SEQUENCE LISTING (114 sequences)

SEQ ID NO: 1 - DNA, 2087 bp, Homo Sapiens
  CDS (222)...(1865)
accacgcgtccgcagcttggtcaacagagctagaccctgtctcaaacacaaaaaataaaaacaaagaaatgggcaagtggctggccccaaggcacaaggccctagtggcaggcccggtctggcctggagtggagtggtagtgactctcaggcaggcagggaggaggaagttgggcgtcaacctaagaccaggctcaccggcttgctgggaaggttaccaagatgctgtgtgggcccgaagttgctcagcctgaagtagacaccaccctgggtcgtgtgcgaggccggcaggtgggcgtgaagggcacagaccgccttgtgaatgtctttctgggcattccatttgcccagccgccactgggccctgaccggttctcagccccacacccagcacagccctgggagggtgtgcgggatgccagcactgcgcccccaatgtgcctacaagacgtggagagcatgaacagcagcagatttgtcctcaacggaaaacagcagatcttctccgtttcagaggactgcctggtcctcaacgtctatagcccagctgaggtccccgcagggtccggtaggccggtcatggtatgggtccatggaggcgctctgataactggcgctgccacctcctacgatggatcagctctggctgcctatggggatgtggtcgtggttacagtccagtaccgccttggggtccttggcttcttcagcactggagatgagcatgcacctggcaaccagggcttcctagatgtggtagctgctttgcgctgggtgcaagaaaacatcgcccccttcgggggtgacctcaactgtgtcactgtctttggtggatctgccggtgggagcatcatctctggcctggtcctgtccccagtggctgcagggctgttccacagagccatcacacagagtggggtcatcaccaccccagggatcatcgactctcacccttggcccctagctcagaaaatcgcaaacaccttggcctgcagctccagctccccggctgagatggtgcagtgccttcagcagaaagaaggagaagagctggtccttagcaagaagctgaaaaatactatctatcctctcaccgttgatggcactgtcttccccaaaagccccaaggaactcctgaaggagaagcccttccactctgtgcccttcctcatgggtgtcaacaaccatgagttcagctggctcatccccaggggctggggtctcctggatacaatggagcagatgagccgggaggacatgctggccatctcaacacccgtcttgaccagtctggatgtgccccctgagatgatgcccaccgtcatagatgaatacctaggaagcaactcggacgcacaagccaaatgccaggcgttccaggaattcatgggtgacgtattcatcaatgttcccaccgtcagtttttcaagataccttcgagattctggaagccctgtctttttctatgagttccagcatcgacccagttcttttgcgaagatcaaacctgcctgggtgaaggctgatcatggggccgagggtgcttttgtgttcggaggtcccttcctcatggacgagagctcccgcctggcctttccagaggccacagaggaggagaagcagctaagcctcaccatgatggcccagtggacccactttgcccggacaggggaccccaatagcaaggctctgcctccttggccccaattcaaccaggcggaacaatatctggagatcaacccagtgccacgggccggacagaagttcagggaggcctggatgcagttctggtcagagacgctccccagcaagatacaacagtggcaccagaagcagaagaacaggaaggcccaggaggacctctgaggccaggcctgaaccttcttggctggggcaaaccactcttcaagtggtggcagagtcccagcacggcagcccgcctctccccctgctgagactttaatctccaccagcccttaaagtgtcggccgctctgtgactggagttatgctcttttgaaatgtcacaaggccgcctcccacctctggggcattgtacaagttcttccctctcaaaaaaaaaaaaaaa

SEQ ID NO: 2 - protein, 547 aa, Homo Sapiens
MLCGPEVAQPEVDTTLGRVRGRQVGVKGTDRLVNVFLGIPFAQPPLGPDRFSAPHPAQPWEGVRDASTAPPMCLQDVESMNSSRFVLNGKQQIFSVSEDCLVLNVYSPAEVPAGSGRPVMVWVHGGALITGAATSYDGSALAAYGDVVVVTVQYRLGVLGFFSTGDEHAPGNQGFLDVVAALRWVQENIAPFGGDLNCVTVFGGSAGGSIISGLVLSPVAAGLFHRAITQSGVITTPGIIDSHPWPLAQKIANTLACSSSSPAEMVQCLQQKEGEELVLSKKLKNTIYPLTVDGTVFPKSPKELLKEKPFHSVPFLMGVNNHEFSWLIPRGWGLLDTMEQMSREDMLAISTPVLTSLDVPPEMMPTVIDEYLGSNSDAQAKCQAFQEFMGDVFINVPTVSFSRYLRDSGSPVFFYEFQHRPSSFAKIKPAWVKADHGAEGAFVFGGPFLMDESSRLAFPEATEEEKQLSLTMMAQWTHFARTGDPNSKALPPWPQFNQAEQYLEINPVPRAGQKFREAWMQFWSETLPSKIQQWHQKQKNRKAQEDL

SEQ ID NO: 3 - DNA, 1644 bp, Homo Sapiens
  CDS (1)...(1644)
atgctgtgtgggcccgaagttgctcagcctgaagtagacaccaccctgggtcgtgtgcgaggccggcaggtgggcgtgaagggcacagaccgccttgtgaatgtctttctgggcattccatttgcccagccgccactgggccctgaccggttctcagccccacacccagcacagccctgggagggtgtgcgggatgccagcactgcgcccccaatgtgcctacaagacgtggagagcatgaacagcagcagatttgtcctcaacggaaaacagcagatcttctccgtttcagaggactgcctggtcctcaacgtctatagcccagctgaggtccccgcagggtccggtaggccggtcatggtatgggtccatggaggcgctctgataactggcgctgccacctcctacgatggatcagctctggctgcctatggggatgtggtcgtggttacagtccagtaccgccttggggtccttggcttcttcagcactggagatgagcatgcacctggcaaccagggcttcctagatgtggtagctgctttgcgctgggtgcaagaaaacatcgcccccttcgggggtgacctcaactgtgtcactgtctttggtggatctgccggtgggagcatcatctctggcctggtcctgtccccagtggctgcagggctgttccacagagccatcacacagagtggggtcatcaccaccccagggatcatcgactctcacccttggcccctagctcagaaaatcgcaaacaccttggcctgcagctccagctccccggctgagatggtgcagtgccttcagcagaaagaaggagaagagctggtccttagcaagaagctgaaaaatactatctatcctctcaccgttgatggcactgtcttccccaaaagccccaaggaactcctgaaggagaagcccttccactctgtgcccttcctcatgggtgtcaacaaccatgagttcagctggctcatccccaggggctggggtctcctggatacaatggagcagatgagccgggaggacatgctggccatctcaacacccgtcttgaccagtctggatgtgccccctgagatgatgcccaccgtcatagatgaatacctaggaagcaactcggacgcacaagccaaatgccaggcgttccaggaattcatgggtgacgtattcatcaatgttcccaccgtcagtttttcaagataccttcgagattctggaagccctgtctttttctatgagttccagcatcgacccagttcttttgcgaagatcaaacctgcctgggtgaaggctgatcatggggccgagggtgcttttgtgttcggaggtcccttcctcatggacgagagctcccgcctggcctttccagaggccacagaggaggagaagcagctaagcctcaccatgatggcccagtggacccactttgcccggacaggggaccccaatagcaaggctctgcctccttggccccaattcaaccaggcggaacaatatctggagatcaacccagtgccacgggccggacagaagttcagggaggcctggatgcagttctggtcagagacgctccccagcaagatacaacagtggcaccagaagcagaagaacaggaaggcccaggaggacctctga

SEQ ID NO: 4 - protein, 554 aa, Mus Musculus
MACLLLIFPTTVIGPKVTQPEVDTPLGRVRGRQVGVKDTDRMVNVFLGIPFAQAPLGPLRFSAPLPPQPWEGVRDASINPPMCLQDVERMSNSRFTLNEKMKIFPISEDCLTLNIYSPTEITAGDKRPVMVWIHGGSLRVGSSTSHDGSALAAYGDVVVVTVQYRLGIFGFLSTGDKHMPGNRGFLDVVAALRWVQGNIAPFGGDPNCVTIFGNSAGGIIVSSLLLSPMSAGLFHRAISQSGVVISKILEDLNAWSEAQNFANSVACGSASPAELVQCLLQKEGKDLITKKNVNISYTVNDSFFPQRPQKLLANKQFPTVPYLLGVTNHEFGWLLLKFWNILDKMEHLSQEDLLENSRPLLAHMQLPPEIMPTVIDEYLDNGSDESATRYALQELLGDITLVIPTLIFSKYLQDAGCPVFLYEFQHTPSSFAKFKPAWVKADHSSENAFVFGGPFLTDESSLLAFPEATEEEKQLSLTMMAQWSQFARTGNPNGKGLPPWPQLNQLEQYLEIGLEPRTGVKLKKGRLQFWTETLPRKIQEWHREQRSRKVPEEL

SEQ ID NO: 5 - protein, 565 aa, Rattus Norvegicus
MRLYPLVWLFLAACTAWGYPSSPPVVNTVKGKVLGKYVNLEGFAQPVAVFLGIPFAKPPLGSLRFAPPQPAEPWNFVKNTTSYPPMCSQDAVGGQVLSELFTNRKENIPLQFSEDCLYLNVYTPADLTKNSRLPVMVWIHGGGLVVGGASTYDGQVLSAHENVVVVTIQYRLGIWGFFSTGDEHSRGNWGHLDQVAALHWVQDNIANFGGNPGSVTIFGESAGGFSVSALVLSPLAKNLFHRAISESGVVLTSALITTDSKPIAKLIATLSGCKTTTSAVMVHCLRQKTEDELLETSLKLNLFKLDLLGNPKESYPFLPTVIDGVVLPKTPEEILAEKSFNTVPYIVGINKQEFGWIIPTLMGYPLSEGKLDQKTAKSLLWKSYPTLKISEKMIPVVAEKYFGGTDDPAKRKDLFQDLVADVIFGVPSVMVSRSHRDAGAPTFMYEFEYRPSFVSAMRPKTVIGDHGDELFSVFGSPFLKDGASEEETNLSKMVMKYWANFARNGSPNGGGLPHWPEYDQKEGYLKIGASTQAAQRLKDKEVAFWSELRAKEAAEEPSHWKHVEL

SEQ ID NO: 6 - protein, 5 aa, Artificial Sequence
  Catalytic Serine Motif
GXSXG

SEQ ID NO: 7 - DNA, 1512 bp, Homo Sapiens
  CDS (342)...(1334)
ccacgcgtccggactagttccatttccacagctcctcctccccggccgcgcgcccctcccgccccgcgcgcgcctcctctttctcgcggccgagttcagcccgggcagccatatgggggatacgccagcaacagacgccggccgccaagatctgcatccctaggccacgctaagaccctggggaagagcgcaggagcccgggagaagggctggaaggaggggactggacgtgcggagaattcccccctaaaaggcagaagcccccgcccccaccctcgagctccgctcgggcagagcgcctgcctgcctgccgctgctgcgggcgcccacctcgcccagccatgccaggcccggccaccgacgcggggaagatccctttctgcgacgccaaggaagaaatccgtgccgggctcgaaagctctgagggcggcggcggcccggagaggccaggcgcgcgcgggcagcggcagaacatcgtctggaggaatgtcgtcctgatgagcttgctccacttgggggccgtgtactccctggtgctcatccccaaagccaagccactcactctgctctgggcctacttctgcttcctcctggccgctctgggtgtgacagctggtgcccatcgcttgtggagccacaggtcctaccgggccaagctgcctctgaggatatttctggctgtcgccaactccatggctttccagaatgacatcttcgagtggtccagggaccaccgagcccaccacaagtactcagagacggatgctgacccccacaatgcccgccggggcttcttcttctcccatattgggtggctgtttgttcgcaagcatcgagatgttattgagaaggggagaaagcttgacgtcactgacctgcttgctgatcctgtggtccggatccagagaaagtactataagatctccgtggtgctcatgtgctttgtggtccccacgctggtgccctggtacatctggggagagagtctgtggaattcctacttcttggcctctattctccgctataccatctcactcaacatcagctggctggtcaacagcgccgcccacatgtatggaaaccggccctatgacaagcacatcagccctcggcagaacccactcgtcgctctgggtgccattggtgaaggcttccataattaccatcacacctttccctttgactactctgcgagtgaatttggcttaaattttaacccaaccacctggttcattgatttcatgtgctggctggggctggccactgaccgcaaacgggcaaccaagccgatgatcgaggcccggaaggccaggactggagacagcagtgcttgaacttggaacagccatcccacatgtctgccgttgcaacctcggttcatggctttggttacaatagctctcttgtacattggatcgtgggagggggcagagggtggggaaggaacgagtcaatgtggtttgggaatgtttttgtttatctcaaaataatgttgaaatacaattatcaatg

SEQ ID NO: 8 - protein, 330 aa, Homo Sapiens
MPGPATDAGKIPFCDAKEEIRAGLESSEGGGGPERPGARGQRQNIVWRNVVLMSLLHLGAVYSLVLIPKAKPLTLLWAYFCFLLAALGVTAGAHRLWSHRSYRAKLPLRIFLAVANSMAFQNDIFEWSRDHRAHHKYSETDADPHNARRGFFFSHIGWLFVRKHRDVIEKGRKLDVTDLLADPVVRIQRKYYKISVVLMCFVVPTLVPWYIWGESLWNSYFLASILRYTISLNISWLVNSAAHMYGNRPYDKHISPRQNPLVALGAIGEGFHNYHHTFPFDYSASEFGLNFNPTTWFIDFMCWLGLATDRKRATKPMIEARKARTGDSSA

SEQ ID NO: 9 - DNA, 990 bp, Homo Sapiens
  CDS (1)...(990)
atgccaggcccggccaccgacgcggggaagatccctttctgcgacgccaaggaagaaatccgtgccgggctcgaaagctctgagggcggcggcggcccggagaggccaggcgcgcgcgggcagcggcagaacatcgtctggaggaatgtcgtcctgatgagcttgctccacttgggggccgtgtactccctggtgctcatccccaaagccaagccactcactctgctctgggcctacttctgcttcctcctggccgctctgggtgtgacagctggtgcccatcgcttgtggagccacaggtcctaccgggccaagctgcctctgaggatatttctggctgtcgccaactccatggctttccagaatgacatcttcgagtggtccagggaccaccgagcccaccacaagtactcagagacggatgctgacccccacaatgcccgccggggcttcttcttctcccatattgggtggctgtttgttcgcaagcatcgagatgttattgagaaggggagaaagcttgacgtcactgacctgcttgctgatcctgtggtccggatccagagaaagtactataagatctccgtggtgctcatgtgctttgtggtccccacgctggtgccctggtacatctggggagagagtctgtggaattcctacttcttggcctctattctccgctataccatctcactcaacatcagctggctggtcaacagcgccgcccacatgtatggaaaccggccctatgacaagcacatcagccctcggcagaacccactcgtcgctctgggtgccattggtgaaggcttccataattaccatcacacctttccctttgactactctgcgagtgaatttggcttaaattttaacccaaccacctggttcattgatttcatgtgctggctggggctggccactgaccgcaaacgggcaaccaagccgatgatcgaggcccggaaggccaggactggagacagcagtgct

SEQ ID NO: 10 - protein, 248 aa, Artificial Sequence
  Consensus Sequence
ILLGALHLGALYLLALLPTELKWKTVIVALLLYVITGGLGITAGYHRLWSHRSYKAKLPLRIFLAIFGTLAVQGSIYEWARDHRAHHKYSDTDADPHDANRGFFFSHVGWLLVKKHPAVKEKGKKLDLSDLKADPVVRFQHRYYIPLMVLMGFILPTLVPGYLWGETFWGGFVWAGFLRLVFVLHATWCVNSAAHKFGYRPYDSRITPRNNWLVALVTFGEGWHNFHHTFPYDYRNAEKWKWEYDLTK

SEQ ID NO: 11 - protein, 248 aa, Artificial Sequence
  Conserved Iron Binding Motif
HXXXXHXXXXXXXXXXXXXXXXXXXXXXXXXXXXXXXXXXXXXXXXXHXXXHHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXHXXXHH

SEQ ID NO: 12 - protein, 15 aa, Artificial Sequence
  Fatty Acid Desaturase Family 1 Signature Sequence
GEXXHNXHHXFPXDY

SEQ ID NO: 13 - protein, 359 aa, Homo Sapiens
MPAHLLQDDISSSYTTTTTITAPPSRVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLLMCFILPTLVPWYFWGETFQNSVFVATFLRYAVVLNATWLVNSAAHLFGYRPYDKNISPRENILVSLGAVGEGFHNYHHSFPYDYSASEYRWHINFTTFFIDCMAALGLAYDRKKVSKAAILARIKRTGDGNYKSG

SEQ ID NO: 14 - protein, 358 aa, Rattus Norvegicus
MPAHMLQEISSSYTTTTTITEPPSGNLQNGREKMKKVPLYLEEDIRPEMREDIHDPSYQDEEGPPPKLEYVWRNIILMALLHVGALYGITLIPSSKVYTLLWGIFYYLISALGITAGAHRLWSHRTYKARLPLRIFLIIANTMAFQNDVYEWARDHRAHHKFSETHADPHNSRRGFFFSHVGWLLVRKHPAVKEKGGKLDMSDLKAEKLVMFQRRYYKPGLLLMCFILPTLVPWYCWGETFLHSLFVSTFLRYTLVLNATWLVNSAAHLYGYRPYDKNIQSRENILVSLGSVGEGFHNYHHAFPYDYSASEYRWHINFTTFFIDCMAALGLAYDRKKVSKAAVLARIKRTGDGSHKSS

SEQ ID NO: 15 - protein, 357 aa, Gallus Gallus
MPAHLLQEEEFSSASSTTTVTSRVTKNGNVIMEKDLLNHDDVAAERGMVDDLFDETYREKEGPKPPLRYVWRNIILMSLLHLGAIIGLTLIPSAKIQTLAWAILCFVLSALGITAGSHRLWSHRSYKATLPLRIFLTIANSMAFQNDIYEWARDHRVHHKFSETHADPHNAMRGYFFSHMAWLLVRKHPDVIEKGQKLDLSDLKADKVVMFQRRYYKPSVVLLCFTLPTLVPWYFWDESIIISFFIPAILRYTLGLNATWLVNSAAHMFGNRPYDQNINPRENPLVSVGALGEGFHNYHHTFPYDYSTSEFGWRFNLTTAFIDLMCLLGLASDRKKVSKEVILARKMRTGDGSHKSG

SEQ ID NO: 16 - DNA, 1327 bp, Homo Sapiens
  CDS (107)...(1096)
cacgcgtccgggaaagagctggttccctggcaggctggagggcaggagctggggccacgctggtctgggatagttgggcagggaggctgtctacctggtctccagaatggacggccctgtggcagagcatgccaagcaggagccctttcacgtggtcacacctctgttggagagctgggcgctgtcccaggtggcgggcatgcctgtcttcctcaagtgtgagaatgtgcagcccagcggctccttcaagattcggggcattgggcatttctgccaggagatggccaagaagggatgcagacacctggtgtgctcctcagggggtaatgcgggcatcgctgctgcctatgctgctaggaagctgggcattcctgccaccatcgtgctccccgagagcacctccctgcaggtggtgcagaggctgcaggcggagggggccgaggttcagctgactggaaaggtctgggacgaggccaatctgagggcgcaagagttggccaagagggacggctgggagaatgtccccccgtttgaccaccccctaatatggaaaggccacgccagcctggtgcaggagctgaaagcagtgctgaggaccccaccaggtgccctggtgctggcagttgggggtgggggtctcctggccggggtggtggctggcctgctggaggtgggctggcagcatgtacccatcattgccatggagacccatggggcacactgcttcaatgcggccatcacagccggcaagctggtcacacttccagacatcaccagtgtggccaagagcctgggtgccaagacggtggccgctcgggccctggagtgcatgcaggtgtgcaagattcactctgaagtggtggaggacaccgaggctgtgagcgctgtgcagcagctcctggatgatgagcgtatgctggtggagcctgcctgtggggcagccttagcagccatctactcaggcctcctgcggaggctccaggccgagggctgcctgcccccttccctgacttcagttgtggtaatcgtgtgtggaggcaacaacatcaacagccgagagctgcaggccttgaaaacccacctgggccaggtctgaggggtcccatcctggccccaaagacccctgagaggcccatggacagtcctgtgtctggatgaggaggactcagtgctggcagatggcagtggaagctgccctgtgcaactgtgctggctgcctcctgaaggaagccctcctggactgcttcttttggctctccgacaactccggccaataaacactttctgaattgagtttgcgaataaaaaaaaaaaaaaaaaaaaaaaa

SEQ ID NO: 17 - protein, 329 aa, Homo Sapiens [sequence omitted]

SEQ ID NO: 18 - DNA, 990 bp, Homo Sapiens
  CDS (1)...(990)
atggacggccctgtggcagagcatgccaagcaggagccctttcacgtggtcacacctctgttggagagctgggcgctgtcccaggtggcgggcatgcctgtcttcctcaagtgtgagaatgtgcagcccagcggctccttcaagattcggggcattgggcatttctgccaggagatggccaagaagggatgcagacacctggtgtgctcctcagggggtaatgcgggcatcgctgctgcctatgctgctaggaagctgggcattcctgccaccatcgtgctccccgagagcacctccctgcaggtggtgcagaggctgcaggcggagggggccgaggttcagctgactggaaaggtctgggacgaggccaatctgagggcgcaagagttggccaagagggacggctgggagaatgtccccccgtttgaccaccccctaatatggaaaggccacgccagcctggtgcaggagctgaaagcagtgctgaggaccccaccaggtgccctggtgctggcagttgggggtgggggtctcctggccggggtggtggctggcctgctggaggtgggctggcagcatgtacccatcattgccatggagacccatggggcacactgcttcaatgcggccatcacagccggcaagctggtcacacttccagacatcaccagtgtggccaagagcctgggtgccaagacggtggccgctcgggccctggagtgcatgcaggtgtgcaagattcactctgaagtggtggaggacaccgaggctgtgagcgctgtgcagcagctcctggatgatgagcgtatgctggtggagcctgcctgtggggcagccttagcagccatctactcaggcctcctgcggaggctccaggccgagggctgcctgcccccttccctgacttcagttgtggtaatcgtgtgtggaggcaacaacatcaacagccgagagctgcaggccttgaaaacccacctgggccaggtctga

SEQ ID NO: 19 - protein, 15 aa, Artificial Sequence
  Serine/Threonine Dehydratase Pyridoxal-Phosphate Attachment Site Conserved Sequence
XXXXXXXXXXKXXXX

SEQ ID NO: 20 - DNA, 3669 bp, Homo Sapiens
  CDS (106)...(2859)
ccacgcgtccggcctggactggaagcgtgcaacactccagagtcgtaggagtgaacactgcacaggaatctctgcccatctcaggagaaaccaaacttggggaaaatgtttgcggtccacttgatggcattttacttcagcaagctgaaggaggaccagatcaagaaggtggacaggttcctgtatcacatgcggctctccgatgacacccttttggacatcatgaggcggttccgggctgagatggagaagggcctggcaaaggacaccaaccccacggctgcagtgaagatgttgcccaccttcgtcagggccattcccgatggttccgaaaatggggagttcctttccctggatctcggagggtccaagttccgagtgctgaaggtgcaagtcgctgaagaggggaagcgacacgtgcagatggagagtcagttctacccaacgcccaatgaaatcatccgcgggaacggcatagagctgtttgaatatgtagctgactgtctggcagatttcatgaagaccaaagatttaaagcataagaaattgccccttggcctaactttttctttcccctgtcgacagactaaactggaagagggtgtcctactttcgtggacaaaaaagtttaaggcacgaggagttcaggacacggatgtggtgagccgtctgaccaaagccatgagaagacacaaggacatggacgtggacatcctggccctggtcaatgacaccgtggggaccatgatgacctgtgcctatgacgacccctactgcgaagttggtgtcatcatcggaactggcaccaatgcgtgttacatggaggacatgagcaacattgacctggtggagggcgacgagggcaggatgtgcatcaacacagagtggggggccttcggggacgacggggccctggaggacattcgcactgagttcgacagggagctggacctcggctctctcaacccaggaaagcaactgttcgagaagatgatcagtggcctgtacctgggggagcttgtcaggcttatcttgctgaagatggccaaggctggcctcctgtttggtggtgagaaatcttctgctctccacactaagggcaagatcgaaacacggcacgtggctgccatggagaagtataaagaaggccttgctaatacaagagagatcctggtggacctgggtctggaaccgtctgaggctgactgcattgccgtccagcatgtctgtaccatcgtctccttccgctcggccaatctctgtgcagcagctctggcggccatcctgacacgcctccgggagaacaagaaggtggaacggctccggaccacagtgggcatggacggcaccctctacaagatacaccctcagtacccaaaacgcctgcacaaggtggtgaggaaactggtcccaagctgtgatgtccgcttcctcctgtcagagagtggcagcaccaagggggccgccatggtgaccgcggtggcctcccgcgtgcaggcccagcggaagcagatcgacagggtgctggctttgttccagctgacccgagagcagctcgtggacgtgcaggccaagatgcgggctgagctggagtatgggctgaagaagaagagccacgggctggccacggtcaggatgctgcccacctacgtctgcgggctgccggacggcacagagaaaggaaagtttctcgccctggatcttgggggaaccaacttccgggtcctcctggtgaagatcagaagtggacggaggtcagtgcgaatgtacaacaagatcttcgccatccccctggagatcatgcagggcactggtgaggagctctttgatcacattgtgcagtgcatcgccgacttcctggactacatgggcctcaagggagcctccctacctttgggcttcacattctcatttccctgcaggcagatgagcattgacaagggaacactcatagggtggaccaaaggtttcaaggccactgactgtgaaggggaggacgtggtggacatgctcagggaagccatcaagaggagaaacgagtttgacctggacattgttgcagtcgtgaatgatacagtggggaccatgatgacctgtggctatgaagatcctaattgtgagattggcctgattgcaggaacaggcagcaacatgtgctacatggaggacatgaggaacatcgagatggtggaggggggtgaagggaagatgtgcatcaatacagagtggggaggatttggagacaatggctgcatagatgacatccggacccgatacgacacggaggtggatgaggggtccttgaatcctggcaagcagagatacgagaaaatgaccagtgggatgtacttgggggagattgtgcggcagatcctgatcgacctgaccaagcagggtctcctcttccgagggcagatttcagagcgtctccggaccaggggcatcttcgaaaccaagttcctgtcccagatcgaaagcgatcggctggcccttctccaggtcaggaggattctgcagcagctgggcctggacagcacgtgtgaggacagcatcgtggtgaaggaggtgtgcggagccgtgtcccggcgggcggcccagctctgcggtgctggcctggccgctatagtggaaaaaaggagagaagaccaggggctagagcacctgaggatcactgtgggtgtggacggcaccctgtacaagctgcaccctcacttttctagaatattgcaggaaactgtgaaggaactagcccctcgatgtgatgtgacattcatgctgtcagaagatggcagtggaaaaggggcagcactgatcactgctgtggccaagaggttacagcaggcacagaaggagaactaggaacccctgggattggacctgatgcatcttggatactgaacagcttttcctctggcagatcagttggtcagagaccaatgggcaccctcctggctgacctcaccttctggatggccgaaagagaaccccaggttctcgggtactcttagtatcttgtactggatttgcagtgacattacatgacatctctatttggtatatttgggccaaaatgggccaacttatgaaatcaaagtgtctgtcctgagagatcccctttcaacacattgttcaggtgaggcttgagctgtcaattctctatggctttcagtcttgtggctgcgggacttggaaatatatagaatctgcccatgtggctggcaggctgtttccccattgggatgcttaagccatctcttataggggattggaccctgtacttgtggatgaacattggagagcaagaggaactcacgttatgaactagggggatctcatctaacttgtccttaacttgccatgttgacttcaaacctgttaagagaacaaagactttgaagtatccagccccagggtgcagagaggttgattgccagggagcactgcaggaatcattgcatgcttaaagcgagttatgtcagcaccctgtaggattttgttccttattaagtgtgtgccatgtggtggggtgctgtctggggcatctgtttttcattttgcctgtggtttgtgttgcagstgttgatagttgttttaaggattgttaggtataggaaatccagtaaattaataaaaaaattttgattttccaataaaaaaaaaaaaaaaaaa

SEQ ID NO: 21 - protein, 917 aa, Homo Sapiens [sequence omitted]

SEQ ID NO: 22 - DNA, 2754 bp, Homo Sapiens
  CDS (1)...(2754)
atgtttgcggtccacttgatggcattttacttcagcaagctgaaggaggaccagatcaagaaggtggacaggttcctgtatcacatgcggctctccgatgacacccttttggacatcatgaggcggttccgggctgagatggagaagggcctggcaaaggacaccaaccccacggctgcagtgaagatgttgcccaccttcgtcagggccattcccgatggttccgaaaatggggagttcctttccctggatctcggagggtccaagttccgagtgctgaaggtgcaagtcgctgaagaggggaagcgacacgtgcagatggagagtcagttctacccaacgcccaatgaaatcatccgcgggaacggcatagagctgtttgaatatgtagctgactgtctggcagatttcatgaagaccaaagatttaaagcataagaaattgccccttggcctaactttttctttcccctgtcgacagactaaactggaagagggtgtcctactttcgtggacaaaaaagtttaaggcacgaggagttcaggacacggatgtggtgagccgtctgaccaaagccatgagaagacacaaggacatggacgtggacatcctggccctggtcaatgacaccgtggggaccatgatgacctgtgcctatgacgacccctactgcgaagttggtgtcatcatcggaactggcaccaatgcgtgttacatggaggacatgagcaacattgacctggtggagggcgacgagggcaggatgtgcatcaacacagagtggggggccttcggggacgacggggccctggaggacattcgcactgagttcgacagggagctggacctcggctctctcaacccaggaaagcaactgttcgagaagatgatcagtggcctgtacctgggggagcttgtcaggcttatcttgctgaagatggccaaggctggcctcctgtttggtggtgagaaatcttctgctctccacactaagggcaagatcgaaacacggcacgtggctgccatggagaagtataaagaaggccttgctaatacaagagagatcctggtggacctgggtctggaaccgtctgaggctgactgcattgccgtccagcatgtctgtaccatcgtctccttccgctcggccaatctctgtgcagcagctctggcggccatcctgacacgcctccgggagaacaagaaggtggaacggctccggaccacagtgggcatggacggcaccctctacaagatacaccctcagtacccaaaacgcctgcacaaggtggtgaggaaactggtcccaagctgtgatgtccgcttcctcctgtcagagagtggcagcaccaagggggccgccatggtgaccgcggtggcctcccgcgtgcaggcccagcggaagcagatcgacagggtgctggctttgttccagctgacccgagagcagctcgtggacgtgcaggccaagatgcgggctgagctggagtatgggctgaagaagaagagccacgggctggccacggtcaggatgctgcccacctacgtctgcgggctgccggacggcacagagaaaggaaagtttctcgccctggatcttgggggaaccaacttccgggtcctcctggtgaagatcagaagtggacggaggtcagtgcgaatgtacaacaagatcttcgccatccccctggagatcatgcagggcactggtgaggagctctttgatcacattgtgcagtgcatcgccgacttcctggactacatgggcctcaagggagcctccctacctttgggcttcacattctcatttccctgcaggcagatgagcattgacaagggaacactcatagggtggaccaaaggtttcaaggccactgactgtgaaggggaggacgtggtggacatgctcagggaagccatcaagaggagaaacgagtttgacctggacattgttgcagtcgtgaatgatacagtggggaccatgatgacctgtggctatgaagatcctaattgtgagattggcctgattgcaggaacaggcagcaacatgtgctacatggaggacatgaggaacatcgagatggtggaggggggtgaagggaagatgtgcatcaatacagagtggggaggatttggagacaatggctgcatagatgacatccggacccgatacgacacggaggtggatgaggggtccttgaatcctggcaagcagagatacgagaaaatgaccagtgggatgtacttgggggagattgtgcggcagatcctgatcgacctgaccaagcagggtctcctcttccgagggcagatttcagagcgtctccggaccaggggcatcttcgaaaccaagttcctgtcccagatcgaaagcgatcggctggcccttctccaggtcaggaggattctgcagcagctgggcctggacagcacgtgtgaggacagcatcgtggtgaaggaggtgtgcggagccgtgtcccggcgggcggcccagctctgcggtgctggcctggccgctatagtggaaaaaaggagagaagaccaggggctagagcacctgaggatcactgtgggtgtggacggcaccctgtacaagctgcaccctcacttttctagaatattgcaggaaactgtgaaggaactagcccctcgatgtgatgtgacattcatgctgtcagaagatggcagtggaaaaggggcagcactgatcactgctgtggccaagaggttacagcaggcacagaaggagaactag

SEQ ID NO: 23 - protein, 482 aa, Artificial Sequence
  Consensus Sequence
ADLLQAVEELLDDFTVSTETLREVTKRFIKEMEKGLSPPKEGGNTASVVKMLPTFVRSTPTGTEKGDFLALDLGGTNFRVLLVKLGGNGKGVEMTQSKYRIPEELMTGENVTGEQLFDFIAECIKDFMDEQFPKGKKEPLPLGFTFSFPCSQTSINEGILIRWTKGFKIGRATNSGVEGHDVVQLLREAIKRRGAFPIDVVAVVNDTVGTLMSCAYTKGRGDPECETVIGLIVGTGTNACYMEEMRNIEKLEGKLKDDIPDEGRMCINMEWGAFGDNGHLDLPRTKYDVVIDEESPNPGQQLFEKMISGMYLGEIVRLILLDLTKEGLLFKGQDSPKLKTRGSFETSVLSRIESDPSENLEDVRAILQTALGLETTDEERKLVRRVCEAVSTRAARLCAAGLAAILKKIRENRGRERLKVTVGVDGSVYKLYPGFKERLAEALRDLLPDCEGSEEDKKVSIIPAEDGSGKGAALVAAVAAKL

SEQ ID NO: 24 - protein, 26 aa, Artificial Sequence
  Hexokinase Signature Domain
LGFTFSFPCXQXSIXXGXLIXWTKGF

SEQ ID NO: 25 - DNA, 3544 bp, Homo Sapiens
  CDS (589)...(2586)
tcctatagggagtcgccccgcgtccgaaaagattataagtaaatactctgctctttcaagtgaaccaaacctatcaaacctgtttagaaaataaaccaggcagaataaaatgatgcgaaatgttcattttaaaaaacttcaggatgggcacaaacacacagaagtgggaaatgaataaaagagtattgataaatttttgaaaattgttgaagctgagtaatgggctttcagtccagtgtaaagctgttggagcgcgggagcaaaggtaaagaatgatgtaatgcgctggctgctccaaagcatcttttgttgtggaatggttattccagtcatctctttatgaatcaaatgtgaggggctgctttgtggacggagtcctttgcaagagcacatcaacgggaaagagaaagagacattcacttggagggctcttgctgaaaatgggtttaactctccttttgccagtcaccaccagcctgacctcatacacttttagtacaatggagtggctgagcctttgagcacaccaccattacatcatcgtggcaaattaaagaaggaggtgggaaaagaggacttattgttgtcatggcccatgagatgattggaactcaaattgttactgagaggttggtggctctgctggaaagtggaacggaaaaagtgctgctaattgatagccggccatttgtggaatacaatacatcccacattttggaagccattaatatcaactgctccaagcttatgaagcgaaggttgcaacaggacaaagtgttaattacagagctcatccagcattcagcgaaacataaggttgacattgattgcagtcagaaggttgtagtttacgatcaaagctcccaagatgttgcctctctctcttcagactgttttctcactgtacttctgggtaaactggagaagagcttcaactctgttcacctgcttgcaggtgggtttgctgagttctctcgttgtttccctggcctctgtgaaggaaaatccactctagtccctacctgcatttctcagccttgcttacctgttgccaacattgggccaacccgaattcttcccaatctttatcttggctgccagcgagatgtcctcaacaaggagctgatgcagcagaatgggattggttatgtgttaaatgccagcaatacctgtccaaagcctgactttatccccgagtctcatttcctgcgtgtgcctgtgaatgacagcttttgtgagaaaattttgccgtggttggacaaatcagtagatttcattgagaaagcaaaagcctccaatggatgtgttctagtgcactgtttagctgggatctcccgctccgccaccatcgctatcgcctacatcatgaagaggatggacatgtctttagatgaagcttacagatttgtgaaagaaaaaagacctactatatctccaaacttcaattttctgggccaactcctggactatgagaagaagattaagaaccagactggagcatcagggccaaagagcaaactcaagctgctgcacctggagaagccaaatgaacctgtccctgctgtctcagagggtggacagaaaagcgagacgcccctcagtccaccctgtgccgactctgctacctcagaggcagcaggacaaaggcccgtgcatcccgccagcgtgcccagcgtgcccagcgtgcagccgtcgctgttagaggacagcccgctggtacaggcgctcagtgggctgcacctgtccgcagacaggctggaagacagcaataagctcaagcgttccttctctctggatatcaaatcagtttcatattcagccagcatggcagcatccttacatggcttctcctcatcagaagatgctttggaatactacaaaccttccactactctggatgggaccaacaagctatgccagttctcccctgttcaggaactatcggagcagactcccgaaaccagtcctgataaggaggaagccagcatccccaagaagctgcagaccgccaggccttcagacagccagagcaagcgattgcattcggtcagaaccagcagcagtggcaccgcccagaggtcccttttatctccactgcatcgaagtgggagcgtggaggacaattaccacaccagcttccttttcggcctttccaccagccagcagcacctcacgaagtctgctggcctgggccttaagggctggcactcggatatcttggccccccagacctctaccccttccctgaccagcagctggtattttgccacagagtcctcacacttctactctgcctcagccatctacggaggcagtgccagttactctgcctacagctgcagccagctgcccacttgcggagaccaagtctattctgtgcgcaggcggcagaagccaagtgacagagctgactcgcggcggagctggcatgaagagagcccctttgaaaagcagtttaaacgcagaagctgccaaatggaatttggagagagcatcatgtcagagaacaggtcacgggaagagctggggaaagtgggcagtcagtctagcttttcgggcagcatggaaatcattgaggtctcctgagaagaaagacacttgtgacttctatagacaatttttttttcttgttcacaaaaaaattccctgtaaatctgaaatatatatatgtacatacatatatatttttggaaaatggagctatggtgtaaaagcaacaggtggatcaacccagttgttactctcttaacatctgcatttgagagatcagctaatacttctctcaacaaaaatggaagggcagatgctagaatcccccctagacggaggaaaaccattttattcagtgaattacacatcctcttgttcttaaaaaagcaagtgtctttggtgttggaggacaaaatcccctaccattttcacgttgtgctactaagagatctcaaatattagtctttgtccggacccttccatagtacaccttagcgctgagactgagccagcttgggggtcaggtaggtagaccctgttagggacagagcctagtggtaaatccaagagaaatgatcctatccaaagctgattcacaaacccacgctcacctgacagccgagggacacgagcatcactctgctggacggaccattaggggccttgccaaggtctaccttagagcaaacccagtacctcagacaggaaagtcggggctttgaccactaccatatctggtagcccattttctaggcattgtgaataggtaggtagctagtcacacttttcagaccaattcaaactgtctatgcacaaaattcccgtgggcctagatggagataatttttttttcttctcagctttatgaagagaagggaaactgtctaggattcagctgaaccaccaggaacctggcaacatcacgatttaagctaaggttgggaggctaacgagtctacctccctctttgtaaatcaaagaattgtttaaaatgggattgtcaatcctttaaataaagatgaacttggtttcaaaaaaaaaaaaaaaaaaaagg

SEQ ID NO: 26 - protein, 665 aa, Homo Sapiens
MAHEMIGTQIVTERLVALLESGTEKVLLIDSRPFVEYNTSHILEAININCSKLMKRRLQQDKVLITELIQHSAKHKVDIDCSQKVVVYDQSSQDVASLSSDCFLTVLLGKLEKSFNSVHLLAGGFAEFSRCFPGLCEGKSTLVPTCISQPCLPVANIGPTRILPNLYLGCQRDVLNKELMQQNGIGYVLNASNTCPKPDFIPESHFLRVPVNDSFCEKILPWLDKSVDFIEKAKASNGCVLVHCLAGISRSATIAIAYIMKRMDMSLDEAYRFVKEKRPTISPNFNFLGQLLDYEKKIKNQTGASGPKSKLKLLHLEKPNEPVPAVSEGGQKSETPLSPPCADSATSEAAGQRPVHPASVPSVPSVQPSLLEDSPLVQALSGLHLSADRLEDSNKLKRSFSLDIKSVSYSASMAASLHGFSSSEDALEYYKPSTTLDGTNKLCQFSPVQELSEQTPETSPDKEEASIPKKLQTARPSDSQSKRLHSVRTSSSGTAQRSLLSPLHRSGSVEDNYHTSFLFGLSTSQQHLTKSAGLGLKGWHSDILAPQTSTPSLTSSWYFATESSHFYSASAIYGGSASYSAYSCSQLPTCGDQVYSVRRRQKPSDRADSRRSWHEESPFEKQFKRRSCQMEFGESIMSENRSREELGKVGSQSSFSGSMEIIEVS

SEQ ID NO: 27 - DNA, 1998 bp, Homo Sapiens
  CDS (1)...(1998)
atggcccatgagatgattggaactcaaattgttactgagaggttggtggctctgctggaaagtggaacggaaaaagtgctgctaattgatagccggccatttgtggaatacaatacatcccacattttggaagccattaatatcaactgctccaagcttatgaagcgaaggttgcaacaggacaaagtgttaattacagagctcatccagcattcagcgaaacataaggttgacattgattgcagtcagaaggttgtagtttacgatcaaagctcccaagatgttgcctctctctcttcagactgttttctcactgtacttctgggtaaactggagaagagcttcaactctgttcacctgcttgcaggtgggtttgctgagttctctcgttgtttccctggcctctgtgaaggaaaatccactctagtccctacctgcatttctcagccttgcttacctgttgccaacattgggccaacccgaattcttcccaatctttatcttggctgccagcgagatgtcctcaacaaggagctgatgcagcagaatgggattggttatgtgttaaatgccagcaatacctgtccaaagcctgactttatccccgagtctcatttcctgcgtgtgcctgtgaatgacagcttttgtgagaaaattttgccgtggttggacaaatcagtagatttcattgagaaagcaaaagcctccaatggatgtgttctagtgcactgtttagctgggatctcccgctccgccaccatcgctatcgcctacatcatgaagaggatggacatgtctttagatgaagcttacagatttgtgaaagaaaaaagacctactatatctccaaacttcaattttctgggccaactcctggactatgagaagaagattaagaaccagactggagcatcagggccaaagagcaaactcaagctgctgcacctggagaagccaaatgaacctgtccctgctgtctcagagggtggacagaaaagcgagacgcccctcagtccaccctgtgccgactctgctacctcagaggcagcaggacaaaggcccgtgcatcccgccagcgtgcccagcgtgcccagcgtgcagccgtcgctgttagaggacagcccgctggtacaggcgctcagtgggctgcacctgtccgcagacaggctggaagacagcaataagctcaagcgttccttctctctggatatcaaatcagtttcatattcagccagcatggcagcatccttacatggcttctcctcatcagaagatgctttggaatactacaaaccttccactactctggatgggaccaacaagctatgccagttctcccctgttcaggaactatcggagcagactcccgaaaccagtcctgataaggaggaagccagcatccccaagaagctgcagaccgccaggccttcagacagccagagcaagcgattgcattcggtcagaaccagcagcagtggcaccgcccagaggtcccttttatctccactgcatcgaagtgggagcgtggaggacaattaccacaccagcttccttttcggcctttccaccagccagcagcacctcacgaagtctgctggcctgggccttaagggctggcactcggatatcttggccccccagacctctaccccttccctgaccagcagctggtattttgccacagagtcctcacacttctactctgcctcagccatctacggaggcagtgccagttactctgcctacagctgcagccagctgcccacttgcggagaccaagtctattctgtgcgcaggcggcagaagccaagtgacagagctgactcgcggcggagctggcatgaagagagcccctttgaaaagcagtttaaacgcagaagctgccaaatggaatttggagagagcatcatgtcagagaacaggtcacgggaagagctggggaaagtgggcagtcagtctagcttttcgggcagcatggaaatcattgaggtctcctga

SEQ ID NO: 28 - DNA, 1114 bp, Homo Sapiens
  CDS (89)...(760)
ctatagggagtcgcccacgcgtccggggcggtggcgcgctgacacctggcggcggcggagggcgggcagaagcccgcgggccagcaccatggaggacgtgaagctggagttcccttcccttccacagtgcaaggaagacgccgaggagtggacctaccctatgagacgagagatgcaggaaattttacctggattgttcttaggcccatattcatctgctatgaaaagcaagctacctgtactacagaaacatggaataacccatataatatgcatacgacaaaatattgaagcaaactttattaaaccaaactttcagcagttatttagatatttagtcctggatattgcagataatccagttgaaaatataatacgttttttccctatgactaaggaatttattgatgggagcttacaaatgggaggaaaagttcttgtgcatggaaatgcagggatctccagaagtgcagcctttgttattgcatacattatggaaacatttggaatgaagtacagagatgcttttgcttatgttcaagaaagaagattttgtattaatcctaatgctggatttgtccatcaacttcaggaatatgaagccatctacctagcaaaattaacaatacagatgatgtcaccactccagatagaaaggtcattatctgttcattctggtaccacaggcagtttgaagagaacacatgaagaagaggatgattttggaaccatgcaagtggcgactgcacagaatggctgacttgaagagcaacatcatagagtgtgaatttctatttgggaaggagaaaatacaagagaaaattataatgtaaaatggtaaaaacataagtagtttttttttcaattacatgttgcttccagacatacttctctgcaacttgttgagcaacattttaagatgttggacttctgcaatagatgacactgatggttttactcctttttttaaaaacacatgcgcgcgcacacacacatgctttacaagttttattataaaccaagaattttggacttgcaaagaggtattattgcaataatgcacttttcatacttgaaatttatttgtatgatataaagttattactttaaacaa

SEQ ID NO: 29 - protein, 223 aa, Homo Sapiens
MEDVKLEFPSLPQCKEDAEEWTYPMRREMQEILPGLFLGPYSSAMKSKLPVLQKHGITHIICIRQNIEANFIKPNFQQLFRYLVLDIADNPVENIIRFFPMTKEFIDGSLQMGGKVLVHGNAGISRSAAFVIAYIMETFGMKYRDAFAYVQERRFCINPNAGFVHQLQEYEAIYLAKLTIQMMSPLQIERSLSVHSGTTGSLKRTHEEEDDFGTMQVATAQNG

SEQ ID NO: 30 - DNA, 672 bp, Homo Sapiens
  CDS (1)...(672)
atggaggacgtgaagctggagttcccttcccttccacagtgcaaggaagacgccgaggagtggacctaccctatgagacgagagatgcaggaaattttacctggattgttcttaggcccatattcatctgctatgaaaagcaagctacctgtactacagaaacatggaataacccatataatatgcatacgacaaaatattgaagcaaactttattaaaccaaactttcagcagttatttagatatttagtcctggatattgcagataatccagttgaaaatataatacgttttttccctatgactaaggaatttattgatgggagcttacaaatgggaggaaaagttcttgtgcatggaaatgcagggatctccagaagtgcagcctttgttattgcatacattatggaaacatttggaatgaagtacagagatgcttttgcttatgttcaagaaagaagattttgtattaatcctaatgctggatttgtccatcaacttcaggaatatgaagccatctacctagcaaaattaacaatacagatgatgtcaccactccagatagaaaggtcattatctgttcattctggtaccacaggcagtttgaagagaacacatgaagaagaggatgattttggaaccatgcaagtggcgactgcacagaatggctga

SEQ ID NO: 31 - protein, 173 aa, Artificial Sequence
  Consensus Sequence
GPSEILPHLYLGSYSTASEANLALLKKLGITHVINVTEEVPNPFELDKKNDRHYTNAYISKNSGFTYLQIPNVDDHIYYHIAWNHETKISKYFDEAVDFIDDARQKGGKVLVHCQAGISRSATLIIAYLMKTRNLSLNEAYDFVYVYHIKERRCPIISPNFGFLRQLIEYERK

SEQ ID NO: 32 - protein, 172 aa, Artificial Sequence
  Consensus Sequence
GPSEILPHLYLGSYSDASEANLALLKKLGITHVINVTEEVPNNFELKKKNDRYYTNEYISKGSGFTYLQIPNVDDIYYHIAWNTETKISKYLEEAVEFIEDAEKKGGKVLVHCQAGVSRSATLVIAYLMKTRNLSLRDAYDFVYVYHIKERRCPIISPNFGFLRQLIEYERK

SEQ ID NO: 33 - protein, 131 aa, Artificial Sequence
  Consensus Sequence
TAGELKALLESAPKLILIDVRSPEFGEEYEYEGGHIPGAVNVPEEEIEALLDRSGILPDIEKLHLLKDPEELAKLFGELGSSKDKRVIVYCRSGRGLLRNRRSALAALLLKKLGYPEVYILKGGYKEWLAK

SEQ ID NO: 34 - protein, 94 aa, Artificial Sequence
  Consensus Sequence
VLEELKLLLNEDVVLLDVRSPEEYEGGHIPGAVNIPLSELLDRLGLDKDKPVIVYCRSGVRSAAKAAWLLRELGFKNVYLLDGGYKEWSAAGPP

SEQ ID NO: 35 - DNA, 1182 bp, Homo Sapiens
  CDS (326)...(1009)
tggaggtagaaaacttttattagcksgtccggttgaggcctacagcggggaaaggacttgccagattttcgccgcaagtcagggccatagcggggggcataacaaggcctcccaaccgaaggtcaagcaagagctccgagcgtcccacacaagtcccgaagggacactgtgacgccgcgctactgaaggcgcctgggttcccggactcggccaccgcctcgccgcttccgcccctcagaagcatggcggccacgtagcccggcccggattggacgttggcggtggacgccaaacagttggcaacacgattggctgctgcggggtgatgacgtcagggggcggtgtcggagtgaatgggggcagcatgaggccgggcggctttttgggcgccggacagcggctgagtagagccatgagccgatgtgttttggagcctcgccccccggggaagcggtggatggtggctggcctggggaatcccggactgcccggcacgcgacacagcgtgggcatggcggtgctggggcagctggcgcggcggctgggtgtggcggagagttggacgcgcgaccggcactgtgccgccgacctcgccctggccccgctgggggatgcccaactggtcctgctccggccacggcggcttatgaacgccaacgggcgcagcgtggcccgggctgcggagctgtttgggctgactgccgaggaagtctacctggtgcatgatgagctggacaagcccctggggagactggctctgaagctggggggcagtgccaggggccacaatggagtccgttcctgcattagctgcctcaactccaatgcaatgccaaggctgcgggtgggtatcgggcgcccggcgcaccctgaggcggttcaggcccatgtgctgggctgcttctcccctgctgagcaggagctgctgcctctgttgctggatcgagccaccgacctgatcttggaccacatccgtgagcgaagccaggggccctcactggggccgtgacactagtggccatggctgcctgcctgactgtagtgcccaccaacccagccactgccacagagctgccacgccagccttggtatctactttttatacaaatctcctctagactgttccaggctgcctgcggattaaagtgggggtgactgtgaaaaaaaaaaaaaaaaaaagga

SEQ ID NO: 36 - protein, 227 aa, Homo Sapiens [sequence omitted]

SEQ ID NO: 37 - DNA, 684 bp, Homo Sapiens
  CDS (1)...(684)
atgacgtcagggggcggtgtcggagtgaatgggggcagcatgaggccgggcggctttttgggcgccggacagcggctgagtagagccatgagccgatgtgttttggagcctcgccccccggggaagcggtggatggtggctggcctggggaatcccggactgcccggcacgcgacacagcgtgggcatggcggtgctggggcagctggcgcggcggctgggtgtggcggagagttggacgcgcgaccggcactgtgccgccgacctcgccctggccccgctgggggatgcccaactggtcctgctccggccacggcggcttatgaacgccaacgggcgcagcgtggcccgggctgcggagctgtttgggctgactgccgaggaagtctacctggtgcatgatgagctggacaagcccctggggagactggctctgaagctggggggcagtgccaggggccacaatggagtccgttcctgcattagctgcctcaactccaatgcaatgccaaggctgcgggtgggtatcgggcgcccggcgcaccctgaggcggttcaggcccatgtgctgggctgcttctcccctgctgagcaggagctgctgcctctgttgctggatcgagccaccgacctgatcttggaccacatccgtgagcgaagccaggggccctcactggggccgtga

SEQ ID NO: 38 - protein, 193 aa, Artificial Sequence
  Consensus Sequence
TIKLIVGLGNPGKQYAETRHNAGFMVLDLLASRLGLSLREEKRFFGLGGKVLVSGKKHCVILLKPRTYMNLSGKAVLALASFYKIKPEEILVVHDDLDLPLGKIRLKQGGGAGRGHNGLKSIISHLGNTNNFNRLRIGIGRPNPGSNDVAEFVLSKFSPAERPLLEKALDKAIEALEMIIEGHGMNKLMNRFN

SEQ ID NO: 39 - DNA, 2629 bp, Homo Sapiens
  CDS (257)...(2545)
gcacgagacacctctccccttcttactgcttccctccggctataacttgccagtcacagcagccagctgctgtagaagaggggaggaaacaagccagtgcaaggggagcaaaagagaaaaggagccaggctgggcttcctgatcccacagcatcgcagagctcgggaggcacagctcacagacacaggaaacacaggactgctattctgctctcctgcccacggtgatctggtgccagctggtggaacagtgggtgatggcgtccctgctgcaagaccagctgaccactgatcaggacttgctgctgatgcaggaaggcatgccgatgcgcaaggtgaggtccaaaagctggaagaagctaagatacttcagacttcagaatgacggcatgacagtctggcatgcacggcaggccaggggcagtgccaagcccagcttctcaatctctgatgtggagacaatacgtaatggccatgattccgagttgctgcgtagcctggcagaggagctccccctggagcagggcttcaccattgtcttccatggccgccgctccaacctggacctgatggccaacagtgttgaggaggcccagatatggatgcgagggctccagctgttggtggatcttgtcaccagcatggaccatcaggagcgcctggaccaatggctgagcgattggtttcaacgtggagacaaaaatcaggatggtaagatgagtttccaagaagttcagcggttattgcacctaatgaatgtggaaatggaccaagaatatgccttcagtctttttcaggcagcagacacgtcccagtctggaaccctggaaggagaagaattcgtacagttctataaggcattgactaaacgtgctgaggtgcaggaactgtttgaaagtttttcagctgatgggcagaagctgactctgctggaatttttggatttcctccaagaggagcagaaggagagagactgcacctctgagcttgctctggaactcattgaccgctatgaaccttcagacagtggcaaactgcggcatgtgctgagtatggatggcttcctcagctacctctgctctaaggatggagacatcttcaacccagcctgcctccccatctatcaggatatgactcaacccctgaaccactacttcatctgctcttctcataacacctacctagtgggggaccagctttgcggccagagcagcgtcgagggatatatacgggccctgaagcgggggtgccgctgcgtggaggtggatgtatgggatggacctagcggggaacctgtcgtttaccacggacacaccctgacctcccgcatcctgttcaaagatgtcgtggccacagtagcacagtatgccttccagacatcagactacccagtcatcttgtccctggagacccactgcagctgggagcagcagcagaccatggcccgtcatctgactgagatcctgggggagcagctgctgagcaccaccttggatggggtgctgcccactcagctgccctcgcctgaggagcttcggaggaagatcctggtgaaggggaagaagttaacacttgaggaagacctggaatatgaggaagaggaagcagaacctgagttggaagagtcagaattggcgctggagtcccagtttgagactgagcctgagccccaggagcagaaccttcagaataaggacaaaaagaagaaatccaagcccatcttgtgtccagccctctcttccctggttatctacttgaagtctgtctcattccgcagcttcacacattcaaaggagcactaccacttctacgagatatcatctttctctgaaaccaaggccaagcgcctcatcaaggaggctggcaatgagtttgtgcagcacaatacttggcagttaagccgtgtgtatcccagcggcctgaggacagactcttccaactacaacccccaggaactctggaatgcaggctgccagatggtggccatgaatatgcagactgcagggcttgaaatggacatctgtgatgggcatttccgccagaatggcggctgtggctatgtgctgaagccagacttcctgcgtgatatccagagttctttccaccctgagaagcccatcagccctttcaaagcccagactctcttaatccaggtgatcagcggtcagcaactccccaaagtggacaagaccaaagaggggtccattgtggatccactggtgaaagtgcagatctttggcgttcgtctagacacagcacggcaggagaccaactatgtggagaacaatggttttaatccatactgggggcagacactatgtttccgggtgctggtgcctgaacttgccatgctgcgttttgtggtaatggattatgactggaaatcccgaaatgactttattggtcagtacaccctgccttggacctgcatgcaacaaggttaccgccacattcacctgctgtccaaagatggcatcagcctccgcccagcttccatctttgtgtatatctgcatccaggaaggcctggagggggatgagtcctgaggtgggcatttcacgggaagggttggtgtgctggctttagacggggagaaacatctggaaggatgctcgagggggggcccgggc

SEQ ID NO: 40 - protein, 762 aa, Homo Sapiens
MASLLQDQLTTDQDLLLMQEGMPMRKVRSKSWKKLRYFRLQNDGMTVWHARQARGSAKPSFSISDVETIRNGHDSELLRSLAEELPLEQGFTIVFHGRRSNLDLMANSVEEAQIWMRGLQLLVDLVTSMDHQERLDQWLSDWFQRGDKNQDGKMSFQEVQRLLHLMNVEMDQEYAFSLFQAADTSQSGTLEGEEFVQFYKALTKRAEVQELFESFSADGQKLTLLEFLDFLQEEQKERDCTSELALELIDRYEPSDSGKLRHVLSMDGFLSYLCSKDGDIFNPACLPIYQDMTQPLNHYFICSSHNTYLVGDQLCGQSSVEGYIRALKRGCRCVEVDVWDGPSGEPVVYHGHTLTSRILFKDVVATVAQYAFQTSDYPVILSLETHCSWEQQQTMARHLTEILGEQLLSTTLDGVLPTQLPSPEELRRKILVKGKKLTLEEDLEYEEEEAEPELEESELALESQFETEPEPQEQNLQNKDKKKKSKPILCPALSSLVIYLKSVSFRSFTHSKEHYHFYEISSFSETKAKRLIKEAGNEFVQHNTWQLSRVYPSGLRTDSSNYNPQELWNAGCQMVAMNMQTAGLEMDICDGHFRQNGGCGYVLKPDFLRDIQSSFHPEKPISPFKAQTLLIQVISGQQLPKVDKTKEGSIVDPLVKVQIFGVRLDTARQETNYVENNGFNPYWGQTLCFRVLVPELAMLRFVVMDYDWKSRNDFIGQYTLPWTCMQQGYRHIHLLSKDGISLRPASIFVYICIQEGLEGDES

SEQ ID NO: 41 - DNA, 2289 bp, Homo Sapiens
  CDS (1)...(2289)
atggcgtccctgctgcaagaccagctgaccactgatcaggacttgctgctgatgcaggaaggcatgccgatgcgcaaggtgaggtccaaaagctggaagaagctaagatacttcagacttcagaatgacggcatgacagtctggcatgcacggcaggccaggggcagtgccaagcccagcttctcaatctctgatgtggagacaatacgtaatggccatgattccgagttgctgcgtagcctggcagaggagctccccctggagcagggcttcaccattgtcttccatggccgccgctccaacctggacctgatggccaacagtgttgaggaggcccagatatggatgcgagggctccagctgttggtggatcttgtcaccagcatggaccatcaggagcgcctggaccaatggctgagcgattggtttcaacgtggagacaaaaatcaggatggtaagatgagtttccaagaagttcagcggttattgcacctaatgaatgtggaaatggaccaagaatatgccttcagtctttttcaggcagcagacacgtcccagtctggaaccctggaaggagaagaattcgtacagttctataaggcattgactaaacgtgctgaggtgcaggaactgtttgaaagtttttcagctgatgggcagaagctgactctgctggaatttttggatttcctccaagaggagcagaaggagagagactgcacctctgagcttgctctggaactcattgaccgctatgaaccttcagacagtggcaaactgcggcatgtgctgagtatggatggcttcctcagctacctctgctctaaggatggagacatcttcaacccagcctgcctccccatctatcaggatatgactcaacccctgaaccactacttcatctgctcttctcataacacctacctagtgggggaccagctttgcggccagagcagcgtcgagggatatatacgggccctgaagcgggggtgccgctgcgtggaggtggatgtatgggatggacctagcggggaacctgtcgtttaccacggacacaccctgacctcccgcatcctgttcaaagatgtcgtggccacagtagcacagtatgccttccagacatcagactacccagtcatcttgtccctggagacccactgcagctgggagcagcagcagaccatggcccgtcatctgactgagatcctgggggagcagctgctgagcaccaccttggatggggtgctgcccactcagctgccctcgcctgaggagcttcggaggaagatcctggtgaaggggaagaagttaacacttgaggaagacctggaatatgaggaagaggaagcagaacctgagttggaagagtcagaattggcgctggagtcccagtttgagactgagcctgagccccaggagcagaaccttcagaataaggacaaaaagaagaaatccaagcccatcttgtgtccagccctctcttccctggttatctacttgaagtctgtctcattccgcagcttcacacattcaaaggagcactaccacttctacgagatatcatctttctctgaaaccaaggccaagcgcctcatcaaggaggctggcaatgagtttgtgcagcacaatacttggcagttaagccgtgtgtatcccagcggcctgaggacagactcttccaactacaacccccaggaactctggaatgcaggctgccagatggtggccatgaatatgcagactgcagggcttgaaatggacatctgtgatgggcatttccgccagaatggcggctgtggctatgtgctgaagccagacttcctgcgtgatatccagagttctttccaccctgagaagcccatcagccctttcaaagcccagactctcttaatccaggtgatcagcggtcagcaactccccaaagtggacaagaccaaagaggggtccattgtggatccactggtgaaagtgcagatctttggcgttcgtctagacacagcacggcaggagaccaactatgtggagaacaatggttttaatccatactgggggcagacactatgtttccgggtgctggtgcctgaacttgccatgctgcgttttgtggtaatggattatgactggaaatcccgaaatgactttattggtcagtacaccctgccttggacctgcatgcaacaaggttaccgccacattcacctgctgtccaaagatggcatcagcctccgcccagcttccatctttgtgtatatctgcatccaggaaggcctggagggggatgagtcctga

SEQ ID NO: 42 - DNA, 2171 bp, Homo Sapiens
  CDS (232)...(2058)
ccacgcgtccgcccacgcgtccgcagccaaccagctatacctcttttgaagattttaagaacttagcctcctgaacagtcttcttcgaaagtgaaaagtggtaacagctgatgagtatcaagaaattattttccgcaaaggggcagagttaattgtatttggaacccatgacagcacctactggggaaagacttctaagtgaggagaaacggctctacaggtcatgaaactatggaaatgagatggtttttgtcaaagattcaggatgacttcagaggtggaaaaattaacctagaaaaaactcagaggttacttgaaaaattagatattcggtgcagttatattcatgtgaaacagatttttaaggacaatgacaggctgaaacaaggaagaatcaccatagaagaatttagagcaatttatcgaattatcacgcacagagaagaaattattgagattttcaacacatattctgaaaaccggaaaattcttttagcaagtaatctggctcaatttctgacacaagaacaatatgcagctgagatgagtaaagctattgcttttgagatcattcagaaatacgagcctatcgaagaagttaggaaagcacaccaaatgtcattagaaggttttacaagatacatggattcacgtgaatgtctactgtttaaaaatgaatgtagaaaagtttatcaagatatgactcatccattaaatgattattttatttcatcttcacataacacatatttggtatctgatcaattattgggaccaagtgacctttggggatatgcaagtgcccttgtgaaaggatgccgttgtttggagattgactgctgggatggagcacaaaatgaacctgttgtatatcatggctacacactcacaagcaaacttctgtttaaaactgttatccaagctatacacaagtatgcattcatgacatctgactacccagtggtgctctctttagaaaatcactgctccactgcccaacaagaagtaatggcagacaatttgcaggctacttttggagagtccttgctttctgatatgcttgatgattttcctgatactctaccatcaccagaggcactaaaattcaaaatattagttaaaaataagaaaataggaaccttaaaggaaacccatgaaagaaaaggttctgataagcatggagacaatcaagacaaggaaacaggggtaaaaaagttacctggagtaatgcttttcaagaaaaagaagaccaggaagctaaaaattgctctggccttatctgatcttgtcatttatacgaaagctgagaaattcaaaagctttcaacattcaagattatatcagcaatttaatgaaaataattctattggggagacacaagcccgaaaactttcaaaattgcgagtccatgagtttatttttcacaccaggaagttcattaccagaatatatcccaaagcaacaagagcagactcttctaattttaatccccaagaattttggaatataggttgtcaaatggtggctttaaatttccagacccctggtctgcccatggatctgcaaaatgggaaatttttggataatggtggttctggatatattttgaaaccacatttcttaagagagagtaaatcatactttaacccaagtaacataaaagagggtatgccaattacacttacaataaggctcatcagtggtatccagttgcctcttactcattcatcatctaacaaaggtgattcattagtaattatagaagtttttggtgttccaaatgatcaaatgaagcagcagactcgtgtaattaaaaaaaatgcttttagtccaagatggaatgaaacattcacatttattattcatgtcccagaattggcattgatacgttttgttgttgaaggtcaaggtttaatagcaggaaatgaatttcttgggcaatatactttgccacttctatgcatgaacaaaggttatcgtcgtattcctctgttttccagaatgggtgagagccttgagcctgcttcactgtttgtttatgtttggtacgtcagataacagctaatgataaatgacatatcattagctatgcatcgcaataaaacagccaaaatgaaaaaaaaaaaaaaaaaaaaaaaattggcggccgcaagcttattccctttagtaag

SEQ ID NO: 43 - protein, 608 aa, Homo Sapiens
MEMRWFLSKIQDDFRGGKINLEKTQRLLEKLDIRCSYIHVKQIFKDNDRLKQGRITIEEFRAIYRIITHREEIIEIFNTYSENRKILLASNLAQFLTQEQYAAEMSKAIAFEIIQKYEPIEEVRKAHQMSLEGFTRYMDSRECLLFKNECRKVYQDMTHPLNDYFISSSHNTYLVSDQLLGPSDLWGYASALVKGCRCLEIDCWDGAQNEPVVYHGYTLTSKLLFKTVIQAIHKYAFMTSDYPVVLSLENHCSTAQQEVMADNLQATFGESLLSDMLDDFPDTLPSPEALKFKILVKNKKIGTLKETHERKGSDKHGDNQDKETGVKKLPGVMLFKKKKTRKLKIALALSDLVIYTKAEKFKSFQHSRLYQQFNENNSIGETQARKLSKLRVHEFIFHTRKFITRIYPKATRADSSNFNPQEFWNIGCQMVALNFQTPGLPMDLQNGKFLDNGGSGYILKPHFLRESKSYFNPSNIKEGMPITLTIRLISGIQLPLTHSSSNKGDSLVIIEVFGVPNDQMKQQTRVIKKNAFSPRWNETFTFIIHVPELALIRFVVEGQGLIAGNEFLGQYTLPLLCMNKGYRRIPLFSRMGESLEPASLFVYVWYVR

SEQ ID NO: 44 - DNA, 1827 bp, Homo Sapiens
  CDS (1)...(1827)
atggaaatgagatggtttttgtcaaagattcaggatgacttcagaggtggaaaaattaacctagaaaaaactcagaggttacttgaaaaattagatattcggtgcagttatattcatgtgaaacagatttttaaggacaatgacaggctgaaacaaggaagaatcaccatagaagaatttagagcaatttatcgaattatcacgcacagagaagaaattattgagattttcaacacatattctgaaaaccggaaaattcttttagcaagtaatctggctcaatttctgacacaagaacaatatgcagctgagatgagtaaagctattgcttttgagatcattcagaaatacgagcctatcgaagaagttaggaaagcacaccaaatgtcattagaaggttttacaagatacatggattcacgtgaatgtctactgtttaaaaatgaatgtagaaaagtttatcaagatatgactcatccattaaatgattattttatttcatcttcacataacacatatttggtatctgatcaattattgggaccaagtgacctttggggatatgcaagtgcccttgtgaaaggatgccgttgtttggagattgactgctgggatggagcacaaaatgaacctgttgtatatcatggctacacactcacaagcaaacttctgtttaaaactgttatccaagctatacacaagtatgcattcatgacatctgactacccagtggtgctctctttagaaaatcactgctccactgcccaacaagaagtaatggcagacaatttgcaggctacttttggagagtccttgctttctgatatgcttgatgattttcctgatactctaccatcaccagaggcactaaaattcaaaatattagttaaaaataagaaaataggaaccttaaaggaaacccatgaaagaaaaggttctgataagcatggagacaatcaagacaaggaaacaggggtaaaaaagttacctggagtaatgcttttcaagaaaaagaagaccaggaagctaaaaattgctctggccttatctgatcttgtcatttatacgaaagctgagaaattcaaaagctttcaacattcaagattatatcagcaatttaatgaaaataattctattggggagacacaagcccgaaaactttcaaaattgcgagtccatgagtttatttttcacaccaggaagttcattaccagaatatatcccaaagcaacaagagcagactcttctaattttaatccccaagaattttggaatataggttgtcaaatggtggctttaaatttccagacccctggtctgcccatggatctgcaaaatgggaaatttttggataatggtggttctggatatattttgaaaccacatttcttaagagagagtaaatcatactttaacccaagtaacataaaagagggtatgccaattacacttacaataaggctcatcagtggtatccagttgcctcttactcattcatcatctaacaaaggtgattcattagtaattatagaagtttttggtgttccaaatgatcaaatgaagcagcagactcgtgtaattaaaaaaaatgcttttagtccaagatggaatgaaacattcacatttattattcatgtcccagaattggcattgatacgttttgttgttgaaggtcaaggtttaatagcaggaaatgaatttcttgggcaatatactttgccacttctatgcatgaacaaaggttatcgtcgtattcctctgttttccagaatgggtgagagccttgagcctgcttcactgtttgtttatgtttggtacgtcagataa

SEQ ID NO: 45 - protein, 85 aa, Artificial Sequence
  Consensus Amino Acid Sequence
VIKEGWLLKKSKSWKKRYFVLFNNVLLYYKDSKKKPKGSIPLSGCQVEKPDKNCFEIRTDRTLLLQAESEEERKEWVKAIQSAIR

SEQ ID NO: 46 - protein, 29 aa, Artificial Sequence
  Consensus Amino Acid Sequence
ELKEAFKEFDKDGDGKISFEEFKAALKKL

SEQ ID NO: 47 - protein, 29 aa, Artificial Sequence
  Consensus Amino Acid Sequence
ELKEAFKEFDKDGDGKISFEEFKAALKKL

SEQ ID NO: 48 - protein, 153 aa, Artificial Sequence
  Consensus Amino Acid Sequence
DMSIPLSHYFISSSHNTYLTGKQLWGKSSVESYRQQLDAGCRCVELDCWDGKPDDEPIIYHGHTLTLEIKLKDVLEAIKDFAFKPTSPYPVILSLENHCNSDDQQRKMAKYFKEIFGDMLLTKPTLDSLTTEPGLPLPSLKDLRGKILLKNKK

SEQ ID NO: 49 - protein, 128 aa, Artificial Sequence
  Consensus Amino Acid Sequence
ELSNLVNYIQSIKFRSFELSGEEKNTSYEISSFSERKVKAKKLLKESPVEFVKYNKRQLSRVYPKGTRVDSSNFMPQVFWNAGCQMVALNFQTSDLPMQINDGMFEYNGGQPDGSFKSGYLLKPEFLR

SEQ ID NO: 50 - protein, 95 aa, Artificial Sequence
  Consensus Amino Acid Sequence
LTVTVIEARNLPKMDKVNGRLSDPYVKVSLLGDKKDLKKFKTKVVKKTNGLNPVWNEETFVFEKVPLPELASKTLRFAVYDEDRFSRDDFIGQVT

SEQ ID NO: 51 - protein, 170 aa, Artificial Sequence
  Consensus Amino Acid Sequence
SPDCNVFDPEHKQVHQDMNQPLSHYFINSSHNTYLTGNQLSSGESSVEMYRQALLKGCRCIELDCWDGKDGDPEPIITHGHTMTTEISFKDCLEAIKEHAFVTSEYPVILSLENHCDSTPQQQAKMAEYCKEVFGDMLFTEPLEESPLEPGKELPSPEELKRKILIKNKK

SEQ ID NO: 52 - protein, 202 aa, Artificial Sequence
  Consensus Amino Acid Sequence
MASQIKKISSTNDCLQFMQKGSELKKVRSNSWKYNRYFTLDDDMQTLWWEPHWFSKKDSEKPKFDISDIKEIRMGKNTETFRNNGKEFQIQEPEDCCFSIIFGENYFHESLDLVANSADVANIWVSGLRYLVDYAKHMLDNYQEQLDQWLREWFQQADRNKDSRMSFREAQNLLKLMNVQMDEEYAFSIFRECDFSQSNTLD

SEQ ID NO: 53 - protein, 119 aa, Artificial Sequence
  Consensus Amino Acid Sequence
PELSDLVNYCQPVKFKGFEMAEEKNTYYHMSSFSENKAEKLVNKEHPKEFVRYNQRNLLRVYPKGTRIDSSNYNPMVFWNHGCQMVALNFQTHGRSMWLNQGMFRANGGCGYVLKPDFL

SEQ ID NO: 54 - protein, 40 aa, Artificial Sequence
  Consensus Amino Acid Sequence
WTCMKHGYRHVSLLSKDGTSLHPASHFVYTCMQEDLDMDE

SEQ ID NO: 55 - protein, 63 aa, Artificial Sequence
  Consensus Amino Acid Sequence
YKPQRAWMHGAQMIALSRQDDKEKLWLMQGMFRANGGCGYVKKPNFLLNAGSSGVFYPTENPV

SEQ ID NO: 56 - protein, 29 aa, Artificial Sequence
  Consensus Amino Acid Sequence
ELKEAFKEFDKDGDGKISFEEFKAALKKL

SEQ ID NO: 57 - protein, 153 aa, Artificial Sequence; identical to SEQ ID NO: 48
  Consensus Amino Acid Sequence

SEQ ID NO: 58 - protein, 128 aa, Artificial Sequence; identical to SEQ ID NO: 49
  Consensus Amino Acid Sequence

SEQ ID NO: 59 - protein, 95 aa, Artificial Sequence; identical to SEQ ID NO: 50
  Consensus Amino Acid Sequence

SEQ ID NO: 60 - protein, 191 aa, Artificial Sequence
  Consensus Amino Acid Sequence
SPDCNVFDPEHKQVHQDMNQPLSHYFINSSHNTYLTGNQLSSGESSVEMYRQALLKGCRCIELDCWDGKDGDPEPIITHGHTMTTEISFKDCLEAIKEHAFVTSEYPVILSLENHCDSTPQQQAKMAEYCKEVFGDMLFTEPLEESPLEPGKELPSPEELKRKILIKNKKLKEHSEEKESEEKKTDEETES

SEQ ID NO: 61 - protein, 128 aa, Artificial Sequence
  Consensus Amino Acid Sequence
SDLVNYCQPVKFKGFEMAEEKNTYYHMSSFSENKAEKLVNKEHPKEFVRYNQRNLLRVYPKGTRIDSSNYNPMVFWNHGCQMVALNFQTHGRSMWLNQGMFRANGGCGYVLKPDFLLKARPNDEVFDP

SEQ ID NO: 62 - protein, 51 aa, Artificial Sequence
  Consensus Amino Acid Sequence
DXDNSILVFYWDENSTGDNQGHRKGPLIVMCDENQSTAGCXXDELIVMFYW

SEQ ID NO: 63 - DNA, 4055 bp, Homo Sapiens
  CDS (210)...(3752)
tactatagggagtcgacccacgcgtccggccgcgccgagcctggtggcccaggtgccccgcccgcgtcagccctgctccagccccgcgctagcccagcgcccctcgccccgggccgtccggaccgcgcccccgcccagggccttgcgcacgccggggcccaggccgagggccgcagcgccggggccggcgatgagcgcgaggagccggcatgagcgcagacagcagccctctcgtgggcagcacgcccaccggttatgggaccctgacgatagggacatcaatagatcccctcagctcctcagtttcatccgtgaggctcagcggctactgtggcagtccatggagggtcatcggctatcacgtcgtggtctggatgatggctgggatccctttgctgctcttccgttggaagcccctgtggggggtgcggctgcggctccggccctgcaacctggcccacgccgaaacactcgttatcgaaataagagacaaagaggatagttcctggcagctcttcactgtccaggtgcagactgaggccatcggcgagggcagcctggagccgtccccacagtcccaggcagaggatggccggagccaggcggcagttggggcggtaccagagggtgcctggaaggatacggcccagctccacaagagcgaggaggcggtgagtgtcggacagaagcgggtgctgcggtattacctcttccagggccagcgctatatctggatcgagacccagcaagccttctaccaggtcagcctcctggaccatggccgctcttgtgacgacgtccaccgctcccgccatggcctcagcctccaggaccaaatggtgaggaaggccatttacggccccaacgtgatcagcataccggtcaagtcctacccccagctgctggtggacgaggcactgaacccctactatgggttccaggccttcagcatcgcgctgtggctggctgaccactactactggtacgccctgtgcatcttcctcatttcctccatctccatctgcctgtcgctgtacaagaccagaaagcaaagccagactctaagggacatggtcaagttgtccatgcgggtgtgcgtgtgccggccagggggagaggaagagtgggtggactccagtgagctagtgcccggagactgcctggtgctgccccaggagggtgggctgatgccctgtgatgccgccctggtggccggcgagtgcatggtgaatgagagctctctgacaggagagagcattccagtgctgaagacggcactgccggaggggctggggccctactgtgcagagacacaccggcggcacacactcttctgcgggaccctcatcttgcaggcccgggcctatgtgggaccgcacgtcctggcagtggtgacccgcacagggttctgcacggcaaaagggggcctggtgagctccatcttgcacccccggcccatcaacttcaagttctataaacacagcatgaagtttgtggctgccctctctgtcctggctctcctcggcaccatctacagcatcttcatcctctaccgaaaccgggtgcctctgaatgagattgtaatccgggctctcgacctggtgaccgtggtggtgccacctgccctgcctgctgccatgactgtgtgcacgctctacgcccagagccgactgcggagacagggcattttctgcatccacccactgcgcatcaacctggggggcaagctgcagctggtgtgtttcgacaagacgggcaccctcactgaggacggcttagacgtgatgggggtggtgcccctgaaggggcaggcattcctgcccctggtcccagagcctcgccgcctgcctgtggggcccctgctccgagcactggccacctgccatgccctcagccggctccaggacacccccgtgggcgaccccatggacttgaagatggtggagtctactggctgggtcctggaggaagagccggctgcagactcagcatttgggacccaggtcttggcagtgatgagacctccactttgggagccccagctgcaggcaatggaggagcccccggtgccagtcagcgtcctccaccgcttccccttctcttcggctctgcagcgcatgagtgtggtggtggcgtggccaggggccactcagcccgaggcctacgtcaaaggctccccggagctggtggcagggctctgcaaccccgagacagtgcccaccgacttcgcccagatgctgcagagctatacagctgctggctaccgtgtcgtggccctggccagcaagccactgcccactgtgcccagcctggaggcagcccagcaactgacgagggacactgtggaaggagacctgagcctcctggggctgctggtcatgaggaacctactgaagccgcagacaacgccagttatccaggctctgcgaaggacccgcatccgcgccgtcatggtgacaggggacaacctgcagacagcggtgactgtggcccggggctgtggcatggtggccccccaggagcatctgatcatcgtccacgccacccaccctgagcggggtcagcctgcctctctcgagttcctgccgatggagtcccccacagccgtgaatggcgttaaggatcctgaccaggctgcaagctacaccgtggagccagacccccgatccaggcacctggccctcagcgggcccacctttggtatcattgtgaagcacttccccaagctgctgcccaaggtcctggtccagggcactgtctttgcccgcatggcccctgagcagaagacagagctggtgtgcgagctacagaagcttcagtactgcgtgggcatgtgcggagacggtgccaatgactgtggggccctgaaggcggctgatgtcggcatctcgctgtcccaggcagaagcctcagtggtctcacccttcacctcgagcatggccagtattgagtgcgtgcccatggtcatcagggaggggcgctgttcccttgacacttcgttcagcgtcttcaagtacatggctctgtacagcctgacccagttcatctccgtcctgatcctctacacgatcaacaccaacctgggtgacctgcagttcctggccatcgacctggtcatcaccaccacagtggcagtgctcatgagccgcacggggccagcgctggtcctgggacgggtacggccaccgggggcgctgctcagcgtgcccgtgctcagcagcctgctgctgcagatggtcctggtgaccggcgtgcagctagggggctacttcctgaccctggcccagccatggttcgtgcctctgaacaggacagtggccgcaccagacaacctgcccaactacgagaacaccgtggtcttctctctgtccagcttccagtacctcatcctggctgcagccgtgtccaagggggcgcccttccgccggccgctctacaccaatgtgcccttcctggtggccctggcgctcctgagctccgtcctggtgggccttgtcctggtccccggcctcctgcaggggccgctggcgctgaggaacatcactgacaccggcttcaagctgctgctgctgggtctggtcaccctcaacttcgtgggggccttcatgctggagagcgtgctagaccagtgcctccccgcctgcctgcgccgcctccggcccaagcgggcctccaagaagcgcttcaagcagctggaacgagagctggccgagcagccctggccaccgctgcccgccggccccctgaggtagtgcaggcccacgggcaccccagacactggaactccctgcctctgagccaccaactggacccctctccagcaacaccaccgccaccacctcccacatccctgaggttggcgactgtctacactcctcccccgagaccacccccaccctggggaagcgttgactactgtcccctaccttggaccatcccgcgtaggggtggcagcccccagctcccctcagtgctgctgtcagtgtagcaaataaagtcatgatattttcctggcaaaaaaaaaaaaaaaaaaaaaaaaaaaaaaaaaaaaaaaa

SEQ ID NO: 64 - protein, 1180 aa, Homo Sapiens
MSADSSPLVGSTPTGYGTLTIGTSIDPLSSSVSSVRLSGYCGSPWRVIGYHVVVWMMAGIPLLLFRWKPLWGVRLRLRPCNLAHAETLVIEIRDKEDSSWQLFTVQVQTEAIGEGSLEPSPQSQAEDGRSQAAVGAVPEGAWKDTAQLHKSEEAVSVGQKRVLRYYLFQGQRYIWIETQQAFYQVSLLDHGRSCDDVHRSRHGLSLQDQMVRKAIYGPNVISIPVKSYPQLLVDEALNPYYGFQAFSIALWLADHYYWYALCIFLISSISICLSLYKTRKQSQTLRDMVKLSMRVCVCRPGGEEEWVDSSELVPGDCLVLPQEGGLMPCDAALVAGECMVNESSLTGESIPVLKTALPEGLGPYCAETHRRHTLFCGTLILQARAYVGPHVLAVVTRTGFCTAKGGLVSSILHPRPINFKFYKHSMKFVAALSVLALLGTIYSIFILYRNRVPLNEIVIRALDLVTVVVPPALPAAMTVCTLYAQSRLRRQGIFCIHPLRINLGGKLQLVCFDKTGTLTEDGLDVMGVVPLKGQAFLPLVPEPRRLPVGPLLRALATCHALSRLQDTPVGDPMDLKMVESTGWVLEEEPAADSAFGTQVLAVMRPPLWEPQLQAMEEPPVPVSVLHRFPFSSALQRMSVVVAWPGATQPEAYVKGSPELVAGLCNPETVPTDFAQMLQSYTAAGYRVVALASKPLPTVPSLEAAQQLTRDTVEGDLSLLGLLVMRNLLKPQTTPVIQALRRTRIRAVMVTGDNLQTAVTVARGCGMVAPQEHLIIVHATHPERGQPASLEFLPMESPTAVNGVKDPDQAASYTVEPDPRSRHLALSGPTFGIIVKHFPKLLPKVLVQGTVFARMAPEQKTELVCELQKLQYCVGMCGDGANDCGALKAADVGISLSQAEASVVSPFTSSMASIECVPMVIREGRCSLDTSFSVFKYMALYSLTQFISVLILYTINTNLGDLQFLAIDLVITTTVAVLMSRTGPALVLGRVRPPGALLSVPVLSSLLLQMVLVTGVQLGGYFLTLAQPWFVPLNRTVAAPDNLPNYENTVVFSLSSFQYLILAAAVSKGAPFRRPLYTNVPFLVALALLSSVLVGLVLVPGLLQGPLALRNITDTGFKLLLLGLVTLNFVGAFMLESVLDQCLPACLRRLRPKRASKKRFKQLERELAEQPWPPLPAGPLR

SEQ ID NO: 65 - DNA, 3540 bp, Homo Sapiens
  CDS (1)...(3540)
atgagcgcagacagcagccctctcgtgggcagcacgcccaccggttatgggaccctgacgatagggacatcaatagatcccctcagctcctcagtttcatccgtgaggctcagcggctactgtggcagtccatggagggtcatcggctatcacgtcgtggtctggatgatggctgggatccctttgctgctcttccgttggaagcccctgtggggggtgcggctgcggctccggccctgcaacctggcccacgccgaaacactcgttatcgaaataagagacaaagaggatagttcctggcagctcttcactgtccaggtgcagactgaggccatcggcgagggcagcctggagccgtccccacagtcccaggcagaggatggccggagccaggcggcagttggggcggtaccagagggtgcctggaaggatacggcccagctccacaagagcgaggaggcggtgagtgtcggacagaagcgggtgctgcggtattacctcttccagggccagcgctatatctggatcgagacccagcaagccttctaccaggtcagcctcctggaccatggccgctcttgtgacgacgtccaccgctcccgccatggcctcagcctccaggaccaaatggtgaggaaggccatttacggccccaacgtgatcagcataccggtcaagtcctacccccagctgctggtggacgaggcactgaacccctactatgggttccaggccttcagcatcgcgctgtggctggctgaccactactactggtacgccctgtgcatcttcctcatttcctccatctccatctgcctgtcgctgtacaagaccagaaagcaaagccagactctaagggacatggtcaagttgtccatgcgggtgtgcgtgtgccggccagggggagaggaagagtgggtggactccagtgagctagtgcccggagactgcctggtgctgccccaggagggtgggctgatgccctgtgatgccgccctggtggccggcgagtgcatggtgaatgagagctctctgacaggagagagcattccagtgctgaagacggcactgccggaggggctggggccctactgtgcagagacacaccggcggcacacactcttctgcgggaccctcatcttgcaggcccgggcctatgtgggaccgcacgtcctggcagtggtgacccgcacagggttctgcacggcaaaagggggcctggtgagctccatcttgcacccccggcccatcaacttcaagttctataaacacagcatgaagtttgtggctgccctctctgtcctggctctcctcggcaccatctacagcatcttcatcctctaccgaaaccgggtgcctctgaatgagattgtaatccgggctctcgacctggtgaccgtggtggtgccacctgccctgcctgctgccatgactgtgtgcacgctctacgcccagagccgactgcggagacagggcattttctgcatccacccactgcgcatcaacctggggggcaagctgcagctggtgtgtttcgacaagacgggcaccctcactgaggacggcttagacgtgatgggggtggtgcccctgaaggggcaggcattcctgcccctggtcccagagcctcgccgcctgcctgtggggcccctgctccgagcactggccacctgccatgccctcagccggctccaggacacccccgtgggcgaccccatggacttgaagatggtggagtctactggctgggtcctggaggaagagccggctgcagactcagcatttgggacccaggtcttggcagtgatgagacctccactttgggagccccagctgcaggcaatggaggagcccccggtgccagtcagcgtcctccaccgcttccccttctcttcggctctgcagcgcatgagtgtggtggtggcgtggccaggggccactcagcccgaggcctacgtcaaaggctccccggagctggtggcagggctctgcaaccccgagacagtgcccaccgacttcgcccagatgctgcagagctatacagctgctggctaccgtgtcgtggccctggccagcaagccactgcccactgtgcccagcctggaggcagcccagcaactgacgagggacactgtggaaggagacctgagcctcctggggctgctggtcatgaggaacctactgaagccgcagacaacgccagttatccaggctctgcgaaggacccgcatccgcgccgtcatggtgacaggggacaacctgcagacagcggtgactgtggcccggggctgtggcatggtggccccccaggagcatctgatcatcgtccacgccacccaccctgagcggggtcagcctgcctctctcgagttcctgccgatggagtcccccacagccgtgaatggcgttaaggatcctgaccaggctgcaagctacaccgtggagccagacccccgatccaggcacctggccctcagcgggcccacctttggtatcattgtgaagcacttccccaagctgctgcccaaggtcctggtccagggcactgtctttgcccgcatggcccctgagcagaagacagagctggtgtgcgagctacagaagcttcagtactgcgtgggcatgtgcggagacggtgccaatgactgtggggccctgaaggcggctgatgtcggcatctcgctgtcccaggcagaagcctcagtggtctcacccttcacctcgagcatggccagtattgagtgcgtgcccatggtcatcagggaggggcgctgttcccttgacacttcgttcagcgtcttcaagtacatggctctgtacagcctgacccagttcatctccgtcctgatcctctacacgatcaacaccaacctgggtgacctgcagttcctggccatcgacctggtcatcaccaccacagtggcagtgctcatgagccgcacggggccagcgctggtcctgggacgggtacggccaccgggggcgctgctcagcgtgcccgtgctcagcagcctgctgctgcagatggtcctggtgaccggcgtgcagctagggggctacttcctgaccctggcccagccatggttcgtgcctctgaacaggacagtggccgcaccagacaacctgcccaactacgagaacaccgtggtcttctctctgtccagcttccagtacctcatcctggctgcagccgtgtccaagggggcgcccttccgccggccgctctacaccaatgtgcccttcctggtggccctggcgctcctgagctccgtcctggtgggccttgtcctggtccccggcctcctgcaggggccgctggcgctgaggaacatcactgacaccggcttcaagctgctgctgctgggtctggtcaccctcaacttcgtgggggccttcatgctggagagcgtgctagaccagtgcctccccgcctgcctgcgccgcctccggcccaagcgggcctccaagaagcgcttcaagcagctggaacgagagctggccgagcagccctggccaccgctgcccgccggccccctgagg

SEQ ID NO: 66 - protein, 1187 aa, Caenorhabditis elegans
MTLESGDHTLTLFAYRTGPFRTILFYALTVLTLGIFRLILHWKQKWDVKMRMVPCTFEAAEYIYIIDNHNVSELQPVLRKSNATIPTENGEMRKVPELRWFVYRKLEYVWIDDLNSDESVDEISDNDNCWKTSFEIANRIPCRSLLAVSESNFGLTLSEISRRLEFYGRNEIVVQLRPILYLLVMEVITPFYVFQIFSVTVWYNDEYAYYASLIVILSLGSIVMDVYQIRTQEIRLRSMVHSTESVEVIREGTEMTIGSDQLVPGDILLIPPHGCLMQCDSVLMNGTVIVNESVLTGESVPITKVALTDETNDSVFNIEKNSKNVLFCGTQVLQTRFYRGKKVKAIVLRTAYSTLKGQLVRSIMYPKPVDFRFTKDLFKFILFLACISGCGFIYTIIVMIMRGNTLRRIIVRSLDIITITVPPALPAAMSVGIINAQLRLKKKEIFCISPSTINTCGAINVVCFDKTGTLTEDGLDFHVVRPVMSAVNQEIQKVKLEKSNRTEFMGEMTELTSRNGLPFDGDLVKAIATCHSLTRINGVLHGDPLDLILFQKTGWTMEEGIEGDIEEETQRFDNVQPSIIKPTDDKSAEYSVIRQFTFSSSLQRMSVIVFDPREDRPDNMMLYSKGSPEMILSLCDPNTVPEDYLLQVNSYAQHGFRLIAVARRPLDLNFNKASKVKRDAVECDLEMLGLIVMENRVKPVTLGVINQLNRANIRTVMVTGDNLLTGLSVARECGIIRPSKRAFLVEHVPGELDEYGRTKIFVKQSVSSSDEVIEDDASVSISMCSSTWKGSSEGDGFSPTNTEVETPNPVTADSLGHLIASSYHLAISGPTFAVIVHEYPELVDQLCSVCDVFARMAPDQKQSLVEQLQQIDYTVAMCGDGANDCAALKAAHAGISLSDAEASIAAPFTSKVPDIRCVPTVISEGRAALVTSFGIFKYMAGYSLTQFVTVMHLYWISNILTDGQFMYIDMFLITMFALLFGNTPAFYRLAHTPPPTRLLSIASMTSVVGQLIIIGVVQFIVFFSTSQQPWFTPYQPPVDDEVEDKRSMQGTALFCVSMFQYIILALVYSKGPPFRGNLWSNKPIYKKKRSIEAIIDYVPTTNSDHIRRPSINGVTSSRTESTLLSAEGQQLHMTTSKNGKGGENPHSSALFERLISRIGGEPTWLTNPIPPHSLSEPEEPEKLERTY

SEQ ID NO: 67 - DNA, 7249 bp, Homo Sapiens
  CDS (225)...(3995)
cacgcgtccgggctgggctgaggcgaggcggcggcggcgacagcggcggccgggtcccccgcggcccctggggctggtccggccgcgagggaggccgcggaggaggcggcgcggcggcggccagtgagcggccccgatctgacagacatccctgaatcttggtgtttggacataggagtgatcttccattgtttgaagcactggacctttaatccactgtaggtatggacagggaagaaaggaagaccatcaatcagggtcaagaagatgaaatggagatttatggttacaatttgagtcgctggaagcttgccatagtttctttaggagtgatttgctctgatgggtttctcctcctcctcctctattggatgcctgagtggcgggtgaaagcgacctgtgtcagagctgcaattaaagactgtgaagtagtgctgctgaggactactgatgaattcaaaatgtggttttgtgcaaaaattcgcgttctttctttggaaacttacccagtttcaagtccaaaatctatgtctaataagctttcaaatggccatgcagtttgtttaattgagaatcccactgaagaaaataggcacaggatcagtaaatattcacagactgaatcacaacagattcgttatttcacccaccatagtgtaaaatatttctggaatgataccattcacaattttgatttcttaaagggactggatgaaggtgtttcttgtacgtcaatttatgaaaagcatagtgcaggactgacaaaggggatgcatgcctacagaaaactgctttatggagtaaatgaaattgctgtaaaagtgccttctgtttttaagcttctaattaaagaggttctcaacccattttacattttccagctgttcagtgttatactgtggagcactgatgaatactattactatgctctagctattgtggttatgtccatagtatcaatcgtaagctcactatattccattagaaagcaatatgttatgttgcatgacatggtggcaactcatagtaccgtaagagtttcagtttgtagagtaaatgaagaaatagaagaaatcttttctaccgaccttgtgccaggagatgtcatggtcattccattaaatgggacaataatgccttgtgatgctgtgcttattaatggtacctgcattgtaaacgaaagcatgttaacaggagaaagtgttccagtgacaaagactaatttgccaaatccttcagtggatgtgaaaggaataggagatgaattatataatccagaaacacataaacgacatactttgttttgtgggacaactgttattcagactcgtttctacactggagaactcgtcaaagccatagttgttagaacaggatttagtacttccaaaggacagcttgttcgttccatattgtatcccaaaccaactgattttaaactctacagagatgcctacttgtttctactatgtcttgtggcagttgctggcattgggtttatctacactattattaatagcattttaaatgaggtacaagttggggtcataattatcgagtctcttgatattatcacaattactgtgccccctgcacttcctgctgcaatgactgctggtattgtgtatgctcagagaagactgaaaaaaatcggtattttctgtatcagtcctcaaagaataaatatttgtggacagctcaatcttgtttgctttgacaagactggaactctaactgaagatggtttagatctttgggggattcaacgagtggaaaatgcacgatttctttcaccagaagaaaatgtgtgcaatgagatgttggtaaaatcccagtttgttgcttgtatggctacttgtcattcacttacaaaaattgaaggagtgctctctggtgatccacttgatctgaaaatgtttgaggctattggatggattctggaagaagcaactgaagaagaaacagcacttcataatcgaattatgcccacagtggttcgtcctcccaaacaactgcttcctgaatctacccctgcaggaaaccaagaaatggagctgtttgaacttccagctacttatgagataggaattgttcgccagttcccattttcttctgctttgcaacgtatgagtgtggttgccagggtgctgggggataggaaaatggacgcctacatgaaaggagcgcccgaggccattgccggtctctgtaaacctgaaacagttcctgtcgattttcaaaacgttttggaagacttcactaaacagggcttccgtgtgattgctcttgcacacagaaaattggagtcaaaactgacatggcataaagtacagaatattagcagagatgcaattgagaacaacatggattttatgggattaattataatgcagaacaaattaaagcaagaaacccctgcagtacttgaagatttgcataaagccaacattcgcaccgtcatggtcacaggtgacagtatgttgactgctgtctctgtggccagagattgtggaatgattctacctcaggataaagtgattattgctgaagcattacctccaaaggatgggaaagttgccaaaataaattggcattatgcagactccctcacgcagtgcagtcatccatcagcaattgacccagaggctattccggttaaattggtccatgatagcttagaggatcttcaaatgactcgttatcattttgcaatgaatggaaaatcattctcagtgatactggagcattttcaagaccttgttcctaagttgatgttgcatggcaccgtgtttgcccgtatggcacctgatcagaagacacagttgatagaagcattgcaaaatgttgattattttgttgggatgtgtggtgatggcgcaaatgattgtggtgctttgaagagggcacacggaggcatttccttatcggagctcgaagcttcagtggcatctccctttacctctaagactcctagtatttcctgtgtgccaaaccttatcagggaaggccgtgctgctttaataacttccttctgtgtgtttaaattcatggcattgtacagcattatccagtacttcagtgttactctgctgtattctatcttaagtaacctaggagacttccagtttctcttcattgatctggcaatcattttggtagtggtatttacaatgagtttaaatcctgcctggaaagaacttgtggcacaaagaccaccttcgggtcttatatctggggcccttctcttctccgttttgtctcagattatcatctgcattggatttcaatctttgggttttttttgggtcaaacagcaaccttggtatgaagtgtggcatccaaaatcagatgcttgtaatacaacaggaagcgggttttggaattcttcacacgtagacaatgaaaccgaacttgatgaacataatatacaaaattatgaaaataccacagtgttttttatttccagttttcagtacctcatagtggcaattgccttttcaaaaggaaaacccttcaggcaaccttgctacaaaaattatttttttgttttttctgtgatttttttatatatttttatattattcatcatgttgtatccagttgcctctgttgaccaggttcttcagatagtgtgtgtaccatatcagtggcgtgtaactatgctcatcattgttcttgtcaatgcctttgtgtctatcacagtggagaacttcttccttgacatggtcctttggaaagttgtgttcaaccgagacaaacaaggagagtatcggttcagcaccacacagccaccgcaggagtcagtggatcggtggggaaaatgctgcttaccctgggccctgggctgtagaaagaagacaccaaaggcaaagtacatgtatctggcgcaggagctcttggttgatccagaatggccaccaaaacctcagacaaccacagaagctaaagctttagttaaggagaatggatcatgtcaaatcatcaccataacatagcagtgaatcagtctcagtggtattgctgatagcagtattcaggaatatgtgattttaggagtttctgatcctgtgtgtcagaatggcactagttcagtttatgtcccttctgatatagtagcttatttgacagctttgctcttccttaaaataaaaacagaaaaatatatcgtcctaacagttaaattaacaatcaatccataaagtcctatatcttcattcagcaacccaaatattacatacatttccagaattttcttgattgttactttcagtgatattctttatattgggtacaggagaagtttggtgtttggtaggtttttcaacattagtttttgagactagtttacctcttcacatttatgctcacaaccctcttgttagaaaagtctgtgtttatatacaggctgtaagtttgtgattgataaaaagaagatgagtgttaattagcctccagtgaaaatatactgaaagcctgttttcatttgattccaatgtttcttccaaagaattctgtataaacatatgccaattccctatgatggtctagagttaggaatgagtgtttatggtgttgcttatagaacaactcaggtaatctccatttctggttttatattttctgtacaaactgcctgggttttatttttctaatcagcaaggtgcttcactgccttcttgagacgcctctcaaagctcttaaatggctcctgtgctatgtgtggtgttggcagtctaatttgcttctgttaaatgttgtagaacctttttcactaggaaataagattcatttctttcggcagtagatgtagattcatcttttaacgtttcttcaaatttgtttctgtcaggctttgtgttattttaaatggttttttaaaattttcttctatgttttcaattacctaaagacataggataatagttttttttaagttagaattttacctcataaaattttttgaggtttgatgtatgtctctgtcttatcaataatgaggcttaaaaaatactggatttgaatggctgccgttttttcaaagcaatatgaatttgatgagtttgttttatgccattaggtggcgccagaggtcagaacatgtctattttgaattggatcgttacaaatgagcatatttgatgcggaaatttctgggagaaaaaaaattgaggaaataaagttaaaaaattgacattcattgagccaaaagagatgtggagaaacatttttcacctttctgtttggcctgattaacatttaaattcttgccaaaattaaggtaacttttaaaaaacaccttttataggtggatccagcagtctggcaacgcccacagttaccacaacacagaaaactgatcgtgctataaaatggacgctaaactatgaaaacagtgtgacattgttctctgttcttccagagccagtaacatgcttgctcgtgctttctacttctagctgatcattcttttcccaacatatatttacaaattaccaaattttacctagaattttaggaccaaatggttctcactctttatgctgcaaagacctggatgatgtttggtaactatagaaaaatagaaattacactcaggatcactgttactgctattgccactgatgattcctgcaaaaatataatcgaagttttccatcaaatgtataatatgctattaatacacattagatgataacagttgttccatgaatgattctatgaagctatgcatcttagacctcttgagctgtgaattagcactattttctatagttacttattctctggatcattttataatttccatattaatttcaaatatgctcgtgttattcttcagtgatttccacaattgtgcattttattctttggtttaagtactgaagcatataatgaaagtaattgctaagtagcagcttaaaaattcaattatccgattgtatttaacatctttaagagcatgatcataaagagctatttttgacaccccccccccacttttttaacatttagagttagtaagggttttatatctcttctgtccatattgttttcaaaggaatgaggtgtttaggtggctggaaaagcatttgtaggaagttagatttgaatatagacaaggtgggttattcacgttgagaatgttatttgaagaatgcctgtgaagccaggtgtgggttctactcagtgccatagatagactgagtcttctctcgtaggttaccattacatagtaattttgattctgaattacacattaaattatttgagtttatacagacctaaattttaaaatctgtacatatattattttgatgtattaagatgaatattgctgatttaaattttatttatgcacatacttaaaggacagaaatgtctgggaaagtaattgttaaataatgatatgtaactttttaactttttaaataaataacaagatttttaatgtgtgtctccctcagggttgtttaaagttttttttctccctcaagtataaatagtggtaactatatgttttgtatcttctagcaccaactgctgtaaagcaatgctgcaaataatgcttgaatacaagtggctaagccaacaacagaataaatacttttatagtagttttataatcctgaaattcgaaagctttcccaattgcacttgcatctaaacaaaactgttgcagtttttactctatttattttgttccccatgtttatgaaagtcctgcacagtttcaaaggcatggtaaataatatatcaatgtttatgtagtctgttacagaaacagctatagataacattatccagtgaagagcaaaattcaagctttagaaaatattcatgcatgcaattttgacatatctaaaaataggtttttgtatatttatggtgggaggtggttgggaacttttaacaaaatggggtgttaatttttgtacagtctgtgggcatttacacatttttaatgtattaaaatttggtaattatgtgtacattaaattaataaaagttacttctagttatgatttgtgaattccctaagaccttggatttttttaagtaactttatatcagaaatgatactgcatctttatatttttaaaattgtattgctgctcaagaatggtaccctcttgtcaaaaaggcatacattcataattgtacattcagcattgtaaataatcttatgaaaccttttttgattgaagctattcaaaataaaaattttaatgaatgaaaaaaaaaaaaaaaa

SEQ ID NO: 68 - protein, 1256 aa, Homo Sapiens
MDREERKTINQGQEDEMEIYGYNLSRWKLAIVSLGVICSDGFLLLLLYWMPEWRVKATCVRAAIKDCEVVLLRTTDEFKMWFCAKIRVLSLETYPVSSPKSMSNKLSNGHAVCLIENPTEENRHRISKYSQTESQQIRYFTHHSVKYFWNDTIHNFDFLKGLDEGVSCTSIYEKHSAGLTKGMHAYRKLLYGVNEIAVKVPSVFKLLIKEVLNPFYIFQLFSVILWSTDEYYYYALAIVVMSIVSIVSSLYSIRKQYVMLHDMVATHSTVRVSVCRVNEEIEEIFSTDLVPGDVMVIPLNGTIMPCDAVLINGTCIVNESMLTGESVPVTKTNLPNPSVDVKGIGDELYNPETHKRHTLFCGTTVIQTRFYTGELVKAIVVRTGFSTSKGQLVRSILYPKPTDFKLYRDAYLFLLCLVAVAGIGFIYTIINSILNEVQVGVIIIESLDIITITVPPALPAAMTAGIVYAQRRLKKIGIFCISPQRINICGQLNLVCFDKTGTLTEDGLDLWGIQRVENARFLSPEENVCNEMLVKSQFVACMATCHSLTKIEGVLSGDPLDLKMFEAIGWILEEATEEETALHNRIMPTVVRPPKQLLPESTPAGNQEMELFELPATYEIGIVRQFPFSSALQRMSVVARVLGDRKMDAYMKGAPEAIAGLCKPETVPVDFQNVLEDFTKQGFRVIALAHRKLESKLTWHKVQNISRDAIENNMDFMGLIIMQNKLKQETPAVLEDLHKANIRTVMVTGDSMLTAVSVARDCGMILPQDKVIIAEALPPKDGKVAKINWHYADSLTQCSHPSAIDPEAIPVKLVHDSLEDLQMTRYHFAMNGKSFSVILEHFQDLVPKLMLHGTVFARMAPDQKTQLIEALQNVDYFVGMCGDGANDCGALKRAHGGISLSELEASVASPFTSKTPSISCVPNLIREGRAALITSFCVFKFMALYSIIQYFSVTLLYSILSNLGDFQFLFIDLAIILVVVFTMSLNPAWKELVAQRPPSGLISGALLFSVLSQIIICIGFQSLGFFWVKQQPWYEVWHPKSDACNTTGSGFWNSSHVDNETELDEHNIQNYENTTVFFISSFQYLIVAIAFSKGKPFRQPCYKNYFFVFSVIFLYIFILFIMLYPVASVDQVLQIVCVPYQWRVTMLIIVLVNAFVSITVENFFLDMVLWKVVFNRDKQGEYRFSTTQPPQESVDRWGKCCLPWALGCRKKTPKAKYMYLAQELLVDPEWPPKPQTTTEAKALVKENGSCQIITIT

SEQ ID NO: 69 - DNA, 3768 bp, Homo Sapiens
  CDS (1)...(3768)
atggacagggaagaaaggaagaccatcaatcagggtcaagaagatgaaatggagatttatggttacaatttgagtcgctggaagcttgccatagtttctttaggagtgatttgctctgatgggtttctcctcctcctcctctattggatgcctgagtggcgggtgaaagcgacctgtgtcagagctgcaattaaagactgtgaagtagtgctgctgaggactactgatgaattcaaaatgtggttttgtgcaaaaattcgcgttctttctttggaaacttacccagtttcaagtccaaaatctatgtctaataagctttcaaatggccatgcagtttgtttaattgagaatcccactgaagaaaataggcacaggatcagtaaatattcacagactgaatcacaacagattcgttatttcacccaccatagtgtaaaatatttctggaatgataccattcacaattttgatttcttaaagggactggatgaaggtgtttcttgtacgtcaatttatgaaaagcatagtgcaggactgacaaaggggatgcatgcctacagaaaactgctttatggagtaaatgaaattgctgtaaaagtgccttctgtttttaagcttctaattaaagaggttctcaacccattttacattttccagctgttcagtgttatactgtggagcactgatgaatactattactatgctctagctattgtggttatgtccatagtatcaatcgtaagctcactatattccattagaaagcaatatgttatgttgcatgacatggtggcaactcatagtaccgtaagagtttcagtttgtagagtaaatgaagaaatagaagaaatcttttctaccgaccttgtgccaggagatgtcatggtcattccattaaatgggacaataatgccttgtgatgctgtgcttattaatggtacctgcattgtaaacgaaagcatgttaacaggagaaagtgttccagtgacaaagactaatttgccaaatccttcagtggatgtgaaaggaataggagatgaattatataatccagaaacacataaacgacatactttgttttgtgggacaactgttattcagactcgtttctacactggagaactcgtcaaagccatagttgttagaacaggatttagtacttccaaaggacagcttgttcgttccatattgtatcccaaaccaactgattttaaactctacagagatgcctacttgtttctactatgtcttgtggcagttgctggcattgggtttatctacactattattaatagcattttaaatgaggtacaagttggggtcataattatcgagtctcttgatattatcacaattactgtgccccctgcacttcctgctgcaatgactgctggtattgtgtatgctcagagaagactgaaaaaaatcggtattttctgtatcagtcctcaaagaataaatatttgtggacagctcaatcttgtttgctttgacaagactggaactctaactgaagatggtttagatctttgggggattcaacgagtggaaaatgcacgatttctttcaccagaagaaaatgtgtgcaatgagatgttggtaaaatcccagtttgttgcttgtatggctacttgtcattcacttacaaaaattgaaggagtgctctctggtgatccacttgatctgaaaatgtttgaggctattggatggattctggaagaagcaactgaagaagaaacagcacttcataatcgaattatgcccacagtggttcgtcctcccaaacaactgcttcctgaatctacccctgcaggaaaccaagaaatggagctgtttgaacttccagctacttatgagataggaattgttcgccagttcccattttcttctgctttgcaacgtatgagtgtggttgccagggtgctgggggataggaaaatggacgcctacatgaaaggagcgcccgaggccattgccggtctctgtaaacctgaaacagttcctgtcgattttcaaaacgttttggaagacttcactaaacagggcttccgtgtgattgctcttgcacacagaaaattggagtcaaaactgacatggcataaagtacagaatattagcagagatgcaattgagaacaacatggattttatgggattaattataatgcagaacaaattaaagcaagaaacccctgcagtacttgaagatttgcataaagccaacattcgcaccgtcatggtcacaggtgacagtatgttgactgctgtctctgtggccagagattgtggaatgattctacctcaggataaagtgattattgctgaagcattacctccaaaggatgggaaagttgccaaaataaattggcattatgcagactccctcacgcagtgcagtcatccatcagcaattgacccagaggctattccggttaaattggtccatgatagcttagaggatcttcaaatgactcgttatcattttgcaatgaatggaaaatcattctcagtgatactggagcattttcaagaccttgttcctaagttgatgttgcatggcaccgtgtttgcccgtatggcacctgatcagaagacacagttgatagaagcattgcaaaatgttgattattttgttgggatgtgtggtgatggcgcaaatgattgtggtgctttgaagagggcacacggaggcatttccttatcggagctcgaagcttcagtggcatctccctttacctctaagactcctagtatttcctgtgtgccaaaccttatcagggaaggccgtgctgctttaataacttccttctgtgtgtttaaattcatggcattgtacagcattatccagtacttcagtgttactctgctgtattctatcttaagtaacctaggagacttccagtttctcttcattgatctggcaatcattttggtagtggtatttacaatgagtttaaatcctgcctggaaagaacttgtggcacaaagaccaccttcgggtcttatatctggggcccttctcttctccgttttgtctcagattatcatctgcattggatttcaatctttgggttttttttgggtcaaacagcaaccttggtatgaagtgtggcatccaaaatcagatgcttgtaatacaacaggaagcgggttttggaattcttcacacgtagacaatgaaaccgaacttgatgaacataatatacaaaattatgaaaataccacagtgttttttatttccagttttcagtacctcatagtggcaattgccttttcaaaaggaaaacccttcaggcaaccttgctacaaaaattatttttttgttttttctgtgatttttttatatatttttatattattcatcatgttgtatccagttgcctctgttgaccaggttcttcagatagtgtgtgtaccatatcagtggcgtgtaactatgctcatcattgttcttgtcaatgcctttgtgtctatcacagtggagaacttcttccttgacatggtcctttggaaagttgtgttcaaccgagacaaacaaggagagtatcggttcagcaccacacagccaccgcaggagtcagtggatcggtggggaaaatgctgcttaccctgggccctgggctgtagaaagaagacaccaaaggcaaagtacatgtatctggcgcaggagctcttggttgatccagaatggccaccaaaacctcagacaaccacagaagctaaagctttagttaaggagaatggatcatgtcaaatcatcaccataaca

SEQ ID NO: 70 - DNA, 3919 bp, Homo Sapiens
  CDS (68)...(3682)
ttaccggaagtaaaacttcggaagtgaggcgttcctctgcccggaagtgagcgccgcgctaggaaagatggcggcagcggcggcggtgggcaacgcggtgccctgcggggcccggccttgcggggtccggcctgacgggcagcccaagcccgggccgcagccgcgcgcgctccttgccgccgggccggcgctcatagcgaacggtgacgagctggtggctgccgtgtggccgtaccggcggttggcgctgttgcggcgcctcacggtgctgccattcgccgggctgctttacccggcctggttgggtgccgcagccgctggctgctggggctggggcagcagttgggtgcagatccccgaagctgcgctgctcgtgcttgccaccatctgcctcgcgcacgcgctcactgtcctctcggggcattggtctgtgcacgcgcattgcgcgctcacctgcaccccggagtacgaccccagcaaagcgacctttgtgaaggtggcgccaacccccaacaatggctccacggagctcgtggccctgcaccgcaatgagggcgaagacgggcttgaggtgctgtccttcgaattccagaagatcaagtattcctacgatgccctggagaagaagcagtttctccccgtggcctttcctgtgggaaacgccttctcatactatcagagcaacagaggcttccaggaagactcagagatccgagcagctgagaagaaatttgggagcaacaaggccgagatggtggtgcctgacttctcggagcttttcaaggagagagccacagcccccttctttgtatttcaggtgttctgtgtggggctctggtgcctggatgagtactggtactacagcgtctttacgctatccatgctggtggcgttcgaggcctcgctggtgcagcagcagatgcggaacatgtcggagatccggaagatgggcaacaagccccacatgatccaggtctaccgaagccgcaagtggaggcccattgccagtgatgagatcgtaccaggggacatcgtctccatcggccgctccccacaggagaacctggtgccatgtgacgtgcttctgctgcgaggccgctgcatcgtagacgaggccatgctcacgggggagtccgtgccacagatgaaggagcccatcgaagacctcagcccagaccgggtgctggacctccaggctgattcccggctgcacgtcatcttcgggggcaccaaggtggtgcagcacatccccccacagaaagccaccacgggcctgaagccggttgacagcgggtgcgtggcctacgtcctgcggaccggattcaacacatctcagggcaagctgctgcgcaccatcctcttcggggtcaagagggtgactgcgaacaacctggagaccttcatcttcatcctcttcctcctggtgtttgccatcgctgcagctgcctatgtatggattgaaggtaccaaggaccccagccggaaccgctacaagctgtttctggagtgcaccctgatcctcacctcggtcgtgcctcctgagctgcccatcgagctgtccctggccgtcaacacctccctcatcgccctggccaagctctacatgtactgcacagagcccttccggatcccctttgctggcaaggtcgaggtgtgctgctttgacaagacggggacgttgaccagtgacagcctggtggtgcgcggtgtggccgggctgagagacgggaaggaggtgaccccagtgtccagcatccctgtagaaacacaccgggccctggcctcgtgccactcgctcatgcagctggacgacggcaccctcgtgggtgaccctctagagaaggccatgctgacggccgtggactggacgctgaccaaagatgagaaagtattcccccgaagtattaaaactcaggggctgaaaattcaccagcgctttcattttgccagtgccctgaagcgaatgtccgtgcttgcctcgtatgagaagctgggctccaccgacctctgctacatcgcggccgtgaagggggcccccgaaactctgcactccatgttctcccagtgcccgcccgactaccaccacatccacaccgagatctcccgggaaggagcccgcgtcctggcgctggggtacaaggagctgggacacctcactcaccagcaggcccgggaggtcaagcgggaggccctggagtgcagcctcaagttcgtcggcttcattgtggtctcctgcccgctcaaggctgactccaaggccgtgatccgggagatccagaatgcgtcccaccgggtggtcatgatcacgggagacaacccgctcactgcatgccacgtggcccaggagctgcacttcattgaaaaggcccacacgctgatcctgcagcctccctccgagaaaggccggcagtgcgagtggcgctccattgacggcagcatcgtgctgcccctggcccggggctccccaaaggcactggccctggagtacgcactgtgcctcacaggcgacggcttggcccacctgcaggccaccgacccccagcagctgctccgcctcatcccccatgtgcaggtgttcgcccgtgtggctcccaagcagaaggagtttgtcatcaccagcctgaaggagctgggctacgtgaccctcatgtgtggggatggcaccaacgacgtgggcgccctgaagcatgctgacgtgggtgtggcgctcttggccaatgcccctgagcgggttgtcgagcggcgacggcggccccgggacagcccaaccctgagcaacagtggcatcagagccacctccaggacagccaagcagcggtcggggctccctccctccgaggagcagccaacctcccagagggaccgcctgagccaggtgctgcgagacctcgaggacgagagtacgcccattgtgaaactgggggatgccagcatcgcagcacccttcacctccaagctctcatccatccagtgcatctgccacgtgatcaagcagggccgctgcacgctggtgaccacgctacagatgttcaagatcctggcgctcaatgccctcatcctggcctacagccagagcgtcctctacctggagggagtcaagttcagtgacttccaggccaccctacaggggctgctgctggccggctgcttcctcttcatctcccgttccaagcccctcaagaccctctcccgagaacggcccctgcccaacatcttcaacctgtacaccatcctcaccgtcatgctccagttctttgtgcacttcctgagccttgtctacctgtaccgtgaggcccaggcccggagccccgagaagcaggagcagttcgtggacttgtacaaggagtttgagccaagcctggtcaacagcaccgtctacatcatggccatggccatgcagatggccaccttcgccatcaattacaaaggcccgcccttcatggagagcctgcccgagaacaagcccctggtgtggagtctggcagtttcactcctggccatcattggcctgctcctcggctcctcgcccgacttcaacagccagtttggcctcgtggacatccctgtggagttcaagctggtcattgcccaggtcctgctcctggacttctgcctggcgctcctggccgaccgcgtcctgcagttcttcctggggaccccgaagctgaaagtgccttcctgagatggcagtgctggtacccactgcccaccctggctgccgctgggcgggaaccccaacagggccccgggagggaaccctgcccccaaccccccacagcaaggctgtacagtctcgcccttggaagactgagctgggacccccacagccatccgctggcttggccagcagaaccagccccaagccagcacctttggtaaataaagcagcatctgagattttaaaaaaaaaaaaaaaaaa

SEQ ID NO: 71 - protein, 1204 aa, Homo Sapiens
MAAAAAVGNAVPCGARPCGVRPDGQPKPGPQPRALLAAGPALIANGDELVAAVWPYRRLALLRRLTVLPFAGLLYPAWLGAAAAGCWGWGSSWVQIPEAALLVLATICLAHALTVLSGHWSVHAHCALTCTPEYDPSKATFVKVAPTPNNGSTELVALHRNEGEDGLEVLSFEFQKIKYSYDALEKKQFLPVAFPVGNAFSYYQSNRGFQEDSEIRAAEKKFGSNKAEMVVPDFSELFKERATAPFFVFQVFCVGLWCLDEYWYYSVFTLSMLVAFEASLVQQQMRNMSEIRKMGNKPHMIQVYRSRKWRPIASDEIVPGDIVSIGRSPQENLVPCDVLLLRGRCIVDEAMLTGESVPQMKEPIEDLSPDRVLDLQADSRLHVIFGGTKVVQHIPPQKATTGLKPVDSGCVAYVLRTGFNTSQGKLLRTILFGVKRVTANNLETFIFILFLLVFAIAAAAYVWIEGTKDPSRNRYKLFLECTLILTSVVPPELPIELSLAVNTSLIALAKLYMYCTEPFRIPFAGKVEVCCFDKTGTLTSDSLVVRGVAGLRDGKEVTPVSSIPVETHRALASCHSLMQLDDGTLVGDPLEKAMLTAVDWTLTKDEKVFPRSIKTQGLKIHQRFHFASALKRMSVLASYEKLGSTDLCYIAAVKGAPETLHSMFSQCPPDYHHIHTEISREGARVLALGYKELGHLTHQQAREVKREALECSLKFVGFIVVSCPLKADSKAVIREIQNASHRVVMITGDNPLTACHVAQELHFIEKAHTLILQPPSEKGRQCEWRSIDGSIVLPLARGSPKALALEYALCLTGDGLAHLQATDPQQLLRLIPHVQVFARVAPKQKEFVITSLKELGYVTLMCGDGTNDVGALKHADVGVALLANAPERVVERRRRPRDSPTLSNSGIRATSRTAKQRSGLPPSEEQPTSQRDRLSQVLRDLEDESTPIVKLGDASIAAPFTSKLSSIQCICHVIKQGRCTLVTTLQMFKILALNALILAYSQSVLYLEGVKFSDFQATLQGLLLAGCFLFISRSKPLKTLSRERPLPNIFNLYTILTVMLQFFVHFLSLVYLYREAQARSPEKQEQFVDLYKEFEPSLVNSTVYIMAMAMQMATFAINYKGPPFMESLPENKPLVWSLAVSLLAIIGLLLGSSPDFNSQFGLVDIPVEFKLVIAQVLLLDFCLALLADRVLQFFLGTPKLKVPS

SEQ ID NO: 72 - DNA, 3612 bp, Homo Sapiens
  CDS (1)...(3612)
atggcggcagcggcggcggtgggcaacgcggtgccctgcggggcccggccttgcggggtccggcctgacgggcagcccaagcccgggccgcagccgcgcgcgctccttgccgccgggccggcgctcatagcgaacggtgacgagctggtggctgccgtgtggccgtaccggcggttggcgctgttgcggcgcctcacggtgctgccattcgccgggctgctttacccggcctggttgggtgccgcagccgctggctgctggggctggggcagcagttgggtgcagatccccgaagctgcgctgctcgtgcttgccaccatctgcctcgcgcacgcgctcactgtcctctcggggcattggtctgtgcacgcgcattgcgcgctcacctgcaccccggagtacgaccccagcaaagcgacctttgtgaaggtggcgccaacccccaacaatggctccacggagctcgtggccctgcaccgcaatgagggcgaagacgggcttgaggtgctgtccttcgaattccagaagatcaagtattcctacgatgccctggagaagaagcagtttctccccgtggcctttcctgtgggaaacgccttctcatactatcagagcaacagaggcttccaggaagactcagagatccgagcagctgagaagaaatttgggagcaacaaggccgagatggtggtgcctgacttctcggagcttttcaaggagagagccacagcccccttctttgtatttcaggtgttctgtgtggggctctggtgcctggatgagtactggtactacagcgtctttacgctatccatgctggtggcgttcgaggcctcgctggtgcagcagcagatgcggaacatgtcggagatccggaagatgggcaacaagccccacatgatccaggtctaccgaagccgcaagtggaggcccattgccagtgatgagatcgtaccaggggacatcgtctccatcggccgctccccacaggagaacctggtgccatgtgacgtgcttctgctgcgaggccgctgcatcgtagacgaggccatgctcacgggggagtccgtgccacagatgaaggagcccatcgaagacctcagcccagaccgggtgctggacctccaggctgattcccggctgcacgtcatcttcgggggcaccaaggtggtgcagcacatccccccacagaaagccaccacgggcctgaagccggttgacagcgggtgcgtggcctacgtcctgcggaccggattcaacacatctcagggcaagctgctgcgcaccatcctcttcggggtcaagagggtgactgcgaacaacctggagaccttcatcttcatcctcttcctcctggtgtttgccatcgctgcagctgcctatgtatggattgaaggtaccaaggaccccagccggaaccgctacaagctgtttctggagtgcaccctgatcctcacctcggtcgtgcctcctgagctgcccatcgagctgtccctggccgtcaacacctccctcatcgccctggccaagctctacatgtactgcacagagcccttccggatcccctttgctggcaaggtcgaggtgtgctgctttgacaagacggggacgttgaccagtgacagcctggtggtgcgcggtgtggccgggctgagagacgggaaggaggtgaccccagtgtccagcatccctgtagaaacacaccgggccctggcctcgtgccactcgctcatgcagctggacgacggcaccctcgtgggtgaccctctagagaaggccatgctgacggccgtggactggacgctgaccaaagatgagaaagtattcccccgaagtattaaaactcaggggctgaaaattcaccagcgctttcattttgccagtgccctgaagcgaatgtccgtgcttgcctcgtatgagaagctgggctccaccgacctctgctacatcgcggccgtgaagggggcccccgaaactctgcactccatgttctcccagtgcccgcccgactaccaccacatccacaccgagatctcccgggaaggagcccgcgtcctggcgctggggtacaaggagctgggacacctcactcaccagcaggcccgggaggtcaagcgggaggccctggagtgcagcctcaagttcgtcggcttcattgtggtctcctgcccgctcaaggctgactccaaggccgtgatccgggagatccagaatgcgtcccaccgggtggtcatgatcacgggagacaacccgctcactgcatgccacgtggcccaggagctgcacttcattgaaaaggcccacacgctgatcctgcagcctccctccgagaaaggccggcagtgcgagtggcgctccattgacggcagcatcgtgctgcccctggcccggggctccccaaaggcactggccctggagtacgcactgtgcctcacaggcgacggcttggcccacctgcaggccaccgacccccagcagctgctccgcctcatcccccatgtgcaggtgttcgcccgtgtggctcccaagcagaaggagtttgtcatcaccagcctgaaggagctgggctacgtgaccctcatgtgtggggatggcaccaacgacgtgggcgccctgaagcatgctgacgtgggtgtggcgctcttggccaatgcccctgagcgggttgtcgagcggcgacggcggccccgggacagcccaaccctgagcaacagtggcatcagagccacctccaggacagccaagcagcggtcggggctccctccctccgaggagcagccaacctcccagagggaccgcctgagccaggtgctgcgagacctcgaggacgagagtacgcccattgtgaaactgggggatgccagcatcgcagcacccttcacctccaagctctcatccatccagtgcatctgccacgtgatcaagcagggccgctgcacgctggtgaccacgctacagatgttcaagatcctggcgctcaatgccctcatcctggcctacagccagagcgtcctctacctggagggagtcaagttcagtgacttccaggccaccctacaggggctgctgctggccggctgcttcctcttcatctcccgttccaagcccctcaagaccctctcccgagaacggcccctgcccaacatcttcaacctgtacaccatcctcaccgtcatgctccagttctttgtgcacttcctgagccttgtctacctgtaccgtgaggcccaggcccggagccccgagaagcaggagcagttcgtggacttgtacaaggagtttgagccaagcctggtcaacagcaccgtctacatcatggccatggccatgcagatggccaccttcgccatcaattacaaaggcccgcccttcatggagagcctgcccgagaacaagcccctggtgtggagtctggcagtttcactcctggccatcattggcctgctcctcggctcctcgcccgacttcaacagccagtttggcctcgtggacatccctgtggagttcaagctggtcattgcccaggtcctgctcctggacttctgcctggcgctcctggccgaccgcgtcctgcagttcttcctggggaccccgaagctgaaagtgccttcc

SEQ ID NO: 73 - protein, 7 aa, Artificial Sequence
  Consensus Amino Acid Sequence
DKTGTXX

SEQ ID NO: 74 - protein, 1157 aa, Caenorhabditis elegans
MGVDQLVETIIPYNLRSIATHLYVPPFTIITAIWTYVWLNIFGYEEYYELGMLGYAAIFVILALVLLFCHWMMPVRCFLMCSKQEDVRIASHVCVIPTQNNGWPELVKLMRTTRDKQTKLWFEFQRVHYTWDEESREFQTKTLDTAKPMVFFQKSHGFEVEEHVKDAKYLLGDNKTEMIVPQFLEMFIERATAPFFVFQVFCVGLWCLEDMWYYSLFTLFMLMTFEATLVKQQMKNMSEIRNMGNKTYMINVLRGKKWQKIKIEELVAGDIVSIGRGAEEECVPCDLLLLRGPCIVDESMLTGESVPQMKEPIEDVEKDKIFDIETDSRLHVIFGGTKIVQHTAPGKAAEGMVKSPDGNCICYVIRTGFNTSQGKLLRTIMFGVKKATANNLETFCFILFLLIFAIAAAAYLWIKGSVDETRSKYKLFLECTLILTSVIPPELPIELSLAVNSSLMALQKLGIFCTEPFRIPFAGKVDICCFDKTGTLTTDNLVVEGVALNNQKEGMIRNAEDLPHESLQVLASCHSLVRFEEDLVGDPLEKACLSWCGWNLTKGDAVMPPKTAAKGISGIKIFHRYHFSSAMKRMTVVAGYQSPGTSDTTFIVAVKGAPEVLRNMYADLPSDYDETYTRLTRQGSRVLAMGIRKLGETRVGELRDKKRENFENDLAFAGFVVISCPLKSDTKTMIREIMDSSHVVAMITGDNPLTACHVSKVLKFTKKSLPTLVLDEPADGVDWMWKSVDGTIELPLKPETKNKMERKAFFNSHEFCLTGSAFHHLVHNEHTFLRELILHVKVFARMAPKQKERIINELKSLGKVTLMCGDGTNDVGALKHANVGVALLTNPYDAEKAAEKEKEKKAKIEEARSLVRSGAQLPQRPGAPGAPPAANAARPRLDNLMKELEEEEKAQVIKLGDASIAAPFTSKYTSIASICHVIKQGRCTLVTTLQMFKILALNALVSAYSLSALYLDGVKFSDTQATIQGLLLAACFLFISKSKPLKTLSRQRPMANIFNAYTLLTVTLQFIVHFSCLLYIVGLAHEANTEKAPVDLEAKFTPNILNTTVYIISMALQVCTFAVNYRGRPFMESLFENKAMLYSIMFSGGAVFTLASGQATDLMIQFELVVLPEALRNALLMCVTADLVICYIIDRGLNFLLGDMF

SEQ ID NO: 75 - protein, 9 aa, Artificial Sequence
  Consensus Amino Acid Sequence
XXXXXXGEX

SEQ ID NO: 76 - protein, 10 aa, Artificial Sequence
  Consensus Amino Acid Sequence
XXXDKTGTXT

SEQ ID NO: 77 - protein, 11 aa, Artificial Sequence
  Consensus Amino Acid Sequence
XGDGXNDXPXL

SEQ ID NO: 78 - protein, 7 aa, Artificial Sequence
  Amino Acid Residues Important for Calcium Transport
IPEGLPA

SEQ ID NO: 79 - protein, 28 aa, Homo Sapiens
DLVTVVVPPALPAAMTVCTLYAQSRLRR

SEQ ID NO: 80 - protein, 28 aa, Homo Sapiens
DIITITVPPALPAAMTAGIVYAQRRLKK

SEQ ID NO: 81 - protein, 28 aa, Homo Sapiens
LILTSVVPPELPIELSLAVNTSLIALAK

SEQ ID NO: 82 - protein, 29 aa, Saccharomyces cerevisiae
DIITIVVPPALPATLTIGTNFALSRLKEK

SEQ ID NO: 83 - protein, 28 aa, Saccharomyces cerevisiae
LIITSVVPPELPMELTMAVNSSLAALAK

SEQ ID NO: 84 - protein, 28 aa, Schizosaccharomyces pombe
VLTILVPPALPATLSVGIANSIARLSRA

SEQ ID NO: 85 - protein, 29 aa, Caenorhabditis elegans
DLVTIVVPPALPAVMGIGIFYAQRRLRQK

SEQ ID NO: 86 - protein, 29 aa, Caenorhabditis elegans
DIITIVVPPALPAAMSVGIINANSRLKKK

SEQ ID NO: 87 - protein, 29 aa, Caenorhabditis elegans
DIITITVPPALPAAMSVGIINAQLRLKKK

SEQ ID NO: 88 - protein, 29 aa, Caenorhabditis elegans
LILTSVIPPELPIELSLAVNSSLMALQKL

SEQ ID NO: 89 - protein, 29 aa, Homo Sapiens
ALAVAAIPEGLPAVITTCLALGTRRMAKK

SEQ ID NO: 90 - protein, 29 aa, Oryctolagus cuniculus
ALAVAAIPEGLPAVITTCLALGTRRMAKK

SEQ ID NO: 91 - protein, 29 aa, Gallus gallus
ALAVAAIPEGLPAVITTCLALGTRRMAKK

SEQ ID NO: 92 - protein, 29 aa, Felis catus
ALAVAAIPEGLPAVITTCLALGTRRMAKK

SEQ ID NO: 93 - protein, 29 aa, Procambarus clarkii
ALAVAAIPEGLPAVITTCLALGTRRMAKK

SEQ ID NO: 94 - protein, 29 aa, Homo Sapiens
ALAVAAIPEGLPAVITTCLALGTRRMAKK

SEQ ID NO: 95 - protein, 29 aa, Homo Sapiens
ALAVAAIPEGLPAVITTCLALGTRRMARK

SEQ ID NO: 96 - protein, 29 aa, Drosophila melanogaster
AVAVAAIPEGLPAVITTCLALGTRRMAKK

SEQ ID NO: 97 - protein, 29 aa, Saccharomyces cerevisiae
SLAVAAIPEGLPIIVTVTLALGVLRMAKR

SEQ ID NO: 98 - protein, 29 aa, Saccharomyces cerevisiae
TVIVVAVPEGLPLAVTLALAFATTRMTKD

SEQ ID NO: 99 - protein, 29 aa, Homo Sapiens
TVLVVAVPEGLPLAVTISLAYSVKKMMKD

SEQ ID NO: 100 - protein, 29 aa, Homo Sapiens
TVLVVAVPEGLPLAVTISLAYSVKKMMKD

SEQ ID NO: 101 - protein, 29 aa, Rattus norvegicus
TVLVVAVPEGLPLAVTISLAYSVKKMMKD

SEQ ID NO: 102 - protein, 29 aa, Homo Sapiens
TVLVVAVPEGLPLAVTISLAYSVKKMMKD

SEQ ID NO: 103 - protein, 29 aa, Homo Sapiens
TVLVVAVPEGLPLAVTISLAYSVKKMMKD

SEQ ID NO: 104 - protein, 29 aa, Saccharomyces cerevisiae
TVLIVSCPCVIGLAVPIVFVIASGVAAKR

SEQ ID NO: 105 - protein, 29 aa, Homo Sapiens
TVLCIACPCSLGLATPTAVMVGTGVGAQN

SEQ ID NO: 106 - protein, 29 aa, Homo Sapiens
TVLCIACPCSLGLATPTAVMVGTGVAAQN

SEQ ID NO: 107 - protein, 29 aa, Drosophila melanogaster
GIIVANVPEGLLATVTVCLTLTAKRMASK

SEQ ID NO: 108 - protein, 29 aa, Hydra vulgaris
GIIVANVPEGLLATVTVCLTLTAKKMAKK

SEQ ID NO: 109 - protein, 29 aa, Bufo marinus
GIIVANVPEGLLATVTVCLTLTAKRMARK

SEQ ID NO: 110 - protein, 29 aa, Homo Sapiens
GIIVANVPEGLLATVTVCLTLTAKRMARK

SEQ ID NO: 111 - protein, 29 aa, Homo Sapiens
GIIVANVPEGLLATVTVCLTLTAKRMARK

SEQ ID NO: 112 - protein, 29 aa, Homo Sapiens
GIIVANVPEGLLATVTVCLTVTAKRMARK

SEQ ID NO: 113 - protein, 29 aa, Homo Sapiens
ILFNNLIPISLLVTLEVVKFTQAYFINWD

SEQ ID NO: 114 - protein, 29 aa, Saccharomyces cerevisiae
ILFSNLVPISLFVTVELIKYYQAFMIGSD

(19) United States
(12) Patent Application Publication
(10) Pub. No.: US 2011/0150860 A1
(43) Pub. Date: Jun. 23, 2011

(54) NOVEL 25869, 25934, 26335, 50365, 21117, 38692, 46508, 16816, 16839, 49937, 49931 AND 49933 MOLECULES AND USES THEREFOR

(75) Inventors: Rory A.J. Curtis, Ashland, MA (US); Thomas Joseph Logan, Springfield, PA (US); Maria Alexandra Glucksmann, Lexington, MA (US); Rachel E. Meyers, Newton, MA (US); Mark J. Williamson, Saugus, MA (US); Laura A. Rudolph-Owen, Medford, MA (US); Miyoung Chun, Santa Barbara, CA (US); Fong-Ying Tsai, Newton, MA (US)

(73) Assignee: Millennium Pharmaceuticals, Inc., Cambridge, MA (US)

(21) Appl. No.: 12/755,449

(22) Filed: Apr. 7, 2010

Related U.S. Application Data
(63) Continuation of application No. 12/150,094, filed on Apr. 24, 2008, now Pat. No. 7,732,182.
Application No. 12/150,094 is a division of application No. 11/445,606, filed on Jun. 2, 2006, now Pat. No. 7,411,054.
Application No. 11/445,606 is a continuation of application No. 10/377,072, filed on Feb. 27, 2003, now abandoned.
Application No. 10/377,072 is a continuation-in-part of application No. 09/895,860, filed on Jun. 29, 2001, now abandoned.
Application No. 10/377,072 is a continuation-in-part of application No. 09/723,806, filed on Nov. 28, 2000, now Pat. No. 6,686,185.
Application No. 10/377,072 is a continuation-in-part of application No. 09/843,297, filed on Apr. 25, 2001, now Pat. No. 6,569,667.
Application No. 10/377,072 is a continuation-in-part of application No. 09/861,801, filed on May 21, 2001, now abandoned.
Application No. 10/377,072 is a continuation-in-part of application No. 09/816,494, filed on Mar. 23, 2001, now Pat. No. 6,664,089.
Application No. 09/816,494 is a continuation-in-part of application No. 09/888,911, filed on Jun. 25, 2001, now abandoned.
Application No. 10/377,072 is a continuation-in-part of application No. 09/908,664, filed on Jul. 17, 2001, now abandoned.
Application No. 10/377,072 is a continuation-in-part of application No. 09/935,291, filed on Aug. 21, 2001, now abandoned.
(60) Provisional application No. 60/215,370, filed on Jun. 29, 2000.
(60) Provisional application No. 60/187,455, filed on Mar. 7, 2000.
(60) Provisional application No. 60/199,801, filed on Apr. 26, 2000.
(60) Provisional application No. 60/205,508, filed on May 19, 2000.
(60) Provisional application No. 60/213,688, filed on Jun. 23, 2000.
(60) Provisional application No. 60/218,675, filed on Jul. 17, 2000.
(60) Provisional application No. 60/250,932, filed on Nov. 30, 2000.
(60) Provisional application No. 60/226,504, filed on Aug. 21, 2000.

(51) Int. Cl. A61K 39/395; C07H 21/04; C07K 14/00; C07K 16/00; C12P 21/06; G01N 33/53; G01N 33/48; G01N 33/50; C12N 5/10; A61K 31/711; A61K 38/16; A61K 31/7088; A61P 35/00; A61P 3/00; A61P 7/00; A61P 19/00; A61P 25/00; A61P 31/12

(52) U.S. Cl. 424/130.1; 536/23.1; 530/350; 530/387.9; 435/69.1; 436/94; 436/86; 435/7.1; 435/325; 514/44 R; 514/21.2; 514/19.3; 514/16.4; 514/17.7; 514/13.3; 514/7.4; 514/9.7; 514/3.7; 514/13.5; 514/18.6; 514/44 A

(57) ABSTRACT

The invention provides isolated nucleic acids molecules, designated 25869, 25934, 26335, 50365, 21117, 38692, 46508, 16816, 16839, 49937, 49931 and 49933 nucleic acid molecules. The invention also provides antisense nucleic acid molecules, recombinant expression vectors containing 25869, 25934, 26335, 50365, 21117, 38692, 46508, 16816, 16839, 49937, 49931 or 49933 nucleic acid molecules, host cells into which the expression vectors have been introduced, and nonhuman transgenic animals in which a 25869, 25934, 26335, 50365, 21117, 38692, 46508, 16816, 16839, 49937, 49931 or 49933 gene has been introduced or disrupted. The invention still further provides isolated 25869, 25934, 26335, 50365, 21117, 38692, 46508, 16816, 16839, 49937, 49931 or 49933 proteins, fusion proteins, antigenic peptides and anti-25869, 25934, 26335, 50365, 21117, 38692, 46508, 16816, 16839, 49937, 49931 or 49933 antibodies. Diagnostic and therapeutic methods utilizing compositions of the invention are also provided.

### RELATED APPLICATIONS

The present application is a continuation of U.S. patent application Ser. No. 12/150,094, filed Apr. 24, 2008 (pending), which is a divisional of U.S. patent application Ser. No. 11/445,606, filed Jun. 2, 2006, now U.S. Pat. No. 7,411,054, which is a continuation of U.S. patent application Ser. No. 10/377,072, filed Feb. 27, 2003 (abandoned), which is a continuation-in-part of U.S. patent application Ser. No. 09/895,860, filed Jun. 29, 2001 (abandoned), which claims the benefit of U.S. Provisional Application Ser. No. 60/215,370, filed Jun. 29, 2000 (abandoned). U.S. patent application Ser. No. 10/377,072 is also a continuation-in-part of U.S. patent application Ser. No. 09/723,806, filed Nov. 28, 2000, now U.S. Pat. No. 6,686,185, which claims the benefit of U.S. Provisional Application Ser. No. 60/187,455, filed Mar. 7, 2000 (abandoned). U.S. patent application Ser. No. 10/377,072 is also a continuation-in-part of U.S. patent application Ser. No. 09/843,297, filed Apr. 25, 2001, now U.S. Pat. No. 6,569,667, which claims the benefit of U.S. Provisional Application Ser. No. 60/199,801, filed Apr. 26, 2000 (abandoned). U.S. patent application Ser. No. 10/377,072 is also a continuation-in-part of U.S. patent application Ser. No. 09/861,801, filed May 21, 2001 (abandoned), which claims the benefit of U.S. Provisional Application Ser. No. 60/205,508, filed May 19, 2000 (abandoned). U.S. patent application Ser. No. 10/377,072 is also a continuation-in-part of U.S. patent application Ser. No. 09/816,494, filed Mar. 23, 2001, now U.S. Pat. No. 6,664,089, which claims the benefit of U.S. patent application Ser. No. 09/815,419, filed Mar. 22, 2001 (abandoned), and also claims the benefit of U.S. Provisional Application Ser. No. 60/191,858, filed Mar. 24, 2000 (abandoned). U.S. patent application Ser. No. 10/377,072 is also a continuation-in-part of U.S. patent application Ser. No. 09/888,911, filed Jun. 25, 2001 (abandoned), which claims the benefit of U.S. Provisional Application Ser. No. 60/213,688, filed Jun. 23, 2000 (abandoned). U.S. patent application Ser. No. 10/377,072 is also a continuation-in-part of U.S. patent application Ser. No. 09/908,664, filed Jul. 17, 2001 (abandoned), which claims the benefit of U.S. Provisional Application Ser. No. 60/218,675, filed Jul. 17, 2000 (abandoned). U.S. patent application Ser. No. 10/377,072 is also a continuation-in-part of U.S. patent application Ser. No. 09/935,291, filed Aug. 21, 2001 (abandoned), which claims the benefit of U.S. Provisional Application Ser. No. 60/250,932, filed Nov. 30, 2000 (abandoned) and also claims the benefit of U.S. Provisional Application Ser. No. 60/226,504, filed Aug. 21, 2000 (abandoned). The entire contents of each of the above-referenced patent applications are incorporated herein by this reference.

### BACKGROUND OF THE INVENTION

The enormous variety of biochemical reactions that comprise life are nearly all mediated by a series of biological catalysts known as enzymes. Enzymes are proteins which possess specific properties that enable them to catalyze a series of reactions, allowing metabolic pathways to degrade and to reconstruct products needed to maintain organisms. By the binding of substrates through geometrically and physically complementary reactions, enzymes are stereospecific in binding substrates as well as in catalyzing reactions. The stringency for this stereospecificity varies as some enzymes are more specific to the identity of their substrates, while others are capable of binding multiple substrates and can catalyze numerous types of reactions.

Examples of enzymes include, for example, carboxylases, fatty acid desaturases, serine/threonine dehydratases, hexokinases, peptidyl tRNA hydrolases, dual specificity phosphatases, phospholipases and transporters. Such enzymes have the ability to, for example: (1) hydrolyze an ester linkage and/or liberate the free acid form of a substrate, e.g., hydrolysis of a triglyceride and/or liberation of free fatty acid(s) and glycerol; (2) catalyze the formation of a double bond, preferably, at positions up to 9 carbons from the carboxyl end of a molecule, e.g., a fatty acid, such as a polyunsaturated fatty acid; (3) catalyze the phosphorylation of a sugar, e.g., an aldohexoses and a ketohexoses (e.g., glucose, mannose, fructose, sorbitol and glucosamine); (4) catalyze sugar metabolism; (5) transfer a phosphate from a phosphate donor (e.g., ATP) to a sugar, e.g., an aldohexoses and a ketohexoses (e.g., glucose, mannose, fructose, sorbitol and glucosamine) to form a phosphorylated sugar, e.g., glucose-6-phosphate; (6) catalyze the removal of a phosphate group attached to a tyrosine residue in a protein target, e.g., a growth factor receptor; (7) catalyze the removal of a phosphate group attached to a serine or threonine residue in a protein e.g., a growth factor receptor; (8) hydrolyze covalent bond between peptide and tRNA within peptidyl-tRNAs; (9) catalyze the hydrolysis of phosphatidyl-inositol-4,5-bisphosphate (PIP2) producing diacylglycerol and inositol 1,4,5-trisphosphate; (10) transport of a substrate or target molecule (e.g., a Ca²⁺ ion) from one side of a biological membrane to the other; and (11) be phosphorylated or dephosphorylated. Accordingly, there exists a need to identify additional human enzymes, for example, for use as disease markers and as targets for identifying various therapeutic modulators.

### SUMMARY OF THE INVENTION

The present invention is based, at least in part, on the discovery of novel nucleic acid molecules and proteins encoded by such nucleic acid molecules, referred to herein as “25869, 25934, 26335, 50365, 21117, 38692, 46508, 16816, 16839, 49937, 49931 or 49933”. The 25869, 25934, 26335, 50365, 21117, 38692, 46508, 16816, 16839, 49937, 49931 or 49933 nucleic acid and protein molecules of the present invention are useful as modulating agents in regulating a variety of cellular processes, e.g., including but not limited to cell proliferation, differentiation, growth and division. In particular, these nucleic acid molecules will be advantageous in the regulation of any cellular function, uncontrolled proliferation and differentiation, such as in cases of cancer. Accordingly, in one aspect, this invention provides isolated nucleic acid molecules encoding 25869, 25934, 26335, 50365, 21117, 38692, 46508, 16816, 16839, 49937, 49931 or 49933 proteins or biologically active portions thereof, as well as nucleic acid fragments suitable as primers or hybridization probes for the detection of 25869, 25934, 26335, 50365, 21117, 38692, 46508, 16816, 16839, 49937, 49931 or 49933-encoding nucleic acids.

The nucleotide sequence of the cDNA encoding 25869, 25934, 26335, 50365, 21117, 38692, 46508, 16816, 16839, 49937, 49931 or 49933, and the amino acid sequence of 25869, 25934, 26335, 50365, 21117, 38692, 46508, 16816, 16839, 49937, 49931 or 49933 polypeptides are depicted in Table 1.

TABLE 1
Sequences of the invention

| | cDNA | Protein | | ATCC |
|---|---|---|---|---|
| Gene | SEQ | SEQ | Coding Region | Accession |
| Name | ID NO: | ID NO: | SEQ ID NO: | Number |
| 25869 | 1 | 2 | 3 | |
| 25934 | 7 | 8 | 9 | |
| 26335 | 16 | 17 | 18 | |
| 50365 | 20 | 21 | 22 | |
| 21117 | 25 | 26 | 27 | |
| 38692 | 28 | 29 | 30 | |
| 46508 | 35 | 36 | 37 | |
| 16816 | 39 | 40 | 41 | |
| 16839 | 42 | 43 | 44 | |
| 49937 | 63 | 64 | 65 | |
| 49931 | 67 | 68 | 69 | |
| 49933 | 70 | 71 | 72 | |

Accordingly, in one aspect, the invention features a nucleic acid molecule which encodes a 25869, 25934, 26335, 50365, 21117, 38692, 46508, 16816, 16839, 49937, 49931 or 49933 protein or polypeptide, e.g., a biologically active portion of the 25869, 25934, 26335, 50365, 21117, 38692, 46508, 16816, 16839, 49937, 49931 or 49933 protein. In a preferred embodiment, the isolated nucleic acid molecule encodes a polypeptide having the amino acid sequence of SEQ ID NO:2, 8, 17, 21, 26, 29, 36, 40, 43, 64, 68 or 71. In other embodiments, the invention provides isolated 25869, 25934, 26335, 50365, 21117, 38692, 46508, 16816, 16839, 49937, 49931 or 49933 nucleic acid molecules having the nucleotide sequence shown in SEQ ID NO:1, 3, 7, 9, 16, 18, 20, 22, 25, 27, 28, 30, 35, 37, 39, 41, 42, 44, 63, 65, 67, 69, 70 or 72. In still other embodiments, the invention provides nucleic acid molecules that are substantially identical (e.g., naturally occurring allelic variants) to the nucleotide sequence shown in SEQ ID NO:1, 3, 7, 9, 16, 18, 20, 22, 25, 27, 28, 30, 35, 37, 39, 41, 42, 44, 63, 65, 67, 69, 70 or 72. In other embodiments, the invention provides a nucleic acid molecule which hybridizes under a stringent hybridization condition as described herein to a nucleic acid molecule comprising the nucleotide sequence of SEQ ID NO:1, 3, 7, 9, 16, 18, 20, 22, 25, 27, 28, 30, 35, 37, 39, 41, 42, 44, 63, 65, 67, 69, 70 or 72, wherein the nucleic acid encodes a full length 25869, 25934, 26335, 50365, 21117, 38692, 46508, 16816, 16839, 49937, 49931 or 49933 protein or an active fragment thereof.

In a related aspect, the invention further provides nucleic acid constructs which include a 25869, 25934, 26335, 50365, 21117, 38692, 46508, 16816, 16839, 49937, 49931 or 49933 nucleic acid molecule described herein. In certain embodiments, the nucleic acid molecules of the invention are operatively linked to native or heterologous regulatory sequences. Also included are vectors and host cells containing the 25869, 25934, 26335, 50365, 21117, 38692, 46508, 16816, 16839, 49937, 49931 or 49933 nucleic acid molecules of the invention e.g., vectors and host cells suitable for producing polypeptides.

In another related aspect, the invention provides nucleic acid fragments suitable as primers or hybridization probes for the detection of 25869, 25934, 26335, 50365, 21117, 38692, 46508, 16816, 16839, 49937, 49931 or 49933-encoding nucleic acids.

In still another related aspect, isolated nucleic acid molecules that are antisense to a 25869, 25934, 26335, 50365, 21117, 38692, 46508, 16816, 16839, 49937, 49931 or 49933 encoding nucleic acid molecule are provided.

In another aspect, the invention features 25869, 25934, 26335, 50365, 21117, 38692, 46508, 16816, 16839, 49937, 49931 or 49933 polypeptides, and biologically active or antigenic fragments thereof that are useful, e.g., as reagents or targets in assays applicable to treatment and diagnosis of 25869, 25934, 26335, 50365, 21117, 38692, 46508, 16816, 16839, 49937, 49931 or 49933-associated disorders. In another embodiment, the invention provides 25869, 25934, 26335, 50365, 21117, 38692, 46508, 16816, 16839, 49937, 49931 or 49933 polypeptides having a 25869, 25934, 26335, 50365, 21117, 38692, 46508, 16816, 16839, 49937, 49931 or 49933 activity.

In other embodiments, the invention provides 25869, 25934, 26335, 50365, 21117, 38692, 46508, 16816, 16839, 49937, 49931 or 49933 polypeptides, e.g., a 25869, 25934, 26335, 50365, 21117, 38692, 46508, 16816, 16839, 49937, 49931 or 49933 polypeptide having the amino acid sequence shown in SEQ ID NO:2, 8, 17, 21, 26, 29, 36, 40, 43, 64, 68 or 71; an amino acid sequence that is substantially identical to the amino acid sequence shown in SEQ ID NO:2, 8, 17, 21, 26, 29, 36, 40, 43, 64, 68 or 71; or an amino acid sequence encoded by a nucleic acid molecule having a nucleotide sequence which hybridizes under a stringent hybridization condition as described herein to a nucleic acid molecule comprising the nucleotide sequence of SEQ ID NO:1, 3, 7, 9, 16, 18, 20, 22, 25, 27, 28, 30, 35, 37, 39, 41, 42, 44, 63, 65, 67, 69, 70 or 72, wherein the nucleic acid encodes a full length 25869, 25934, 26335, 50365, 21117, 38692, 46508, 16816, 16839, 49937, 49931 or 49933 protein or an active fragment thereof.

In a related aspect, the invention further provides nucleic acid constructs which include a 25869, 25934, 26335, 50365, 21117, 38692, 46508, 16816, 16839, 49937, 49931 or 49933 nucleic acid molecule described herein.

In a related aspect, the invention provides 25869, 25934, 26335, 50365, 21117, 38692, 46508, 16816, 16839, 49937, 49931 or 49933 polypeptides or fragments operatively linked to non-25869, 25934, 26335, 50365, 21117, 38692, 46508, 16816, 16839, 49937, 49931 or 49933 polypeptides to form fusion proteins.

In another aspect, the invention features antibodies and antigen-binding fragments thereof, that react with, or more preferably specifically or selectively bind 25869, 25934, 26335, 50365, 21117, 38692, 46508, 16816, 16839, 49937, 49931 or 49933 polypeptides.

In another aspect, the invention provides methods of screening for compounds that modulate the expression or activity of the 25869, 25934, 26335, 50365, 21117, 38692, 46508, 16816, 16839, 49937, 49931 or 49933 polypeptides or nucleic acids.

In still another aspect, the invention provides a process for modulating 25869, 25934, 26335, 50365, 21117, 38692, 46508, 16816, 16839, 49937, 49931 or 49933 polypeptide or nucleic acid expression or activity, e.g., using the compounds identified in the screens described herein. In certain embodiments, the methods involve treatment of conditions related to aberrant activity or expression of the 25869, 25934, 26335, 50365, 21117, 38692, 46508, 16816, 16839, 49937, 49931 or 49933 polypeptides or nucleic acids, such as conditions or disorders involving aberrant or deficient 25869, 25934, 26335, 50365, 21117, 38692, 46508, 16816, 16839, 49937, 49931 or 49933 expression. Examples of such disorders include, but are not limited to cellular proliferative and/or differentiative disorders, angiogenic disorders, brain disorders, neurological disorders, blood vessel disorders, breast disorders, colon disorders, kidney disorders, lung disorders, ovarian disorders, prostate disorders, hematopoeitic disorders, pancreatic disorders, skeletal muscle disorders, skin disorders, hormonal disorders, immune e.g., inflammatory, disorders, cardiovascular disorders, lipid homeostasis disorders, endothelial cell disorders, liver disorders, disorders of the small intestine, pain disorders, viral diseases, metabolic disorders, bone metabolism disorders or platelet disorders.

The invention also provides assays for determining the activity of or the presence or absence of 25869, 25934, 26335, 50365, 21117, 38692, 46508, 16816, 16839, 49937, 49931 or 49933 polypeptides or nucleic acid molecules in a biological sample, including for disease diagnosis.

In a further aspect, the invention provides assays for determining the presence or absence of a genetic alteration in a 25869, 25934, 26335, 50365, 21117, 38692, 46508, 16816, 16839, 49937, 49931 or 49933 polypeptide or nucleic acid molecule, including for disease diagnosis.

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a 25869, 25934, 26335, 50365, 21117, 38692, 46508, 16816, 16839, 49937, 49931 or 49933 molecule. In one embodiment, the capture probe is a nucleic acid, e.g., a probe complementary to a 25869, 25934, 26335, 50365, 21117, 38692, 46508, 16816, 16839, 49937, 49931 or 49933 nucleic acid sequence. In another embodiment, the capture probe is a polypeptide, e.g., an antibody specific for 25869, 25934, 26335, 50365, 21117, 38692, 46508, 16816, 16839, 49937, 49931 or 49933 polypeptides. Also featured is a method of analyzing a sample by contacting the sample to the aforementioned array and detecting binding of the sample to the array.

Other features and advantages of the invention will be apparent from the following detailed description, and from the claims.

### DETAILED DESCRIPTION OF THE INVENTION

### Human 25869

### COE-1

The present invention is based, at least in part, on the discovery of novel carboxylesterase family members, referred to herein as “25869”, “Carboxylesterase” or “COE-1” nucleic acid and protein molecules. These novel molecules are capable of hydrolyzing ester-, thioester-, and amide-containing compounds (e.g., triglycerides) to their respective free acids, are upregulated in marmoset monkeys treated with cholestyramine, a cholesterol and/or lipid lowering drug, and play a role in or function in a variety of cellular processes, including lipid homeostasis, hydrolysis of endogenous and/or exogenous compounds; detoxification and/or activation of drugs, pro-drugs, toxins, and/or carcinogens; intra- or inter-cellular signaling; gene expression; and/or cellular growth and/or differentiation.

Carboxylesterases comprise a family of enzymes which catalyze the hydrolysis of a variety of ester-, thioester-, and amide-containing chemicals, as well as drugs (including prodrugs) to their respective free acids. Carboxylesterases catalyze the hydrolysis of endogenous compounds such as short- and long-chain acyl-glycerols, long-chain acylcarnitine, and long-chain acyl-CoA esters (Satoh, T. and Hosokawa, M. (1998) Annu. Rev. Pharmacol. Toxicol. 38:257-88). The general enzymatic reaction that carboxylesterases catalyze is:

a carboxylic ester+H₂O<=>an alcohol+a carboxylic anion

The hydrolytic activity of carboxylesterases is dependent on the presence of a “catalytic triad” of amino acid residues that are non-contiguous in the primary sequence but adjacent in the tertiary structure (Ollis, D. L. et al. (1992) Protein Eng. 5:197-211; Cygler, M. et al. (1993) Protein Sci. 2:366-382). In carboxylesterases, the catalytic triad residues are Ser/His/Glu (Satoh and Hosokawa (1998) supra; Oakeshott, J. G. et al. (1999) Bioessays 21:1031-1042).

The first step of the hydrolysis reaction catalyzed by carboxylesterases liberates the alcohol moiety of the substrate and forms a covalent linkage between the remaining acid moiety of the substrate and the serine residue of the catalytic triad. The second step cleaves this linkage and liberates the acid moiety of the substrate, largely through the action of the histidine residue of the catalytic triad (Oakeshott et al. (1999) supra).

While many well-studied carboxylesterases have catalytic triads, several members of the carboxyl/cholinesterase multigene family have been reported to lack a functional catalytic triad (Hortsch, M. et al. (1990) Development 110:1327-1340; Auld, V. J. (1995) Cell 81:757-767; Ichtchenko, K. et al. (1995) Cell 81:435-443; Oakeshott, J. G. et al. (1995) Trends. Ecol. Evol. 10:103-110) and, thus, to lack carboxylesterase activity. However, some of these enzymes have ligand-binding functions involved in signal transduction.

Carboxylesterases are responsible for the hydrolysis of many exogenous compounds, resulting in both the inactivation of drugs and the activation of pro-drugs (Satoh, T. (1987) Reviews in Biochem. Toxicol. 8:155-81; Heymann, E. (1980) Enzymatic Basis of Detoxification 2:291-323; Heymann, E. (1982) Metabolic Basis of Detoxification 1:229-45; Leinweber, F.-J. (1987) Drug Metab. Rev. 18:379-439). Human liver and plasma carboxylesterase converts lovastatin to its active form (Tang, B. K. and Kalow, W. (1995) Eur. J. Clin. Pharmacol. 47:449-51) and converts an inactive prodrug form of prostaglandin F2α to its active metabolite (Cheng-Bennett, A. et al. (1994) Br. J. Opthalmol. 78:560-67). A significant number of drugs and endogenous compounds are substrates of carboxylesterases, including dipivefrin hydrochloride (Nakamura, M. et al. (1993) Ophthalmic Res. 25:46-51), carbonates (McCracken, N. W. et al. (1993) Biochem. Pharmacol. 45:31-36; Huang, T. L. et al. (1993) Pharmacol. Res. 10:639-48), cocaine (Dean, R. A. (1995) J. Pharmacol. Exp. Ther. (1995) 275:965-71; Brzezinski, M. R. et al. (1994) Biochem. Pharmacol. 48:1747-55), salicylates (White, K. N. et al. (1994) Biochem. Soc. Trans. 22:220S), capsaicin (Park, Y. H. and Lee, S. S. (1994) Biochem. Mol. Biol. Int. 34:351-60), palmitoyl-coenzyme A (Hosokawa, M. et al. (1987) Mol. Pharmacol. 31:579-84; Hosokawa, M. et al. (1990) Arch. Biochem. Biophys. 277:219-27; Tsujita, T. and Okuda, H. (1993) J. Lipid Res. 34:1773-81; Mentlein, R. et al. (1984) Arch. Biochem. Biophys. 234:612-21), haloperidol (Nambu, K. et al. (1987) Biochem. Pharmacol. 36:1715-22), imidapril (Yamada, Y. et al. (1992) Arzneimittel Forsch. 42:507-12), pyrrolizidine alkaloids (Dueker, S. R. et al. (1992) Toxicol. Appl. Pharmacol. 117:116-21; Dueker, S. R. et al. (1995) Arch. Toxicol. 69:725-28; Dueker, S. R. et al. (1992) Drug. Metab. Dispos. 20:275-80), and steroids (Lund-Pero, M. et al. (1994) Clin. Chim. Acta. 224:9-20).

The novel COE-1 molecules of the instant invention are members of the lipase subfamily of carboxylesterases and show significant homology to mouse and rat lipases. Analysis of the activity of a recently characterized hepatic microsomal lipase, ES-10, showed that increased expression of ES-10 in hepatic cell lines resulted in an increase in the rate of depletion of intracellular triacylglycerol stores, indicating that ES-10 is capable of hydrolysis of stored triacylglycerol. In addition, hepatocytes expressing ES-10 exhibited increased levels of apo-B 100 in the VLDL fraction (Lehner, R. and Vance, D. E. (1999) Biochem. J. 343:1-10; Robbi, M. et al. (1990) Biochem. J. 269:451-458).

Because the COE-1 molecules of the instant invention show significant homology to ES-10, COE-1 may function as an intracellular lipase, the activity of which may contribute to the mobilization of intracellular triacylglycerol stores, which can be used for lipoprotein assembly. Accordingly, modulation of COE-1 activity may result in the modulation of serum lipoprotein and/or triglyceride levels. For example, inhibition of COE-1 activity may have positive effects on serum lipoprotein and triglyceride profiles. In view of the foregoing activities, the COE-1 molecules of the present invention provide novel diagnostic targets and therapeutic agents to control carboxylesterase-associated disorders.

As used herein, a “carboxylesterase-associated disorder” includes a disorder, disease or condition which is caused or characterized by a misregulation (e.g., downregulation or upregulation) of carboxylesterase activity. Carboxylesterase-associated disorders can detrimentally affect cellular functions such as lipid homeostasis; cellular proliferation, growth, differentiation, or migration; inter- or intra-cellular communication; tissue function, such as cardiac function or musculoskeletal function; systemic responses in an organism, such as nervous system responses, hormonal responses (e.g., insulin response), or immune responses; and protection of cells from toxic compounds (e.g., carcinogens, toxins, or mutagens).

In a preferred embodiment, a carboxylesterase-associated disorder is a “lipid homeostasis disorder”. Other examples of carboxylesterase-associated disorders include cardiovascular disorders, neurological (CNS) disorders, cellular proliferation, growth, differentiation, or migration disorders, hormonal disorders, immune disorders and disorders affecting tissues in which COE-1 protein is expressed, e.g., the kidney, colon, liver, brain, small intestine, and skeletal muscle, as assessed by TaqMan analysis.

Members of the COE-1 family of proteins, for example, include at least one “carboxylesterase domain” in the protein or corresponding nucleic acid molecule. As used herein, the term “carboxylesterase domain” includes a protein domain having at least about 440-600 amino acid residues and a bit score of at least 440 when compared against a carboxylesterase Hidden Markov Model (HMM), e.g., PFAM Accession Number PF00135. Preferably, a carboxylesterase domain includes a protein having an amino acid sequence of about 460-580, 480-560, 500-540, or more preferably about 519 amino acid residues, and a bit score of at least 470, 480, 490, 500, or more preferably, 516.6. To identify the presence of a carboxylesterase domain in a COE-1 protein, and make the determination that a protein of interest has a particular profile, the amino acid sequence of the protein is searched against a database of known protein domains (e.g., the HMM database). The carboxylesterase domain (HMM) has been assigned the PFAM Accession number PF00135 (see the PFAM website, available online through Washington University in St. Louis). A search was performed against the HMM database resulting in the identification of a carboxylesterase domain in the amino acid sequence of human COE-1 at about residues 5-523 of SEQ ID NO:2.

A description of the Pfam database can be found in Sonhammer et al. (1997) Proteins 28:405-420, and a detailed description of HMMs can be found, for example, in Gribskov et al. (1990) Methods Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference.

In another embodiment, a COE-1 family member of the present invention is identified based on the presence of a “catalytic triad” in the protein or corresponding nucleic acid molecule. As used herein, the term “catalytic triad” includes a group of three amino acid residues which are non-contiguous in the primary sequence but which are adjacent in the tertiary structure of a protein and which actively participate in an enzymatic reaction catalyzed by a carboxylesterase. In the carboxylesterases of the present invention, the catalytic triad residues are typically serine, histidine, and glutamic acid (Satoh and Hosokawa (1998) supra; Oakeshott et al. (1999) supra). The amino acid residues of the catalytic triad may also be referred to herein as the “catalytic residues” (e.g., the “catalytic serine” or “catalytic histidine”) or the “active site residues” (e.g., the “active site serine” or “active site histidine”). In a preferred embodiment, the serine residue of a catalytic triad of the COE-1 molecules of the present invention is contained within a catalytic serine motif, as defined herein. In a further preferred embodiment, the serine residue of a catalytic triad of the COE-1 molecules of the present invention is contained within a carboxylesterases type-B serine active site. An alignment of the COE-1 amino acid sequence (SEQ ID NO:2), using the GAP program in the GCG software package (Blosum 62 matrix) and a gap weight of 12 and a length weight of 4, with a mouse liver carboxylesterase precursor (GenBank Accession No. Q63880; SEQ ID NO:4), identified two of the catalytic triad residues as serine²⁰⁵ and histidine⁴²⁶ of human COE-1.

In another embodiment, a COE-1 family member of the present invention is identified based on the presence of a “catalytic serine motif” in the protein or corresponding nucleic acid molecule. As used herein, a “catalytic serine motif” includes a group of five amino acid residues having the consensus sequence G-X-S-X-G (SEQ ID NO:6), wherein X indicates any amino acid residue. Catalytic serine motifs are found in all known lipases (Lehner, R. and Vance, D. E. (1999) Biochem. J. 343:1-10). In a preferred embodiment, a catalytic serine motif includes a catalytic serine, as defined herein. In a further preferred embodiment, a catalytic serine motif is contained within a carboxylesterases type-B serine active site. An alignment of the COE-1 amino acid sequence (SEQ ID NO:2), using the GAP program in the GCG software package (Blosum 62 matrix) and a gap weight of 12 and a length weight of 4, with a mouse liver carboxylesterase precursor (GenBank Accession No. Q63880; SEQ ID NO:4), identified a catalytic serine motif at about residues 203-207 of human COE-1 (SEQ ID NO:2).

In another embodiment, a COE-1 protein of the present invention is identified based on the presence of a “carboxylesterases type-B serine active site” in the protein or corresponding nucleic acid molecule. A carboxylesterases type-B serine active site functions as part of the catalytic active site of a carboxylesterase. The carboxylesterases type-B serine active site has been assigned ProSite Accession Number PS00122. To identify the presence of a carboxylesterases type-B serine active site in a COE-1 protein, and to make the determination that a protein of interest has a particular profile, the amino acid sequence of the protein may be searched against a database of known protein domains (e.g., the ProSite database) using the default parameters (available online through the Swiss Institute of Bioinformatics (SIB)). In a preferred embodiment, a carboxylesterases type-B serine active site comprises a catalytic serine motif, which further comprises a catalytic serine. A search was performed against the ProSite database resulting in the identification of a carboxylesterases type-B serine active site in the amino acid sequence of human COE-1 (SEQ ID NO:2) at about residues 192-207.

In another embodiment, a COE-1 family member of the present invention is identified based on the presence of an “ER retention signal” in the protein or corresponding nucleic acid molecule. As used herein, an “ER retention signal” includes a group of four amino acid residues located at the C-terminus of a polypeptide sequence which targets a protein to the lumen of the endoplasmic reticulum. Based on homology to other proteins known to have ER retention signals, an ER retention signal was identified in the amino acid sequence of human COE-1 (SEQ ID NO:2) at about residues 544-547.

Isolated proteins of the present invention, preferably COE-1 proteins, have an amino acid sequence sufficiently homologous to the amino acid sequence of SEQ ID NO:2, or are encoded by a nucleotide sequence sufficiently homologous to SEQ ID NO:1 or 3. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains having at least 50%, 55%, 60%, 65%, 70%, 75%, 80%, 85%, 85%, 88%, 89%, 90%, 95%, 96%, 97%, 98%, 99%, 99.1%, 99.2%, 99.3%, 99.4%, 99.5%, 99.6%, 99.7%, 99.8%, 99.9% or more homology or identity across the amino acid sequences of the domains and contain at least one and preferably two structural domains or motifs, are defined herein as sufficiently homologous. Furthermore, amino acid or nucleotide sequences which share at least 50%, 55%, 60%, 65%, 70%, 75%, 80%, 85%, 85%, 88%, 89%, 90%, 95%, 96%, 97%, 98%, 99%, 99.1%, 99.2%, 99.3%, 99.4%, 99.5%, 99.6%, 99.7%, 99.8%, 99.9% or more homology or identity and share a common functional activity are defined herein as sufficiently homologous.

In a preferred embodiment, a COE-1 protein includes at least one or more of the following domains, motifs, and/or amino acid residues: a carboxylesterase domain, a catalytic triad, a catalytic serine, a catalytic histidine, a catalytic serine motif, a carboxylesterases type-B serine active site, and/or an ER retention signal, and has an amino acid sequence at least about 50%, 55%, 60%, 65%, 70%, 75%, 80%, 85%, 85%, 88%, 89%, 90%, 95%, 96%, 97%, 98%, 99%, 99.1%, 99.2%, 99.3%, 99.4%, 99.5%, 99.6%, 99.7%, 99.8%, 99.9% or more homologous or identical to the amino acid sequence of SEQ ID NO:2. In yet another preferred embodiment, a COE-1 protein includes at least one or more of the following domains, motifs, and/or amino acid residues: a carboxylesterase domain, a catalytic triad, a catalytic serine, a catalytic histidine, a catalytic serine motif, a carboxylesterases type-B serine active site, and/or an ER retention signal, and is encoded by a nucleic acid molecule having a nucleotide sequence which hybridizes under stringent hybridization conditions to a complement of a nucleic acid molecule comprising the nucleotide sequence of SEQ ID NO:1 or 3. In another preferred embodiment, a COE-1 protein includes at least one or more of the following domains, motifs, and/or amino acid residues: a carboxylesterase domain, a catalytic triad, a catalytic serine, a catalytic histidine, a catalytic serine motif, a carboxylesterases type-B serine active site, and/or an ER retention signal, and has a COE-1 activity.

As used interchangeably herein, a “COE-1 activity”, “biological activity of COE-1” or “functional activity of COE-1”, includes an activity exerted or mediated by a COE-1 protein, polypeptide or nucleic acid molecule on a COE-1 responsive cell or on a COE-1 substrate, as determined in vivo or in vitro, according to standard techniques. In one embodiment, a COE-1 activity is a direct activity, such as an association with a COE-1 target molecule. As used herein, a “target molecule” or “binding partner” is a molecule with which a COE-1 protein binds or interacts in nature, such that COE-1-mediated function is achieved. A COE-1 target molecule can be a non-COE-1 molecule or a COE-1 protein or polypeptide of the present invention. In an exemplary embodiment, a COE-1 target molecule is a COE-1 substrate or ligand, e.g., a triglyceride. A COE-1 activity can also be an indirect activity, such as a cellular signaling activity mediated by interaction of the COE-1 protein with a COE-1 substrate or ligand.

In a preferred embodiment, a COE-1 activity is at least one of the following activities: (i) interaction with a COE-1 substrate or target molecule (e.g., a triglyceride); (ii) conversion of a COE-1 substrate or target molecule to a product (e.g., hydrolysis of an ester linkage and/or liberation of the free acid form of the substrate, e.g., hydrolysis of a triglyceride and/or liberation of free fatty acid(s) and glycerol); (iii) modulation of lipolysis; (iv) modulation of lipid uptake by a cell (e.g., a liver cell); (v) modulation of lipid synthesis and/or secretion; (vi) modulation of intracellular lipid release and/or turnover; (vii) modulation of intracellular lipid and/or triglyceride mass; (viii) modulation of secreted lipid and/or triglyceride mass; (ix) modulation of serum lipid, lipoprotein, and/or triglyceride levels; (x) modulation of lipid homeostasis; (xi) direct or indirect modulation of lipoprotein assembly; (xii) interaction with and/or hydrolysis of a second non-COE-1 protein; (xiii) activation/deactivation of a COE-1 substrate or target molecule (e.g., activation/deactivation of a carcinogen); (xiv) metabolism and/or detoxification of a drug; (xv) modulation of cellular signaling and/or gene transcription (e.g., either directly or indirectly); and/or (xvi) modulation of cellular proliferation and/or differentiation.

Isolation of the Human 25869 or COE-1 cDNA

The invention is based, at least in part, on the discovery of genes encoding novel members of the carboxylesterase family. The entire sequence of human clone Fbh25869 was determined and found to contain an open reading frame termed human “25869” or “COE-1.”

The nucleotide sequence encoding the human COE-1 is set forth as SEQ ID NO:1. The human COE-1 gene, which is approximately 2087 nucleotides in length, encodes a protein having a molecular weight of approximately 60.2 kD and which is approximately 547 amino acid residues in length as set forth as SEQ ID NO:2. The coding region (open reading frame) of SEQ ID NO:1 is set forth as SEQ ID NO:3. Clone Fbh25869 comprises the coding region of human COE-1.

### Analysis of the Human 25869 or COE-1 Molecules

The amino acid sequence of human COE-1 was analyzed using the program PSORT (available online) to predict the localization of the proteins within the cell. This program assesses the presence of different targeting and localization of amino acid sequences within the query sequence. The results of the analyses show that human COE-1 may be localized to the cytoplasm, to the nucleus, or to the mitochondria.

Searches of the amino acid sequence of human COE-1 were performed against the HMM database. These searches resulted in the identification of a “carboxylesterase domain” at about residues 5-523 of SEQ ID NO:2 (score=516.6).

Searches of the amino acid sequence of human COE-1 were further performed against the Prosite database. These searches resulted in the identification in the amino acid sequence of human COE-1 of a potential N-glycosylation site and a number of potential protein kinase C phosphorylation sites, casein kinase II phosphorylation sites, and N-myristoylation sites. These searches further resulted in the identification of a carboxylesterases type-B serine active site at about residues 192-207 of SEQ ID NO:2.

A “catalytic triad” was also identified in the human COE-1. An alignment of human COE-1 (SEQ ID NO:2) with a mouse liver carboxylesterase precursor (GenBank Accession No. Q63880; SEQ ID NO:4), identified two of the catalytic triad amino acids, namely Ser²⁰⁵ and His⁴²⁶ within SEQ ID NO:2.

A “catalytic serine motif” was also identified in the human COE-1. An alignment of human COE-1 (SEQ ID NO:2), using the GAP program in the GCG software package (Blosum 62 matrix) and a gap weight of 12 and a length weight of 4, with a mouse liver carboxylesterase precursor (GenBank Accession No. Q63880; SEQ ID NO:4), identified a catalytic serine motif at about residues 203-207 of human COE-1 (SEQ ID NO:2).

An “ER retention signal” was also identified in the human COE-1. Based on homology to other proteins known to have ER retention signals, an ER retention signal was identified in the amino acid sequence of human COE-1 (SEQ ID NO:2) at about residues 544-547.

Global alignments (using the GAP program in the GCG software package (Blosum 62 matrix) and a gap weight of 12 and a length weight of 4) of the human COE-1 amino acid sequence (SEQ ID NO:2) revealed that human COE-1 has a 67.3% identity with the amino acid sequence of a mouse liver carboxylesterase precursor (SEQ ID NO:4; GenBank Accession No. Q63880) and a 46% identity with the amino acid sequence of rat liver carboxylesterase 10 precursor (also referred to as ES-10; SEQ ID NO:5; GenBank Accession No. P16303).

Tissue Distribution of COE-1 mRNA

This example describes the tissue distribution of human COE-1 mRNA, as may be determined using in situ hybridization analysis. For in situ analysis, various tissues, e.g., tissues obtained from brain, are first frozen on dry ice. Ten-micrometer-thick sections of the tissues are postfixed with 4% formaldehyde in DEPC-treated 1× phosphate-buffered saline at room temperature for 10 minutes before being rinsed twice in DEPC 1× phosphate-buffered saline and once in 0.1 M triethanolamine-HCl (pH 8.0). Following incubation in 0.25% acetic anhydride-0.1 M triethanolamine-HCl for 10 minutes, sections are rinsed in DEPC 2×SSC (1×SSC is 0.15 M NaCl plus 0.015 M sodium citrate). Tissue is then dehydrated through a series of ethanol washes, incubated in 100% chloroform for 5 minutes, and then rinsed in 100% ethanol for 1 minute and 95% ethanol for 1 minute and allowed to air dry.

Hybridizations are performed with ³⁵S-radiolabeled (5×10⁷ cpm/ml) cRNA probes. Probes are incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1×Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides are washed with 2×SSC. Sections are then sequentially incubated at 37° C. in TNE (a solution containing 10 mM Tris-HCl (pH 7.6), 500 mM NaCl, and 1 mM EDTA), for 10 minutes, in TNE with 10 μg of RNase A per ml for 30 minutes, and finally in TNE for 10 minutes. Slides are then rinsed with 2×SSC at room temperature, washed with 2×SSC at 50° C. for 1 hour, washed with 0.2×SSC at 55° C. for 1 hour, and 0.2×SSC at 60° C. for 1 hour. Sections are then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

### Analysis of Human COE-1 Expression Using the TaqMan Procedure

TAQMAN™ analysis on a panel of: (1) normal artery; (2) normal vein; (3) aortic smooth muscle cells—early; (4) coronary smooth muscle cells; (5) human umbilical vein endothelial cells (HUVECs)—static; (6) human umbilical vein endothelial cells (HUVECs)—shear; (7) normal heart; (8) heart—congestive heart failure (CHF); (9) kidney; (10) skeletal muscle; (11) normal adipose tissue; (12) pancreas; (13) primary osteoblasts; (14) differentiated osteoclasts; (15) normal skin; (16) normal spinal cord; (17) normal brain cortex; (18) brain—hypothalamus; (19) nerve; (20) dorsal root ganglion (DRG); (21) glial cells (astrocytes); (22) glioblastoma; (23) normal breast; (24) breast tumor; (25) normal ovary; (26) ovary tumor; (27) normal prostate; (28) prostate tumor; (29) epithelial cells (prostate); (30) normal colon; (31) colon tumor; (32) normal lung; (33) lung tumor; (34) lung—chronic obstructive pulmonary disease (COPD); (35) colon—inflammatory bowel disease (IBD); (36) normal liver; (37) liver—fibrosis; (38) dermal cells—fibroblasts; (39) normal spleen; (40) normal tonsil; (41) lymph node; (42) small intestine; (43) skin—decubitus; (44) synovium; (45) bone marrow mononuclear cells (BM-MNC); and (46) activated peripheral blood mononuclear cells (PBMCs), revealed that human 25869 or COE-1 was highly expressed in the kidney, colon, liver, brain, small intestine, and skeletal muscle. Expression of human COE-1 was also upregulated in marmoset monkeys treated with cholestyramine, a drug which lowers cholesterol and/or lipids in the blood.

### Analysis of Human 25869 or COE-1 Activity

The activity, e.g., the lipase activity, of COE-1 molecules of the present invention may be determined by the use of any of the following assays. All of the following assays are performed as described in Lehner, R. and Vance, D. E. (1999) Biochem. J. 343:1-10, the contents of which are incorporated herein by reference.

### Materials

Oleic acid, essentially fatty acid-free bovine serum albumin (BSA), p-nitrophenyl fatty acyl esters and Protein A-Sepharose CL 4B are purchased from Sigma (St. Louis, Mo., U.S.A.). [9,10-3H]Triolein (28 mCi/mmol) is from Dupont NEN (Boston, Mass., U.S.A.). [1,3-3H]glycerol (2.6 Ci/mmol), [U-¹⁴C]glycerol (149 mCi/mmol), [9,10-3H]oleic acid (10 Ci/mmol), L-[4,5-3H]leucine (57 Ci/mmol) and ECL Western blotting reagents are obtained from Amersham Canada (Oakville, Ontario, Canada). Dulbecco's modified Eagle's medium (DMEM), sodium pyruvate, penicillin/streptomycin, fetal bovine and horse sera and Geneticin (G-418 sulfate) are from Gibco BRL (Life Technologies Inc., Grand Island, N.Y., U.S.A.). Triascin C is purchased from Biomol Research Laboratories, Inc. (Plymouth Meeting, Pa., U.S.A.). All other chemicals and solvents are of reagent or better quality and are obtained from any number of suppliers known to those of skill in the art. Sheep anti-[human apolipoprotein (apo)B] IgG is from Boehringer-Mannheim.

### Cell Culture

Primary hepatocytes are isolated from male Sprague-Dawley rats (body weight 125-150 g), fed ad libitum, by collagenase perfusion of the liver. The cells are cultured in DMEM containing 15% (v/v) fetal bovine serum, as described in Yao, Z. and Vance, D. E. (1988) J. Biol. Chem. 263:449-509. HepG2 cells, obtained from A.T.C.C., are cultured in minimal Eagle's medium containing 0.1 mM nonessential amino acids, 1 mM sodium pyruvate, penicillin (10 units/ml), streptomycin (100 μg/ml) and 10% fetal bovine serum. McArdle RH7777 cells obtained from A.T.C.C., are cultured in DMEM containing pyruvate, antibiotics, 10% fetal bovine serum, and 10% (v/v) horse serum. All cultures are maintained in 100 mm dishes (Corning) at 37° C. in humidified air (89-91% saturation) containing 5% CO₂.

Generation of Stable Cell Lines Expressing COE-1 cDNA

Cells, e.g., McArdle RH7777 cells, are transfected with 10 μg of pBK-CMV plasmid vector (Stratagene) with or without COE-1 cDNA insert using a calcium precipitation procedure, as described in Chen, C. and Okayama, H. (1987) Mol. Cell. Biol. 7:2745-2752. Cells stably expressing either the empty vector or the vector containing the COE-1 cDNA are selected for resistance to the antibiotic G-418 (0.08% w/v). Transfected McArdle RH7777 cells are grown in DMEM supplemented with 10% horse serum and 10% fetal bovine serum, penicillin/streptomycin (40 units/ml) and 0.02% (w/v) G-418. Cells are maintained at 37° C. in humidified air containing 5% CO₂.

### Preparation of Microsomal Membranes

Cells, e.g., McArdle RH7777 cells, from ten 100 mm diameter culture dishes (approximately 50 mg of protein) are harvested into 5 ml of 10 mM Tris/HCl, pH 7.4, containing 250 mM sucrose and 5 mM EDTA. Cells are homogenized with a Polytron, and the microsomal membranes are isolated by ultracentrifugation from a post-mitochondrial supernatant, as described in Lehner, R. and Kuksis, A. (1993) J. Biol. Chem. 268:8781-8786.

### Lipase Assay

Lipolytic activities in microsomal membranes (50 μg of protein) isolated from ‘mock’ transfected (empty vector) and COE-1 cDNA transfected cells (e.g., McArdle RH7777 cells) are assessed using either radiolabeled triolein or a chromogenic substrate (p-nitrophenyl laurate) as described in Lehner, R. and Verger, R. (1997) Biochemistry 36:1861-1868.

### Immunoblot Analysis

Cells, e.g., McArdle RH7777 cells (non-transfected, ‘mock’ transfected, and COE-1 cDNA transfected) are harvested in phosphate-buffered saline (PBS) and disrupted by brief sonication. Cell homogenates (35 μg of protein) are electrophoresed on an SDS/12% (w/v) polyacrylamide gel, transferred to a nitrocellulose membrane, and the expression of COE-1 is analyzed by blotting with anti-COE-1 antibodies using standard methods.

### Lipid Uptake by Transfected Cells

Cells, e.g., McArdle RH7777 cells (at approximately 80% confluency in 60 mm diameter culture dishes) are incubated with 2 ml of serum-free DMEM containing 0.5% BSA and 100 μCi [³H]oleic acid for 1 hour. The medium is aspirated, the cells are washed with DMEM/0.5% BSA, and subsequently the cells are incubated with 5 ml of DMEM/0.5% BAS for 2 hours. The medium is removed, diluted with DMEM/0.5% BSA and 5 ml aliquots are added to the cells, e.g., McArdle RH7777 cells, stably transfected with either pBK-CMV vector or with pBK-CMV containing COE-1 cDNA. Aliquots of medium (1 ml) are taken after 2, 4, and 6 hours of incubation. At the end of the incubation period, the cells are washed with ice-cold PBS, harvested in the same buffer, and dispersed by brief sonication. Cellular and medium lipids are extracted as described in Folch, J. et al. ((1957) J. Biol. Chem. 226:449-509) in the presence of non-labeled lipid carriers. The lipids are applied to TLC plates and developed to one-third the height with chloroform/methanol/acetic acid/water (25:15:4:2, by volume) to separate glycerophospholipids, followed by development in heptane/isopropyl ether/acetic acid (60:40:4, by volume) to separate neutral lipids. Lipids are made visible by exposure to iodine; bands corresponding to various lipid classes are scraped, and the associated radioactivity is determined by scintillation counting.

### Effect of Triascin C on Glycerolipid Synthesis and Secretion

Cells, e.g., McArdle RH7777 cells, stably transfected with pBK-CMV or pBK-CMV containing COE-1 cDNA are grown to approximately 70% confluency in 60 mm diameter dishes and incubated for 24 hours with serum-free DMEM containing 0.1 mM oleic acid complexed with BSA (0.13% final concentration). The medium is aspirated and the cells are incubated for 1 hour with DMEM containing 0.1 mM oleic acid/BSA and various concentrations of triascin C in DMSO. The final concentration of DMSO is 0.4%. After a 1 hour incubation triascin C, 10 μCi/dish of [³H]glycerol is added. Cells and medium are collected after 2 hours, lipids are extracted, and the radioactivity associated with phosphatidylcholine and triacylglycerol is analyzed as described above.

### Intracellular Lipid Turnover in Transfected Cells

Cells, e.g., McArdle RH7777 cells, stably transfected with pBK-CMV or pBK-CMV containing COE-1 cDNA (grown to approximately 70% confluency) are incubated with serum-free DMEM containing 0.1 mM oleic acid complexed with BSA (0.13% final concentration) and 20 μCi [³H]glycerol for 15 hours. After 13 hours, triascin C (20 μM final concentration) is added. After an additional 2 hours of labeling, medium is removed, cells are washed and incubated for 6 hours±triascin C (chase). Cells are harvested, lipids are extracted and separated by TLC, and the amount of radioactivity in phosphatidylcholine and triacylglycerol is determined.

### Utilization of Intracellular Triacylglycerol Stores for Lipoprotein Assembly and Secretion

### Contribution of [¹⁴C]Glycerol-Labeled Intracellular Triacylglycerol Pools in the Presence of Exogenous Oleate: Pulse-Chase Protocol

Cells are incubated for 2 hours with 0.375 mM oleic acid/0.5% BSA to increase the triacylglycerol stores. Cells are then washed and incubated for 1 hour with DMEM followed by 2 hours with 0.375 mM oleic acid/0.5% BSA and 0.25 μCi [¹⁴C]glycerol. Radioactivity in cellular and medium phosphatidylcholine and triacylglycerol is analyzed following lipid extraction and TLC as described above.

### Secretion of [³H]Glycerol-Prelabeled Intracellular Triacylglycerol Pools in the Absence of Oleate

Cells are incubated for 16 hours with serum-free DMEM containing 0.375 mM oleic acid/0.5% BSA and 0.25 μCi [¹⁴C]glycerol, washed and incubated with DMEM in the absence of extracellular oleate for up to 8 hours. Radioactivity in cellular medium lipids is analyzed.

Lipid Secretion from Transfected Cells

Cells, e.g., McArdle RH7777 cells, at 60-70% confluency are washed with DMEM and incubated in the absence of serum for 2 hours. One set of dishes is incubated for 4 hours with serum-free DMEM containing 0.5% BSA and 10 μCi [³H]glycerol. The other set of dishes is incubated for 4 hours with serum-free DMEM containing 0.375 mM oleic acid/0.5% BSA and 10 μCi [³H]glycerol. At the end of a 4 hour pulse, a set of oleate-treated cells is washed with DMEM and incubated for various times with serum-free DMEM containing 0.5% BSA (chase). Medium and cells are collected, and lipids are extracted in the presence of non-labeled lipid carriers. Lipids are separated by TLC and made visible by exposure to iodine, and the radioactivity associated with phosphatidylcholine and triacylglycerol is determined.

Triacylglycerol and Phosphatidylcholine Mass Secretion from Transfected Cells

The experimental design for determination of the mass of triacylglycerol and phosphatidylcholine secreted is identical to that described above, except that cells are grown in 100 mm diameter dishes, and medium from three dishes is combined. Lipids are extracted by TLC, and the mass of phosphatidylcholine is determined as described in Chalvardjian, A. and Rudnicki, E. (1970) Anal. Biochem. 36:225-226. The mass of triacylglycerol is determined according to the methods of Snyder, F. and Stephens, N. ((1959) Biochim. Biophys. Acta 34:244-245) using trioleoylglycerol as an authentic standard.

### Determination of Intracellular and Secreted Triacylglycerol Mass

Cells (e.g., freshly prepared primary rat hepatocytes seeded at (5-6)×10⁶ cells/60 mm diameter dish; McArdle RH7777 cells at 80% confluency; or HepG2 cells) are incubated for 12 hours in their respective growth media supplemented with 0.375 mM oleate/0.5% BSA. Cells are then washed with DMEM and incubated for 2 hours with DMEM containing 0.375 mM oleate/0.5% BSA. The media are then collected and triacylglycerol mass is analyzed (triacylglycerol secretion at 2 hours of supplementation with oleate). Cells are washed and incubated for 2 hours with DMEM in the absence of oleate. Medium is removed, the cells washed, and fresh DMEM replenished. The procedure is repeated every 2 hours. Media and cell lipids are extracted and triacylglycerol mass determined as described above.

### ApoB Secretion

Cells stably transfected with either pBK-CMV or pBK-CMV containing COE-1 cDNA grown to approximately 70% confluency in 60 mm diameter dishes are incubated for 16 hours with serum-free DMEM. Cells are then incubated for 2 hours with DMEM containing 0.375 mM oleic acid/0.5% BSA, washed for 1 hour with leucine-free medium±oleic acid/BSA and incubated for 2 hours with 2 ml of either leucine-free DMEM containing 0.5% BSA and 250 μCi of [³H]leucine, or leucine-free DMEM containing 0.375 mM oleic acid/0.5% BSA and 250 μCi [³H]leucine. Medium is removed and briefly centrifuged to remove cellular debris. A 0.1 ml aliquot of 10× immunoprecipitation buffer (1.5 M NaCl, 0.5 M Tris/HCl, pH 7.4, 50 mM EDTA, 5% (v/v) Triton X-100, 1% (w/v) SDS; Wu, X. et al. (1996) J. Lipid Res. 37:1198-1206) is added to the culture medium (0.9 ml containing 1 mM final concentration of benzamidine). Anti (human apoB) IgG (10 μl) is then added. The mixture is incubated for 12 hours at 4° C., then 50 μl of Protein A-Sepharose is added, and the mixture is incubated for 3 more hours. The beads are pelleted by brief centrifugation, washed three times with an excess of immunoprecipitation buffer, then 0.1 μg of rat VLDL protein is added, followed by electrophoresis sample buffer (125 mM Tris/HCl, pH 6.8, 4% SDS, 20% (v/v) glycerol, 10% (v/v) β-mercaptoethanol, 0.02% Bromophenol Blue; Wu et al. (1996) supra). Samples are boiled and electrophoresed through 5% (w/v) polyacrylamide gels containing 0.1% SDS. Gels are silver stained (Rabilloud, T. et al. (1988) Electrophoresis 9:288-291), and bands corresponding to apoB48 and apoB 100 are excised, dissolved at 60° C. in 0.2 ml of 60% (v/v) perchloric acid followed by 0.4 ml of 30% (v/v) hydrogen peroxide (Mahin, D. T. and Lofberg, R. T. (1966) Anal. Biochem. 16:500-509), and the radioactivity associated with apoB48 and apoB100 is determined using Hionic-Fluor scintillation cocktail (Packard Instrument Co., Meriden, Conn., U.S.A.).

For detection of apoB in VLDL, density centrifugation is performed according to Chung et al. ((1980) J. Lipid Res. 21:284-291). To 1.2 ml of medium (containing 1 mM benzamidine) are added 100 μl of freshly prepared rat plasma and 0.7 grams KBr (4 M final concentration of KBr, density 1.3 g/ml). The mixture is placed into 5.5 ml Quick-Seal centrifuge tubes (Beckman), carefully overlayed with 0.9% NaCl, and centrifuged for 45 minutes at 41,600 g (65,000 revolutions/minute; VTi 65.2 rotor). Fractions of 0.5 ml are collected from the bottom of the tubes, and the densities are determined. Top fractions containing VLDL have a density of <1.013 g/ml. Fractions are adjusted to 0.9 ml with water, and apoB is immunoprecipitated and analyzed as described above.

### Other Methods

7.5 mM oleic acid/10% (w/v) BSA stock solution is prepared by dissolving fatty acid-free BSA in DMEM. The solution is heated to 56° C., added to 0.106 grams oleic acid, stirred until the solution clarifies, and sterilized by filtration.

Protein concentration is determined using the Bio-Rad Protein Assay kit using BSA as a protein standard.

### Human 25934

The human 25934 sequence (SEQ ID NO:7), which is approximately 1512 nucleotides long including untranslated regions, contains a predicted methionine-initiated coding sequence of about 990 nucleotides (nucleotides 342-1334 of SEQ ID NO:7; SEQ ID NO:9). The coding sequence encodes a 330 amino acid protein (SEQ ID NO:8).

Human 25934 contains the following regions or other structural features: a desaturase domain located at about amino acid residues 51 to 295 of SEQ ID NO:8; two transmembrane regions at about amino acids 50-93 and 194-235 of SEQ ID NO:8; three cytoplasmic domains at about amino acids 1-49, 94-193, and 236-330 of SEQ ID NO:8; one predicted N-glycosylation site (PS00001) at about amino acids 233 to 236 of SEQ ID NO:8; one predicted cAmp and cGMP dependent protein kinase phosphorylation site (PS00004) at about amino acids 311 to 314 of SEQ ID NO:8; four predicted Protein Kinase C phosphorylation sites (PS00005) at about amino acids 98 to 100, 101 to 103, 255 to 257 and 308 to 310 of SEQ ID NO:8; two predicted Casein Kinase II phosphorylation sites (PS00006) located at about amino acids 138 to 141 and 283-286 of SEQ ID NO:8; four predicted N-myristoylation sites (PS00008) from about amino acids 23 to 28, 40 to 45, 59 to 64, and 88 to 93 of SEQ ID NO:8; one predicted amidation site (PS00009) from about amino acid 170 to 173 of SEQ ID NO:8; and one predicted fatty acid desaturase family 1 signature (PS00476) from about amino acid 268 to 282 of SEQ ID NO:8.

For general information regarding PFAM identifiers, PS prefix and PF prefix domain identification numbers, refer to Sonnhammer et al. (1997) Protein 28:405-420.

A plasmid containing the nucleotide sequence encoding human 25934 was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The 25934 protein contains a significant number of structural characteristics in common with members of the desaturase family.

Based on sequence homology, 25934 polypeptide is predicted to be a member of the desaturase family of enzymes, specifically the stearoyl-Co desaturase family (SCD family, EC 1.14.99.5).

Fatty acid desaturases are critical regulatory enzymes of unsaturated fatty acid biosynthesis and catalyze the conversion of a single bond between two carbon atoms (C—C) to a double bond (C═C) in a fatty acyl chain. The resultant double bond is often referred to as an unsaturated bond. Eukaryotic fatty acid desaturases, typically, are iron-containing enzymes that catalyze the NAD-(P)H and O₂-dependent introduction of double bonds into methylene-interrupted fatty acid chains. Examination of the deduced amino acid sequence from mammals, fungi, insects, higher plants and cyanobacteria has revealed three regions of conserved primary sequence containing HX(3 or 4)H, HX(2 or 3), and HX(2 or 3)HH. This motif is also present in the bacterial membrane enzymes alkaline hydroxylase (omega-hydroxylase) and xylene monooxygenase.

There are three types of eukaryotic fatty acid desaturases, acyl-CoA, acyl-ACP, and acyl-lipid desaturases (Ntambi et al., Biochem. and Biophys. Res. Com. 266:1-4, 1999). In plants and cyanobacteria, acyl-lipid desaturases catalyzing most desaturation reactions and introduce unsaturated bonds into fatty acids that are in a lipid-bound form. Acyl-ACP desaturases are present in the plastids of plant cells and insert a double bond into fatty acids that are bound to acyl carrier protein (ACP). In animals, yeast and fungal cells, Acyl-CoA introduce unsaturated bonds into fatty acids that are bound to coenzyme A (CoA). A gene cloned from this family is stearoyl-CoA desaturase and this gene has been identified in many organisms including mice, rats, humans, yeast, ovines, and hamsters.

Fatty acid desaturases can introduce an unsaturated bond at a specific position in a fatty acyl chain, for example, at the −6, −9, or −12 position. Desaturases are typically integral membrane proteins induced in the endoplasmic reticulum by dietary manipulations and then rapidly degraded (Ozols, J. (1997) MBC Vol. 8 (11): 2281-2290). Unsaturated fatty acids can be formed from a variety of fatty acids including palmitate and stearate resulting in the formation of unsaturated fatty acids palmitoleate (16:1), and oleate (18:1).

In mammals, the rate limiting step in the biosynthesis of monounsaturated fatty acids is the insertion of an unsaturated bond by stearoyl-CoA desaturase (SCD) in the −9 position of the fatty acid. SCD preferentially catalyzes the synthesis of oleic acid. Oleate enriched low density lipoprotein (LDL) exhibits increased affinity for the vessel wall, and is therefore pro-atherogenic (Rudel, L. L. et al. (1997) J. Clin. Invest. 1:100(1):74-83). SCD involvement in generating atherogenic LDL variants and in regulating triglyceride synthesis is further supported by the finding that polyunsaturated fatty acids (PUFA), which protect against atherosclerosis, negatively regulate the expression of the SCD gene (Rudel, L L et al. (1995) Atheroscler. Thromb. Vasc. Biol. 15(12):2101-10; Ntambi, J M (1999) J. Lipid Res. 40(9): 1549-58). Moreover, a mouse deficient for SCD exhibits significant reduction in triglycerides (Miyazaki, M. et al. (2000) J. Biol. Chem., in press).

SCD enzymes are structurally and functionally homologous to one another, and can convert a single bond to a double bond in a fatty acyl chain. SCD enzymes utilize oxygen and electrons from cytochrome b₅ for catalysis. Similar to other enzymes such as ribonucleotide reductases and methane monoxygenases, stearoyl-CoA desaturases can have a conserved iron binding motif which includes eight histidines (Shanklin et al. (1997) Proc. Natl. Acad. Sci. USA 94:2981-2986), “H-X(3-4)-H-X(7-41)-H-X(2-3)-H-H-X(61-189)-H-X(2-3)-H-H (SEQ ID NO:11).” The eight histidine residues common to desaturase family members are typically divided among three regions of the protein: region Ia (H-X(3-4)-H); region Ib (the first H-X(2-3)-H-H sequence); and region II (the second H-X(2-3)-H-H sequence) (Shanklin et al. (1994) Biochemistry 33:12787-94).

SCDs typically contain two or three long hydrophobic domains termed “transmembrane regions,” each of which is capable of spanning the membrane two times (Shanklin et al. (1994) Biochemistry 33:12787-94). Because a transmembrane region is capable of traversing the membrane twice, amino acid residues flanking a transmembrane region reside on the same side of the membrane (Stukey et al. (1990) J. Biol. Chem. 265:20144-49). Thus, when region I (regions Ia and Ib) and region II are divided by a transmembrane region in a desaturase family member, the regions will typically reside on the same side of the membrane, e.g., the cytoplasmic face of the endoplasmic reticulum membrane.

A 25934 polypeptides include a “desaturase domain” or regions homologous with a “desaturase domain”. As used herein, the term “desaturase domain” includes an amino acid sequence of about 25 to 600 amino acid residues in length and having a bit score for the alignment of the sequence to the fatty acid desaturase domain (HMM) of at least 50. Preferably, a desaturase domain includes at least about 50-500 amino acids, more preferably about 100-400 amino acid residues, or about 200-250 amino acids and has a bit score for the alignment of the sequence to the desaturase domain (HMM) of at least 60, 80, 100, 150, 200, 250, 300, 450, 500 or greater.

In a preferred embodiment, 25934 polypeptide or protein has a “desaturase domain” or a region which includes at least about 50-500 amino acids, more preferably about 100-400 amino acid residues, or about 200-250 amino acid residues and has at least about 70% 80% 90% 95%, 99%, or 100% homology with a “desaturase domain,” e.g., the desaturase domain of human 25934 (e.g., residues 51-295 of SEQ ID NO:8). Preferably, the desaturase domain of a 25934 polypeptide includes at least one, two, three, four, five, six, seven and preferably eight conserved histidines. Preferably, the histidines form an eight-histidine motif, which binds two iron atoms in the catalytic center. For example, a 25934 polypeptide contains histidine residues at about amino acids 94, 99, 131, 134, 135, 272, 275, and 276 of SEQ ID NO:8.

To identify the presence of a “desaturase” domain in a 25934 protein sequence, and make the determination that a polypeptide or protein of interest has a particular profile, the amino acid sequence of the protein can be searched against a database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a “desaturase” domain in the amino acid sequence of human 25934 at about residues 51-295 of SEQ ID NO:8 (the identified desaturase consensus domain is set forth in SEQ ID NO:10).

A 25934 family member includes a desaturase domain and optionally also a fatty acid desaturase family 1 signature, i.e., a motif that matches the ProSite motif PS00476, “G-E-X-[FY]-H-N-[FY]-H-H-X-F-P-X-D-Y (SEQ ID NO:12),” e.g., this motif is found at about residues 268 to 282 of SEQ ID NO:8.

In one embodiment, a 25934 protein includes at least one, preferably two, transmembrane regions. As used herein, the term “transmembrane region” includes an amino acid sequence of about 20 amino acid residues in length that spans a phospholipid membrane, e.g., an endoplasmic reticulum membrane, twice. More preferably, a transmembrane region includes about at least 22, 24, 25, 30, 35, 40, 45, 50, 55, 60, 65, or 70 amino acid residues and spans a phospholipid membrane twice. Transmembrane regions are rich in hydrophobic residues, and typically have an a-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane regions are described in, for example, pfam.wustl.edu/cgi-bin/getdesc?name=7tm-1, and Zagotta W. N. et al, (1996) Annual Rev. Neuronsci. 19: 235-63, the contents of which are incorporated herein by reference.

In a preferred embodiment, a 25934 polypeptide or protein has at least one transmembrane region or a region which includes at least 20, 22, 24, 25, 30, 35, 40, 45, 50, 55, 60, 65, or 70 amino acid residues and has at least about 60%, 70% 80% 90% 95%, 99%, or 100% homology with a “transmembrane region,” e.g., at least one transmembrane region of human 25934 (e.g., amino acid residues 50-93 or 194-235 of SEQ ID NO:8).

In one embodiment, a 25934 protein includes at least one cytoplasmic domain. When located at the N-terminal domain the cytoplasmic domain is referred to herein as an “N-terminal cytoplasmic domain”. As used herein, an “N-terminal cytoplasmic domain” includes an amino acid sequence having about 1-200, preferably about 10-100, preferably about 20-90, more preferably about 30-80, more preferably about 35-70, more preferably about 40-60, or even more preferably about 45-55 amino acid residues in length and is located in the cytoplasm of a cell. The C-terminal amino acid residue of a “N-terminal cytoplasmic domain” is adjacent to an N-terminal amino acid residue of a transmembrane region in a 25934 protein. For example, an N-terminal cytoplasmic domain is located at about amino acid residues 1-49 of SEQ ID NO:8.

In a preferred embodiment, a 25934 polypeptide or protein has at least one N-terminal cytoplasmic domain or a region which includes at least about 5, preferably about 40-60, or even more preferably about 45-55 amino acid residues and has at least about 60%, 70% 80% 90% 95%, 99%, or 100% homology with an “N-terminal cytoplasmic domain,” e.g., at least one N-terminal cytoplasmic domain of human 25934 (e.g., residues 1-49 of SEQ ID NO:8).

In another embodiment, a 25934 protein includes a “cytoplasmic loop” in the sequence of the protein. As used herein, a “cytoplasmic loop” includes an amino acid sequence having a length of at least about 10, preferably about 20-250, preferably about 30-150, more preferably about 80-120 amino acid residues and is located within the cytoplasm of a cell. Accordingly, the N-terminal amino acid residue of a “cytoplasmic loop” is adjacent to a C-terminal amino acid residue of a transmembrane region and the C-terminal residue of a “cytoplasmic loop” is adjacent to a N-terminal amino acid residue of a transmembrane region in a 25934 protein. For example, a cytoplasmic loop is found at about amino acid residues 94-193 of SEQ ID NO:8.

In a preferred embodiment, a 25934 polypeptide or protein has a cytoplasmic loop or a region which includes at least about 10, preferably about 20-250, preferably about 30-150, more preferably about 80-120 amino acid residues and has at least about 60%, 70% 80% 90% 95%, 99%, or 100% homology with an “cytoplasmic loop,” e.g., the cytoplasmic loop of human 25934 (e.g., residues 94-193 of SEQ ID NO:8).

In another embodiment, a 25934 protein includes a “C-terminal cytoplasmic domain”, also referred to herein as a C-terminal cytoplasmic tail, in the sequence of the protein. As used herein, a “C-terminal cytoplasmic domain” includes an amino acid sequence having a length of at least about 30, preferably about 50-150, preferably about 60-200, more preferably about 80-110 amino acid residues and is located within the cytoplasm of a cell. Accordingly, the N-terminal amino acid residue of a “C-terminal cytoplasmic domain” is adjacent to a C-terminal amino acid residue of a transmembrane region in a 25934 protein. For example, a C-terminal cytoplasmic domain is found at about amino acid residues 236-330 of SEQ ID NO:8.

In a preferred embodiment, a 25934 polypeptide or protein has a C-terminal cytoplasmic domain or a region which includes at least about 30, preferably about 50-150, preferably about 60-200, more preferably about 80-110 amino acid residues and has at least about 60%, 70% 80% 90% 95%, 99%, or 100% homology with an “C-terminal cytoplasmic domain,” e.g., the C-terminal cytoplasmic domain of human 25934 (e.g., residues 236-330 of SEQ ID NO:8).

25934 polypeptides of the invention include 25934 fragments which include: all or part of a hydrophobic sequence e.g., all or part of the sequence from about residue 71 to about residue 91 of SEQ ID NO:8; all or part of a hydrophilic fragment; or other fragments that include a cysteine or a glycosylation site.

A multiple sequence alignment of the 25934 amino acid sequence with the human (SEQ ID NO:13), rat (SEQ ID NO:14), and chicken (SEQ ID NO:15) delta-9 desaturase proteins revealed 63.6%, 58.4%, and 58.4% identity between the 25934 amino acid sequence and the chicken, human and rat amino acid sequences, respectively.

As the 25934 polypeptides of the invention may modulate 25934-mediated activities, they may be useful as of for developing novel diagnostic and therapeutic agents for 25934-mediated or related disorders, as described below.

As used herein, a “25934 activity”, “biological activity of 25934” or “functional activity of 25934”, refers to an activity exerted by a 25934 protein, polypeptide or nucleic acid molecule on e.g., a 25934-responsive cell or on a 25934 substrate, e.g., a protein substrate, as determined in vivo or in vitro. In one embodiment, a 25934 activity is a direct activity, such as an association with a 25934 target molecule. A “target molecule” or “binding partner” is a molecule with which a 25934 protein binds or interacts in nature. In an exemplary embodiment, the binding partner is a fatty acid, e.g., myristic, palmitic or stearic acid. The 25934 proteins of the present invention can have one or more of the following activities: (1) catalyzing the formation of a double bond, preferably, at positions up to 9 carbons from the carboxyl end of a molecule, e.g., a fatty acid, such as a polyunsaturated fatty acid; (2) modulating the synthesis of monounsaturated fatty acids, e.g., modulating the synthesis of a fatty acid synthesized in an animal, e.g., oleic acid, palmitoyl- and stearoyl-CoA; (3) modulating the desaturation of a fatty acid, e.g., a polyunsaturated fatty acids; (4) modulating cellular lipid composition, e.g., modulating the ratio of saturated and unsaturated fatty acids; (5) modulating the energy state of adipocytes; (6) modulating membrane fluidity; (7) modulating lipid storage; (8) modulating proliferation and/or differentiation; (9) modulating lipoprotein (e.g., LDL) composition and/or concentration; (10) regulating triglyceride synthesis; (11) altering the HDL/LDL ration; or (12) modulating fatty acid metabolism.

Based on the above-described sequence similarities, the 25934 molecules of the present invention are predicted to have similar biological activities as other desaturase family members, and in particular, stearoyl CoA desaturases (SCD). For example, the 25934 polypeptide or a domain therein, e.g., desaturase domain, may function to catalyze the conversion of a single bond between two carbon atoms (C—C) to a double bond (C═C) in a fatty acid chain. This modification is expected to occur at the n9 position of the fatty acid. Desaturases are predicted to contribute to an unfavorable LDL content state, e.g., by increasing LDL-oleate, which is atherogenic (Rudel, L L. et al. (1997) J. Clin Invest. 1:100(1):74-83) as well as by playing a role in triglyceride metabolism and/or biosynthesis. As shown by TAQMAN™ analysis, Niacin treatment in the marmoset model results in significant repression of 25934 in the liver. Niacin has been shown to alter the composition of LDL and HDL to a favorable state, to cause a significant reduction in triglycerides, and to increase HDL concentration (Goldberg, A. (2000) Am. J. Cardiol. 85(9):1100-5. Moreover, a mouse deficient for SCD exhibits significant reduction in triglycerides (Miyazaki, M. et al. (2000) J. Biol. Chem. 275(39):30132-8). Accordingly, the 25934 molecules can act as novel diagnostic targets and therapeutic agents for controlling disorders associated with abnormal or aberrant desaturase activity and/or triglyceride levels. In particular, it is predicted that targeting the inhibition of 25934 nucleic acids and polypeptides will results in the favorable modification, and possible reduction, of LDL content and/or reduction of triglycerides. Thus, the 25934 molecules can act as novel targets for treating and/or diagnosing fatty acid metabolic disorders (e.g., desaturation of fatty acids) such as obesity and/or diabetes and more generally, cardiovascular disorders.

The term “cardiovascular disorders” or “disease” includes heart disorders, as well as disorders of the blood vessels of the circulation system caused by, e.g., abnormally high concentrations of lipids in the blood vessels.

In some embodiments, the therapeutic and prophylactic uses of the compositions of the invention, further include the administration of cholesterol lowering agents as a combination drug therapies. The term “combination therapy” as used herein refers to the administration to a subject (concurrently or sequentially) of two or more cholesterol lowering agents. Current combination therapy therapies using combinations of niacin and statins are being used with positive results to treat hyperlipidemia (Guyton, J R. (1999) Curr Cardiol Rep. 1(3):244-250; Otto, C. et al. (1999) Internist (Berl) 40(12):1338-45). Other useful drug combinations include those derived by addition of fish oil, bile acid binding resins, or stanol esters, as well as nonstatin combinations susn as niacin-resin or fibrate-niacin (Guyton, J R. (1999) supra). For examples of dosages and administration schedules of the cholesterol lowering agents, the teachings of Guyton, J R. (1999) supra, Otto, C. et al. (1999) supra, Guyton, J R et al. (1998) Am J Cardiol 82(12A):82U-86U; Guyton, J R et al. (1998) Am J Cardiol. 82(6):737-43; Vega, G L et al. (1998) Am J. Cardiol. 81(4A):36B-42B; Schectman, G. (1996) Ann Intern Med. 125(12):990-1000; Nakamura, H. et al. (1993) Nippon Rinsho 51(8):2101-7; Goldberg, A. et al. (2000) Am J Cardiol 85(9):1100-5; Morgan, J M et al. (1996) J Cardiovasc. Pharmac. Ther. 1(3):195-202; Stein, E A et al. (1996) J Cardiovasc Pharmacol Ther 1(2):107-116; and Goldberg, A C (1998) Am Cardiol 82(12A):35U-41U, are expressly incorporated by reference.

The 25934 molecules can also be used to treat, diagnose or prevent lipid disorders. Examples of lipid disorders include those disorders which affect fatty acid metabolism. Fatty acids are synthesized from acetyl-CoA, which is derived from carbohydrate, protein and other non-lipid sources, and the pathway produces saturated fatty acids, predominantly palmitic acid (10:0). In mammals, the fatty acids may be elongated and desaturated. Desaturation is catalyzed by desaturases which function by inserting one or more double bonds at positions up to 9 carbons from the carboxyl end of a fatty acid molecule.

The degree of fatty acid desaturation in cell membrane lipids determines membrane fluidity. The activity of the desaturase enzyme is critical for maintaining the ratio of saturated and unsaturated fatty acids in cell membranes. Alterations in this ratio can, e.g., alter the physical properties of membranes. Moreover, alterations in the ratio of fatty acids have been implicated in a range of diseases including diabetes, obesity, hypertension, cancer, developmental disorders, immune disorders and neurological and the above-described heart diseases. For example, tumor tissue and virus-transformed cells have a higher content of unsaturated fatty acids, especially oleic acid. Such shifts increase the metabolic rates of many lipid-dependent enzymes and are associated with a higher capacity for cell division.

As assessed by TAQMAN™ analysis, the 25934 mRNA is found in the brain, ovary, kidney and liver, therefore the molecules of the invention can be used to develop novel agents or compounds to treat, prevent and/or diagnose disorders involving aberrant activities of those cells. For example, the molecules of the invention can be used to treat, present and/or diagnose neurological disorders, brain disorders, ovarian disorders, kidney disorders and liver disorders, as described below.

Identification and Characterization of Human 25934 cDNA

The human 25934 sequence (SEQ ID NO:7), which is approximately 1512 nucleotides long including untranslated regions, contains a predicted methionine-initiated coding sequence of about 990 nucleotides (nucleotides 342 to 1334 of SEQ ID NO:7; SEQ ID NO:9). The coding sequence encodes a 330 amino acid protein (SEQ ID NO:8).

Tissue Distribution of 25934 mRNA

Endogenous human 25934 gene expression was determined using the Perkin-Elmer/ABI 7700 Sequence Detection System which employs TAQMAN™ technology. To determine the level of 25934 in various human tissues a primer/probe set was designed using Primer Express (Perkin-Elmer) software and primary cDNA sequence information. Total RNA was prepared from a series of human tissues using an RNEASY™ kit from Qiagen. First strand cDNA was prepared from 1 μg total RNA using an oligo-dT primer and SUPERSCRIPT II™ reverse transcriptase (Gibco/BRL). cDNA obtained from approximately 50 ng total RNA was used per TAQMAN™ reaction.

25934 mRNA levels were analyzed in a variety of samples isolated from the human fetal heart, spinal cord, brain (cortex, hypothalamus, glial cells), ovary, kidney, liver, endothelial cells and smooth muscle cells (SMC). The highest relative 25934 mRNA expression, i.e., greater than 200 relative units, was observed in spinal cord, brain and ovary. High level mRNA expression, i.e., greater than 100 relative units was observed in the kidney, endothelial cells and human umbilical vein endothelial cells (HUVEC). Expression in liver (a target organ for 25934) was positive but lower relative to other tissues

The relative 25934 mRNA expression levels were determined using an expanded TAQMAN™ panel of human liver tissues and then this panel was used to compare the expression of 25934 mRNA and stearoyl CoA desaturase (SCD) mRNA. Expression of 25934 mRNA in human liver is equivalent to the relative expression of the known SCD gene.

Further TAQMAN™ analyses also demonstrate an inhibition of 25934 mRNA expression in the marmoset animal model. Niacin treatment in the marmoset model results in significant repression of 25934 in the liver.

### Human 26335 (DHY)

The present invention is based, at least in part, on the discovery of novel molecules, referred to herein as “26335”, “dehydratase” or “DHY” nucleic acid and protein molecules, which are novel members of a family of enzymes possessing dehydratase activity. These novel molecules are capable of delaminating serine or threonine to pyruvate or 2-oxobutyrate, respectively, by catalyzing a two-step reaction of dehydration of the amino acid, followed by hydrolysis of the resulting imine. These novel molecules may thus play a role in or function in a variety of cellular processes, e.g., cellular proliferation, growth, differentiation, migration, and inter- or intra-cellular communication.

The biosynthesis and metabolism of amino acids is of critical importance in many metabolic and catabolic pathways in cells, and is fundamental to the production of cellular proteins. A wide array of enzymes facilitate the synthesis, interconversion, and degradation of amino acids, including transaminases, oxidases, reductases, dehydrogenases, and kinases, among many others. One such family of enzymes, the serine and threonine dehydratases, catalyze the irreversible deamination of serine or threonine to pyruvate or 2-oxobutyrate, respectively.

The reaction mechanism for these enzymes has been characterized (Snell and Di Mari (1970) The Enzymes (Boyer, P. D., ed.), Academic Press: 3^{rd} ed. Vol. 2: 335-370; and Ogawa et al. (1989) Biochim. Biophys. Acta 996: 139-141). First, a Schiff base is formed between a pyridoxal-5′phosphate cofactor and a specific lysine residue which is strictly conserved within the serine and threonine dehydratase family. A new Schiff base is subsequently formed between the cofactor and the hydroxyamino acid by transimination, catalyzing the removal of the α-proton through stabilization of the resulting carbanion by the planar π-system of the prosthetic group. The hydroxyl group is eliminated, and the resultant enamine is freed by a second transimination. A tautomerization step results in the formation of a ketimine, which hydrolyses to the 2-oxoacid and ammonia (Gabowski et al. (1993) Trends in Biological Sciences 18: 297-300). A subclass of the serine dehydratases found in anaerobic bacteria substitutes an iron-sulfur cofactor for pyridoxal-5′-phosphate, and exhibits an altered reaction mechanism with similarities to the mechanism of aconitase (Hofmeister et al. (1993) Eur J Biochem 215(2):341-9). Threonine dehydratases, in general, are able to deaminate either threonine or serine, while the serine dehydratases have been found to be specific for the deamination of serine (Grabowski et al. (1992) Eur. J. Biochem. 199:89-94; and Alfoldi et al. (1968) J. Bacteriol. 96:1512-1518).

Members of the serine and threonine dehydratase family are found in nearly all organisms, from bacteria to yeast to mammals. Alignments of the amino acid sequences of family members from disparate organisms have revealed two conserved regions, termed C1 and C2. The conserved C1 domain is located approximately 50 amino acid residues from the N-terminus of the enzyme, and includes the consensus sequence (G)S(F)K(I)RG (Datta et al. (1987) Proc. Natl. Acad. Sci. USA 84: 393-397). This region of the protein has been shown to bind the cofactor, pyridoxal-5′-phosphate, at the conserved lysine residue (Schlitz and Schmitt (1981) FEBS Lett. 134:57-62). Conserved region C2 is located in the central region of the amino acid sequences of these enzymes, and is predicted to have a beta sheet-coil-beta sheet structure (Datta et al., supra). C2 is rich in glycine, and is thought to be involved in the catalytic activity of the enzymes (Marceau et al. (1988) J. Biol. Chem. 263: 16926-16933).

As used herein, the term “dehydratase” includes a molecule which is involved in the metabolism and catabolism of biochemical molecules necessary for energy metabolism, for intra- or intercellular signaling, and for metabolism or catabolism of metabolically important biomolecules. Typically, dehydratases are involved in the deamination of amino acids, e.g., serine or theronine. Examples of dehydratases include serine and threonine dehydratases. Thus, the DHY molecules of the present invention provide novel diagnostic targets and therapeutic agents to control dehydratase-associated disorders.

As used herein, a “dehydratase-associated disorder” includes a disorder, disease or condition which is caused or characterized by a misregulation (e.g., downregulation or upregulation) of dehydratase activity. Misregulation of dehydratase activity can result in the overproduction or lack of production of one or more amino acids or biologically important metabolic precursor molecules (e.g., pyruvate or 2-oxobutyrate), and, by extension, aberrant metabolite, energy molecules, and/or protein production in the cell as a whole. Proteins produced by the cell not only include those involved in normal cellular functioning (e.g., enzymes, receptors, chaperoning, and transcription factors), but also important signaling molecules (e.g., growth factors, cytokines, and neuropeptides). Dehydratase-associated disorders, therefore, can detrimentally affect cellular functions such as cellular proliferation, growth, differentiation, or migration, inter- or intra-cellular communication; and tissue function, such as cardiac function or musculoskeletal function. Examples of dehydratase-associated disorders include cellular proliferation, growth, differentiation, or migration disorders, CNS disorders, cardiovascular disorders and disorders affecting tissues in which DHY protein is expressed.

As used herein, a “dehydratase-mediated activity” includes an activity which involves the deamination of one or more amino acids, e.g., threonine or serine. Dehydratase-mediated activities include the production of biochemical molecules necessary for energy metabolism, for intra- or intercellular signaling (e.g., the production of growth factors), and for metabolism or catabolism of metabolically important biomolecules (e.g., isoleucine and protein production).

The family of DHY proteins comprises at least one “transmembrane domain”. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al., (1996) Annual Rev. Neurosci. 19: 235-263, the contents of which are incorporated herein by reference. Amino acid residues 67-83, 167-187, 270-288, and 295-311 of the native DHY protein are predicted to comprise transmembrane domains. Accordingly, DHY proteins having at least 50-60% homology, preferably about 60-70%, more preferably about 70-80%, or about 80-90% homology with a transmembrane domain of human DHY are within the scope of the invention.

In another embodiment, a DHY molecule of the present invention is identified based on the presence of a “serine/threonine dehydratase pyridoxal-phosphate attachment site” in the protein or corresponding nucleic acid molecule. As used herein, the term “serine/threonine dehydratase pyridoxal-phosphate attachment site” includes a protein domain having an amino acid sequence of about 10-20 amino acid residues. Preferably, a serine/threonine dehydratase pyridoxal-phosphate attachment site has about 14 residues and the following consensus sequence: [DESH]-x(4,5)-[STVG]-x-[AS]-[FYI]-K-[DLIFSA]-[RVMF]-[GA]-[LIVMGA] (SEQ ID NO:19) (Datta et al. (1987) Proc. Natl. Acad. Sci. USA 84:393-397; and Ogawa et al. (1989) Biochim. Biophys. Acta 996:139-141). To identify the presence of a serine/threonine dehydratase pyridoxal-phosphate attachment site in a DHY protein, and make the determination that a protein of interest has a particular profile, the amino acid sequence of the protein may be searched against a database of known protein domains (e.g., the ProSite database). The serine/threonine dehydratase pyridoxal-phosphate attachment site has been assigned ProSite accession number PS00165. A search was performed against the ProSite database resulting in the identification of a serine/threonine dehydratase pyridoxal-phosphate attachment site in the amino acid sequence of human DHY (SEQ ID NO:17) at about residues 39-52 of SEQ ID NO:17.

In another embodiment, a DHY molecule of the present invention is identified based on the presence of a “serine/threonine dehydratase domain” in the protein or corresponding nucleic acid molecule. As used herein, the term “serine/threonine dehydratase domain” includes a protein domain having an amino acid sequence of about 200-400 amino acid residues and a bit score of about 50, 60, 70, 80, 90, 100, 110, 120, 130, 140, 150, 160, 170, 180, 190, 200, 210, or 220 or more. Preferably, a serine/threonine dehydratase domain includes at least about 250-350, or more preferably about 301 amino acid residues, and a bit score of at least 229. To identify the presence of a serine/threonine dehydratase domain in a DHY protein, and make the determination that a protein of interest has a particular profile, the amino acid sequence of the protein may be searched against a database of known protein domains (e.g., the HMM database). A search was performed against the HMM database resulting in the identification of a serine/threonine dehydratase domain in the amino acid sequence of human DHY (SEQ ID NO:17) at about residues 11-311 of SEQ ID NO:17.

In another embodiment, a DHY molecule of the present invention is identified based on the presence of a “pyridoxal phosphate-dependent lyase synthase domain” in the protein or corresponding nucleic acid molecule. As used herein, the term “pyridoxal phosphate-dependent lyase synthase domain” includes a protein domain having an amino acid sequence of about 200-300 amino acid residues and having a bit score for the alignment of the sequence to the pyridoxal phosphate-dependent lyase synthase domain of at least 10, 20, 30, 40, 50, 60, 70, 80 or higher. Preferably, a pyridoxal phosphate-dependent lyase synthase domain includes at least about 240-275, or more preferably about 265 amino acid residues, and has a bit score for the alignment of the sequence to the pyridoxal phosphate-dependent lyase synthase domain of at least 88. The pyridoxal phosphate-dependent lyase synthase domain has been assigned ProDom entry 206. To identify the presence of a pyridoxal phosphate-dependent lyase synthase domain in a DHY protein, and to make the determination that a protein of interest has a particular profile, the amino acid sequence of the protein may be searched against a database of known protein domains (e.g., the ProDom database) using the default parameters. A search was performed against the ProDom database resulting in the identification of a pyridoxal phosphate-dependent lyase synthase domain in the amino acid sequence of human DHY (SEQ ID NO:17) at about residues 18-282 of SEQ ID NO:17.

In a preferred embodiment, the DHY molecules of the invention include at least one or more of the following domains: a transmembrane domain, a serine/threonine dehydratase pyridoxal-phosphate attachment site, a serine/threonine dehydratase domain, and/or a pyridoxal phosphate-dependent lyase synthase domain.

Isolated proteins of the present invention, preferably DHY proteins, have an amino acid sequence sufficiently identical to the amino acid sequence of SEQ ID NO:17, or are encoded by a nucleotide sequence sufficiently identical to SEQ ID NO:16 or 18. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, an “DHY activity”, “biological activity of DHY” or “functional activity of DHY”, refers to an activity exerted by a DHY protein, polypeptide or nucleic acid molecule on a DHY responsive cell or tissue, or on a DHY protein substrate, as determined in vivo, or in vitro, according to standard techniques. In one embodiment, a DHY activity is a direct activity, such as an association with a DHY-target molecule. As used herein, a “target molecule” or “binding partner” is a molecule with which a DHY protein binds or interacts in nature, such that DHY-mediated function is achieved. A DHY target molecule can be a non-DHY molecule or a DHY protein or polypeptide of the present invention (e.g., pyridoxal-5′-phosphate). In an exemplary embodiment, a DHY target molecule is a DHY ligand (e.g., serine or threonine). Alternatively, a DHY activity is an indirect activity, such as a cellular signaling activity mediated by interaction of the DHY protein with a DHY ligand. The biological activities of DHY are described herein. For example, the DHY proteins of the present invention can have one or more of the following activities: 1) modulate metabolism and catabolism of biochemical molecules necessary for energy production or storage (e.g., amino acids, such as serine or threonine); 2) modulate intra- or intercellular signaling; 3) modulate metabolism or catabolism of metabolically important biomolecules; 4) modulate cellular growth and differentiation; 5) modulate cellular proliferation; and 6) modulate production of growth factors and cytokines.

Accordingly, another embodiment of the invention features isolated DHY proteins and polypeptides having a DHY activity. Other preferred proteins are DHY proteins having one or more of the following domains: a transmembrane domain, a serine/threonine dehydratase pyridoxal-phosphate attachment site, a serine/threonine dehydratase domain, and/or a pyridoxal phosphate-dependent lyase synthase domain and, preferably, a DHY activity.

Additional preferred proteins have one or more of the following domains: a transmembrane domain, a serine/threonine dehydratase pyridoxal-phosphate attachment site, a serine/threonine dehydratase domain, and/or a pyridoxal phosphate-dependent lyase synthase domain, and are, preferably, encoded by a nucleic acid molecule having a nucleotide sequence which hybridizes under stringent hybridization conditions to a nucleic acid molecule comprising the nucleotide sequence of SEQ ID NO:16 or 18.

Isolation of the 26335 or DHY cDNA

The invention is based, at least in part, on the discovery of a human gene encoding a novel protein, referred to herein as DHY. The entire sequence of human clone Fbh26335 was determined and found to contain an open reading frame termed human “26335” or “DHY”, set forth in SEQ ID NO:16 and SEQ ID NO:18. The human DHY gene, which is approximately 1327 nucleotides in length, encodes a protein having a molecular weight of approximately 36.2 kD, which is approximately 329 amino acid residues in length and which is set forth in SEQ ID NO:17. The coding region (open reading frame) of SEQ ID NO:16, is set forth as SEQ ID NO:18.

### Analysis of the Human DHY Molecule

The amino acid sequence of human DHY was analyzed using the program PSORT to predict the localization of the protein within the cell. This program assesses the presence of different targeting and localization amino acid sequences within the query sequence. The results of the analysis show that human DHY (SEQ ID NO:17) may be localized to the cytoplasm, to the mitochondrion, to golgi, to the endoplasmic reticulum, extracellular to the cell or to the cell wall, to vacuoles, to the nucleus, or to secretory vesicles.

A search of the amino acid sequence of DHY was performed against the MEMSAT database. This search resulted in the identification of four transmembrane domains in the amino acid sequence of human 26335 or DHY (SEQ ID NO:17) at about residues 67-83, 167-187, 270-288, and 295-311.

A search of the amino acid sequence of DHY was also performed against the ProSite database. This search resulted in the identification of a “serine/threonine dehydratase pyridoxal-phosphate attachment site” in the amino acid sequence of DHY (SEQ ID NO:17) at about residues 39-52.

A search of the amino acid sequence of DHY was also performed against the HMM database. This search resulted in the identification of a “serine/threonine dehydratase domain” in the amino acid sequence of DHY (SEQ ID NO:17) at about residues 11-311 (score=229.5).

A search of the amino acid sequence of DHY was also performed against the ProDom database. This search resulted in the identification of a “pyridoxal phosphate-dependent lyase synthase domain” in the amino acid sequence of human DHY (SEQ ID NO:17) at about residues 18-282 (score=88).

Tissue Distribution of Human 26335 or DHY mRNA Using TaqMan™ Analysis

Highest expression of DHY mRNA was detected in HMVECL, U937/A10P10, bronchial epithelium, astrocytes, primary osteoblasts, keratinocytes, bronchial epithelium mix (BEA8-2B), congestive heart failure (CHF) heart tissue, the pituitary gland, fetal kidney tissue, fetal liver tissue, mesangial, T24Ctl, T24 (treated), adrenal gland tissue, Burkitt's Lymphoma tissue, mammary epithelium, WT LNCap+casodex, A549 IL-1, SCC25 CDDP-tongue squamous cell carcinoma tissue, testes, K563 (red blood cell line), A459 control (random-primed), liver tissue, prostate tissue, normal colon tissue, HMC-1 (mast cell line), normal megakarocytes, colon to liver metastasis (CHT128), colon to liver metastasis (CHT133), normal breast tissue, PTH osteo, lung squamous cell carcinoma tissue PIT299, and d8 dendritic cells.

Lesser expression was also detected in HUVECL, HL60/S, prostate epithelium, coronary smooth muscle cells, fetal lung tissue, fetal thymus tissue, congestive heart failure (CHF) heart tissue, prostate smooth muscle tissue, thyroid tissue, LPS 24 hour osteoblasts, uterine smooth muscle tissue (treated), bronchial smooth muscle tissue, umbilical smooth muscle tissue (treated), A2780 WT, fetal liver tissue, fetal skin, fetal adrenal gland tissue, midterm placental tissue, lung carcinoma tissue, embryonic keratinocytes, testes, skin, adipose, placental tissue (random-primed), kidney tissue (random-primed), HPK (random primed), salivary gland, heart tissue, the thymus, stomach tissue, spleen tissue, small intestine tissue, normal breast epithelia, normal ovarian epithelia, colon carcinoma tissue, ovarian ascites, serum starved embryonic lung tissue, lung squamous cell carcinoma tissue, brain subcortical white matter, normal prostate tissue (ziplox), HUVEC L (umbilical endothelium).

No expression was detected in U937/A10p50, CaCo, Hela cells, HL60/Adr, fetal brain tissue, melanocytes, cerebellum, aortic endothelial cells, prostate fibroblast tissue, mammary gland tissue, natural killer cells, LPS 1 hr. osteoblasts, LPS 6 hr. osteoblasts, WT LNCap+ testosterone, A2780ADR, fetal spleen tissue, the esophagus, p65 con+/+, p65 IL-1+/+, pulmonary artery smooth muscle tissue, erythroleukemia cells, SCC25 WT-tongue squamous cell carcinoma tissue, fetal hypothalamus, T cells (CD3 treated), T cells (CD3 IL-4/IL-10 treated), T cells (CD3 IFNg/TFNa treated), trachea tissue, ME 180 IL-1 cervical carcinoma tissue, ME 180 control, MCP-1 mast cell line, HPKII, lung tissue (random primed), heart tissue (random primed), fetal brain tissue (random primed), testes (random primed), RAJI (Burkitt's lymphoma B cell), ST 486 (lymphoma B cell), HL60 (acute promyelocytic leukemia), umbilical cord smooth muscle tissue (treated, random primed), uterine smooth muscle (treated, random primed), mammary gland tissue (random primed), small intestine tissue (random primed), fetal liver tissue (random primed), skeletal liver tissue (random primed), stomach tissue (random primed), spleen tissue (random primed), liver tissue (random primed), brain tissue (random primed), uterine tissue, uterine tissue (random primed), thymus tissue (random primed), 9 week fetus, lung tissue, skeletal muscle, retinal pigmentosa epithelial tissue, retinal tissue, bone marrow, Th-1 induced T cell, Th-2 induced T cell, colon carcinoma tissue (NDR 109), colon carcinoma tissue (NDR82), fetal dorsal spinal cord tissue, lung adenocarcinoma tissue (PIT245), megakaryocytes, BMCD34+, IBD colon tissue, cervical cancer tissue, spinal cord, dorsal root ganglia, and ovarian epithelium tumor tissue.

### Human 50365

The human 50365 sequence (SEQ ID NO:20, as recited below), which is approximately 3669 nucleotides long including untranslated regions, contains a predicted methionine-initiated coding sequence of about 2754 nucleotides, including the termination codon. The coding sequence encodes a 917 amino acid protein (SEQ ID NO:21, as recited below).

Human 50365 contains the following regions or other structural features: two hexokinase domains located at about amino acids 16 to 463 and 464 to 910 of SEQ ID NO:21, the latter of which includes a predicted hexokinase signature domain (PFAM Accession Number PS00378) from about amino acid residue 597 to about amino acid residue 622 of SEQ ID NO:21; two N-glycosylation sites (PS00001) from about amino acid 208 to about 211, and from about amino acid 655 to about 658, of SEQ ID NO:21; one glycosaminoglycan attachment site (PS00002) from about amino acid 896 to about 899 of SEQ ID NO:21; one cAMP- and cGMP-dependent protein kinase site (PS00004) from about amino acid 500 to 503 of SEQ ID NO:21; twelve protein kinase C phosphorylation sites (PS00005) from about amino acid 172 to 174, 379 to 381, 449 to 451, 508 to 510, 523 to 525, 547 to 549, 551 to 553, 772 to 774, 791 to 793, 826 to 828, 877 to 879, and 896 to 898, of SEQ ID NO:21; thirteen casein kinase II sites (PS00006) from about amino acid 35 to 38, 114 to 117, 161 to 164, 243 to 246, 275 to 278, 364 to 367, 569 to 572, 625 to 628, 722 to 725, 726 to 729, 787 to 790, 810 to 813, and 877 to 880, of SEQ ID NO:21; two tyrosine kinase phosphorylation sites (PS00007) from about amino acid 20 to 27, and 490 to 497, of SEQ ID NO:21; twenty-five N-myristylation sites (PS00008) from about amino acid 74 to 79, 151 to 156, 166 to 171, 179 to 184, 212 to 217, 227 to 232, 233 to 238, 299 to 304, 317 to 322, 348 to 353, 360 to 365, 411 to 416, 448 to 453, 518 to 523, 589 to 594, 613 to 618, 659 to 664, 674 to 679, 680 to 685, 746 to 751, 779 to 784, 807 to 812, 834 to 839, 858 to 863, and 895 to 900, of SEQ ID NO:21; and two amidation sites (PS00009) from amino acid 100 to 103, and amino acid 547 to 550 of SEQ ID NO:21.

Human 50365 is predicted to be a soluble, cytoplasmic polypeptide.

For general information regarding PFAM identifiers, PS prefix and PF prefix domain identification numbers, refer to Sonnhammer et al. (1997) Protein 28:405-420.

The 50365 protein contains a significant number of structural characteristics in common with members of the hexokinase family. Hexokinases are a family of sugar phosphorylating enzymes which carry out the phosphorylation of hexoses, for example, glucose, mannose, fructose, sorbitol and glucosamine, at the 6′-position. The phosphoryl donor can be MgATP, ITP, or dATP. Both α- and β-D-hexoses can be phosphorylated, although with different kinetic constants.

Four major isoenzymes are found in vertebrates: types I, II, III, and IV. The liver hexokinase isoenzyme (type IV) is also misleadingly known as glucokinase, and is expressed only in the liver and pancreatic β-cells. This isoenzyme has an important role in modulating insulin secretion. Structurally, the enzymes typically include a small N-terminal hydrophobic region, followed by two similar hexokinase domains of about 450 residues each. The second such region has catalytic activity, while the first has a regulatory role.

Hexokinases are present in nearly all cells. These enzymes have been identified as important for normal glycolytic activity. Irregularities in their function can lead to disorders such as diabetes and hemolytic anemia arising from hexokinase deficiency.

A 50365 polypeptide can include a “hexokinase domain” or regions homologous with a “hexokinase domain”. Type I, II, and III mammalian hexokinase polypeptides typically include two hexokinase domains. Each domain can form a structural unit that includes features of an α/β sandwich. Each domain can include amino acids with regulatory and/or catalytic functions, e.g., including a pocket for ATP and hexose substrates.

As used herein, the term “hexokinase domain” includes an amino acid sequence of about 300 to about 600 amino acid residues in length and having a bit score for the alignment of the sequence to the hexokinase domain (HMM) of at least 300. Preferably, a hexokinase domain includes at least about 350 to about 500 amino acids, more preferably about 400 to about 490 amino acid residues and has a bit score for the alignment of the sequence to the hexokinase domain (HMM) of at least 500, 600, 700, 800 or greater. The hexokinase domain (HMM) has been assigned the PFAM Accession PF00349.

In a preferred embodiment 50365 polypeptide or protein has a “hexokinase domain” or a region which includes at least about 500 to about 1200, more preferably about 550 to about 1100 or about 600 to about 1000 amino acid residues and has at least about 60%, 70% 80% 90% 95%, 99%, or 100% homology with a “hexokinase domain,” e.g., the hexokinase domain of human 50365 (e.g., residues 597 to 622 of SEQ ID NO:21).

Preferably, the hexokinase domain includes a “hexokinase signature domain”. This term refers to a protein domain having an amino acid sequence of at least about 15 to about 30 more preferably about 20 to about 30 or about 24 to about 28 amino acid residues which includes the following amino acid sequence: “L-G-F-T-F-S-F-P-C-x-Q-x-S-I-x-x-G-x-L-I-x-W-T-K-G-F” (SEQ ID NO:24). Preferably, a 50365 polypeptide or protein has a “hexokinase signature domain” or a region which includes and has at least about 60%, 70% 80% 90% 95%, 99%, or 100% homology with a “hexokinase signature domain,” e.g., the hexokinase signature domain of human 50365 (e.g., residues 597 to 622 of SEQ ID NO:21).

To identify the presence of a “hexokinase” domain in a 50365 protein sequence, and make the determination that a polypeptide or protein of interest has a particular profile, the amino acid sequence of the protein can be searched against the Pfam database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of two “hexokinase domains” in the amino acid sequence of human 50365 at about residues 16 to 463 and 464 to 910 of SEQ ID NO:21, the identified hexokinase domain consensus sequence is set forth in SEQ ID NO:23.

A 50365 family member can include at least one hexokinase domain. Furthermore, a 50365 family member can include at least one, preferably two N-glycosylation sites (PS00001); at least one glycosaminoglycan attachment site (PS00002); at least one cAMP- and cGMP-dependent protein kinase site (PS00004); at least one, two, three, four, five, six, seven, eight, nine, ten, eleven, or preferably twelve protein kinase C phosphorylation sites (PS00005); at least one, two, three, four, five, six, seven, eight, nine, ten, eleven, twelve, or preferably thirteen predicted casein kinase II phosphorylation sites (PS00006); at least one, or preferably two tyrosine kinase phosphorylation sites (PS00007); at least one, two, three, four, five, six, seven, eight, nine, ten, eleven, twelve, thirteen, fourteen, fifteen, sixteen, seventeen, eighteen, twenty, twenty-three, or preferably twenty-five predicted N-myristylation sites (PS00008); and at least one, preferably two amidation sites (PS00009).

50365 polypeptides of the invention include fragments which include: all or part of a hydrophobic sequence, e.g., the sequence of from about amino acid residue 365 to about amino acid residue 380, or from about amino acid residue 645 to about amino acid residue 655, of SEQ ID NO:21; all or part of a hydrophilic sequence, e.g., the sequence of from about amino acid residue 98 to about amino acid residue 120, or from about amino acid residue 715 to about amino acid residue 745 of SEQ ID NO:21.

As the 50365 polypeptides of the invention may modulate 50365-mediated activities, they may be useful as of for developing novel diagnostic and therapeutic agents for 50365-mediated or related disorders, as described below.

As used herein, a “50365 activity”, “biological activity of 50365” or “functional activity of 50365”, refers to an activity exerted by a 50365 protein, polypeptide or nucleic acid molecule on e.g., a 50365-responsive cell or on a 50365 substrate, e.g., a protein substrate, as determined in vivo or in vitro. In one embodiment, a 50365 activity is a direct activity, such as an association with a 50365 target molecule. A “target molecule” or “binding partner” is a molecule with which a 50365 protein binds or interacts in nature. In an exemplary embodiment, is a 50365 substrate, e.g., an aldohexose or ketohexose (e.g., glucose, mannose, fructose, sorbitol and glucosamine), or a phosphate-containing molecule, e.g., ITP, dATP, or MgATP as phosphoryl donor.

A 50365 activity can also be an indirect activity, e.g., a cellular signaling activity mediated by interaction of the 50365 protein with a 50365 substrate. For example, the 50365 proteins of the present invention can have one or more of the following activities: (1) it can catalyze the phosphorylation of a sugar, e.g., an aldohexoses and a ketohexoses (e.g., glucose, mannose, fructose, sorbitol and glucosamine); (2) it can catalyze sugar metabolism; (3) it can transfer a phosphate from a phosphate donor (e.g., ATP) to a sugar, e.g., an aldohexoses and a ketohexoses (e.g., glucose, mannose, fructose, sorbitol and glucosamine) to form a phosphorylated sugar, e.g., glucose-6-phosphate; (4) it can modulate glycolytic activities in a cell or tissue, e.g., a tissue in which a 50365 protein is expressed, e.g., muscle tissue and colon; or (5) it can modulate sugar metabolism; and/or (6) it can modulate cellular proliferation and/or differentiation.

Based on its structural features, the 50365 molecules of the present invention can have similar biological activities as hexokinase family members.

Expression of 50365 mRNA is modulated in a number of cancerous tissue samples. For example, 50365 mRNA is elevated in a number of colon tumors and colonic liver metastases (see, e.g., section entitled “Tissue Distribution of 50365 mRNA by TaqMan Analysis” below). Thus, the 50365 molecules can act as novel diagnostic targets and therapeutic agents for controlling disorders of neoplasia, e.g., cancer, a cell differentiative disorder, or a cell proliferative disorder as well as colon and lung disorders. 50365 molecules can also act as indicators and an agent for metabolic disorders, e.g., disorders of sugar metabolism and glycolysis.

Identification and Characterization of Human 50365 cDNA

The human 50365 nucleic acid sequence is recited as follows:

(SEQ ID NO: 20)
CCACGCGTCCGGCCTGGACTGGAAGCGTGCAACACTCCAGAGTCGTAGG
AGTGAACACTGCACAGGAATCTCTGCCCATCTCAGGAGAAACCAAACTT
GGGGAAAATGTTTGCGGTCCACTTGATGGCATTTTACTTCAGCAAGCTG
AAGGAGGACCAGATCAAGAAGGTGGACAGGTTCCTGTATCACATGCGGC
TCTCCGATGACACCCTTTTGGACATCATGAGGCGGTTCCGGGCTGAGAT
GGAGAAGGGCCTGGCAAAGGACACCAACCCCACGGCTGCAGTGAAGATG
TTGCCCACCTTCGTCAGGGCCATTCCCGATGGTTCCGAAAATGGGGAGT
TCCTTTCCCTGGATCTCGGAGGGTCCAAGTTCCGAGTGCTGAAGGTGCA
AGTCGCTGAAGAGGGGAAGCGACACGTGCAGATGGAGAGTCAGTTCTAC
CCAACGCCCAATGAAATCATCCGCGGGAACGGCATAGAGCTGTTTGAAT
ATGTAGCTGACTGTCTGGCAGATTTCATGAAGACCAAAGATTTAAAGCA
TAAGAAATTGCCCCTTGGCCTAACTTTTTCTTTCCCCTGTCGACAGACT
AAACTGGAAGAGGGTGTCCTACTTTCGTGGACAAAAAAGTTTAAGGCAC
GAGGAGTTCAGGACACGGATGTGGTGAGCCGTCTGACCAAAGCCATGAG
AAGACACAAGGACATGGACGTGGACATCCTGGCCCTGGTCAATGACACC
GTGGGGACCATGATGACCTGTGCCTATGACGACCCCTACTGCGAAGTTG
GTGTCATCATCGGAACTGGCACCAATGCGTGTTACATGGAGGACATGAG
CAACATTGACCTGGTGGAGGGCGACGAGGGCAGGATGTGCATCAACACA
GAGTGGGGGGCCTTCGGGGACGACGGGGCCCTGGAGGACATTCGCACTG
AGTTCGACAGGGAGCTGGACCTCGGCTCTCTCAACCCAGGAAAGCAACT
GTTCGAGAAGATGATCAGTGGCCTGTACCTGGGGGAGCTTGTCAGGCTT
ATCTTGCTGAAGATGGCCAAGGCTGGCCTCCTGTTTGGTGGTGAGAAAT
CTTCTGCTCTCCACACTAAGGGCAAGATCGAAACACGGCACGTGGCTGC
CATGGAGAAGTATAAAGAAGGCCTTGCTAATACAAGAGAGATCCTGGTG
GACCTGGGTCTGGAACCGTCTGAGGCTGACTGCATTGCCGTCCAGCATG
TCTGTACCATCGTCTCCTTCCGCTCGGCCAATCTCTGTGCAGCAGCTCT
GGCGGCCATCCTGACACGCCTCCGGGAGAACAAGAAGGTGGAACGGCTC
CGGACCACAGTGGGCATGGACGGCACCCTCTACAAGATACACCCTCAGT
ACCCAAAACGCCTGCACAAGGTGGTGAGGAAACTGGTCCCAAGCTGTGA
TGTCCGCTTCCTCCTGTCAGAGAGTGGCAGCACCAAGGGGGCCGCCATG
GTGACCGCGGTGGCCTCCCGCGTGCAGGCCCAGCGGAAGCAGATCGACA
GGGTGCTGGCTTTGTTCCAGCTGACCCGAGAGCAGCTCGTGGACGTGCA
GGCCAAGATGCGGGCTGAGCTGGAGTATGGGCTGAAGAAGAAGAGCCAC
GGGCTGGCCACGGTCAGGATGCTGCCCACCTACGTCTGCGGGCTGCCGG
ACGGCACAGAGAAAGGAAAGTTTCTCGCCCTGGATCTTGGGGGAACCAA
CTTCCGGGTCCTCCTGGTGAAGATCAGAAGTGGACGGAGGTCAGTGCGA
ATGTACAACAAGATCTTCGCCATCCCCCTGGAGATCATGCAGGGCACTG
GTGAGGAGCTCTTTGATCACATTGTGCAGTGCATCGCCGACTTCCTGGA
CTACATGGGCCTCAAGGGAGCCTCCCTACCTTTGGGCTTCACATTCTCA
TTTCCCTGCAGGCAGATGAGCATTGACAAGGGAACACTCATAGGGTGGA
CCAAAGGTTTCAAGGCCACTGACTGTGAAGGGGAGGACGTGGTGGACAT
GCTCAGGGAAGCCATCAAGAGGAGAAACGAGTTTGACCTGGACATTGTT
GCAGTCGTGAATGATACAGTGGGGACCATGATGACCTGTGGCTATGAAG
ATCCTAATTGTGAGATTGGCCTGATTGCAGGAACAGGCAGCAACATGTG
CTACATGGAGGACATGAGGAACATCGAGATGGTGGAGGGGGGTGAAGGG
AAGATGTGCATCAATACAGAGTGGGGAGGATTTGGAGACAATGGCTGCA
TAGATGACATCCGGACCCGATACGACACGGAGGTGGATGAGGGGTCCTT
GAATCCTGGCAAGCAGAGATACGAGAAAATGACCAGTGGGATGTACTTG
GGGGAGATTGTGCGGCAGATCCTGATCGACCTGACCAAGCAGGGTCTCC
TCTTCCGAGGGCAGATTTCAGAGCGTCTCCGGACCAGGGGCATCTTCGA
AACCAAGTTCCTGTCCCAGATCGAAAGCGATCGGCTGGCCCTTCTCCAG
GTCAGGAGGATTCTGCAGCAGCTGGGCCTGGACAGCACGTGTGAGGACA
GCATCGTGGTGAAGGAGGTGTGCGGAGCCGTGTCCCGGCGGGCGGCCCA
GCTCTGCGGTGCTGGCCTGGCCGCTATAGTGGAAAAAAGGAGAGAAGAC
CAGGGGCTAGAGCACCTGAGGATCACTGTGGGTGTGGACGGCACCCTGT
ACAAGCTGCACCCTCACTTTTCTAGAATATTGCAGGAAACTGTGAAGGA
ACTAGCCCCTCGATGTGATGTGACATTCATGCTGTCAGAAGATGGCAGT
GGAAAAGGGGCAGCACTGATCACTGCTGTGGCCAAGAGGTTACAGCAGG
CACAGAAGGAGAACTAGGAACCCCTGGGATTGGACCTGATGCATCTTGG
ATACTGAACAGCTTTTCCTCTGGCAGATCAGTTGGTCAGAGACCAATGG
GCACCCTCCTGGCTGACCTCACCTTCTGGATGGCCGAAAGAGAACCCCA
GGTTCTCGGGTACTCTTAGTATCTTGTACTGGATTTGCAGTGACATTAC
ATGACATCTCTATTTGGTATATTTGGGCCAAAATGGGCCAACTTATGAA
ATCAAAGTGTCTGTCCTGAGAGATCCCCTTTCAACACATTGTTCAGGTG
AGGCTTGAGCTGTCAATTCTCTATGGCTTTCAGTCTTGTGGCTGCGGGA
CTTGGAAATATATAGAATCTGCCCATGTGGCTGGCAGGCTGTTTCCCCA
TTGGGATGCTTAAGCCATCTCTTATAGGGGATTGGACCCTGTACTTGTG
GATGAACATTGGAGAGCAAGAGGAACTCACGTTATGAACTAGGGGGATC
TCATCTAACTTGTCCTTAACTTGCCATGTTGACTTCAAACCTGTTAAGA
GAACAAAGACTTTGAAGTATCCAGCCCCAGGGTGCAGAGAGGTTGATTG
CCAGGGAGCACTGCAGGAATCATTGCATGCTTAAAGCGAGTTATGTCAG
CACCCTGTAGGATTTTGTTCCTTATTAAGTGTGTGCCATGTGGTGGGGT
GCTGTCTGGGGCATCTGTTTTTCATTTTGCCTGTGGTTTGTGTTGCAGS
TGTTGATAGTTGTTTTAAGGATTGTTAGGTATAGGAAATCCAGTAAATT
AATAAAAAAATTTTGATTTTCCAATAAAAAAAAAAAAAAAAAA.

The human 50365 sequence (SEQ ID NO:20) is approximately 3669 nucleotides long. The nucleic acid sequence includes an initiation codon (ATG) and a termination codon (TAA) which are underscored and bolded above. The region between and inclusive of the initiation codon and the termination codon is a methionine-initiated coding sequence of about 2754 nucleotides (SEQ ID NO:22), including the termination codon. The coding sequence encodes a 917 amino acid protein (SEQ ID NO:21), which is recited as follows:

(SEQ ID NO: 21)
MFAVHLMAFYFSKLKEDQIKKVDRFLYHMRLSDDTLLDIMRRFRAEMEK
GLAKDTNPTAAVKMLPTFVRAIPDGSENGEFLSLDLGGSKFRVLKVQVA
EEGKRHVQMESQFYPTPNEIIRGNGIELFEYVADCLADFMKTKDLKHKK
LPLGLTFSFPCRQTKLEEGVLLSWTKKFKARGVQDTDVVSRLTKAMRRH
KDMDVDILALVNDTVGTMMTCAYDDPYCEVGVIIGTGTNACYMEDMSNI
DLVEGDEGRMCINTEWGAFGDDGALEDIRTEFDRELDLGSLNPGKQLFE
KMISGLYLGELVRLILLKMAKAGLLFGGEKSSALHTKGKIETRHVAAME
KYKEGLANTREILVDLGLEPSEADCIAVQHVCTIVSFRSANLCAAALAA
ILTRLRENKKVERLRTTVGMDGTLYKIHPQYPKRLHKVVRKLVPSCDVR
FLLSESGSTKGAAMVTAVASRVQAQRKQIDRVLALFQLTREQLVDVQAK
MRAELEYGLKKKSHGLATVRMLPTYVCGLPDGTEKGKFLALDLGGTNFR
VLLVKIRSGRRSVRMYNKIFAIPLEIMQGTGEELFDHIVQCIADFLDYM
GLKGASLPLGFTFSFPCRQMSIDKGTLIGWTKGFKATDCEGEDVVDMLR
EAIKRRNEFDLDIVAVVNDTVGTMMTCGYEDPNCEIGLIAGTGSNMCYM
EDMRNIEMVEGGEGKMCINTEWGGFGDNGCIDDIRTRYDTEVDEGSLNP
GKQRYEKMTSGMYLGEIVRQILIDLTKQGLLFRGQISERLRTRGIFETK
FLSQIESDRLALLQVRRILQQLGLDSTCEDSIVVKEVCGAVSRRAAQLC
GAGLAAIVEKRREDQGLEHLRITVGVDGTLYKLHPHFSRILQETVKELA
PRCDVTFMLSEDGSGKGAALITAVAKRLQQAQKEN.

Tissue Distribution of 50365 mRNA by TAQMAN™ Analysis

Endogenous human 50365 gene expression was determined using the Perkin-Elmer/ABI 7700 Sequence Detection System which employs TAQMAN™ technology.

To determine the level of 50365 in various human tissues a primer/probe set was designed. Total RNA was prepared from a series of human tissues using an RNEASY™ kit from Qiagen. First strand cDNA was prepared from 1 μg total RNA using an oligo-dT primer and SUPERSCRIPT II™ reverse transcriptase (Gibco/BRL). cDNA obtained from approximately 50 ng total RNA was used per TAQMAN™ reaction. Tissues tested include the human tissues and several cell lines shown in Tables 2 and 3 below. 50365 mRNA was detected in colon and liver tissue, and upregulated in colonic liver metastases (Table 2). In addition, 50365 mRNA was also detectable in adenomas and adenocarcinomas. 50365 expression was also found to a lesser extent in some lung tumor and ovary tumor tissues (Table 3).

TABLE 2

| Tissue Type | Expression |
|---|---|
| CHT 410 Colon Normal | 0.32 |
| CHT 425 Colon Normal | 0.41 |
| CHT 371 Colon Normal | 1.06 |
| PIT 281 Colon Normal | 0.00 |
| NDR 211 Colon Normal | 0.22 |
| CHT 122 Adenomas | 0.21 |
| CHT 887 Adenomas | 1.65 |
| CHT 414 Colonic Adenocarcinoma-B | 0.47 |
| CHT 841 Colonic Adenocarcinoma-B | 0.05 |
| CHT 890 Colonic Adenocarcinoma-B | 0.58 |
| CHT 910 Colonic Adenocarcinoma-B | 3.85 |
| CHT 377 Colonic Adenocarcinoma-B | 0.00 |
| CHT 520 Colonic Adenocarcinoma-C | 0.80 |
| CHT 596 Colonic Adenocarcinoma-C | 0.77 |
| CHT 907 Colonic Adenocarcinoma-C | 2.41 |
| CHT 372 Colonic Adenocarcinoma-C | 2.09 |
| NDR 210 Colonic Adenocarcinoma-C | 0.95 |
| CHT 1365 Colonic Adenocarcinoma-C | 2.54 |
| CLN 740 Liver Normal | 0.00 |
| CLN 741 Liver Normal | 0.00 |
| NDR 165 Liver Normal | 0.00 |
| NDR 150 Liver Normal | 0.14 |
| PIT 236 Liver Normal | 0.00 |
| CHT 1878 Liver Normal | 0.00 |
| CHT 119 Colon Liver Metastasis | 7.52 |
| CHT 131 Colon Liver Metastasis | 0.77 |
| CHT 218 Colon Liver Metastasis | 5.45 |
| CHT 739 Colon Liver Metastasis | 10.53 |
| CHT 755 Colon Liver Metastasis | 3.64 |
| CHT 215 Colon Abdominal Metastasis | 0.24 |
| PIT 337 Colon Normal | 0.29 |
| CHT 807 Colonic Adenocarcinoma-B | 61.64 |
| CHT 382 Colonic Adenocarcinoma-B | 57.11 |
| CHT 077 Colon Liver Metastasis | 180.49 |

The mRNA expression data for 50365 mRNA tabulated in Table 2 indicate that 50365 expression is upregulated in some adenomas and adenocarcinomas, and in most colonic liver metastases (see “Relative Expression” values). Relative expression in Table 2 is relative to expression of β2-macroglobulin.

TABLE 3

| Tissue Type | Expression |
|---|---|
| PIT 400 Breast Normal | 0.00 |
| PIT 372 Breast Normal | 0.00 |
| CHT 559 Breast Normal | 0.00 |
| MDA 236-Breast Tumor: PD-IDC(ILC?) | 0.00 |
| MDA 304 Breast Tumor: MD-IDC | 0.00 |
| CHT 2002 Breast Tumor: IDC | 0.00 |
| CHT 562 Breast Tumor: IDC | 0.00 |
| NDR 138 Breast Tumor ILC (LG) | 0.00 |
| CHT 1841 Lymph node (Breast Metastasis) | 0.00 |
| PIT 58 Lung (Breast Metastasis) | 0.00 |
| CHT 620 Ovary Normal | 0.00 |
| PIT 208 Ovary Normal | 0.00 |
| CLN 012 Ovary Tumor | 0.00 |
| CLN 07 Ovary Tumor | 0.05 |
| CLN 17 Ovary Tumor | 1.38 |
| MDA 25 Ovary Tumor | 0.00 |
| MDA 216 Ovary Tumor | 0.00 |
| PIT 298 Lung Normal | 0.00 |
| MDA 185 Lung Normal | 0.00 |
| CLN 930 Lung Normal | 0.00 |
| MPI 215 Lung Tumor--SmC | 0.00 |
| MDA 259 Lung Tumor-PDNSCCL | 0.00 |
| CHT 832 Lung Tumor-PDNSCCL | 0.97 |
| MDA 262 Lung Tumor-Small Cell Carcinoma | 0.00 |
| CHT 793 Lung Tumor-Adenocarcinoma | 0.03 |
| CHT 331 Lung Tumor-Adenocarcinoma | 0.00 |
| CHT 405 Colon Normal | 0.16 |
| CHT 523 Colon Normal | 0.65 |
| CHT 371 Colon Normal | 2.38 |
| CHT 382 Colon Tumor: MD | 0.88 |
| CHT 528 Colon Tumor: MD | 7.84 |
| CLN 609 Colon Tumor | 2.21 |
| NDR 210 Colon Tumor: MD-PD | 0.84 |
| CHT 340 Colon-Liver Metastasis | 3.23 |
| NDR 100 Colon-Liver Metastasis | 1.11 |
| PIT 260 Liver Normal (female) | 1.17 |
| CHT 1653 Cervix Squamous CC | 0.00 |
| CHT 569 Cervix Squamous CC | 0.00 |
| A24 HMVEC-Arr | 0.00 |
| C48 HMVEC-Prol | 0.00 |
| Pooled Hemangiomas | 0.00 |
| HCT116N22 Normal Ox.ygenation | 0.97 |
| HCT116H22 Hypoxic | 0.00 |

50365 mRNA was analyzed by TaqMan in a number of cell lines derived from normal and tumor cells (Table 3). Relative expression in Table 3 is relative to expression of β2-macroglobulin. Elevated 50365 mRNA expression levels were detected in some colon cell lines, e.g., normal colon, colon tumor; colonic liver metastases; some lung cell lines, e.g., lung tumor-PDNSCCL (poorly differentiated non-small cell carcinoma of the lung), lung tumor-adenocarcinoma; and an ovary tumor cell line. 50365 mRNA was also detected under normal oxygenation conditions.

### Human 21117

The human 21117 sequence (SEQ ID NO:25), which is approximately 3544 nucleotides long including untranslated regions, contains a predicted methionine-initiated coding sequence of about 1998 nucleotides (nucleotides 589 to 2586 of SEQ ID NO:25; SEQ ID NO:27). The coding sequence encodes a 665 amino acid protein (SEQ ID NO:26).

Human 21117 contains the following regions or other structural features: a dual specificity phosphatase catalytic domain (PF00782) located from about amino acid residue 158 to 297 of SEQ ID NO:26; a rhodanese-like domain (PF00581) located from about amino acid residue 11 to 131 of SEQ ID NO:26; and one tyrosine specific protein phosphatase active site (PS00383) at amino acids 242 to 254 of SEQ ID NO:26.

The 21117 protein additionally includes: six predicted N-glycosylation sites (PS00001) at amino acids 38 to 41, 49 to 52, 190 to 193, 212 to 215, 300 to 303, and 640 to 643 of SEQ ID NO:26; two predicted cAMP and cGMP-dependent protein kinase phosphorylation sites (PS00004) at amino acids 277 to 280 and 624 to 627 of SEQ ID NO:26; twelve predicted Protein Kinase C sites (PS00005) at about amino acids 12 to 14, 23 to 25, 72 to 74, 82 to 84, 393 to 395, 439 to 441; 473 to 475, 481 to 483, 486 to 488, 596 to 598, 604 to 606, and 609 to 611 of SEQ ID NO:26; thirteen casein kinase II phosphorylation sites (PS00006) at amino acids 21 to 24, 91 to 94, 214 to 217, 266 to 269, 369 to 372, 421 to 424, 434 to 437, 458 to 461, 508 to 511, 589 to 592, 612 to 615, 617 to 620, and 642 to 645 of SEQ ID NO:26; and seven predicted N-myristoylation sites (PS00008) from about amino acid 134 to 139, 247 to 252, 329 to 334, 382 to 387, 520 to 525, 574 to 579, and 650 to 655 of SEQ ID NO:26.

21117 polypeptides of the invention include 21117 fragments that include: all or part of a hydrophobic sequence e.g., all or part of the sequence from about residue 91 to about residue 106 of SEQ ID NO:26; and/or all or part of a hydrophilic sequence e.g., all or part of the sequence from about residue 592 to about residue 633 of SEQ ID NO:26. Other fragments include a cysteine residue or a glycosylation site.

### Human 38692

The human 38692 sequence (SEQ ID NO:28), which is approximately 1114 nucleotides long including untranslated regions, contains a predicted methionine-initiated coding sequence of about 672 nucleotides (nucleotides 89 to 760 of SEQ ID NO:28; SEQ ID NO:30). The coding sequence encodes a 223 amino acid protein (SEQ ID NO:29).

Human 38692 contains the following regions or other structural features: a dual specificity phosphatase catalytic domain (PF00782) located from about amino acid residue 28 to 173 of SEQ ID NO:29; one predicted Protein Kinase C phosphorylation site (PS00005) at about amino acids 201 to 203 of SEQ ID NO:29; one predicted casein kinase II phosphorylation site (PS0006) at amino acids 205 to 208 of SEQ ID NO:29; two predicted N-myristoylation sites (PS00008) from about amino acid 123 to 128 and 197 to 202 of SEQ ID NO:29; and two tyrosine kinase phosphorylation sites (PS00007) at amino acids 15 to 23 and 142 to 149 of SEQ ID NO:29.

38692 polypeptides of the invention include 38692 fragments that include: all or part of a hydrophobic sequence, e.g., all or part of the sequence from about residue 31 to about residue 41 of SEQ ID NO:29; and/or all or part of a hydrophilic fragment e.g., all or part of the sequence from about residue 200 to about residue 212 of SEQ ID NO:29. Other fragments include cysteine residues.

### 21117 and 38692 Dual Specificity Phosphatase Proteins

The intracellular phosphorylation of proteins is critical for a plethora of regulatory and signalling pathways in eukaryotic cells. Phosphorylation events can govern a wide range of cellular processes, including cell proliferation, differentiation, transcription, and morphology. An essential component of these signalling pathways is the ability of the cell to desensitize, recycle, and counteract phosphorylation signals. The cell primarily utilizes enzymes, termed phosphatases, which remove the phosphate on tyrosine, serine, and threonine side chains. The protein phosphatases are divided into three groups according to catalytic function: (1) protein phosphatases that dephosphorylate serine and threonine residues; (2) protein phosphatases which dephosphorylate tyrosine residues; and (3) dual specificity protein phosphatases which dephosphorylate serine, threonine and tyrosine residues.

Serine/threonine protein phosphatases are associated with the regulation of cholesterol biosynthesis, glycogen metabolism, muscle contractility, calcium ion channels, protein synthesis, regulation of the G2 to M transition of the cell cycle, regulation of glycolysis (6-phosphofructo-2-kinase and pyruvate kinase), glycogenolysis (phosphorylase kinase subunit), gluconeogenesis (fructose-2,6-bisphosphatase and pyruvate kinase), amino-acid degradation (phenylalanine hydroxylase), lipid metabolism (acetyl-CoA carboxylase), catecholamine synthesis (tyrosine hydroxylase) and protein synthesis (elongation factor 2).

Protein tyrosine phosphatases (PTPs) are a family of intracellular and integral membrane phosphatases that dephosphorylate tyrosine residues in proteins. PTPs have been identified in mammals, Drosophila and Schiz. pombe and are implicated in the control of normal and neoplastic growth and proliferation. They have also been found encoded by plasmids in bacteria of the genus Yersinia, where they are implicated in pathogenicity.

Dual specificity phosphatases hydrolyze phosphotyrosine, phosphothreonine, and phosphoserine residues (for a review, see, e.g., Fauman and Saper (1996) Trends in Biochem. 21:412). This class of proteins is exemplified by the VH1 or vaccinia virus late H1 gene protein, whose catalytic activity is required for vaccinia virus replication. A human homolog of VH1, VHR, has also been identified. VH1-like dual specificity phosphatase can also include the phosphatases PAC-1 and CL100/MKP-1, hVH-2/MKP-2, hVH-3, MKP-3, MKP-X, MKP-4, hVH-5, and M3/6 proteins. The PAC-1 and CL100 proteins hydrolyze phosphothreonine and phosphotyrosine residues on phosphorylated MAP (mitogen activated protein) kinases. In order to modulate signalling events, the activity and expression of dual specificity phosphatases can be finely regulated. For example, the PAC-1 and CL100 phosphatase can be induced by growth factors (Keyse, S (1995) Biochim. Biophys. Acta 1265:152-160).

The 21117 and 38692 proteins contain a significant number of structural characteristics in common with members of the dual specificity phosphatase family.

Dual specificity phosphatase proteins are characterized by a common fold. Examples of members of the dual specificity phosphatase family include MAP kinase phosphatase-1 (MKP-1), which dephosphorylates MAP kinase on both threonine and tyrosine residues and a human, vaccinia H1-related phosphatase (VHR), which also removes the phosphate from phosphothreonine and phosphotyrosine residues. Dual specificity phosphatases are exemplified by the VH1 or vaccinia virus late H1 gene protein, which hydrolyzes both phosphotyrosine, phosphothreonine, and phosphoserine. VH1 catalytic activity is required for viral replication. A human homolog of VH1, VHR, has been identified. The three dimensional structure of this family is based on models from x-ray crystallographic data of protein tyrosine phosphatases, and human VHR. The VHR structure includes a core domain consisting of a five-stranded mixed β-sheet and six α-helices. This structure closely superimposes on the structure of phosphotyrosine protein phosphatases. However, dual specificity phosphatases lack the KNRY motif, and the N-terminal structures of tyrosine protein phosphatases which endow these enzymes with a deep active site specific for aryl phosphates. Thus, dual specificity phosphatases have a shallower active site relative to tyrosine protein phosphatases and can accommodate phosphoserine and phosphothreonine substrates. Even so dual specificity phosphatases can have a greater than 50-fold faster rate of phosphatase activity for phosphotyrosine substrates than phosphothreonine or phosphoserine substrates.

Similar to the broader class of phosphatases, dual specificity phosphatases have a highly conserved active site including three catalytic residues, a cysteine, an arginine, and an aspartic acid. The active site cysteine and arginine are found in the “C-X₅-R” motif of the tyrosine phosphatase signature (Prosite PS00383). This motif forms a binding pocket for three of the phosphate oxyanions. The cysteine acts as a nucleophile to accept the PO₃ group. The reaction transiently generates a phospho-cysteine intermediate before the phosphate is transferred to water. The active site arginine stabilizes the transition-state by hydrogen bonding to phosphate oxygens. In addition the histidine preceding the active site cysteine and the serine or threonine following the active site arginine are responsible for lowering the pKₐ of the cysteine to stabilize a negative charge on the cysteine. The active site aspartic acid accelerates the reaction by donating a protein to generate an uncharged hydroxyl (for a review, see Fauman and Saper (1996) Trends in Biochem. 21:412). A C-X₅-R motif is found in the 21117 protein at about amino acids 242 to 254 of SEQ ID NO:26.

The 21117 and 38692 proteins of the present invention show significant homology to members of the dual specificity phosphatase family. Dual specificity phosphatases are known to play critical roles in growth factor signaling. For example, vaccinia H1-related (VHR)-like phosphatases are known to dephosphorylate growth factor receptors and thereby eliminate their signaling. MAP-kinase phosphatases terminate MAP-kinase activity, thus leading to inhibition of growth factor-mediated mitogenic signaling. Thus, dual specificity phosphatases play a key role in inhibiting proliferation and stimulating the differentiation of cells. As the 21117 and 38692 proteins show homology to dual specificity phosphatases, these proteins are likely to be involved in modulating (e.g., inhibiting) the proliferation and (e.g., stimulating) the differentiation of the cells in which they are expressed, e.g., hematopoietic cells such as eythroid cells, myeloid cells, monocytes, or megakaryocytes. Accordingly, the 21117 and 38692 molecules of the invention may be useful for developing novel diagnostic and therapeutic agents for 21117 and 38692-mediated or related disorders, as described below.

A 21117 or 38692 polypeptide of the invention can include a “dual specificity phosphatase catalytic domain” or regions homologous with a “dual specificity phosphatase catalytic domain”. As used herein, the term “dual specificity phosphatase catalytic domain” refers to an amino acid sequence having about 50 to 250, preferably about 100 to 200, more preferably about 120 to 160 amino acid residues and having a bit score for the alignment of the sequence to the dual specificity phosphatase domain (HMM) of at least 50, preferably 100, more preferably 120, 200, or more. The dual specificity phosphatase catalytic domain (HMM) has been assigned the PFAM Accession Number PF00782.

A dual specificity phosphatase domain preferably includes the conserved active site residues cysteine and arginine in a C-X₅-R motif found at about amino acids 242 to 254 of SEQ ID NO:26 (the 21117 protein). Preferably, a dual specificity phosphatase domain includes a conserved general amino acid, e.g., aspartic acid. For example, a 21117 protein has an aspartic acid located at about residue 213 of SEQ ID NO:26 and a 38692 protein has an aspartic acid located at about residue 89 of SEQ ID NO:29. Typically, dual specificity phosphatases are able to dephosphorylate tyrosine residues and serine/threonine residues.

In a preferred embodiment, a 21117 or 38692 polypeptide or protein has a “dual specificity phosphatase catalytic domain” or a region that includes at least about 50 to 250, preferably about 100 to 200, more preferably about 120 to 160, and even more preferably about 130 to 150 amino acid residues and has at least about 70% 80% 90% 95%, 99%, or 100% homology with a “dual specificity phosphatase catalytic domain,” e.g., the dual specificity phosphatase catalytic domain of human 21117 (e.g., residues 158 to 297 of SEQ ID NO:26) or 38692 (e.g., residues 28 to 173 of SEQ ID NO:29).

To identify the presence of a “dual specificity phosphatase catalytic domain” in a 21117 or 38692 protein sequence and to make the determination that a polypeptide or protein of interest has a particular profile, the amino acid sequence of the protein can be searched against a database of HMMs (e.g., the Pfam database, release 2.1) using default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a “dual specificity phosphatase catalytic domain” e.g., the dual specificity phosphatase catalytic domain of human 21117 (amino acids 158 to 297 of SEQ ID NO:26) or human 38692 (amino acids 28 to 173 of SEQ ID NO:29). The identified dual specificity phosphatase catalytic domain consensus sequences of human 21117 and of human 38692 are set forth in SEQ ID NO:31 and 32, respectively.

Human 21117 also contains a “rhodanese-like” domain (PF00581) from about amino acid 11 to 131 of SEQ ID NO:26. The rhodanese-like domain is occasionally found in a single copy in phosphatases, such as Cdc25 phosphatase, a dual-specificity phosphatase. Rhodanese is about 300 amino acids in length and has a conserved domain at the N-terminus and at the C-terminus. A cysteine residue is part of the active site of the enzyme. In a preferred embodiment, a 21117 polypeptide or protein has a “rhodanese-like domain” or a region that includes at least about 80 to 300 amino acids, preferably about 100 to 150 amino acid residues, and has at least about 70% 80% 90% 95%, 99%, or 100% homology with a “rhodanese-like domain,” e.g., the rhodanese-like domain of human 21117 (e.g., residues 11 to 131 of SEQ ID NO:26). A search was performed against the HMM database resulting in the identification of two “rhodanese-like domains” in human 21117 (amino acids 11 to 131 and amino acids 12 to 134 of SEQ ID NO:26). The identified rhodanese-like domain consensus sequences of human 21117 are set forth in SEQ ID NO:33 and 34.

As used herein, a “21117 or 38692 activity”, “biological activity of 21117 or 38692” or “functional activity of 21117 or 38692”, refers to an activity exerted by a 21117 or 38692 protein, polypeptide or nucleic acid molecule on e.g., a 21117 or 38692-responsive cell or on a 21117 or 38692 substrate, e.g., a protein substrate, as determined in vivo or in vitro. In one embodiment, an 21117 or 38692 activity is a direct activity, such as an association with a 21117 or 38692 target molecule. A “target molecule” or “binding partner” is a molecule with which a 21117 or 38692 protein binds or interacts in nature. A 21117 or 38692 activity can also be an indirect activity, e.g., a cellular signaling activity mediated by interaction of the 21117 or 38692 protein with an 21117 or 38692 receptor.

Based on the above-described sequence similarities, the 21117 or 38692 molecules of the present invention are predicted to have similar biological activities as dual specificity phosphatase family members, probably of the VHR-type. Since VHR-phosphatases inhibit growth factor signaling by dephosphorylating, e.g., growth factor receptors, the 21117 or 38692 molecules of the invention are predicted to have one or more of the following activities: (1) catalyze the removal of a phosphate group attached to a tyrosine residue in a protein target, e.g., a growth factor receptor; (2) catalyze the removal of a phosphate group attached to a serine or threonine residue in a protein e.g., a growth factor receptor; (3) modulate growth factor activity; (4) modulate an intracellular signaling pathway, e.g., a MAP kinase or ERK kinase pathway; (5) modulate (e.g., stimulate) cell differentiation, e.g., differentiation of a 38692- or a 21117-expressing cell, e.g., a breast, colon, lung, or adipose cell, a bone cell, an endothelial cell, a liver cell, or a hematopoietic cell (e.g., a myeloid (neutrophil) cell, a monocyte, an erythroid cell, a bone marrow cell, a CD34-expressing cell, a megakaryocyte); (6) stimulate hematopoiesis; (7) modulate cell proliferation, e.g., proliferation of a 38692- or a 21117-expressing cell, e.g., a breast, colon, lung, or adipose cell, a bone cell, an endothelial cell, a liver cell, or a hematopoietic cell (e.g., a myeloid (neutrophil) cell, a monocyte, an erythroid cell, a bone marrow cell, a CD34-expressing cell, a megakaryocyte); (8) inactivate cell surface growth factor receptors, e.g., tyrosine kinase receptors; or (9) modulate apoptosis, of a cell, e.g., a cancer cell, e.g., a leukemic cell.

As assessed by TAQMAN™ analysis described herein, 38692 mRNA is expressed in hematopoietic cells, and in particular, in erythroid cell lineages, therefore the molecules of the invention can be used to develop novel agents or compounds to treat and/or diagnose disorders involving aberrant activities of those cells e.g., hematopoietic, and in particular, erythroid disorders, as described below. For example, 38692 polypeptide is expressed in megakaryocytes, fetal liver CD34+ cells, erythroid progenitor cells (e.g., bone marrow glycophorin A positive cells (BM GPA+)), and Bone Marrow Glycophorin A (BM GPA) low CD71+.

As used herein, a “CD34-positive cell” refers to a cell that expresses detectable levels of the CD34 antigen, preferably human CD34 antigen. The sequence for human CD34 is provided in SwissProt Accession Number P28906. The CD34 antigen is typically present on immature hematopoietic precursor cells and hematopoietic colony-forming cells in the bone marrow, including unipotent (CFU-GM, BFU-E) and pluripotent progenitors (CFU-GEMM, CFU-Mix and CFU-blast). The CD34 is also expressed on stromal cell precursors. Terminal deoxynucleotidyl transferase (TdT)-positive B- and T-lymphoid precursors in normal bone also are CD34+. The CD34 antigen is typically present on early myeloid cells that express the CD33 antigen, but lack the CD14 and CD15 antigens and on early erythroid cells that express the CD71 antigen and dimly express the CD45 antigen. The CD34 antigen is also found on capillary endothelial cells and approximately 1% of human thymocytes. Normal peripheral blood lymphocytes, monocytes, granulocytes and platelets do not express the CD34 antigen. CD34 antigen density is highest on early haematopoietic progenitor cells and decreases as the cells mature. The antigen is undetectably on fully differentiated haematopoietic cells. Approximately 60% of acute B-lymphoid leukemia's and acute myeloid leukemia express the CD34 antigen. The antigen is not expressed on chronic lymphoid leukemia (B or T lineage) or lymphomas.

As the 38692 polypeptides of the invention may modulate 38692-mediated activities, they may be useful as of for developing novel diagnostic and therapeutic agents for 38692-mediated or related disorders, e.g., hematopoietic related disorders, or erythroid-associated disorders. As assessed by TAQMAN™ analysis, 38692 is expressed at high levels in fetal liver, HepG2.2.15-A liver cells, and Hep3B hypoxia cells, therefore the molecules of the invention can be used to develop novel agents or compounds to treat and/or diagnose liver related disorders.

Further TAQMAN™ analyses have demonstrated that 21117 mRNA is expressed in normal breast, normal colon, normal adipose tissue, prostate tumor and lung chronic obstructive pulmonary disorder (COPD) tissue. Thus, diagnostic and therapeutic methods of using the 21117 molecules of the invention to treat/diagnose breast, colon, adipose, prostate, and lung disorders are also contemplated by the present invention.

Identification and Characterization of Human 38692 and 21117 cDNA

The human 21117 sequence (SEQ ID NO:25), which is approximately 3544 nucleotides long including untranslated regions, contains a predicted methionine-initiated coding sequence of about 1998 nucleotides (nucleotides 589 to 2586 of SEQ ID NO:25; SEQ ID NO:27). The coding sequence encodes a 666 amino acid protein (SEQ ID NO:26).

The human 38692 sequence (SEQ ID NO:28), which is approximately 1114 nucleotides long including untranslated regions, contains a predicted methionine-initiated coding sequence of about 672 nucleotides (nucleotides 89 to 760 of SEQ ID NO:28; SEQ ID NO:30). The coding sequence encodes a 224 amino acid protein (SEQ ID NO:29).

Tissue Distribution of 38692 or 21117 mRNA

Endogenous human 21117 and 38692 gene expression was determined using the Perkin-Elmer/ABI 7700 Sequence Detection System which employs TAQMAN™ technology.

To determine the level of 21117 mRNA in various human tissues a primer/probe set was designed using Primer Express (Perkin-Elmer) software and primary cDNA sequence information. Total RNA was prepared from a series of tissues using an RNEASY™ kit from Qiagen. First strand cDNA was prepared from 1 μg total RNA using an oligo-dT primer and SUPERSCRIPT II™ reverse transcriptase (Gibco/BRL). cDNA obtained from approximately 50 ng total RNA was used per TAQMAN™ reaction.

21117 mRNA levels were analyzed in a variety of tissue samples, both normal and diseased, including (1) Aorta/normal; (2) Fetal heart/normal; (3) Heart normal; (4) Heart/Coronary heart failure (CHF); (5) Vein/Normal; (6) SMC (Aortic); (7) Spinal cord/Normal; (8) Brain cortex/Normal; (9) Brain hypothalamus/Normal; (10) Glial cells (Astrocytes); (11) Brain/Glioblastoma; (12) Breast/Normal; (13) Breast tumor/IDC; (14) ovary/Normal; (15) ovary/Tumor; (16) Pancreas; (17) Prostate/Normal; (18) Prostate/Tumr; (19) Colon/normal; (20) Colon/tumor; (21) Colon/IBD (inflammatory bowel disease); (22) Kidney/normal; (23) Liver/normal; (24) Liver fibrosis; (25) Fetal Liver/normal; (26) Lung/normal; (27) Lung/tumor; (28) Lung/chronic obstructive pulmonary disease (COPD); (29) Spleen/normal; (30) Tonsil/normal; (31) Lymph node/normal; (32) Thymus/normal; (33) Epithelial Cells (prostate); (34) Endothelial Cells (aortic); (35) Skeletal Muscle/Normal; (36) Fibroblasts (Dermal); (37) Skin/normal; (38) Adipose/Normal; (39) Osteoblasts(primary); (40) Osteoblasts (undifferentiated); (41) Osteoblasts (differentiated); (42) Osteoclasts; (43) Aortic Smooth Muscle Cells (SMC) Early; (44) Aortic SMC Late; (45) Osteoclasts (undiff); (46) shear human umbilical vein endothelial cells (HUVEC). High relative levels of expression were detected in normal colon, normal breast, chronic obstructive pulmonary disease lung tissue, normal adipose tissue, and undifferentiated osteoblasts.

38692 expression was determined by TAQMAN™ assays on mRNA derived from various tissues and cell lines, including (1) Lung; (2) Kidney; (3) Spleen; (4) Fetal Liver; (5) Granulocytes; (6) NHDF resting; (7) NHDF/TGF-treated for 48 hr; (8) NHLF/CTN-treated for 48 hr; (9) NHLF/TGF-treated for 48 hr; (10) NC Heps; (11) Passage Stellates; (12) Liver Pool; (13) LF/CHT 339; (14) LF/NDR 191; (15) LF/NDR 079; (16) Lymph Nodes NDR 173; (17) Tonsils; (18) TH124 hr; (19) CD4; (20) CD14 Resting; (21) CD19; (22) CD3 Resting; (23) bone marrow mononuclear cells (BM MNC) LP26; (24) mPB CD34+; (25) adult bone marrow (ABM) CD34+; (26) Cord Blood CD34+; (27) Erythroid; (28) Megakaryocytes LP16; (29) Neutrophils d14; (30) NBM CD15+/CD14−/34+; (31) mBM CD15+/CD11b−; (32) BM/glycophorin A (GPA); (33) Hepatocyte (Hep)G2-A; (34) HepG2.2.15-A; (35) HBV-Liver MAI-1; (36) HL60; (37) leukemia cell line K562; (38) Molt 4; (39) liver cell line Hep3B Nor; (40)Hep3B Hypoxia, as well as various hematopoietic cell lines, including, (1) Lung; (2) Colon; (3) Heart; (4) Spleen; (5) Kidney; (6) Liver NDR 200; (7) Fetal Liver; (8) Skeletal Muscle; (9) m BM (bone marrow) mononuclear cells (MNC); (10) mBM MNC LP7; (11) mBM CD34+ LP92; (12) mobilized peripheral blood (mPB) CD34+ LF41; (13) mPB CD34+ LF48; (14) adult bone marrow (ABM) CD34+ LP91; (15) ABM CD34+ LP29; (16) Cord Blood CD34+ LF109; (17) Fetal Liver CD34+ LP93; (18) Fetal Liver CD34+ LP45; (19) Bone Marrow Glycophorin A positive (BM GPA+) LP85; (20) BM GPA+ LP34-1; (21) BM GPA low CD71+ LF38; (22) BM GPA low CD71+ LP85-2; (23) mobilized peripheral blood (mPB) CD41+/CD14− LP94; (24) BM CD41+/C D14− LP78; (25) mBM CD15+ LP15; (26) mBM CD15+/CD11b− LP7-4; (27) mBM CD15+/CD11b+ LP15-2; (28) BM CD15+/CD11b− LF80-4; (29) BM CD15+/CD11b− LP23-2; (30) BM CD15+/CD34− LP27-2; (31) BM CD15+/CD34− LP41-1; (32) Erythrocyte (Ery) d6 LP25-1; (33) Ery d6 LP3′-1; (34) Ery d10 LP24-4; (35) Ery d12 LF24-8; (36) Ery d12 LF24-9; (37) Ery d14 GPA+ LP31-4; (38) Ery d14 CD36+ LP31-7; (39) Megakaryocyte (Meg) 24 hr LF23-2; (40) Meg 44 hr LF6-2; (41) Meg d7 LP31-2; (42) Meg d12 LF26; (43) Meg d14 LP31-5; (44) Neutrophil d4 LF30; (45) Neutrophil d6 LF26; (46) Neutrophil d6 LP27; (47) Neutrophil d7 LP31-3; (48) Neutrophil d12 LP27; (49) Neutrophil d12 LP26B; (50) Neutroph d14 LP31-6. In some samples, mRNA expression was detected at the indicated times in culture (e.g., 24 hrs., 48 hrs., days in culture).

High relative levels of 38692 expression were found in hepatic tissues (e.g., fetal liver cells, HepG2.2.15-A liver cells, fetal liver CD34+ cells), and in hematopoietic cells such as K562 cells, Bone Marrow Glycophorin A (BM GPA) low CD71+ LF38; and BM GPA low CD71+ LP85-2.

### Human 46508

The human 46508 sequence (SEQ ID NO:35), which is approximately 1182 nucleotides long including untranslated regions, contains a predicted methionine-initiated coding sequence of about 684 nucleotides, including the termination codon. The coding sequence encodes a 227 amino acid protein (SEQ ID NO:36).

Human 46508 contains the following regions or other structural features: a peptidyl-tRNA hydrolase domain (PFAM Accession PF01195) located at about amino acid residues 44 to 221 of SEQ ID NO:36; two Protein Kinase C sites (PS00005) at about amino acids 13 to 15, and 150 to 152 of SEQ ID NO:36; two Casein Kinase II sites (PS00006) located at about amino acids 125 to 128, and 194 to 197 of SEQ ID NO:36; seven N-myristoylation sites (PS00008) located at about amino acids 4 to 9, 17 to 22, 23 to 28, 53 to 58, 74 to 79, 149 to 154, and 156 to 161 of SEQ ID NO:36; one amidation site (PS00009) located at about amino acid 40 to 43 of SEQ ID NO:36; and one glycosaminoglycan attachment site (PS00002) located at about amino acids 3 to 6 of SEQ ID NO:36.

For general information regarding PFAM identifiers, PS prefix and PF prefix domain identification numbers, refer to Sonnhammer et al. (1997) Protein 28:405-420.

The 46508 protein contains a significant number of structural characteristics in common with members of the peptidyl-tRNA hydrolase family.

Peptidyl-tRNA hydrolases are a family of important enzymes which hydrolyze ester linkages between the peptide moiety and the tRNA of peptidyl-tRNAs (Kössel, H (1969) Biochim. Biophys. Acta 204:191-202; Garcia-Villegas, M. R. (1991) EMBO J. 10:3549-3555). The esterase activity of peptidyl-tRNA hydrolases cleaves the covalent bond between the nascent peptide and the tRNA. Such cleavage results in the recycling of tRNA. The peptidyl-tRNA hydrolase from E. coli is well characterized, and homologous proteins are found in many eubacterial species. In E. coli, the gene encoding peptidyl-tRNA hydrolase is essential. Further, the required level of peptidyl-tRNA hydrolase activity for viability is escalated under conditions that increase premature translational termination such as exposure to antibiotics (Menninger and Coleman (1993) Antimicrob. Agents Chemother. 37:2027-2029.) and reduced when tRNAs particularly prone to dissociate from the ribosome are supplied in excess (Heurgue-Hamard et al. (1996) EMBO J. 15:2826-2833).

The x-ray crystal structure of E. coli peptidyl-tRNA hydrolase was determined at high resolution (Schmitt et al. (1997) EMBO J. 16:4760-4769). The monomeric protein contains single monomeric α/β globular domain of seven α-strands and six α-helices. The peptidyl-tRNA hydrolase enzyme structure has structural similarity to an aminopeptidase from Aeromonas proteolytica (GenPept:640150) (Chevrier, B. et al. (1994) Structure 2:283-291) and to a lesser extent to bovine purine nucleoside phosphorylase (GenPept:2624420) (Koellner, G. et al. (1997) J. Mol. Biol. 265:202-216.). Genetic data and structural analysis indicate that three residues, asparagine 10, histidine 20, and aspartic acid 93 in the E. coli enzyme are critical residues for catalysis. In addition, asparagine 68 and asparagine 114 of the E. coli enzyme are poised to make favorable electrostatic contacts with the peptide region of the peptidyl-tRNA substrate whereas arginine 133 of the E. coli sequence may contact the tRNA portion of the substrate. The amino acid identities of these positions are conserved in alignments of eubacterial peptidyl-tRNA transferases.

The 46508 polypeptide (SEQ ID NO:36) has the three conserved residues important for catalysis, namely: an asparagine at position 51, a histidine at position 59, and an aspartic acid at position 134 of SEQ ID NO:36. In addition, conserved asparagines and a conserved arginine, residues 68, 114, and 133 of the E. coli peptidyl-tRNA hydrolase, respectively, contribute to the specificity of substrate recognition. The 46508 polypeptide (SEQ ID NO:36) also has these three conserved residues, namely, an asparagine at position 109, an asparagine at position 155, and an arginine at position 173 of SEQ ID NO:36.

Cells which utilize the translation machinery more intensely than quiescent cells, e.g. rapidly growing cells, environmentally stressed cells, and virally infected cells, are likely to produce more peptidyl-tRNA substrates. Further, because of the increased translation activity, such cells also require larger tRNA pools than quiescent cells either in their cytoplasm or mitochondria, or both. Accordingly the activity of peptidyl-tRNA hydrolase enzymes may be required by such cells. Thus, inhibition of 46508 activity might be a successful route to treatment of a variety of disorders, including but not limited to, cell proliferation, cell differentiation, viral infection, and metabolism.

A 46508 polypeptide can include a “peptidyl-tRNA hydrolase domain” or regions homologous with a “peptidyl-tRNA hydrolase domain”. A 46508 polypeptide can optionally further include at least one glycosaminoglycan attachment site; at least one, preferably two, protein kinase C phosphorylation sites; at least one, preferable two, casein kinase II phosphorylation sites; at least one, two, three, four, five, six, preferably seven, N-myristoylation sites; and at least one amidation site.

As used herein, the term “peptidyl-tRNA hydrolase domain” includes an amino acid sequence of about 160 to 240 amino acid residues in length and having a bit score for the alignment of the sequence to the peptidyl-tRNA hydrolase domain profile (Pfam HMM) of at least 80. Preferably, the peptidyl-tRNA hydrolase domain has an amino acid sequence of about 170 to about 200 amino acids, more preferable about 170 to 190 amino acids, or about 177 amino acids, and has a bit score for the alignment of the sequence to the peptidyl-tRNA hydrolase domain (HMM) of at least 100, preferably of at least 120, more preferably of at least 130 or greater. Preferably, the peptidyl-tRNA hydrolase domain further includes the following highly conserved residues: one, preferably two, more preferably three asparagine residues, a histidine residue, an aspartic acid, and an arginine corresponding respectively to asparagine 51, asparagine 109, asparagine 155, histidine 59, aspartic acid 134, and arginine 173 of SEQ ID NO:36. The peptidyl-tRNA hydrolase domain (HMM) has been assigned the PFAM Accession.

In a preferred embodiment 46508 polypeptide or protein has a “peptidyl-tRNA hydrolase domain” or a region which includes at least about 120 to about 200 amino acids, more preferably about 160 to 190, 170 to 180, or about 177 amino acid residues and has at least about 60%, 70% 80% 90% 95%, 99%, or 100% homology with a “peptidyl-tRNA hydrolase domain,” e.g., the peptidyl-tRNA hydrolase domain of human 46508 (e.g., residues 44 to 221 of SEQ ID NO:36).

To identify the presence of a “peptidyl-tRNA hydrolase” domain in a 46508 protein sequence, and make the determination that a polypeptide or protein of interest has a particular profile, the amino acid sequence of the protein can be searched against the Pfam database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a “peptidyl-tRNA hydrolase” domain in the amino acid sequence of human 46508 at about residues 44 to about 221 of SEQ ID NO:36. The identified peptidyl-tRNA hydrolase domain consensus sequence is set forth as SEQ ID NO:38.

A 46508 family member can include at least one peptidyl-tRNA hydrolase domain or regions homologous with a peptidyl-tRNA hydrolase domain. Furthermore, a 46508 family member can include at least one, preferably two, protein kinase C phosphorylation sites (PS00005); at least one, preferable two, casein kinase II phosphorylation sites (PS00006); at least one, two, three, four, five, six, preferably seven, N-myristoylation sites; and at least one amidation site.

46508 polypeptides of the invention include fragments which include: all or part of a hydrophobic sequence, e.g., the sequence from about amino acid 60 to 70, from about 86 to 102, and from about 189 to 195 of SEQ ID NO:36; all or part of a hydrophilic sequence, e.g., the sequence of from about amino acid 77 to 85, from about 217 to 224 of SEQ ID NO:36, a sequence which includes a Cys, or a glycosylation site, of SEQ ID NO:36.

As the 46508 polypeptides of the invention may modulate 46508-mediated activities, they may be useful as of for developing novel diagnostic and therapeutic agents for 46508-mediated or related disorders, as described below.

As used herein, a “46508 activity”, “biological activity of 46508” or “functional activity of 46508”, refers to an activity exerted by a 46508 protein, polypeptide or nucleic acid molecule. For example, a 46508 activity can be an activity exerted by 46508 in a physiological milieu on, e.g., a 46508-responsive cell or on a 46508 substrate, e.g., a protein substrate. A 46508 activity can be determined in vivo or in vitro. In one embodiment, a 46508 activity is a direct activity, such as an association with a 46508 target molecule. A “target molecule” or “binding partner” is a molecule with which a 46508 protein binds or interacts in nature. In an other embodiment, 46508 activity can also be an indirect activity, e.g. a cellular signaling activity mediated by interaction of the 46508 protein with a second protein or with a nucleic acid.

The features of the 46508 molecules of the present invention can provide similar biological activities as peptidyl-tRNA hydrolase family members. For example, the 46508 proteins of the present invention can have one or more of the following activities: (1) ability to bind tRNA; (2) ability to bind peptide fragments; (3) ability to bind peptidyl-tRNAs; (4) ability to hydrolyze covalent bond between peptide and tRNA within peptidyl-tRNAs; or (5) ability to modulate translational efficiency. The 46508 polypeptide may perform one or more of these properties in the milieu of the cell cytoplasm and/or of the cell mitochondria.

As shown below, increased 46508 mRNA expression is detected in a variety of malignant and non-malignant tissues, including cardiovascular tissues (e.g., endothelial cells, coronary smooth muscle cells), pancreas, neural tissues (e.g., brain, hypothalamus, DRG), skin, immune, e.g., erythroid cells, as well as a number of primary and metastatic tumors, e.g., ovarian, breast, prostate and lung tumors. Thus, the 46508 molecules can act as novel diagnostic targets and therapeutic agents for controlling disorders of involving aberrant activity of those cells, e.g., cell proliferative disorders (e.g., cancer), cardiovascular disorders, neurological disorders, pain disorders, pancreatic disorders, breast disorders, colon disorders, ovarian disorders, lung disorders, skin and immune, e.g., erythroid, disorders.

High transcriptional expression of 46508 was observed in tumor samples compared to normal organ control samples. For example, high expression was observed in 5/5 primary ovarian tumor samples, 4/4 primary colon tumor samples, 2/2 colon to liver metastases, and 3/6 primary lung tumor samples. Additionally, high expression was observed in proliferating HMVEC cells when compared to arrested HMVEC cells. Therefore, 46508 may mediate or be involved in cellular proliferative and/or differentiative disorders.

Moderate expression of 46508 was observed in normal heart tissue samples, and in both normal and tumor breast tissue samples, and thus 46508 may mediate disorders involving the heart, e.g. cardiovascular disorders; and it may mediate disorders of the breast, e.g. breast disorders. High to moderate expression of 46508 was also observed in normal pancreas tissue, in the skin and in the normal brain cortex and hypothalamus, therefore, 46508 may mediate disorders involving the pancreas, e.g. pancreatic disorders; it may mediate disorders involving the skin, e.g., skin disorders; and it may mediate disorders involving the brain cortex or hypothalamus, e.g. disorders of the brain.

46508 mRNA expression was also detected in the dorsal root ganglia (DRG). Therefore, 46508-associated disorders can detrimentally affect regulation and modulation of the pain response; and vasoconstriction, inflammatory response and pain therefrom. Examples of such disorders in which the 46508 molecules of the invention may be directly or indirectly involved include pain, pain syndromes, and inflammatory disorders, including inflammatory pain.

Normal and tumorous samples of ovarian tissue also showed expression of 46508 mRNA. Various data indicates that 46508 is highly expressed in several ovarian cell lines, including SKOV3/Var, A2780, MDA 2774 and ES-2. Thus 46508 may mediate diseases involving the ovary.

Moderate expression of 46508 mRNA was also noted in normal colon tissue, in normal and tumor prostate samples, in fibrotic liver tissue samples, in both normal and lung tumor samples and high expression was noted in colon tumor samples. Thus 46508 may mediate diseases involving the colon, e.g. colon disorders; it may mediate diseases involving the prostate, e.g. prostate disorders; it may mediate diseases involving the liver, e.g. liver disorders; and it may mediate diseases involving the lung, e.g. lung disorders;

Identification and Characterization of Human 46508 cDNA

The human 46508 sequence (SEQ ID NO:35), which is approximately 1180 nucleotides long, including untranslated regions, contains a predicted methionine-initiated coding sequence of about 684 nucleotides, including the termination codon (nucleotides indicated as “coding” of SEQ ID NO:35; SEQ ID NO:37). The coding sequence encodes a 227 amino acid protein (SEQ ID NO:36).

Tissue Distribution of 46508 mRNA by TAQMAN™ Analysis

Endogenous human 46508 gene expression was determined using the Perkin-Elmer/ABI 7700 Sequence Detection System which employs TAQMAN™ technology.

To determine the level of 46508 in various human tissues a primer/probe set was designed. Total RNA was prepared from a series of human tissues using an RNEASY™ kit from Qiagen. First strand cDNA was prepared from 1 μg total RNA using an oligo-dT primer and SUPERSCRIPT II™ reverse transcriptase (Gibco/BRL). cDNA obtained from approximately 50 ng total RNA was used per TAQMAN™ reaction. Tissues tested include the human tissues and several cell lines shown in Tables 4, 5 and 6.

Table 4 below shows expression of 46508 mRNA in various normal and diseased tissues, detected using TAQMAN™ analysis. The highest transcriptional expression of 46508 was noted in HUVEC cell line, with moderate to high expression found in normal pancreas, brain, hypothalamus, skeletal muscle, DRG (dorsal root ganglion) and skin. Moderate expression also noted in normal and tumor pairs of breast, ovarian, prostate, colon and lung tissue along with fibrotic liver, normal heart and diseased heart (CHF, congestive heart failure) samples.

TABLE 4
Tissue Distribution of 46508 mRNA by TAQMAN ™ Analysis

| Tissue Type | Expression |
|---|---|
| Artery normal | 8.6685 |
| Aorta diseased | 5.2992 |
| Vein normal | 1.7725 |
| Coronary SMC | 17.1577 |
| HUVEC | 49.0365 |
| Hemangioma | 5.0834 |
| Heart normal | 8.2009 |
| Heart CHF | 7.1393 |
| Kidney | 9.4204 |
| Skeletal Muscle | 16.6308 |
| Adipose normal | 3.9608 |
| Pancreas | 20.3335 |
| primary osteoblasts | 2.7431 |
| Osteoclasts (diff) | 0.8955 |
| Skin normal | 13.0031 |
| Spinal cord normal | 5.1365 |
| Brain Cortex normal | 24.2647 |
| Brain Hypothalamus normal | 25.2951 |
| Nerve | 7.8942 |
| DRG (Dorsal Root Ganglion) | 16.5159 |
| Breast normal | 6.5016 |
| Breast tumor | 11.4382 |
| Ovary normal | 10.0965 |
| Ovary Tumor | 6.7542 |
| Prostate Normal | 10.273 |
| Prostate Tumor | 11.0485 |
| Salivary glands | 2.1822 |
| Colon normal | 3.5327 |
| Colon Tumor | 14.9885 |
| Lung normal | 4.9273 |
| Lung tumor | 7.3655 |
| Lung COPD | 5.0658 |
| Colon IBD | 3.14 |
| Liver normal | 9.3229 |
| Liver fibrosis | 8.6385 |
| Spleen normal | 2.4129 |
| Tonsil normal | 2.83 |
| Lymph node normal | 5.3176 |
| Small intestine normal | 2.0573 |
| Macrophages | 1.3526 |
| Synovium | 2.3388 |
| BM-MNC | 0.2563 |
| Activated PBMC | 1.5538 |
| Neutrophils | 1.5755 |
| Megakaryocytes | 1.57 |
| Erythroid | 15.3566 |
| positive control | 21.1969 |

Table 5 below also shows expression of 46508 mRNA in various normal and diseased tissues, detected using TAQMAN™ analysis. Table 5 shows the high transcriptional expression of 46508 in tumor samples compared to normal organ matched controls. High transcriptional expression was noted in 5/5 primary ovarian tumors, 4/4 primary colon tumors, 2/2 colon to liver metastases, 3/6 primary lung tumors and proliferating HMVEC cells when compared to arrested HMVEC cells.

TABLE 5
Expression of 46508 mRNA in Normal and Cancerous Tissues

| Tissue Type | Expression |
|---|---|
| PIT 400 Breast Normal | 28.36 |
| PIT 372 Breast Normal | 36.40 |
| CHT 1228 Breast Normal | 8.88 |
| MDA 304 Breast Tumor: MD-IDC | 8.34 |
| CHT 2002 Breast Tumor: IDC | 3.55 |
| MDA 236-Breast Tumor: PD-IDC | 3.21 |
| CHT 562 Breast Tumor: IDC | 13.51 |
| NDR 138 Breast Tumor ILC (LG) | 25.30 |
| CHT 1841 Lymph node (Breast met) | 7.19 |
| PIT 58 Lung (Breast met) | 12.22 |
| CHT 620 Ovary Normal | 14.83 |
| CHT 619 Ovary Normal | 7.09 |
| CLN 012 Ovary Tumor | 40.53 |
| CLN 07 Ovary Tumor | 28.26 |
| CLN 17 Ovary Tumor | 94.40 |
| MDA 25 Ovary Tumor | 80.21 |
| CLN 08 Ovary Tumor | 28.07 |
| PIT 298 Lung Normal | 2.14 |
| MDA 185 Lung Normal | 6.05 |
| CLN 930 Lung Normal | 12.13 |
| MPI 215 Lung Tumor--SmC | 9.69 |
| MDA 259 Lung Tumor-PDNSCCL | 13.94 |
| CHT 832 Lung Tumor-PDNSCCL | 8.64 |
| MDA 262 Lung Tumor-SCC | 68.87 |
| CHT 793 Lung Tumor-ACA | 20.83 |
| CHT 331 Lung Tumor-ACA | 8.14 |
| CHT 405 Colon Normal | 2.50 |
| CHT 1685 Colon Normal | 2.10 |
| CHT 371 Colon Normal | 0.95 |
| CHT 382 Colon Tumor: MD | 69.11 |
| CHT 528 Colon Tumor: MD | 54.79 |
| CLN 609 Colon Tumor | 15.25 |
| NDR 210 Colon Tumor: MD-PD | 121.16 |
| CHT 340 Colon-Liver Met | 15.25 |
| CHT 1637Colon-Liver Met | 10.82 |
| PIT 260 Liver N (female) | 1.46 |
| CHT 1653 Cervix Squamous CC | 19.51 |
| CHT 569 Cervix Squamous CC | 1.31 |
| A24 HMVEC-Arrested | 11.56 |
| C48 HMVEC-Proliferating | 31.80 |
| Pooled Hemangiomas | 1.16 |
| HCT116N22 Normoxic | 76.42 |
| HCT116H22 Hypoxic | 68.39 |

Table 6 indicates that 46508 mRNA is highly expressed in several ovarian cell lines including SKOV3/Var, A2780, MDA2774 and ES-2. The table allows comparisons between two normal ovarian surface epithelium samples (MDA 127 Normal Ovary and MDA 224 Normal Ovary) and two ovarian ascites (MDA 124 Ovarian Ascites and MDA 126 Ovarian Ascites) samples. Expression of 46508 mRNA is upregulated in one of the ascites samples. The table also shows an experiment where the ovarian cancer cell line, HEY, was serum starved for 24 hours. Time points were taken at 0, 1, 3, 6, 9 and 12 hours after the addition of 10% serum (HEY 0 hr, HEY 1 hr, HEY 3 hr, HEY 6 hr, HEY 9 hr, and HEY 12 hr, respectively). Since cMyc protein is highly upregulated at 1 hour after addition of serum and phosphorylated at 6 hours, the experiment is a good model for identifying targets that are downstream of cMyc. These data indicate that 46508 mRNA may be regulated in a manner similar to cMyc since the expression increases from 1 to 9 hours after the addition of serum.

Also shown are data involving the ovarian cancer cell lines SKOV3 and SKOV3/Variant. These cell lines were grown in three different cellular environments: on plastic, in soft agar, and as a subcutaneous tumor in nude mice (all cells grown in 10% serum). The plastic sample was used as the “control” in each experiment. The SKOV3/Var cell line is a variant of the parental cell line SKOV3 which is resistant to cisplatin. These data indicate that 46508 mRNA is upregulated in environments that may be more similar to the tumor in vivo (the soft agar and subcutaneous tumor) compared to growth on plastic.

TABLE 6
Expression in Various Ovarian Cells

| Tissue Type | Expression |
|---|---|
| SKOV-3 No GF | 40.67 |
| SKOV-3 EGF ′15 | 41.52 |
| SKOV-3 EGF ′30 | 46.23 |
| SKOV-3 EGF ′60 | 38.88 |
| SKOV-3 Hrg ′15 | 36.02 |
| SKOV-3 Hrg ′30 | 41.67 |
| SKOV-3 Hrg ′60 | 48.87 |
| SKOV-3 Serum ′30 | 54.98 |
| SKOV-3var No GF | 151.25 |
| SKOV-3var EGF ′15 | 140.63 |
| SKOV-3var EGF ′30 | 126.74 |
| SKOV-3var EGF ′60 | 125.43 |
| SKOV-3var Hrg ′15 | 141.61 |
| SKOV-3var Hrg ′30 | 170.76 |
| SKOV-3var Hrg ′60 | 140.15 |
| SKOV-3var Serum ′30 | 190.78 |
| HEY Plastic | 50.94 |
| HEY Soft Agar | 22.96 |
| SKOV-3 | 35.65 |
| SKOV-3var | 122.00 |
| A2780 | 159.87 |
| A2780-ADR | 60.79 |
| OVCAR-3 | 54.98 |
| OVCAR-4 | 59.75 |
| MDA2774 | 123.28 |
| DOV13 | 36.52 |
| Caov-3 | 18.14 |
| ES-2 | 101.18 |
| HEY 0 hr | 55.17 |
| HEY 1 hr | 62.50 |
| HEY 3 hr | 74.84 |
| HEY 6 hr | 69.59 |
| HEY 9 hr | 77.75 |
| HEY 12 hr | 68.63 |
| SKOV-3 SubQ Tumor | 18.01 |
| SKOV-3 Variant Plastic | 152.30 |
| SKOV-3 Var SubQ Tumor | 9.39 |
| MDA 127 Normal Ovary | 11.44 |
| MDA 224 Normal Ovary | 17.10 |
| MDA 124 Ovarian Ascites | 18.84 |
| MDA 126 Ovarian Ascites | 41.67 |
| HEY | 63.81 |
| SKOV-3 Plastic | 72.80 |

### Human 16816

The human 16816 sequence (SEQ ID NO:39), which is approximately 2629 nucleotides long including untranslated regions, contains a predicted methionine-initiated coding sequence of about 2289 nucleotides (nucleotides 257-2545 of SEQ ID NO:39; SEQ ID NO:41), including the terminal codon. The coding sequence encodes a 762 amino acid protein (SEQ ID NO:40).

This mature protein form is approximately 762 amino acid residues in length (from about amino acid 1 to amino acid 762 of SEQ ID NO:40). Human 16816 contains the following regions or other structural features: one predicted phosphatidylinositol-specific phospholipase C domain X (PFAM Accession Number PF00388) located at about amino acid residues 291-436 of SEQ ID NO:40; one predicted phosphatidylinositol-specific phospholipase C domain Y (PFAM Accession Number PF00387) located at about amino acid residues 492-609 of SEQ ID NO:40; two predicted EF hand domains (PFAM Accession Number PF00036) located at about amino acid residues 138-166 and 174-202 of SEQ ID NO:40; one predicted C2 domain (PFAM Accession Number PF00168) located at about amino acid residues 291-436 and 492-609 of SEQ ID NO:40; two cAMP- and cGMP-dependent protein kinase phosphorylation sites (PS00004) located at about amino acids 435-438 and 482-485 of SEQ ID NO:40; nine predicted protein kinase C phosphorylation sites (PS00005) located at about amino acids 31-33, 56-58, 68-70, 203-205, 257-259, 355-357, 504-506, 666-668 and 741-743 of SEQ ID NO:40; seventeen predicted casein kinase II phosphorylation sites (PS00006) located at about amino 11-14, 62-65, 80-83, 100-103, 108-111, 127-130, 155-158, 223-226, 318-321, 410-413, 422-425, 438-441, 463-466, 467-470, 522-525, 649-652 and 710-713 of SEQ ID NO:40; four predicted N-myristoylation sites (PS00008) located at about amino acids 188-193, 219-224, 414-419 and 684-689 of SEQ ID NO:40; two predicted amidation sites (PS00009) located at about amino acids 96-99 and 433-436 of SEQ ID NO:40; one RGD cell attachment sequence (PS00016) located at about amino acids 145-147 of SEQ ID NO:40; and/or two EF hand calcium-binding domains (PS00018) located at about amino acids 147-159 and 183-195 of SEQ ID NO:40.

In one embodiment, a 16816 family member can include at least one phosphatidylinositol-specific phospholipase C domain X (PFAM Accession Number PF00388); at least one phosphatidylinositol-specific phospholipase C domain Y (PFAM Accession Number PF00387); at least one preferably two EF hand domains (PFAM Accession Number PF00036 or PS00018); at least one predicted C2 domain (PFAM Accession Number PF00168). Furthermore, a 16816 family member can include at least one and preferably two cAMP- and cGMP-dependent protein kinase phosphorylation sites; at least one, two, three, four, five, six, seven, eight, and preferably nine protein kinase C phosphorylation sites (PS00005); at least one, two, three, four, five, six, seven, eight, nine, ten, eleven, twelve, thirteen, fourteen, fifteen, sixteen and preferably seventeen casein kinase II phosphorylation sites (PS00006); at least one, two, three, and preferably four N-myristolyation sites (PS00008); at least one and preferably two predicted amidation sites; or at least one RGD cell attachment sequence.

16816 polypeptides of the invention include fragments which include: all or part of a hydrophobic sequence, e.g., the sequence from about amino acid 485 to 500, from about 650 to 660, and from about 685 to 700 of SEQ ID NO:40; all or part of a hydrophilic sequence, e.g., the sequence from about amino acid 125 to 150, from about 465 to 480, and from about 665 to 680 of SEQ ID NO:40; a sequence which includes a Cys, or a glycosylation site.

### Human 16839

The human 16839 sequence (SEQ ID NO:42), which is approximately 2171 nucleotides long including untranslated regions, contains a predicted methionine-initiated coding sequence of about 1827 nucleotides (nucleotides 232-2058 of SEQ ID NO:42; SEQ ID NO:44), including the terminal codon. The coding sequence encodes a 608 amino acid protein (SEQ ID NO:43).

This mature protein form is approximately 608 amino acid residues in length (from about amino acid 1 to amino acid 608 of SEQ ID NO:43). Human 16839 contains the following regions or other structural features: one EF hand domain (PFAM Accession Number PF00036) located at about amino acids 39 to 67 of SEQ ID NO:43; one phosphatidylinositol-specific phospholipase C domain X (PFAM Accession Number PF00388) located at about amino acids 156 to 300 of SEQ ID NO:43; one phosphatidylinositol-specific phospholipase C domain Y (PFAM Accession Number PF00387) located at about amino acids 348 to 465 of SEQ ID NO:43; one C2 domain (PFAM Accession Number PF00168) located at about amino acids 484 to 572 of SEQ ID NO:43; two N-glycosylation sites (PS00001) located at about amino acids 376-379 and 537-540 of SEQ ID NO:43; three cAMP- and cGMP-dependent protein kinase phosphorylation sites (PS00004) located at about amino acids 310-313, 337-340 and 385-388 of SEQ ID NO:43; ten predicted protein kinase C phosphorylation sites (PS00005) located at about amino acids 24-26, 68-70, 220-222, 303-305, 313-315, 340-342, 399-401, 485-487, 501-503 and 533-535 of SEQ ID NO:43; eight predicted casein kinase II phosphorylation sites (PS00006) located at about amino 56-59, 68-71, 79-82, 267-270, 303-306, 356-359, 378-381 and 411-414 of SEQ ID NO:43; and three predicted N-myristoylation sites (PS00008) located at about amino acids 16-21, 479-484 and 560-565 of SEQ ID NO:43.

In one embodiment, a 16839 family member can include at least one EF hand domain (PFAM Accession Number PF00036); at least one phosphatidylinositol-specific phospholipase C domain X (PFAM Accession Number PF00388); at least one phosphatidylinositol-specific phospholipase C domain Y (PFAM Accession Number PF00387); at least one C2 domain (PFAM Accession Number PF00168). Furthermore, a 16839 family member can include at least one N-glycosylation site (PS00001); at least one, two and preferably three cAMP- and cGMP-dependent protein kinase phosphorylation sites; at least one, two, three, four, five, six, seven, eight, nine and preferably ten protein kinase C phosphorylation sites (PS00005); at least one, two, three, four, five, six, seven, and preferably eight casein kinase II phosphorylation sites (PS00006); at least one, two, and preferably three N-myristolyation sites (PS00008).

16839 polypeptides of the invention include fragments which include: all or part of a hydrophobic sequence, e.g., the sequence from about amino acid 340 to 350, from about 480 to 490, and from about 540 to 560 of SEQ ID NO:43; all or part of a hydrophilic sequence, e.g., the sequence from about amino acid 300 to 325, from about 360 to 390, and from about 405 to 420 of SEQ ID NO:43; a sequence which includes a Cys, or a glycosylation site.

### 16816 and 16839 Phospholipase C Proteins

The 16816 and 16839 protein contain a significant number of structural characteristics in common with members of the phospholipase C family.

Phospholipase C (PLC) belongs to a family of enzymes, also known as disulfide isomerases, which play an important role in mediating signal transduction pathways. Many extracellular signaling molecules including hormones, growth factors, neurotransmitters, and immunoglobulins bind to their respective cell surface receptors and activate PLCs. Activated PLCs then catalyze the hydrolysis of phosphatidyl-inositol-4,5-bisphosphate (PIP2), a component of the plasma membrane, to produce diacylglycerol and inositol 1,4,5-trisphosphate (IP3).

In their respective biochemical pathways, IP3 and diacylglycerol serve as second messengers and trigger a series of intracellular responses. IP3 induces the release of calcium from internal cellular storage, and diacylglycerol activates protein kinase C (PKC). Both pathways are part of transmembrane signal transduction mechanisms, which regulate numerous cellular processes, including secretion, neural activity, metabolism, and proliferation.

PLC molecules have been found in a broad spectrum of organisms including bacteria, simple eukaryotes, plants and animals (Munnik et al., Biochim. Biophys. Acta. 1389:222-272, (1998)). Several distinct isoforms of PLC have been identified in animals and are categorized as PLC-beta, PLC-gamma, and PLC-delta. Subtypes are designated by adding Arabic numbers after the Greek letters, e.g., PLC-beta-1. PLCs have a molecular mass of 62-68 kDa, and their amino acid sequences show two regions of significant similarity.

The present invention is based, at least in part, on the discovery of novel molecules, referred to herein as “phospholipase C” or “16816” or “16839” nucleic acid and polypeptide molecules, which play a role in or function in modulating signal transduction pathways.

PLC molecules have been found in a broad spectrum of organisms including bacteria, simple eukaryotes, plants and animals. Members of a family can also have common functional characteristics. Members of the PLC family share one or more common domains such as a pleckstrin homology domain, an EF hand domain, a phosphatidylinositol-specific phospholipase domain X (PLC-X) domain, a phosphatidylinositol-specific phospholipase domain Y (PLC-Y) domain or a C2 domain. Members of this family can also have common functional characteristics, e.g., the ability to hydrolyze phosphatidylinositols.

A 16816 polypeptide can include a “pleckstrin homology (PH) domain” or regions homologous with a “PH domain”. As used herein, the term “PH domain” refers to a protein domain having an amino acid sequence of about 10 to 200, preferably about 50 to 150, more preferably about 108 amino acid residues. By “PH domain” is meant a domain that can function as a recognition site for a phosphatidylinositol, e.g., a 3, 4, 5-trisphosphate (PIP3) or another kinase ligand product, and can function as a means to localize PLC to the cytoplasmic face of the plasma membrane.

As used herein, the term “PH domain” includes an amino acid sequence of about 108 amino acid residues in length and having a bit score for the alignment of the sequence to the PH domain (HMM) of at least 10. Preferably, a PH domain includes at least about 10-200 amino acids, more preferably about 50-150 amino acid residues, or about 75-110 amino acids and has a bit score for the alignment of the sequence to the PH domain (HMM) of at least 20, 30, or greater.

In a preferred embodiment a 16816 polypeptide or protein has a “PH domain” or a region which includes at least about 10-200 amino acids, more preferably about 50-150 amino acid residues, or about 107 amino acid residues and has at least about 60%, 70% 80% 90% 95%, 99%, or 100% homology with a “PH domain,” e.g., the PH domain of human 16816 (e.g., residues 17-124 of SEQ ID NO:40). The identified PH domain consensus sequence is set forth as SEQ ID NO:45.

A 16816 or 16839 polypeptide can also include an “EF hand domain” or regions homologous with an “EF hand domain”. As used herein, the term “EF hand domain” refers to a protein domain having an amino acid sequence of about 5 to 50, preferably about 5 to 40, more preferably about 28-29 amino acid residues. By “EF hand domain” is meant a type of calcium-binding domain that consists of a twelve residue loop flanked on both sides by a twelve residue alpha-helical domain. In an EF-hand loop the calcium ion is coordinated in a pentagonal bipyramidal configuration. The six residues involved in the binding are in positions 1, 3, 5, 7, 9 and 12; these residues are denoted by X, Y, Z, -Y, -X and -Z. The invariant Glu or Asp at position 12 provides two oxygens for liganding Ca (bidentate ligand). Preferably, the EF hand domain includes the following amino acid consensus sequence having Prosite signatures as PS00018, or sequences homologous thereto. D-x-[DNS]-{ILVFYW}-[DENSTG]-[DNQGHRK]-{GP}-[LIVMC]-[DENQSTAGC]-x(2)-[DE]-[LIVMFYW] (SEQ ID NO:62). In the above conserved motif, and other motifs described herein, the standard IUPAC one-letter code for the amino acids is used. Each element in the pattern is separated by a dash (-); square brackets ([ ]) indicate the particular residues that are accepted at that position; x indicates that any residue is accepted at that position; and numbers in parentheses (( )) indicate the number of residues represented by the accompanying amino acid. The EF hand domains are located in mostly hydrophilic regions of the molecule of human 16816 or 16839 polypeptide and which corresponds to about amino acids 138-166 and 172-202 of SEQ ID NO:40; or amino acids 39-67 of SEQ ID NO:43. The EF hand domain (HMM) has been assigned the PFAM Accession Number PF00036.

The “EF hand domain” includes an amino acid sequence of about 28 amino acid residues in length and can have a bit score for the alignment of the sequence to the EF hand (HMM) of at least 5. Preferably, an EF hand domain includes at least about 5-50 amino acids, or at least about 5-40, or about 28 amino acids and has a bit score for the alignment of the sequence to the EF hand (HMM) of at least 5, 10, 15, 20, or greater.

In a preferred embodiment, the 16816 or 16839 polypeptide or protein has an “EF hand domain” or a region which includes at least about 5-50, more preferably about 5-40 or 28-29 amino acid residues and has at least about 60%, 70% 80% 90% 95%, 99%, or 100% homology with an “EF hand domain,” e.g., the EF hand domain of human 16816 or 16839 (e.g., residues 138-166 and 172-202 of SEQ ID NO:40; or amino acids 39-67 of SEQ ID NO:43). The identified EF hand domain consensus sequences within 16816 are set forth as SEQ ID NO:46 and 47, and the identified EF hand domain consensus sequence within 16839 is set forth as SEQ ID NO:56.

A 16816 or 16839 polypeptide can also include a “phosphatidylinositol-specific phospholipase C domain X (referred to herein as “PLC-X domain”)” or regions homologous with a “PLC-X domain”. As used herein, the term “PLC-X domain” refers to a protein domain having an amino acid sequence of about 8 to 200, preferably about 15 to 170, more preferably about 145 amino acid residues. By “PLC-X domain” is meant a subdomain that composes the catalytic site of the phospholipase, e.g., PLC-X subdomain can fold together with another subdomain, e.g., phosphatidylinositol-specific phospholipase C domain Y such that a functioning catalytic site that hydrolyzes a phosphatidylinositol is formed, e.g., phosphatidylinositol 4,5-bisphosphate, is formed.

The “PLC-X domain” includes an amino acid sequence of about 145 amino acid residues in length and can have a bit score for the alignment of the sequence to the phosphatidylinositol-specific phospholipase-C domain X (HMM) of at least 50. Preferably, a PLC-X domain includes at least about 15-170 amino acids, or at least about 20-150, or about 145 amino acids and has a bit score for the alignment of the sequence to the phosphatidylinositol-specific phospholipase-C domain X (HMM) of at least 60, 70, 80, 90, 100, 150, 200, 250, or greater.

In a preferred embodiment, the 16816 or 16839 polypeptide or protein has a “PLC-X” or a region which includes at least about 8-200, more preferably about 15-170 or 20-150 amino acid residues and has at least about 60%, 70% 80% 90% 95%, 99%, or 100% homology with a “PLC-X domain,” e.g., the PLC-X domain of human 16816 or 16839 (e.g., residues 291-436 of SEQ ID NO:40; or residues 156-300 of SEQ ID NO:43). The identified PLC-X domain consensus sequence of 16816 is set forth in SEQ ID NO:48 and the identified PLC-X domain consensus sequence of 16839 is set forth in SEQ ID NO:57.

A 16816 or 16839 polypeptide can include a “phosphatidylinositol-specific phospholipase C domain Y (referred to herein as PLC-Y domain)” or regions homologous with a “PLC-Y domain”. As used herein, the term “PLC-Y domain” refers to a protein domain having an amino acid sequence of about 8 to 200, preferably about 15 to 170, more preferably about 117 amino acid residues. By “PLC-Y domain” is meant a subdomain that composes the catalytic site of the phospholipase, e.g., the subdomain can fold together with another subdomain, e.g., PLC-X domain such that a functioning catalytic site that hydrolyzes a phosphatidylinositol, e.g., phosphatidylinositol 4,5-bisphosphate, is formed.

The “PLC-Y domain” includes an amino acid sequence of about 117 amino acid residues in length and can have a bit score for the alignment of the sequence to the PLC-Y domain (HMM) of at least 50. Preferably, a PLC-Y domain includes at least about 15-170 amino acids, or at least about 20-150, or about 117 amino acids and has a bit score for the alignment of the sequence to the PLC-Y domain (HMM) of at least 60, 70, 80, 90, 100, 110, 120, 140, 160, 180, or greater.

In a preferred embodiment 16816 or 16839 polypeptide or protein has a “PLC-Y domain” or a region which includes at least about 8-200, more preferably about 15-170 or 20-150 amino acid residues and has at least about 60%, 70% 80% 90% 95%, 99%, or 100% homology with a “PLC-Y domain” e.g., PLC-Y domain of human 16816 or 16839 (e.g., residues 492-609 of SEQ ID NO:40; or residues 348-465 of SEQ ID NO:43). The identified PLC-Y domain consensus sequence of 16816 is set forth in SEQ ID NO:49 and the identified PLC-Y domain consensus sequence of 16839 is set forth in SEQ ID NO:58.

A 16816 or 16839 polypeptide can include a “calcium (Ca2+) binding domain (referred to as C2 domain”) or regions homologous with a “C2 domain”. As used herein, the term “C2” refers to a protein domain having an amino acid sequence of about 8 to 200, preferably about 15 to 170, more preferably about 20 to 100, or still more preferably about 90 amino acid residues. By “C2 domain” is meant a domain that can mediate interaction with calcium or phospholipids.

The “C2 domain” includes an amino acid sequence of about 90 amino acid residues in length and can have a bit score for the alignment of the sequence to the C2 domain (HMM) of at least 50. Preferably, a C2 includes at least about 8-200, or at least about 15-170, or at least 20-100, or about 90 amino acids and has a bit score for the alignment of the sequence to the C2 domain (HMM) of at least 60, 70, 80, 85, or greater.

In a preferred embodiment, a 16816 or 16839 polypeptide or protein has a “C2” or a region which includes at least about 10-200, more preferably about 15-170 or 20-100 amino acid residues and has at least about 60%, 70% 80% 90% 95%, 99%, or 100% homology with a “C2,” e.g., the C2 domain of human 16816 or 16839 (e.g., residues 629-719 of SEQ ID NO:40; or residues 484-572 of SEQ ID NO:43). The identified C2 domain consensus sequence of 16816 is set forth in SEQ ID NO:50 and the identified C2 domain consensus sequence of 16839 is set forth in SEQ ID NO:59.

To identify the presence of a “PH domain,” “EF hand,” “PLC-X domain,” “PLC-Y domain,” or a “C2 domain” in a 16816 or 16839 protein sequence, and make the determination that a polypeptide or protein of interest has a particular profile, the amino acid sequence of the protein can be searched against a database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference.

A search was performed against the HMM database resulting in the identification of a “PH domain” in the amino acid sequence of human 16816 at about residues 17-124 of SEQ ID NO:40. The identified PH domain consensus sequence of 16816 is set forth in SEQ ID NO:45. This search also resulted in the identification of “EF domain(s)” in the amino acid sequence of human 16816 or 16839 at about residues 138-166 and 172-202 of SEQ ID NO:40 or amino acids 39-67 of SEQ ID NO:43 respectively. The identified EF domain consensus sequences of 16816 are set forth in SEQ ID NO:46 and 47 and the identified EF domain consensus sequence of 16839 is set forth in SEQ ID NO:56. This search also resulted in the identification of a “C2 domain” in the amino acid sequence of human 16816 or 16839 at about residues 629-719 of SEQ ID NO:40 and residues 484-572 of SEQ ID NO:43, respectively. The identified C2 domain consensus sequence of 16816 is set forth in SEQ ID NO:50 and the identified C2 domain consensus sequence of 16839 is set forth in SEQ ID NO:59. This search also resulted in the identification of a “PLC-Y domain” in the amino acid sequence of human 16816 or 16839 at about residues 492-609 of SEQ ID NO:40 and residues 348-465 of SEQ ID NO:43. The identified PLC-Y domain consensus sequence of 16816 is set forth in SEQ ID NO:49 and the identified PLC-Y domain consensus sequence of 16839 is set forth in SEQ ID NO:58. This search additionally resulted in the identification of a “PLC-X domain” in the amino acid sequence of human 16816 or 16839 at about residues 291-436 of SEQ ID NO:40 and residues 156-300 of SEQ ID NO:43. The identified PLC-X domain consensus sequence of 16816 is set forth in SEQ ID NO:48 and the identified PLC-X domain consensus sequence of 16839 is set forth in SEQ ID NO:57.

To identify the presence of a “phospholipase C” domain in a 16816 or 16839 protein sequence, and make the determination that a polypeptide or protein of interest has a particular profile, the amino acid sequence of the protein can be searched against a database of domains, e.g., the ProDom database (Corpet et al. (1999), Nucl. Acids Res. 27:263-267). The ProDom protein domain database consists of an automatic compilation of homologous domains. Current versions of ProDom are built using recursive PSI-BLAST searches (Altschul S F et al. (1997) Nucleic Acids Res. 25:3389-3402; Gouzy et al. (1999) Computers and Chemistry 23:333-340) of the SWISS-PROT 38 and TREMBL protein databases. The database automatically generates a consensus sequence for each domain. A BLAST search was performed against the HMM database resulting in the identification of a “phospholipase C” domain in the amino acid sequence of human 16816 or 16839.

The phospholipase C domain is homologous to ProDom family PD001214 (“phospholipase phosphodiesterase hydrolase phosphoinositide-specific 1-phosphatidylinositol-45-bisphosphate degradation transducer lipid beta” SEQ ID NO:51, ProDomain Release 2001.1). An alignment of the phospholipase C domain (amino acids 275 to 436 of SEQ ID NO:40) of human 16816 with a consensus amino acid sequence (SEQ ID NO:51) derived from a hidden Markov model shows that the consensus sequence for SEQ ID NO:51 is 54% identical over amino acids 275 to 436 of SEQ ID NO:40.

The phospholipase C domain is also homologous to ProDom family PD186804 (“phospholipase C delta calcium-binding PLC-III hydrolase phosphodiesterase lipid PLC-delta-1 1-phosphatidylinositol-45-bisphosphate” SEQ ID NO:52, ProDomain Release 2001.1. An alignment of the phospholipase C domain (amino acids 1 to 191 of SEQ ID NO:40) of human 16816 with a consensus amino acid sequence (SEQ ID NO:52) derived from a hidden Markov model shows that the consensus sequence for SEQ ID NO:52 is 44% identical over amino acids 1 to 191 of SEQ ID NO:40.

The phospholipase C domain is also homologous to ProDom family PD001202 (“phospholipase phosphodiesterase hydrolase phosphoinositide-specific 1-phosphatidylinositol-45-bisphosphate degradation lipid transducer beta” SEQ ID NOs:53 and 61, ProDomain Release 2001.1. An alignment of the phospholipase C domain (amino acids 491 to 608 of SEQ ID NO:40) of human 16816 with a consensus amino acid sequence (SEQ ID NO:53) derived from a hidden Markov model demonstrates that the consensus sequence for SEQ ID NO:53 is 57% identical over amino acids 491 to 608 of SEQ ID NO:40. An alignment of the phospholipase C domain (amino acids 350 to 473 of SEQ ID NO:43) of human 16839 with a consensus amino acid sequence (SEQ ID NO:61) derived from a hidden Markov model demonstrates that the consensus sequence for SEQ ID NO:61 is 47% identical over amino acids 350 to 473 of SEQ ID NO:43.

The phospholipase C domain is also homologous to ProDom family PD033204 (“C phospholipase delta-4 delta4 phospholipase” SEQ ID NO:54, ProDomain Release 2001.1. An alignment of the phospholipase C domain (amino acids 722 to 761 of SEQ ID NO:40) of human 16816 with a consensus amino acid sequence (SEQ ID NO:54) derived from a hidden Markov model demonstrates that the consensus sequence for SEQ ID NO:54 is 70% identical over amino acids 722 to 761 of SEQ ID NO:40.

The phospholipase C domain is also homologous to ProDom family PD270355 (“1-phosphatidylinositol-4 phosphodiesterase-like bisphosphate” SEQ ID NO:55, ProDomain Release 2001.1. An alignment of the phospholipase C domain (amino acids 562 to 621 of SEQ ID NO:40) of human 16816 with a consensus amino acid sequence (SEQ ID NO:55) derived from a hidden Markov model demonstrates that the consensus sequence for SEQ ID NO:55 is 46% identical over amino acids 562 to 621 of SEQ ID NO:40.

The phospholipase C domain is also homologous to ProDom family PD001214 (“phospholipase phosphodiesterase hydrolase phosphoinositide-specific 1-phosphatidylinositol-45-bisphosphate degradation transducer lipid beta” SEQ ID NO:60, ProDomain Release 2001.1. An alignment of the phospholipase C domain (amino acids 140 to 324 of SEQ ID NO:43) of human 16839 with a consensus amino acid sequence (SEQ ID NO:60) derived from a hidden Markov model shows that the consensus sequence for SEQ ID NO:60 is 48% identical over amino acids 140 to 324 of SEQ ID NO:43.

As the 16816 or 16839 polypeptide of the invention may modulate 16816 or 16839-mediated activity, they may be useful as of for developing novel diagnostic and therapeutic agents for 16816 or 16839-mediated or related disorders, as described below. As used herein, a “16816 or 16839 activity”, “biological activity of 16816 or 16839” or “functional activity of 16816 or 16839”, refers to an activity exerted by a 16816 or 16839 protein, polypeptide or nucleic acid molecule on e.g., a 16816 or 16839-responsive cell or on a 16816 or 16839 substrate, e.g., a protein substrate, as determined in vivo or in vitro. In one embodiment, a 16816 or 16839 activity is a direct activity, such as an association with a 16816 or 16839 target molecule. A “target molecule” or “binding partner” is a molecule with which a 16816 or 16839 protein binds or interacts with in nature. In an exemplary embodiment, it is a receptor, e.g., a tyrosine kinase receptor. In another embodiment, 16816 or 16839 can associate with a second messenger molecule such as a specialized adaptor molecule; with inositol phosphates and inositol lipids; membrane proteins; or with a guanine nucleotide binding-regulatory protein (G-protein). A 16816 or 16839 activity can also be an indirect activity, e.g., a cellular signaling activity mediated by interaction of the 16816 or 16839 protein with a receptor or another signaling molecule. For example, the 16816 or 16839 proteins of the present invention can have one or more of the following activities: (1) transduction of transmembrane signals; (2) lipid-metabolizing activity, e.g., 16816 or 16839 can catalyze the hydrolysis of phosphatidyl-inositol-4,5-bisphosphate (PIP2) producing diacylglycerol and inositol 1,4,5-trisphosphate; (3) the regulation of transmission of signals from cellular receptors such as hormones such as serotonin, growth factors such as platelet-derived growth factor (PDGF), fibroblast growth factor (FGF), and nerve growth factor (NGF), neurotransmitters and immunoglobulins; (4) modulation of cell proliferation; (5) modulation of cell differentiation; (6) modulation of cell migration; (7) modulation of fertilization; and (8) modulation of hypertension.

Based on the above-described sequence similarities, the 16816 or 16839 molecules of the present invention are predicted to have similar biological activities as members of the PLC family. Members of the PLC family play a very important role in transmembrane signal transduction. Extracellular signaling molecules including hormones, growth factors, neurotransmitters, and immunoglobulins bind to their respective cell surface receptors and activate phospholipase-C. PLC molecules have many functions including: glycogenolysis in liver cells, histamine secretion by mast cells, serotonin release by blood platelets, aggregation by blood platelets, insulin release by pancreatic islet cells, epinephrine secretion by adrenal chromaffin cells, and smooth muscle contraction. In general, biological systems that are activated by receptor tyrosine kinase cause the activation of phospholipase-C. The role of an activated PLC is to catalyze the hydrolysis of phosphatidyl-inositol-4,5-bisphosphate (PIP2), a minor component of the plasma membrane, to produce diacylglycerol and inositol 1,4,5-trisphosphate (IP3). Inositol trisphosphate releases calcium from intracellular stores and increases the influx of calcium from the extracellular fluid. The calcium ions directly regulate target enzymes and indirectly affect other enzymes by functioning as a second messenger and interacting with calcium-binding proteins, such as troponin C and calmodulin. For example, calcium ions regulate muscle contraction, glycogen breakdown and exocytosis. Diacylglycerol, a product of the hydrolysis by PLCs, acts as a second messenger by activating protein kinase C. Activated protein kinase C phosphorylates a great number of intracellular proteins at the serine and threonine residues and modulates different signaling pathways. For example, the phosphorylation of glycogen synthase by protein kinase C stops the synthesis of glycogen. Moreover, protein kinase C controls cell division and proliferation. Both pathways are part of transmembrane signal transduction mechanisms, which regulate cellular processes, which include secretion, neural activity, metabolism, differentiation and proliferation.

Both 16816 and 16839 proteins are homologous to the phospholipase C molecule, PLC1. Stimulation of 16816 or 16839 activity is desirable in situations in which 16816 or 16839 is abnormally downregulated and/or in which increased 16816 or 16839 activity is likely to have a beneficial effect. For example, research on chromosome 20q has associated this gene locus with tumor suppressor activity. Deletions and mutations of the 20q chromosome have been associated with myelodysplasia and myeloproliferative disorders. PLC1 is one of the genes present at this locus, and has been found to deleted in these cases. (Asimakopoulos et al. (1994) Blood 84(9):3086-94). As such, 16816 and 16839 may play a role in preventing or treating myeloid disorders.

Likewise, inhibition of 16816 or 16839 activity is desirable in situations in which 16816 or 16839 is abnormally upregulated and/or in which decreased 16816 or 16839 activity is likely to have a beneficial effect. It has been shown that PLC1 overexpression is associated with hepatocellular carcinoma. One antibody, k-PLC1, was shown to react with PLC1 (Wiedmann et al. (1987) Hepatology 7(3):543-50). As such, inhibitors such as 16816- or 16839-specific antibodies may be useful to reduce the quantity of PLC1 in such situations, and potentially decrease the severity and/or occurrence of hepatocellular carcinoma. Thus, the 16816 or 16839 molecules can act as novel diagnostic targets and therapeutic agents for controlling disorders caused by abnormal or aberrant PLC activity. Evidence indicates that a high percentage of primary human mammary carcinomas concomitantly show abnormally high levels of PLC-gamma-1 (Kassis et al., Clin Cancer Res., August; 5(8):2251-60, 1999). Likewise, studies on spontaneous hypertensive rats have suggested that one of the main causes for the hypertension is an abnormal activation of PLC-delta-1 resulting from point mutations in the X and Y regions of the PLC amino acid sequence (Sanada et al., Hypertension 33(4):1036-42, 1999). Therefore, the 16816 or 16839 molecules can act as novel diagnostic targets and therapeutic agents for controlling one or more of cellular proliferative and/or differentiative disorders, disorders associated with bone metabolism, immune disorders, hematopoietic disorders, cardiovascular disorders, liver disorders, viral diseases, pain or metabolic disorders.

As the 16816 or 16839 polypeptides of the invention may modulate 16816 or 16839-mediated activities, they may be useful for developing novel diagnostic and therapeutic agents for 16816 or 16839-mediated or related disorders, as described below.

Accordingly, 16816 or 16839 protein may mediate various disorders, including cellular proliferative and/or differentiative disorders, brain disorders, heart disorders, blood vessel disorders, and platelet disorders.

Identification and Characterization of Human 16816 or 16839 cDNAs

The human 16816 or 16839 sequence (SEQ ID NO:39 or SEQ ID NO:42), which is approximately 2629 or 2171 nucleotides long, respectively, including untranslated regions, contains a predicted methionine-initiated coding sequence of about 2289 or 1827 nucleotides, respectively (nucleotides 257-2545 of SEQ ID NO:39; SEQ ID NO:41; or nucleotides 232-2058 of SEQ ID NO:42; SEQ ID NO:44). The coding sequence encodes a 762 or 608 amino acid protein (SEQ ID NO:40 or SEQ ID NO:43), respectively.

### Gene Expression Analysis

Total RNA was prepared from various human tissues by a single step extraction method using RNA STAT-60 according to the manufacturer's instructions (TelTest, Inc). Each RNA preparation was treated with DNase I (Ambion) at 37° C. for 1 hour. DNAse I treatment was determined to be complete if the sample required at least 38 PCR amplification cycles to reach a threshold level of fluorescence using β-2 microglobulin as an internal amplicon reference. The integrity of the RNA samples following DNase I treatment was confirmed by agarose gel electrophoresis and ethidium bromide staining. After phenol extraction cDNA was prepared from the sample using the SUPERSCRIPT™ Choice System following the manufacturer's instructions (GibcoBRL). A negative control of RNA without reverse transcriptase was mock reverse transcribed for each RNA sample.

Human 16816 or 16839 expression was measured by TAQMAN™ quantitative PCR (Perkin Elmer Applied Biosystems) in cDNA prepared from a variety of normal and diseased (e.g., cancerous) human tissues or cell lines. Probes were designed by PrimerExpress software (PE Biosystems) based on the sequence of the human 16816 or 16839 gene. Each human 16816 or 16839 gene probe was labeled using FAM (6-carboxyfluorescein), and the β2-microglobulin reference probe was labeled with a different fluorescent dye, VIC. The differential labeling of the target gene and internal reference gene thus enabled measurement in same well. Forward and reverse primers and the probes for both β2-microglobulin and target gene were added to the TAQMAN™ Universal PCR Master Mix (PE Applied Biosystems). Although the final concentration of primer and probe could vary, each was internally consistent within a given experiment. A typical experiment contained 200 nM of forward and reverse primers plus 100 nM probe for β-2 microglobulin and 600 nM forward and reverse primers plus 200 nM probe for the target gene. TAQMAN™ matrix experiments were carried out on an ABI PRISM 7700 Sequence Detection System (PE Applied Biosystems). The thermal cycler conditions were as follows: hold for 2 min at 50° C. and 10 min at 95° C., followed by two-step PCR for 40 cycles of 95° C. for 15 sec followed by 60° C. for 1 min.

The following method was used to quantitatively calculate human 16816 or 16839 gene expression in the various tissues relative to β-2 microglobulin expression in the same tissue. The threshold cycle (Ct) value is defined as the cycle at which a statistically significant increase in fluorescence is detected. A lower Ct value is indicative of a higher mRNA concentration. The Ct value of the human 16816 or 16839 gene is normalized by subtracting the Ct value of the β-2 microglobulin gene to obtain a ΔCt value using the following formula: ΔCt=Ct_{human 59914 and 59921}−Ct_{β-2 microglobulin}. Expression is then calibrated against a cDNA sample showing a comparatively low level of expression of the human 16816 or 16839 gene. The ΔCt value for the calibrator sample is then subtracted from ΔCt for each tissue sample according to the following formula: ΔΔCt=ΔCt-ₛₐₘₚₗₑ−ΔCt-_{calibrator}. Relative expression is then calculated using the arithmetic formula given by 2-ΔΔCt. Expression of the target human 16816 or 16839 gene in each of the tissues tested is then graphically represented as discussed in more detail below.

TAQMAN™ real-time quantitative RT-PCR is used to detect the presence of RNA transcript corresponding to human 16816 relative to a no template control in a panel of human tissues or cells. It is found that the highest expression of 16816 orthologs are expressed in skeletal muscle tissue as shown in Table 7.

TABLE 7

| | mean | mean | | |
|---|---|---|---|---|
| Tissue Type | 16816 | βeta 2 | ∂Ct | expression |
| Aorta/normal | 38.87 | 23.105 | 15.765 | 0 |
| Fetal heart/normal | 33.43 | 20.82 | 12.61 | 0.159960132 |
| Heart normal | 34.62 | 18.985 | 15.635 | 0.019651514 |
| Heart/CHF | 35.64 | 20.47 | 15.17 | 0 |
| Vein/Normal | 33.81 | 19.15 | 14.66 | 0.038627826 |
| Spinal cord/Normal | 33.175 | 19.43 | 13.745 | 0.072835434 |
| Brain cortex/Normal | 30.42 | 21.2 | 9.22 | 1.676885618 |
| Brain hypothalamus/ | 30.415 | 20.155 | 10.26 | 0.815515546 |
| Normal | | | | |
| Glial cells (Astrocytes) | 33.495 | 21.58 | 11.915 | 0.258956968 |
| Brain/Glioblastoma | 29.77 | 18.41 | 11.36 | 0.380451455 |
| Breast/Normal | 34.145 | 19.195 | 14.95 | 0.031593778 |
| Breast tumor/IDC | 33.215 | 18.82 | 14.395 | 0.046416588 |
| OVARY/Normal | 35.47 | 21.23 | 14.24 | 0 |
| OVARY/Tumor | 37.815 | 19.785 | 18.03 | 0 |
| Pancreas | 35.73 | 18.125 | 17.605 | 0 |
| Prostate/Normal | 30.895 | 19.255 | 11.64 | 0.313336401 |
| Prostate/Tumor | 31.695 | 18.255 | 13.44 | 0.089982252 |
| Colon/normal | 31.035 | 17.45 | 13.585 | 0.081378093 |
| Colon/tumor | 36.88 | 18.84 | 18.04 | 0 |
| Colon/IBD | 35.185 | 18.445 | 16.74 | 0 |
| Kidney/normal | 30.255 | 21.005 | 9.25 | 1.642375811 |
| Liver/normal | 36.48 | 19.18 | 17.3 | 0 |
| Liver fibrosis | 35.16 | 20.015 | 15.145 | 0 |
| Fetal Liver/normal | 37.02 | 22.14 | 14.88 | 0 |
| Lung/normal | 33.93 | 18.02 | 15.91 | 0.016241 |
| Lung/tumor | 34.845 | 18.955 | 15.89 | 0.016467716 |
| Lung/COPD | 33.345 | 18.13 | 15.215 | 0.026292302 |
| Spleen/normal | 39.23 | 20.19 | 19.04 | 0 |
| Tonsil/normal | 35.72 | 17.955 | 17.765 | 0 |
| Lymph node/normal | 34.55 | 18.485 | 16.065 | 0.014586568 |
| Thymus/normal | 33.475 | 19.675 | 13.8 | 0.070110984 |
| Epithelial Cells (prostate) | 31.825 | 20.57 | 11.255 | 0.409173406 |
| Endothelial Cells (aortic) | 32.815 | 20.775 | 12.04 | 0.237464587 |
| Skeletal Muscle/Normal | 24.19 | 19.34 | 4.85 | 34.674046 |
| Fibroblasts (Dermal) | 31.89 | 18.915 | 12.975 | 0.124204064 |
| Skin/normal | 34.805 | 20.945 | 13.86 | 0.067254951 |
| Adipose/Normal | 32.94 | 18.88 | 14.06 | 0.058548835 |
| Osteoblasts (primary) | 32.595 | 20.175 | 12.42 | 0.182476715 |
| Osteoblasts (Undiff) | 31.05 | 19.025 | 12.025 | 0.239946435 |
| Osteoblasts(Diff) | 30.815 | 18.315 | 12.5 | 0.172633492 |
| Osteoclasts | 34.17 | 17.725 | 16.445 | 0.011208867 |
| Aortic SMC Early | 34.705 | 20.23 | 14.475 | 0.043912768 |
| Aortic SMC Late | 34.18 | 22.97 | 11.21 | 0.42213732 |
| shear HUVEC | 31.315 | 20.955 | 10.36 | 0.76090291 |
| static HUVEC | 31.465 | 20.875 | 10.59 | 0.64877237 |

TAQMAN™ real-time quantitative RT-PCR is used to detect the presence of RNA transcript corresponding to human 16839 relative to a no template control in a panel of human tissues or cells. It is found that 16839 orthologs are expressed in teste as shown in the Table 8 and DRG as shown in Table 9.

TABLE 8

| Tissue Type | 16839.20 | β2.803 | ∂Ct | Expression |
|---|---|---|---|---|
| Adrenal Gland | 40.00 | 19.21 | 20.80 | 0.00 |
| Brain | 40.00 | 21.01 | 18.99 | 0.00 |
| Heart | 40.00 | 18.97 | 21.04 | 0.00 |
| Kidney | 39.90 | 18.98 | 20.92 | 0.00 |
| Liver | 40.00 | 18.61 | 21.40 | 0.00 |
| Lung | 40.00 | 17.12 | 22.88 | 0.00 |
| Mammary Gland | 40.00 | 17.94 | 22.07 | 0.00 |
| Pancreas | 40.00 | 20.50 | 19.50 | 0.00 |
| Placenta | 40.00 | 17.83 | 22.18 | 0.00 |
| Prostate | 40.00 | 17.20 | 22.80 | 0.00 |
| Salivary Gland | 40.00 | 18.48 | 21.52 | 0.00 |
| Muscle | 40.00 | 20.56 | 19.45 | 0.00 |
| Sm. Intestine | 40.00 | 18.82 | 21.19 | 0.00 |
| Spleen | 40.00 | 16.64 | 23.36 | 0.00 |
| Stomach | 40.00 | 18.52 | 21.48 | 0.00 |
| Teste | 26.97 | 20.05 | 6.92 | 1.69 |
| Thymus | 40.00 | 17.66 | 22.35 | 0.00 |
| Trachea | 40.00 | 18.93 | 21.07 | 0.00 |
| Uterus | 40.00 | 18.80 | 21.20 | 0.00 |
| Spinal Cord | 40.00 | 18.94 | 21.07 | 0.00 |
| DRG | 40.00 | 19.50 | 20.51 | 0.00 |
| Skin | 39.10 | 18.44 | 20.66 | 0.00 |

TABLE 9

| Tissue Type | Mean | β 2 Mean | ∂∂ Ct | Expression |
|---|---|---|---|---|
| Artery normal | 40 | 19.38 | 20.62 | 0 |
| Vein normal | 39.38 | 18.47 | 20.91 | 0 |
| Aortic SMC EARLY | 38.88 | 19.23 | 19.64 | 0 |
| Coronary SMC | 39.92 | 20.55 | 19.38 | 0 |
| Static HUVEC | 39.06 | 18.66 | 20.4 | 0 |
| Heart normal | 40 | 17.21 | 22.79 | 0 |
| Heart CHF | 39.26 | 17.18 | 22.07 | 0 |
| Kidney | 34.14 | 17.21 | 16.93 | 0.008 |
| Skeletal Muscle | 40 | 20.26 | 19.74 | 0 |
| Adipose normal | 40 | 18.7 | 21.31 | 0 |
| Pancreas | 39.41 | 19.23 | 20.18 | 0 |
| primary osteoblasts | 37.65 | 17.54 | 20.11 | 0 |
| Osteoclasts (diff) | 35.13 | 15.82 | 19.31 | 0 |
| Skin normal | 39.13 | 19.16 | 19.98 | 0 |
| Spinal cord normal | 40 | 18.59 | 21.41 | 0 |
| Brain Cortex normal | 38.95 | 18.59 | 20.36 | 0 |
| Brain Hypothalamus normal | 40 | 19.43 | 20.57 | 0 |
| Nerve | 40 | 22.34 | 17.66 | 0 |
| DRG (Dorsal Root Ganglion) | 34.58 | 19.82 | 14.76 | 0.0362 |
| Glial Cells (Astrocytes) | 38.84 | 20.34 | 18.5 | 0 |
| Glioblastoma | 38.91 | 16.52 | 22.39 | 0 |
| Breast normal | 39.6 | 18.93 | 20.67 | 0 |
| Breast tumor | 39.98 | 17.11 | 22.88 | 0 |
| Ovary normal | 38.79 | 18.03 | 20.76 | 0 |
| Ovary Tumor | 40 | 18.23 | 21.77 | 0 |
| Prostate Normal | 38.15 | 18.3 | 19.85 | 0 |
| Prostate Tumor | 38.36 | 16.03 | 22.33 | 0 |
| Epithelial Cells (Prostate) | 39.45 | 19.29 | 20.16 | 0 |
| Colon normal | 39.44 | 16.6 | 22.84 | 0 |
| Colon Tumor | 38.73 | 16.22 | 22.51 | 0 |
| Lung normal | 38.99 | 16.12 | 22.87 | 0 |
| Lung tumor | 37.2 | 16.58 | 20.63 | 0 |
| Lung COPD | 38.73 | 16.72 | 22 | 0 |
| Colon IBD | 38.3 | 15.77 | 22.54 | 0 |
| Liver normal | 39.88 | 17.98 | 21.91 | 0 |
| Liver fibrosis | 37.62 | 19.28 | 18.34 | 0 |
| Dermal Cells-fibroblasts | 37.84 | 17.66 | 20.18 | 0 |
| Spleen normal | 37.22 | 18.06 | 19.16 | 0 |
| Tonsil normal | 37.55 | 15.44 | 22.11 | 0 |
| Lymph node | 39.7 | 17.23 | 22.47 | 0 |
| Resting PBMC | 40 | 18.8 | 21.2 | 0 |
| Skin-Decubitus | 36.45 | 18.82 | 17.64 | 0 |
| Synovium | 37.98 | 17.66 | 20.32 | 0 |
| BM-MNC | | | | |
| (Bone marrow | 38.51 | 14.83 | 23.68 | 0 |
| mononuclear cells) | | | | |
| Activated PBMC | 37.06 | 13.93 | 23.13 | 0 |
| Shear HUVEC | 38 | 17.99 | 20.01 | 0.0009 |

Expression of 16839 was also detected in a panel of tissues and liver cell lines as shown in Table 10.

TABLE 10

| Tissue Type | Mean | 18S Mean | ∂∂ Ct | Expression |
|---|---|---|---|---|
| PIT 278/Heart | 33.06 | 13.77 | 19.29 | 0.0016 |
| PIT 351/Kidney | 30.36 | 12.56 | 17.81 | 0.0044 |
| PIT 915/Skeletal | 38.28 | 11.87 | 26.41 | 0 |
| Muscle | | | | |
| NDR 63/Liver | 33.23 | 11.69 | 21.54 | 0.0003 |
| NDR 242/Liver | 34.26 | 12.63 | 21.63 | 0.0003 |
| PIT 260/Liver | 33.58 | 11.77 | 21.81 | 0.0003 |
| CHT 756/Liver | 31.77 | 11.65 | 20.13 | 0.0009 |
| MPI 155/Liver | 34.91 | 14.68 | 20.23 | 0.0008 |
| MPI 146/Liver | 30.91 | 11.95 | 18.97 | 0.002 |
| CHT 902/Liver | 35.72 | 12.49 | 23.22 | 0 |
| PIT 45/Liver | 33.97 | 12.14 | 21.84 | 0.0003 |
| PIT 292/Liver | 35.46 | 13.65 | 21.82 | 0 |
| CLN 784/Liver | 32.56 | 11.76 | 20.8 | 0.0005 |
| NDR 752/Liver | 31.09 | 11.04 | 20.06 | 0.0009 |
| CHT 1679/Liver | 35.38 | 11.7 | 23.68 | 0 |
| CHT 1420/Liver | 33.06 | 11.15 | 21.91 | 0.0003 |
| CHT 339/Liver | 35.68 | 11.23 | 24.45 | 0 |
| CHT 1237/Liver | 33.41 | 11.02 | 22.4 | 0.0002 |

Expression of 16839 was also detected in a panel of breast tumor cell lines as shown in Table 11.

TABLE 11

| | | β2 | | |
|---|---|---|---|---|
| Tissue Type | Mean 16839.4 | Mean | ∂∂ Ct | Expression |
| MCF10MS | 35.1 | 18.84 | 16.25 | 0 |
| MCF10A | 33.88 | 18.81 | 15.07 | 0.03 |
| MCF10AT.cl1 | 34.2 | 18.88 | 15.32 | 0.02 |
| MCF10AT.cl3 | 35.19 | 18.22 | 16.98 | 0 |
| MCF10AT1 | 36.74 | 19.19 | 17.56 | 0 |
| MCF10AT3B | 36.23 | 18.93 | 17.3 | 0 |
| MCF10CA1a.cl1 | 36.94 | 16.34 | 20.59 | 0 |
| MCF10AT3B Agar | 37.76 | 25.07 | 12.68 | 0 |
| MCF10CA1a.cl1 Agar | 37.93 | 23.13 | 14.8 | 0 |
| MCF10A.m25 Plastic | 32.39 | 23.05 | 9.35 | 1.54 |
| MCF10CA Agar | 33.63 | 20.71 | 12.93 | 0.13 |
| MCF10CA Plastic | 35.98 | 20.56 | 15.43 | 0 |
| MCF3B Agar | 37.34 | 21 | 16.34 | 0 |
| MCF3B Plastic | 32.67 | 20.54 | 12.14 | 0.22 |
| MCF10A EGF 0 hr | 28.23 | 16.57 | 11.66 | 0.31 |
| MCF10A EGF 0.5 hr | 28.84 | 16.61 | 12.23 | 0.21 |
| MCF10A EGF 1 hr | 28.68 | 16.79 | 11.9 | 0.26 |
| MCF10A EGF 2 hr | 29.54 | 16.88 | 12.66 | 0.15 |
| MCF10A EGF 4 hr | 31.21 | 16.97 | 14.24 | 0.05 |
| MCF10A EGF 8 hr | 31.16 | 16.65 | 14.51 | 0.04 |
| MCF10A IGF1A 0 hr | 30.54 | 20.41 | 10.13 | 0.90 |
| MCF10A IGF1A 0.5 hr | 30.89 | 21.36 | 9.52 | 1.36 |
| MCF10A IGF1A 1 hr | 29.7 | 20.84 | 8.86 | 2.15 |
| MCF10A IGF1A 3 hr | 30.14 | 21.09 | 9.05 | 1.89 |
| MCF10A IGF1A 24 hr | 28.53 | 20.43 | 8.1 | 3.66 |
| MCF10AT3B.cl5 Plastic | 36.57 | 20.54 | 16.03 | 0 |
| MCF10AT3B.cl6 Plastic | 38.18 | 20.7 | 17.48 | 0 |
| MCF10AT3B.cl3 Plastic | 39.13 | 20.8 | 18.34 | 0 |
| MCF10AT3B.cl1 Plastic | 35.92 | 21.07 | 14.85 | 0 |
| MCF10AT3B.cl4 Plastic | 38.1 | 20.95 | 17.15 | 0 |
| MCF10AT3B.cl2 Plastic | 36.49 | 20.75 | 15.74 | 0 |
| MCF10AT3B.cl5 Agar | 39.22 | 22.68 | 16.54 | 0 |
| MCF10AT3B.cl6 Agar | 35.7 | 23.13 | 12.57 | 0 |
| MCF-7 | 37.46 | 22.25 | 15.22 | 0 |
| ZR--75 | 37.54 | 21.25 | 16.3 | 0 |
| T47D | 37.19 | 20.72 | 16.47 | 0 |
| MDA-231 | 33.55 | 19.7 | 13.85 | 0.07 |
| MDA-435 | 34.73 | 19.39 | 15.35 | 0.02 |
| SkBr3 | 37.16 | 19.86 | 17.3 | 0 |
| Hs578Bst | 36.52 | 18.93 | 17.59 | 0 |
| Hs578T | 31.36 | 19 | 12.35 | 0.19 |

Expression of 16839 was also detected in an oncology phase panel as shown in Table 12 and shows highest relative expression in lung tumor (CHT 832 lung T-PDNCSCCL) and upregulation of 16839 was found in 3/7 lung tumor tissue or cell samples.

TABLE 12

| | Mean | | | |
|---|---|---|---|---|
| Tissue Type | 16839.4 | β2 Mean | ∂∂ Ct | Expression |
| PIT 400 Breast N | 35.4 | 19.18 | 16.23 | 0 |
| PIT 372 Breast N | 35.38 | 19.66 | 15.72 | 0 |
| CHT 558 Breast N | 36.38 | 19.02 | 17.36 | 0 |
| CLN 168 Breast T: IDC | 38.45 | 19.84 | 18.62 | 0 |
| MDA 304 Breast T: MD-IDC | 37.04 | 17.84 | 19.2 | 0 |
| NDR 58 Breast T: IDC | 38.83 | 17.84 | 20.99 | 0 |
| NDR 05 Breast T: IDC | 35.22 | 20.57 | 14.65 | 0 |
| CHT 562 Breast T: IDC | 34.08 | 18.71 | 15.37 | 0.02 |
| NDR 12 Breast T | 35.52 | 21.39 | 14.13 | 0 |
| PIT 208 Ovary N | 37.06 | 18.56 | 18.5 | 0 |
| CHT 620 Ovary N | 36.05 | 19.2 | 16.85 | 0 |
| CLN 03 Ovary T | 40 | 19.83 | 20.17 | 0 |
| CLN 17 Ovary T | 39.49 | 20 | 19.5 | 0 |
| MDA 25 Ovary T | 38.42 | 21.65 | 16.78 | 0 |
| MDA 216 Ovary T | 38.04 | 20.11 | 17.93 | 0 |
| CLN 012 Ovary T | 39.27 | 21.3 | 17.98 | 0 |
| MDA 185 Lung N | 40 | 19.43 | 20.57 | 0 |
| CLN 930 Lung N | 37.99 | 20.29 | 17.7 | 0 |
| MDA 183 Lung N | 39.65 | 18.11 | 21.54 | 0 |
| MPI 215 Lung T--SmC | 31.34 | 18.62 | 12.72 | 0.15 |
| MDA 259 Lung T-PDNSCCL | 37.42 | 19.93 | 17.49 | 0 |
| CHT 832 Lung T-PDNSCCL | 28.18 | 18.86 | 9.32 | 1.56 |
| MDA 253 Lung T-PDNSCC | 33.39 | 18.05 | 15.35 | 0.02 |
| MDA 262 Lung T-SCC | 38.79 | 22.28 | 16.51 | 0 |
| CHT 211 Lung T-AC | 39.1 | 19.29 | 19.82 | 0 |
| CHT 793 Lung T-ACA | 35.74 | 18.55 | 17.19 | 0 |
| CHT 396 Colon N | 39.82 | 17.7 | 22.13 | 0 |
| CHT 523 Colon N | 39.99 | 18.52 | 21.47 | 0 |
| CHT 452 Colon N | 40 | 17.5 | 22.5 | 0 |
| CHT 382 Colon T: MD | 38.42 | 17.97 | 20.45 | 0 |
| CHT 528 Colon T: MD | 34.85 | 17.54 | 17.32 | 0.01 |
| CLN 609 Colon T | 38.1 | 18.82 | 19.29 | 0 |
| CHT 372 Colon T: MD-PD | 37.83 | 19.24 | 18.59 | 0 |
| CHT 340 Colon-Liver Met | 37.93 | 19.99 | 17.94 | 0 |
| NDR 100 Colon-Liver Met | 36.52 | 18.26 | 18.27 | 0 |
| PIT 260 Liver N (female) | 35.31 | 16.93 | 18.37 | 0 |
| ONC 102 Hemangioma | 36.36 | 19.04 | 17.32 | 0 |
| A24 HMVEC-Arr | 35.31 | 18.75 | 16.55 | 0 |
| C48 HMVEC-Prol | 35.12 | 19.18 | 15.94 | 0 |

### Human 49937, 49931 and 49933

The present invention is based, at least in part, on the discovery of novel calcium transporter family members, referred to interchangeably herein as “P-type ATPase”, “E1-E2 ATPase”, “human E1-E2 ATPase”, or “HEAT” nucleic acid and protein molecules (e.g., HEAT-1 (49937), HEAT-2 (49931) and HEAT-3 (49933)). These novel molecules are members of the E1-E2 ATPase superfamily and are highly expressed in human vessels, endothelial cells, and vascular smooth muscle cells, e.g., coronary vascular smooth muscle cells.

The E1-E2 ATPase family is a large superfamily of cation transport enzymes that contains at least 80 members found in diverse organisms such as bacteria, archaea, and eukaryotes (Palmgren, M. G. and Axelsen, K. B. (1998) Biochim. Biophys. Acta. 1365:37-45). These enzymes are involved in ATP hydrolysis-dependent transmembrane movement of a variety of inorganic cations (e.g., H⁺, Na⁺, K⁺, Ca²⁺, Cu²⁺, Cd⁺, and Mg²⁺ ions) across a concentration gradient, whereby the enzyme converts the free energy of ATP hydrolysis into electrochemical ion gradients. E1-E2 ATPases are also known as “P-type” ATPases, referring to the existence of a covalent high-energy phosphoryl-enzyme intermediate in the chemical reaction pathway of these transporters. The superfamily contains four major groups: Ca²⁺ transporting ATPases; Na⁺/K⁺- and gastric H⁺/K⁺ transporting ATPases; plasma membrane H⁺ transporting ATPases of plants, fungi, and lower eukaryotes; and all bacterial P-type ATPases (Kuhlbrandt et al. (1998) Curr. Opin. Struct. Biol. 8:510-516).

The E1-E2 ATPases are involved in ATP hydrolysis-dependent transmembrane movement of inorganic cations (e.g., Ca²⁺ ions) across a concentration gradient. E1-E2 ATPases are phosphorylated at a highly conserved DKTG sequence. Phosphorylation at this site is thought to control the enzyme's substrate affinity. Most E1-E2 ATPases contain ten alpha-helical transmembrane domains, although additional domains may be present. Members of the E1-E2 ATPase superfamily are able to generate electrochemical ion gradients which enable a variety of processes in the cell such as absorption, secretion, transmembrane signaling, nerve impulse transmission, excitation/contraction coupling, and growth and differentiation (Scarborough (1999) Curr. Opin. Cell Biol. 11:517-522).

As indicated in herein, the HEAT molecules of the present invention, e.g., HEAT-2, are up-regulated during shear, proliferation, and tube formation of endothelial cells and, thus, are believed to be involved in angiogenesis. Calcium ions are involved in the regulation of many cellular activities. In vascular smooth muscle cells, transient increases in intracellular calcium levels mediate contraction. Thus, maintenance of a low steady-state level of calcium is critical to maintaining proper cell function. Additionally, since the main determinant of the contraction-relaxation cycle of smooth muscle is calcium, calcium concentration is an important factor in the regulation of vascular tone. The normal concentration of calcium in the cell is in the submicromolar range, while the concentration in the extracellular compartment is in the millimolar range. In order to maintain intracellular calcium concentration in the submicromolar range, several mechanisms are operative in most cells. In smooth muscle cells, these regulatory mechanisms include calcium extrusion via Ca²⁺-transporting E1-E2 ATPases at the plasma membrane and at the sarcoplasmic/endoplasmic reticulum.

Thus, as the HEAT molecules of the present invention are Ca²⁺-transporting E1-E2 ATPases, and are highly expressed in vessels, endothelial cells, and vascular smooth muscle cells, these molecules are believed to be involved in vasotone regulation of vascular smooth muscle cells, e.g., coronary vascular smooth muscle cells. For example, activation of a HEAT molecule of the invention, e.g., HEAT-3, may result in decreased cytosolic calcium concentrations, thus reducing vascular tone. Inhibition of a HEAT molecule of the invention, e.g., HEAT-3, may result in decreased intracellular calcium store, which may subsequently lower the calcium release by vasopressor stimulation, thereby reducing vascular smooth muscle tone.

Accordingly, the HEAT molecules of the present invention provide novel diagnostic targets and therapeutic agents for cardiovascular disorders. The HEAT molecules of the present invention further provide novel diagnostic targets and therapeutic agents for cellular proliferation, growth, or differentiation disorders. Additional disorders that may be treated using the molecules of the present invention include disorders affecting tissues in which HEAT protein is expressed (e.g., vessels, endothelial cells, and vascular smooth muscle cells).

The family of HEAT proteins of the present invention comprises at least one “transmembrane domain,” preferably at least 2, 3, or 4 transmembrane domains, more preferably 5, 6, 7, 8, or 9 transmembrane domains, even more preferably 10 or 11 transmembrane domains, and most preferably, 12 transmembrane domains. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta, W. N. et al. (1996) Annu. Rev. Neurosci. 19:235-263, the contents of which are incorporated herein by reference. Amino acid residues 8-25, 47-65, 231-253, 256-276, 428-448, 464-484, 936-954, 963-987, 994-1015, 1049-1065, 1079-1102, and 1118-1134 of the human HEAT-1 protein (SEQ ID NO:64) are predicted to comprise transmembrane domains. Amino acid residues 29-50, 211-227, 234-253, 294-317, 410-434, 449-469, 941-960, 968-985, 1000-1020, 1076-1092, 1105-1129, 1144-1160 of the human HEAT-2 protein (SEQ ID NO:68) are predicted to comprise transmembrane domains. Amino acid residues 65-89, 99-116, 242-258, 265-281, 445-464, 493-509, 990-1007, 1015-1031, 1049-1073, 1103-1119, 1134-1151, 1171-1187 of the human HEAT-3 protein (SEQ ID NO:71) are also predicted to comprise transmembrane domains.

In another embodiment, members of the HEAT family of proteins include at least one “E1-E2 ATPase domain” in the protein or corresponding nucleic acid molecule. As used herein, the term “E1-E2 ATPase” domain includes a protein domain having at least about 70-110 amino acid residues and a bit score of at least 30 when compared against an E1-E2 ATPase Hidden Markov Model (HMM), e.g., PFAM Accession Number PF00122. Preferably, an E1-E2 ATPase domain includes a protein having an amino acid sequence of about 80-100, or more preferably about 87, 89, or 90 amino acid residues, and a bit score of at least 35, 40, 50, or more preferably, 37.0, 51.4, or 53.4. To identify the presence of an E1-E2 ATPase domain in a HEAT protein, and make the determination that a protein of interest has a particular profile, the amino acid sequence of the protein is searched against a database of known protein motifs and/or domains (e.g., the HMM database). The E1-E2 ATPase domain (HMM) has been assigned the PFAM Accession number PF00122 (see the PFAM website, available online through Washington University in Saint Louis). A search was performed against the HMM database resulting in the identification of an E1-E2 ATPase domain in the amino acid sequence of human HEAT-1 at about residues 299-387 of SEQ ID NO:64. A search was also performed against the HMM database resulting in the identification of an E1-E2 ATPase domain in the amino acid sequence of human HEAT-2 at about residues 278-365 of SEQ ID NO:68. A search was further performed against the HMM database resulting in the identification of an E1-E2 ATPase domain in the amino acid sequence of human HEAT-3 at about residues 302-392 of SEQ ID NO:71.

A description of the Pfam database can be found in Sonhammer et al. (1997) Proteins 28:405-420, and a detailed description of HMMs can be found, for example, in Gribskov et al. (1990) Methods Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference.

Preferably an E1-E2 ATPase domain is at least about 70-110 amino acid residues and has an “E1-E2 ATPase activity”, for example, the ability to interact with a HEAT substrate or target molecule (e.g., ATP or a cation such as Ca²⁺); to transport a HEAT substrate or target molecule (e.g., a cation such as Ca²⁺) from one side of a biological membrane to the other; to adopt an E1 conformation or an E2 conformation; to convert a HEAT substrate or target molecule to a product (e.g., to hydrolyze ATP); to interact with a second non-HEAT protein; to modulate intra- or inter-cellular signaling and/or gene transcription (e.g., either directly or indirectly); to modulate vascular smooth muscle tone; to modulate cellular growth and/or proliferation; and/or to modulate angiogenesis. Accordingly, identifying the presence of an “E1-E2 ATPase domain” can include isolating a fragment of a HEAT molecule (e.g., a HEAT polypeptide) and assaying for the ability of the fragment to exhibit one of the aforementioned E1-E2 ATPase domain activities.

In another embodiment, a HEAT molecule of the present invention may also be identified based on its ability to adopt an E1 conformation or an E2 conformation. As used herein, an “E1 conformation” of a HEAT protein includes a 3-dimensional conformation of a HEAT protein which does not exhibit HEAT activity (e.g., the ability to transport Ca²⁺), as defined herein. An E1 conformation of a HEAT protein usually occurs when the HEAT protein is unphosphorylated. As used herein, an “E2 conformation” of a HEAT protein includes a 3-dimensional conformation of a HEAT protein which exhibits HEAT activity (e.g., the ability to transport c Ca²⁺), as defined herein. An E2 conformation of a HEAT protein usually occurs when the HEAT protein is phosphorylated.

In another embodiment, a HEAT protein of the present invention is identified based on the presence of an “E1-E2 ATPases phosphorylation site” in the protein or corresponding nucleic acid molecule. An E1-E2 ATPases phosphorylation site functions in accepting a phosphate moiety and has the following consensus sequence: D-K-T-G-T-[LIVM]-[TI] (SEQ ID NO:73), wherein D is phosphorylated. The use of amino acids in brackets indicates that the amino acid at the indicated position may be any one of the amino acids within the brackets, e.g., [TI] indicates any of one of either T (threonine) or I (isoleucine). The E1-E2 ATPases phosphorylation site has been assigned ProSite Accession Number PS00154. To identify the presence of an E1-E2 ATPases phosphorylation site in a HEAT protein, and to make the determination that a protein of interest has a particular profile, the amino acid sequence of the protein may be searched against a database of known protein domains (e.g., the ProSite database) using the default parameters (available online through the Swiss Institute for Bioinformatics). A search was performed against the ProSite database resulting in the identification of an E1-E2 ATPases phosphorylation site in the amino acid sequence of human HEAT-1 (SEQ ID NO:64) at about residues 513-519. A similar search resulted in the identification of an E1-E2 phosphorylation site in the amino acid sequence of human HEAT-2 (SEQ ID NO:68) at about residues 498-504 and in the amino acid sequence of human HEAT-3 (SEQ ID NO:71) at about residues 533-539.

Preferably an E1-E2 ATPase phosphorylation site has a “phosphorylation site activity,” for example, the ability to be phosphorylated; to be dephosphorylated; to regulate the E1-E2 conformational change of the HEAT protein in which it is contained; to regulate transport of Ca²⁺ across a biological membrane by the HEAT protein in which it is contained; and/or to regulate the activity (as defined herein) of the HEAT protein in which it is contained. Accordingly, identifying the presence of an “E1-E2 ATPases phosphorylation site” can include isolating a fragment of a HEAT molecule (e.g., a HEAT polypeptide) and assaying for the ability of the fragment to exhibit one of the aforementioned phosphorylation site activities.

The family of HEAT proteins of the present invention also comprises at least one “large extramembrane domain” in the protein or corresponding nucleic acid molecule. As used herein, a “large extramembrane domain” includes a domain having greater than 20 amino acid residues that is found between transmembrane domains, preferably on the cytoplasmic side of the plasma membrane, and does not span or traverse the plasma membrane. A large extramembrane domain preferably includes at least one, two, three, four or more motifs or consensus sequences characteristic of P-type or E1-E2 ATPases, i.e., includes one, two, three, four, or more “P-type ATPase consensus sequences or motifs”. As used herein, the phrase “P-type ATPase consensus sequences or motifs” includes any consensus sequence or motif known in the art to be characteristic of P-type ATPases, including, but not limited to, the P-type ATPase sequence 1 motif (as defined herein), the P-type ATPase sequence 2 motif (as defined herein), the P-type ATPase sequence 3 motif (as defined herein), and the E1-E2 ATPases phosphorylation site (as defined herein).

In one embodiment, the family of HEAT proteins of the present invention comprises at least one “N-terminal” large extramembrane domain in the protein or corresponding nucleic acid molecule. As used herein, an “N-terminal” large extramembrane domain is found in the N-terminal ⅓^{rd} of the protein, preferably between the fourth and fifth transmembrane domains of a HEAT protein, and includes about 50-270, 50-250, 60-230, 70-210, 80-190, 90-170, or preferably, 92, 151, or 163 amino acid residues. In a preferred embodiment, an N-terminal large extramembrane domain includes at least one P-type ATPase sequence 1 motif (as described herein). An N-terminal large extramembrane domain was identified in the amino acid sequence of human HEAT-1 at about residues 277-427 of SEQ ID NO:64. An N-terminal large extramembrane domain was also identified in the amino acid sequence of human HEAT-2 at about residues 318-409 of SEQ ID NO:68 and in the amino acid sequence of human HEAT-3 at about residues 282-444 of SEQ ID NO:71.

The family of HEAT proteins of the present invention also comprises at least one “C-terminal” large extramembrane domain in the protein or corresponding nucleic acid molecule. As used herein, a “C-terminal” large extramembrane domain is found in the C-terminal ⅔^{rds} of the protein, preferably between the sixth and seventh transmembrane domains of a HEAT protein and includes about 340-590, 360-570, 380-550, 400-530, 420-510, 440-490, or preferably, 451, 471, or 480 amino acid residues. In a preferred embodiment, a C-terminal large extramembrane domain includes at least one or more of the following motifs: a P-type ATPase sequence 2 motif (as described herein), a P-type ATPase sequence 3 motif (as defined herein), and/or an E1-E2 ATPases phosphorylation site (as defined herein). A C-terminal large extramembrane domain was identified in the amino acid sequence of human HEAT-1 at about residues 485-935 of SEQ ID NO:64, in the amino acid sequence of human HEAT-2 at about residues 470-940 of SEQ ID NO:68, and in the amino acid sequence of human HEAT-3 at about residues 510-989 of SEQ ID NO:71.

In another embodiment, a HEAT protein of the present invention includes at least one “P-type ATPase sequence 1 motif” in the protein or corresponding nucleic acid molecule. As used herein, a “P-type ATPase sequence 1 motif” is a conserved sequence motif diagnostic for P-type ATPases (Tang, X. et al. (1996) Science 272:1495-1497; Fagan, M. J. and Saier, M. H. (1994) J. Mol. Evol. 38:57). A P-type ATPase sequence 1 motif is involved in the coupling of ATP hydrolysis with transport (e.g., transport of Ca²⁺). The consensus sequence for a P-type ATPase sequence 1 motif is [DNS]-[QENR]-[SA]-[LIVSAN]-[LIV]-[TSN]-G-E-[SN] (SEQ ID NO:75). The use of amino acids in brackets indicates that the amino acid at the indicated position may be any one of the amino acids within the brackets, e.g., [SA] indicates any of one of either S (serine) or A (alanine). In a preferred embodiment, a P-type ATPase sequence 1 motif is contained within an N-terminal large extramembrane domain. In another preferred embodiment, a P-type ATPase sequence 1 motif in the HEAT proteins of the present invention has at least 1, 2, 3, 4, 5, 6, 7, 8 or more amino acid resides which match the consensus sequence for a P-type ATPase sequence 1 motif. A P-type ATPase sequence 1 motif was identified in the amino acid sequence of human HEAT-1 at about residues 341-349 of SEQ ID NO:64, in the amino acid sequence of human HEAT-2 at about residues 318-326 of SEQ ID NO:68, and in the amino acid sequence of human HEAT-3 at about residues 348-356 of SEQ ID NO:71.

In another embodiment, a HEAT protein of the present invention includes at least one “P-type ATPase sequence 2 motif” in the protein or corresponding nucleic acid molecule. As used herein, a “P-type ATPase sequence 2 motif” is a conserved sequence motif diagnostic for P-type ATPases (Tang, X. et al. (1996) Science 272:1495-1497; Fagan, M. J. and Saier, M. H. (1994) J. Mol. Evol. 38:57). Preferably, a P-type ATPase sequence 2 motif overlaps with and/or includes an E1-E2 ATPases phosphorylation site (as defined herein). The consensus sequence for a P-type ATPase sequence 2 motif is [LIV]-[CAML]-[STFL]-D-K-T-G-T-[LI]-T (SEQ ID NO:76). The use of amino acids in brackets indicates that the amino acid at the indicated position may be any one of the amino acids within the brackets, e.g., [LI] indicates any of one of either L (leucine) or I (isoleucine). In a preferred embodiment, a P-type ATPase sequence 2 motif is contained within a C-terminal large extramembrane domain. In another preferred embodiment, a P-type ATPase sequence 2 motif in the HEAT proteins of the present invention has at least 1, 2, 3, 4, 5, 6, 7, 8, 9 or more amino acid resides which match the consensus sequence for a P-type ATPase sequence 2 motif. A P-type ATPase sequence 2 motif was identified in the amino acid sequence of human HEAT-1 at about residues 510-519 of SEQ ID NO:64, in the amino acid sequence of human HEAT-2 at about residues 495-504 of SEQ ID NO:68, and in the amino acid sequence of human HEAT-3 at about residues 530-539 of SEQ ID NO:71.

In yet another embodiment, a HEAT protein of the present invention includes at least one “P-type ATPase sequence 3 motif” in the protein or corresponding nucleic acid molecule. As used herein, a “P-type ATPase sequence 3 motif” is a conserved sequence motif diagnostic for P-type ATPases (Tang, X. et al. (1996) Science 272:1495-1497; Fagan, M. J. and Saier, M. H. (1994) J. Mol. Evol. 38:57). A P-type ATPase sequence 3 motif is involved in ATP binding. The consensus sequence for a P-type ATPase sequence 3 motif is [TIV]-G-D-G-X-N-D-[ASG]-P-[ASV]-L (SEQ ID NO:77). X indicates that the amino acid at the indicated position may be any amino acid (i.e., is not conserved). The use of amino acids in brackets indicates that the amino acid at the indicated position may be any one of the amino acids within the brackets, e.g., [TIV] indicates any of one of either T (threonine), I (isoleucine), or V (valine). In a preferred embodiment, a P-type ATPase sequence 3 motif is contained within a C-terminal large extramembrane domain. In another preferred embodiment, a P-type ATPase sequence 3 motif in the HEAT proteins of the present invention has at least 1, 2, 3, 4, 5, 6, 7, 8 or more amino acid resides (including the amino acid at the position indicated by “X”) which match the consensus sequence for a P-type ATPase sequence 3 motif. A P-type ATPase sequence 3 motif was identified in the amino acid sequence of human HEAT-1 at about residues 876-886 of SEQ ID NO:64, in the amino acid sequence of human HEAT-2 at about residues 881-891 of SEQ ID NO:68, and in the amino acid sequence of human HEAT-3 at about residues 862-872 of SEQ ID NO:71.

Isolated HEAT proteins of the present invention have an amino acid sequence sufficiently homologous to the amino acid sequence of SEQ ID NO:64, 68, or 71, or are encoded by a nucleotide sequence sufficiently homologous to SEQ ID NO:63, 65, 67, 69, 70, or 72. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains having at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more homology or identity across the amino acid sequences of the domains and contain at least one and preferably two structural domains or motifs, are defined herein as sufficiently homologous. Furthermore, amino acid or nucleotide sequences which share at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more homology or identity and share a common functional activity are defined herein as sufficiently homologous.

In a preferred embodiment, a HEAT protein includes at least one or more of the following domains or motifs: a transmembrane domain, an E1-E2 ATPase domain, an E1-E2 ATPases phosphorylation site, an N-terminal large extramembrane domain, a C-terminal large extramembrane domain, a P-type ATPase sequence 1 motif, a P-type ATPase sequence 2 motif, and/or a P-type ATPase sequence 3 motif, and has an amino acid sequence at least about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more homologous or identical to the amino acid sequence of SEQ ID NO:64, 68, or 71. In yet another preferred embodiment, a HEAT protein includes at least one or more of the following domains or motifs: a transmembrane domain, an E1-E2 ATPase domain, an E1-E2 ATPases phosphorylation site, an N-terminal large extramembrane domain, a C-terminal large extramembrane domain, a P-type ATPase sequence 1 motif, a P-type ATPase sequence 2 motif, and/or a P-type ATPase sequence 3 motif, and is encoded by a nucleic acid molecule having a nucleotide sequence which hybridizes under stringent hybridization conditions to a complement of a nucleic acid molecule comprising the nucleotide sequence of SEQ ID NO:63, 65, 67, 69, 70, or 72. In another preferred embodiment, a HEAT protein includes at least one or more of the following domains or motifs: a transmembrane domain, an E1-E2 ATPase domain, an E1-E2 ATPases phosphorylation site, an N-terminal large extramembrane domain, a C-terminal large extramembrane domain, a P-type ATPase sequence 1 motif, a P-type ATPase sequence 2 motif, and/or a P-type ATPase sequence 3 motif, and has a HEAT activity.

As used interchangeably herein, a “HEAT activity”, “biological activity of HEAT” or “functional activity of HEAT”, includes an activity exerted or mediated by a HEAT protein, polypeptide or nucleic acid molecule on a HEAT responsive cell or on a HEAT substrate, as determined in vivo or in vitro, according to standard techniques. In one embodiment, a HEAT activity is a direct activity, such as an association with a HEAT target molecule. As used herein, a “target molecule” or “binding partner” is a molecule with which a HEAT protein binds or interacts in nature, such that HEAT-mediated function is achieved. A HEAT target molecule can be a non-HEAT molecule or a HEAT protein or polypeptide of the present invention. In an exemplary embodiment, a HEAT target molecule is a HEAT substrate (e.g., a Ca²⁺ ion; ATP; or a non-HEAT protein). A HEAT activity can also be an indirect activity, such as a cellular signaling activity mediated by interaction of the HEAT protein with a HEAT substrate (e.g., regulation of vascular smooth muscle tone, cellular growth and/or proliferation, and/or angiogenesis).

In a preferred embodiment, a HEAT activity is at least one of the following activities: (i) interaction with a HEAT substrate or target molecule (e.g., a Ca²⁺ ion; ATP; or a non-HEAT protein); (ii) transport of a HEAT substrate or target molecule (e.g., a Ca²⁺ ion) from one side of a biological membrane to the other; (iii) the ability to be phosphorylated or dephosphorylated; (iv) adoption of an E1 conformation or an E2 conformation; (v) conversion of a HEAT substrate or target molecule to a product (e.g., hydrolysis of ATP to ADP and free phosphate); (vi) interaction with a second non-HEAT protein; (vii) modulation of intra- or inter-cellular signaling and/or gene transcription (e.g., either directly or indirectly); (viii) modulation of vascular smooth muscle tone; (ix) modulation of cellular growth and/or proliferation; and/or (x) modulation of angiogenesis.

Isolation of the Human HEAT cDNAs

The invention is based, at least in part, on the discovery of genes encoding novel members of the E1-E2 ATPase family. The entire sequence of human clones Fbh49937, Fbh49931, and Fbh49933 were determined and found to contain open reading frames termed human “HEAT-1,” human “HEAT-2,” and human “HEAT-3,” respectively.

The nucleotide sequence encoding the human HEAT-b 1 gene, which is approximately 4055 nucleotides in length, is set forth as SEQ ID NO:63. The protein encoded by this nucleic acid molecule has a molecular weight of approximately 129.8 kD, it comprises about 1180 amino acids and it has the amino acid sequence set forth as SEQ ID NO:64. The coding region (open reading frame) of SEQ ID NO:63 is set forth as SEQ ID NO:65.

The nucleotide sequence encoding the human HEAT-2 gene, which is approximately 7249 nucleotides in length, is set forth as SEQ ID NO:67. The protein encoded by this nucleic acid molecule has a molecular weight of approximately 138.2 kD, it comprises about 1256 amino acids and it has the amino acid sequence set forth as SEQ ID NO:68. The coding region (open reading frame) of SEQ ID NO:67 is set forth as SEQ ID NO:69.

The nucleotide sequence encoding the human HEAT-3 gene, which is approximately 3919 nucleotides in length, is set forth as SEQ ID NO:70. The protein encoded by this nucleic acid molecule has a molecular weight of approximately 132.5 kD, it comprises about 1204 amino acids and it has the amino acid sequence set forth as SEQ ID NO:71. The coding region (open reading frame) of SEQ ID NO:70 is set forth as SEQ ID NO:72.

### Analysis of the Human HEAT Molecules

The amino acid sequences of human HEAT-1, HEAT-2, and HEAT-3 were analyzed using the program PSORT (available online; see Nakai, K. and Kanehisa, M. (1992) Genomics 14:897-911) to predict the localization of the proteins within the cell. This program assesses the presence of different targeting and localization of amino acid sequences within the query sequence. The results of the analyses show that human HEAT-1 may be localized to the endoplasmic reticulum, mitochondria, secretory vesicles, or vacuoles. The results of these analyses further show that human HEAT-2 may be localized to the endoplasmic reticulum or the mitochondria and that human HEAT-3 may be localized to endoplasmic reticulum, the mitochondria, or vacuoles.

Analyses of the amino acid sequence of human HEAT-1, HEAT-2, and HEAT-3 were performed using MEMSAT. These analyses resulted in the identification of twelve possible transmembrane domains in the amino acid sequence of human HEAT-2 at residues 29-50, 211-227, 234-253, 294-317, 410-434, 449-469, 941-960, 968-985, 1000-1020, 1076-1092, 1105-1129, and 1144-1160 of SEQ ID NO:68. These analyses further resulted in the identification of twelve possible transmembrane domains in the amino acid sequence of human HEAT-3 at residues 65-89, 99-116, 242-258, 265-281, 445-464, 493-509, 990-1007, 1015-1031, 1049-1073, 1049-1073, 1103-1119, 1134-1151, and 1171-1187 of SEQ ID NO:71. Further analysis of the amino acid sequence of SEQ ID NO:71 (e.g., a Clustal W (1.74) multiple sequence alignment, for example, of the human HEAT-3 amino acid sequence with a known C. elegans cation-transporting ATPase (YE56elegans; SEQ ID NO:74; GenBank Accession No. P90747)) clearly identifies the twelve transmembrane domains of the HEAT-3 gene. The analysis of human HEAT-1 predicted twelve possible transmembrane domains in the amino acid sequence of human HEAT-1 (SEQ ID NO:64) at about residues 8-25, 47-65, 256-276, 428-448, 464-484, 900-920, 936-954, 963-987, 994-1015, 1049-1065, 1079-1102, and 1118-1134. The potential transmembrane domain at about residues 900-920 has a notably low score of only 0.4 by MEMSAT analysis. Further analysis of the amino acid sequence of SEQ ID NO:64 (e.g., alignment with, for example, a known C. elegans E1-E2 ATPase cation transporter (SEQ ID NO:66)) resulted in the identification of a twelfth transmembrane domain at about amino acid residues 231-253 of SEQ ID NO:64. Accordingly, the human HEAT-1 protein of SEQ ID NO:64 is predicted to have at least twelve transmembrane domains, for example, at about residues 8-25, 47-65, 231-253, 256-276, 428-448, 464-484, 936-954, 963-987, 994-1015, 1049-1065, 1079-1102, and 1118-1134.

Searches of the amino acid sequences of human HEAT-1, HEAT-2, and HEAT-3 were also performed against the HMM database. These searches resulted in the identification of an “E1-E2 ATPase” domain in the amino acid sequence of HEAT-1 at about residues 299-387 (score=51.4) of SEQ ID NO:64. These searches also resulted in the identification of an “E1-E2 ATPase” domain in the amino acid sequence of human HEAT-2 at about residues 278-365 (score=53.4) of SEQ ID NO:68. These searches further resulted in the identification of an “E1-E2 ATPase” domain in the amino acid sequence of human HEAT-3 at about residues 302-392 (score=37.0) of SEQ ID NO:71.

Searches of the amino acid sequence of human HEAT-b 1 were performed against the Prosite database. These searches resulted in the identification of an “E1-E2 ATPases phosphorylation site” at about residues 513-519 of SEQ ID NO:64. These searches also resulted in the identification in the amino acid sequence of human HEAT-1 of a number of potential N-glycosylation sites, cAMP- and cGMP-dependent protein kinase phosphorylation sites, protein kinase C phosphorylation sites, casein kinase II phosphorylation sites, and N-myristoylation sites.

Searches of the amino acid sequence of human HEAT-2 were also performed against the Prosite database. These searches resulted in the identification of an “E1-E2 ATPases phosphorylation site” at about residues 498-504 of SEQ ID NO:68. These searches also resulted in the identification in the amino acid sequence of human HEAT-2 of a number of potential N-glycosylation sites, cAMP- and cGMP-dependent protein kinase phosphorylation sites, protein kinase C phosphorylation sites, casein kinase II phosphorylation sites, tyrosine phosphorylation sites, and N-myristoylation sites.

Searches of the amino acid sequence of human HEAT-3 were further performed against the Prosite database. These searches resulted in the identification of an “E1-E2 ATPases phosphorylation site” at about residues 533-539 of SEQ ID NO:71. These searches also resulted in the identification in the amino acid sequence of human HEAT-3 of a number of potential N-glycosylation sites, cAMP- and cGMP-dependent protein kinase phosphorylation sites, protein kinase C phosphorylation sites, casein kinase II phosphorylation sites, and N-myristoylation sites.

The amino acid sequence of human HEAT-2 was used as a database query using the BLASTP program. This search established that human HEAT-2 has the highest homology to a putative yeast Ca²⁺-transporting ATPase (high score=798, probability=2.9e-87).

Table 13 depicts an alignment of a region important in calcium binding from HEAT-1, HEAT-2, HEAT-3 with similar sequences from a number of E1-E2 ATPases of various substrate specificities from a number of different organisms. This region includes the sixth transmembrane domain from each of HEAT-1, HEAT-2, and HEAT-3, as well as a number of amino acid residues adjacent to the sixth transmembrane domain. Amino acid residues determined to be important for calcium binding by mutagenesis of a SERCA calcium-transporting E1-E2 ATPase are indicated (“SERCA mutagenesis”). Amino acid residues in this region that are critical for calcium binding are indicated in bold. Substrate specificities are as follows: Type V (calcium), Ca²⁺ (calcium), Cu²⁺ (copper), Na⁺/K⁺ (sodium/potassium), and PL (phospholipid).

TABLE 13

| | GenBank | | SEQ | Substrate |
|---|---|---|---|---|
| Gene Name | Acc No. | Sequence | ID NO: | Specificity |
| SERCA mutagenesis | | IPEGLPA | 78 | |
| Fbh49937FL (HEAT-1) | | DLVTVVVPPALPAAMTVCTLYAQSRLRR | 79 | |
| Fbh49931FL (HEAT-2) | | DIITITVPPALPAAMTAGIVYAQRRLKK | 80 | |
| Fbh49933FL (HEAT-3) | | LILTSVVPPELPIELSLAVNTSLIALAK | 81 | |
| ATC9_Yeast_yor291 | Q12697 | DIITIVVPPALPATLTIGTNFALSRLKEK | 82 | Type V |
| ATC6_Yeast_SPF1_ye1031w | P39986 | LIITSVVPPELPMELTMAVNSSLAALAK | 83 | Type V |
| ATCY_SCHPO_sp_0140220 | 014022 | VLTILVPPALPATLSVGIANSIARLSRA | 84 | Type V |
| Gp_7324471_CE | AAF59622 | DLVTIVVPPALPAVMGIGIFYAQRRLRQK | 85 | Type V |
| Sp_Q21286_YBF7_CAEEL | Q21286 | DIITIVVPPALPAAMSVGIINANSRLKKK | 86 | Type V |
| YH2M_CAEEL_sp_q27533 | Q27533 | DIITITVPPALPAAMSVGIINAQLRLKKK | 87 | Type V |
| YE56_CAEEL_CAB05683 | CAB05683 | LILTSVIPPELPIELSLAVNSSLMALQKL | 88 | Type V |
| ATP2A1_H_AAB53112 | AAB53112 | ALAVAAIPEGLPAVITTCLALGTRRMAKK | 89 | Ca²⁺ |
| Rabbit SERCA1 | P04191 | ALAVAAIPEGLPAVITTCLALGTRRMAKK | 90 | Ca²⁺ |
| ATCB_Chick_SERCA1 | P13585 | ALAVAAIPEGLPAVITTCLALGTRRMAKK | 91 | Ca²⁺ |
| ATC2_FELCA_SERCA2 | Q00779 | ALAVAAIPEGLPAVITTCLALGTRRMAKK | 92 | Ca²⁺ |
| SERCA Procambarus clarkii | AAB82291 | ALAVAAIPEGLPAVITTCLALGTRRMAKK | 93 | Ca²⁺ |
| hSERCA2 | P16615 | ALAVAAIPEGLPAVITTCLALGTRRMAKK | 94 | Ca²⁺ |
| hSERCA3 | Q93084 | ALAVAAIPEGLPAVITTCLALGTRRMARK | 95 | Ca²⁺ |
| ATCB_DROME_P22700_158416 | P22700 | AVAVAAIPEGLPAVITTCLALGTRRMAKK | 96 | Ca²⁺ |
| ATC1_Yeast_PMR1 | P13586 | SLAVAAIPEGLPIIVTVTLALGVLRMAKR | 97 | Ca²⁺ |
| Y_PMC1 | P38929 | TVIVVAVPEGLPLAVTLALAFATTRMTKD | 98 | Ca²⁺ |
| hPMCA1 | P20020 | TVLVVAVPEGLPLAVTISLAYSVKKMMKD | 99 | Ca²⁺ |
| hPMCA2 | Q01814 | TVLVVAVPEGLPLAVTISLAYSVKKMMKD | 100 | Ca²⁺ |
| Rat PMCA1 | | TVLVVAVPEGLPLAVTISLAYSVKKMMKD | 101 | Ca²⁺ |
| PMCA3_H | Q16720 | TVLVVAVPEGLPLAVTISLAYSVKKMMKD | 102 | Ca²⁺ |
| PMCA4_H | P23634 | TVLVVAVPEGLPLAVTISLAYSVKKMMKD | 103 | Ca²⁺ |
| Pca1p_Yeast | P38360 | TVLIVSCPCVIGLAVPIVFVIASGVAAKR | 104 | Cu²⁺ |
| AT7A_Human | Q04656 | TVLCIACPCSLGLATPTAVMVGTGVGAQN | 105 | Cu²⁺ |
| AT7B_Human | P35670 | TVLCIACPCSLGLATPTAVMVGTGVAAQN | 106 | Cu²⁺ |
| ATNA_DROME_P13607_732656 | P13607 | GIIVANVPEGLLATVTVCLTLTAKRMASK | 107 | Na⁺/K⁺ |
| ATNA_HYDAT | P35317 | GIIVANVPEGLLATVTVCLTLTAKKMAKK | 108 | Na⁺/K⁺ |
| ATN1_BUFMA | P30714 | GIIVANVPEGLLATVTVCLTLTAKRMARK | 109 | Na⁺/K⁺ |
| ATN1 Human | P05023 | GIIVANVPEGLLATVTVCLTLTAKRMARK | 110 | Na⁺/K⁺ |
| ATN2 Human | P50993 | GIIVANVPEGLLATVTVCLTLTAKRMARK | 111 | Na⁺/K⁺ |
| ATN3 Human | P13637 | GIIVANVPEGLLATVTVCLTVTAKRMARK | 112 | Na⁺/K⁺ |
| ATPP2_H_AAD34706 | AAD34706 | ILFNNLIPISLLVTLEVVKFTQAYFINWD | 113 | PL |
| DRS2_ATC4_yeast | P39524 | ILFSNLVPISLFVTVELIKYYQAFMIGSD | 114 | PL |

Tissue Expression Analysis of HEAT-1, HEAT-2, and HEAT-3 mRNA Using Transcriptional Profiling and TAQMAN™ Analysis

This example describes the tissue distribution of human HEAT-2 mRNA, as determined using transcriptional profiling analysis and the TAQMAN™ procedure. For transcriptional profiling analysis, an array of several thousand cDNA clones are spotted onto a nylon membrane and probed with a complex probe prepared by radiolabeling cDNA made from mRNA from, for example, normal tissue, and another, separate probe made from mRNA from another tissue, for example, diseased tissue. Expression levels of each gene in the first (e.g., normal) and the second (e.g., diseased) tissue are then compared. Transcriptional profiling thus allows assessment of the expression level of several thousand genes in an mRNA sample at the same time.

### Endothelial Cell Paradigms

To induce tube formation, human microvascular endothelial cells isolated from the lung (HMVECs) were plated on Matrigel to induce capillary-like tube formation. At 5 hours, the cells were actively forming tubes, and RNA was harvested. Additional RNA samples were prepared from cells 25 hours after plating on Matrigel when tube formation was complete, and from actively proliferating and confluent HMVECs grown on plastic.

Cells were also treated with laminar shear stress (LSS) of 7 dyn/cm² for 24-30 hours, LSS plus one or six additional hours of 12 dyn/cm² (“1 h up” or “6 h up”), or LSS plus one or six additional hours of 2 dyn/cm² (“1 h down” or “6 h down”).

### HEAT-1

The expression levels of human HEAT-b 1 mRNA in various human and monkey cell types and tissues was first determined using the TAQMAN™ procedure. The tissues and cells tested correspond to (1) normal artery; (2) normal vein; (3) aortic smooth muscle cells (early); (4) coronary smooth muscle cells; (5) umbilical vein endothelial cells (static); (6) umbilical vein endothelial cells (shear); (7) normal heart; (8) heart (congestive heart failure); (9) kidney; (10) skeletal muscle; (11) normal adipose tissue; (12) pancreas; (13) primary osteoblasts; (14) differentiated osteoclasts; (15) normal skin; (16) normal spinal cord; (17) normal brain cortex; (18) normal brain hypothalamus; (19) nerve; (20) dorsal root ganglion; (21) glial cells (astrocytes); (22) glioblastoma; (23) normal breast; (24) breast tumor; (25) normal ovary; (26) ovarian tumor; (27) normal prostate; (28) prostate tumor; (29) epithelial cells (prostate); (30) normal colon; (31) colon tumor; (32) normal lung; (33) lung tumor; (34) lung (chronic obstructive pulmonary disease); (35) colon (inflammatory bowel disease); (36) normal liver; (37) liver fibrosis; (38) dermal cells (fibroblasts); (39) normal spleen; (40) normal tonsil; (41) lymph node; (42) small intestine; (43) skin (decubitus); (44) synovium; (45) bone marrow mononuclear cells; and (46) activated peripheral blood mononuclear cells. As assessed by this TAQMAN™ analysis, HEAT-1 is highly expressed in coronary artery vascular smooth muscle cells, prostate epithelial cells, pancreas, and brain (including cortex, hypothalamus, dorsal root ganglion cells, and glial cells/astrocytes).

The expression levels of human HEAT-1 mRNA in various human vascular rich organs was then determined using the TAQMAN™ procedure, the samples tested include (1) confluent microvascular endothelial cells; (2) aortic smooth muscle cells; (3) fetal heart; (4) normal heart atrium; (5) normal heart atrium; (6) normal heart ventricle; (7) normal heart ventricle; (8) normal heart ventricle; (9) normal heart ventricle; (10) normal heart ventricle; (11) diseased heart ventricle; (12) diseased heart ventricle; (13) diseased heart ventricle; (14) normal kidney; (15) normal kidney; (16) normal kidney; (17) normal kidney; (18) normal kidney; (19) hypertensive kidney; (20) hypertensive kidney; (21) hypertensive kidney; (22) hypertensive kidney; (23) skeletal muscle; (24) skeletal muscle; (25) liver; (26) liver; (27) normal fetal adrenal gland; (28) Wilms tumor; (29) Wilms tumor; (30) normal spinal cord; and (31) diseased cartilage. As assessed by this TAQMAN™ panel, HEAT-1 is highly expressed in Wilms' tumor, normal spinal cord, and microvascular endothelial cells.

In another TAQMAN™ experiment, the expression levels of human HEAT-b 1 mRNA in various human and monkey vessels, namely (1) human aortic smooth muscle cells; (2) human microvascular endothelial cells; (3) human adipose tissue; (4) human normal carotid artery; (5) human normal carotid artery; (6) human normal muscular artery; (7) human diseased iliac artery; (8) human diseased tibial artery; (9) human diseased aorta; (10) human normal saphenous vein; (11) human normal saphenous vein; (12) human normal saphenous vein; (13) human normal saphenous vein; (14) human diseased saphenous vein; (15) human normal vein; (16) human normal vein; (17) human normal vein; (18) monkey normal coronary artery; (19) monkey normal coronary artery; (20) monkey normal coronary artery; (21) monkey normal coronary artery; (22) monkey normal vein; and (23) no transcriptase control, was determined. This TAQMAN™ panel demonstrated that HEAT-1 is highly expressed in vessels such as arteries and veins.

The expression levels of human HEAT-1 mRNA in various human coronary vascular cell types, namely (1) aortic smooth muscle cells; (2) aortic smooth muscle cells; (3) aortic smooth muscle cells; (4) coronary smooth muscle cells; (5) coronary smooth muscle cells; (6) coronary smooth muscle cells; (7) coronary smooth muscle cells; (8) macrophages; (9) macrophages treated with IFNγ; (10) macrophages treated with CD40; (11) macrophages treated with LPS; (12) umbilical vein endothelial cells; (13) microvascular endothelial cells; (14) aortic endothelial cells; (15) aortic endothelial cells; (16) cortex renal epithelium; (17) renal proximal tubule epithelium; (18) mesangial cells; (19) skeletal muscle; (20) skeletal muscle; and (21) lung fibroblasts was also determined using the TAQMAN™ procedure. This TAQMAN™ panel demonstrated that HEAT-1 is highly expressed in coronary and vascular smooth muscle cells, as compared to other cell types.

The expression levels of human HEAT-1 mRNA in various human endothelial cell paradigms was determined using the TAQMAN™ procedure. These experiments demonstrated that human HEAT-1 is upregulated during shear stress of endothelial cells and that human HEAT-1 is upregulated during proliferation and tube formation of endothelial cells. These data strongly link human HEAT-1 to a role in angiogenesis.

### HEAT-2

The expression levels of human HEAT-2 mRNA in various human and monkey cell types and tissues was first determined using transcriptional profiling. The samples tested include (1) human aortic smooth muscle cells; (2) human coronary artery smooth muscle cells; (3) human umbilical vein endothelial cells; (4) human microvascular endothelial cells (lung); (5) monkey aorta; (6) monkey vein; (7) monkey heart; and (8) monkey liver. As shown in these experiments, HEAT-2 is highly expressed in coronary artery vascular smooth muscle cells, as compared to other tissues such as aortic vascular smooth muscle cells, umbilical vein endothelial cells, microvascular endothelial cells, heart, liver, aorta, and vein. The expression levels of human HEAT-2 mRNA in various human cell types and tissues was then confirmed in a second experiment using the TAQMAN™ procedure using a panel consisting of the following tissues and samples: (1) normal aorta; (2) normal fetal heart; (3) normal heart; (4) heart (congestive heart failure); (5) normal vein; (6) aortic smooth muscle cells; (7) normal spinal cord; (8) brain (normal cortex); (9) brain (hypothalamus); (10) glial cells (astrocytes); (11) brain (glioblastoma); (12) normal breast; (13) breast tumor (infiltrating ductal carcinoma); (14) normal ovary; (15) ovarian tumor; (16) pancreas; (17) normal prostate; (18) prostate tumor; (19) normal colon; (20) colon tumor; (21) colon (inflammatory bowel disease); (22) normal kidney; (23) normal liver; (24) fibrotic liver; (25) normal fetal liver; (26) normal lung; (27) lung tumor; (28) lung (chronic obstructive pulmonary disease); (29) normal spleen; (30) normal tonsil; (31) normal lymph node; (32) normal thymus; (33) epithelial cells (from prostate); (34) aortic endothelial cells; (35) skeletal muscle; (36) dermal fibroblasts; (37) normal skin; (38) normal adipose tissue; (39) primary osteoblasts; (40) undifferentiated osteoblasts; (41) differentiated osteoblasts; (42) osteoclasts; (43) aortic smooth muscle cells (early); (44) aortic smooth muscle cells (late); (45) human umbilical vein endothelial cells (shear); and (46) human umbilical vein endothelial cells (static).

The expression levels of human HEAT-2 mRNA in various human vascular rich organs, namely (1) normal human heart; (2) normal human heart; (3) normal human heart; (4) normal human heart; (5) normal human heart; (6) normal human heart; (7) normal human heart; (8) normal human heart; (9) diseased human heart; (10) diseased human right ventricle; (11) diseased human left ventricle; (12) normal monkey heart; (13) normal monkey heart; (14) normal monkey heart; (15) normal human kidney; (16) normal human kidney; (17) normal human kidney; (18) normal human kidney; (19) normal human kidney; (20) human hypertensive kidney; (21) human hypertensive kidney; (22) human hypertensive kidney; (23) human hypertensive kidney; (24) human hypertensive kidney; (25) human liver; (26) human liver; (27) human liver; (28) human skeletal muscle; (29) human skeletal muscle; and (30) human skeletal muscle, was then determined using the TAQMAN™ procedure. These experiments demonstrated that HEAT-2 is highly expressed in the heart.

In another experiment, the expression levels of human HEAT-2 mRNA in various human and monkey vessels, namely (1) human adipose tissue; (2) human normal artery; (3) human normal artery; (4) human carotid artery; (5) human carotid artery; (6) human normal artery; (7) human diseased artery; (8) human diseased artery; (9) human diseased artery; (10) human normal vein; (11) human normal vein; (12) human vein; (13) human vein; (14) human normal vein; (15) human varicose vein; (16) confluent human microvascular endothelial cells; (17) human aortic smooth muscle cells; (18) monkey aorta; (19) monkey aorta; (20) monkey aorta; (21) monkey artery; (22) monkey artery; (23) monkey renal artery; (24) monkey renal artery; (25) monkey renal artery; (26) monkey renal artery; (27) monkey renal artery; (28) monkey coronary artery; (29) monkey coronary artery; (30) monkey coronary artery; (31) monkey coronary artery; (32) monkey coronary artery; (33) monkey coronary artery; and (34) monkey coronary artery, was determined using the TAQMAN™ procedure and in situ hybridization. These experiments demonstrated that HEAT-2 is highly expressed in vessels such as arteries and veins.

The expression levels of human HEAT-2 mRNA in various human coronary vascular cell types, namely (1) aortic smooth muscle cells; (2) aortic smooth muscle cells; (3) aortic smooth muscle cells; (4) aortic smooth muscle cells; (5) coronary smooth muscle cells; (6) coronary smooth muscle cells; (7) coronary smooth muscle cells; (8) coronary smooth muscle cells; (9) macrophages; (10) macrophages treated with IFNγ; (11) macrophages treated with CD40; (12) macrophages treated with LPS; (13) umbilical vein endothelial cells; (14) microvascular endothelial cells; (15) aortic endothelial cells; (16) coronary artery endothelial cells; (17) coronary artery endothelial cells; (18) cortex renal epithelium; (19) renal proximal tubule epithelium; (20) mesangial cells; (21) skeletal muscle; (22) skeletal muscle; and (23) lung fibroblasts, was also determined using the TAQMAN™ procedure. As determined by these experiments, HEAT-2 is highly expressed in coronary vascular smooth muscle cells, as compared to other cell types.

The expression levels of human HEAT-2 mRNA in various human endothelial cell paradigms was determined using the TAQMAN™ procedure. The samples tested include (1) umbilical vein endothelial cells (static); (2) umbilical vein endothelial cells (shear regulated); (3) umbilical vein endothelial cells (proliferating); (4) umbilical vein endothelial cells (confluent); (5) umbilical vein endothelial cells (without growth factor treatment); (6) umbilical vein endothelial cells (Interleukin-1 stimulated); (7) microvascular endothelial cells (proliferating); (8) microvascular endothelial cells (confluent); (9) microvascular endothelial cells (proliferating); (10) microvascular endothelial cells (confluent); (11) microvascular endothelial cells (proliferating); (12) microvascular endothelial cells (confluent); (13) microvascular endothelial cells (without growth factor treatment); (14) coronary microvascular endothelial cells (proliferating); (15) coronary microvascular endothelial cells (confluent); (16) microvascular endothelial cells (5% serum plus growth factors); (17) microvascular endothelial cells (5% serum without growth factors); (18) microvascular endothelial cells (HEGF treated); (19) microvascular endothelial cells (VEGF treated); (20) microvascular endothelial cells (bFGF treated); (21) microvascular endothelial cells (IGF treated); (22) 293 cells; (23) umbilical vein endothelial cells (static 25 h); (24) umbilical vein endothelial cells (laminar shear stress); (25) umbilical vein endothelial cells (laminar shear stress+1 h up); (26) umbilical vein endothelial cells (laminar shear stress+1 h down); (27) umbilical vein endothelial cells (static 30 h); (28) umbilical vein endothelial cells (laminar shear stress); (29) umbilical vein endothelial cells (laminar shear stress+6 h up); (30) umbilical vein endothelial cells (static 30 h); (31) umbilical vein endothelial cells (laminar shear stress); and (32) umbilical vein endothelial cells (laminar shear stress+6 h down). These experiments demonstrate that HEAT-2 is upregulated during shear and proliferation of endothelial cells. These data strongly link HEAT-2 to a role in angiogenesis.

Human HEAT-2 was also shown to be upregulated during tube formation of endothelial cells. The expression level of human HEAT-2 is 5-fold higher in the 5-hour Matrigel sample than in any other sample, indicating that expression is significantly induced during the process of capillary-like tube formation. There is also significantly higher expression in proliferating HMVECs than in confluent HMVECs grown on plastic. These results indicate a pro-angiogenic function for human HEAT-2.

Human HEAT-2 mRNA expression was also detected by in situ hybridization analysis in human endothelial cells and myocytes in the heart and in endothelial cells and inflammatory cells in ApoE knockout mouse diseased aortic roots.

### HEAT-3

The expression levels of human HEAT-3 mRNA in various human and monkey cell types and tissues, namely (1) normal aorta; (2) normal fetal heart; (3) normal heart; (4) heart (congestive heart failure); (5) normal vein; (6) normal spinal cord; (7) normal brain cortex; (8) normal brain hypothalamus; (9) glial cells (astrocytes); (10) glioblastoma (brain); (11) normal breast; (12) breast tumor (infiltrating ductal carcinoma); (13) normal ovary; (14) ovarian tumor; (15) pancreas; (16) normal prostate; (17) prostate tumor; (18) normal colon; (19) colon tumor; (20) colon (inflammatory bowel disease); (21) normal kidney; (22) normal liver; (23) liver fibrosis; (24) normal fetal liver; (25) normal lung; (26) lung tumor; (27) lung (chronic obstructive pulmonary disease); (28) normal spleen; (29) normal tonsil; (30) normal lymph node; (31) normal thymus; (32) epithelial cells (prostate); (33) endothelial cells (aortic); (34) normal skeletal muscle; (35) fibroblasts (dermal); (36) normal skin; (37) normal adipose tissue; (38) primary osteoblasts; (39) undifferentiated osteoblasts; (40) differentiated osteoblasts; (41) osteoclasts; (42) aortic smooth muscle cells (early); (43) aortic smooth muscle cells (late); (44) umbilical vein endothelial cells (laminar shear stress); (45) umbilical vein endothelial cells (static); and (46) undifferentiated osteoclasts, was first determined using the TAQMAN™ procedure. These results demonstrate that HEAT-3 is highly expressed in coronary artery vascular smooth muscle cells, prostate epithelial cells, pancreas, and brain (including cortex, hypothalamus, and glial cells/astrocytes).

The expression levels of human HEAT-3 mRNA in various human vascular rich organs, namely (1) normal heart; (2) normal heart; (3) normal heart; (4) normal heart; (5) normal heart; (6) normal heart; (7) normal heart; (8) normal heart; (9) diseased heart; (10) diseased right ventricle; (11) normal fetal heart; (12) normal kidney; (13) normal kidney; (14) normal kidney; (15) normal kidney; (16) normal kidney; (17) hypertensive kidney; (18) hypertensive kidney; (19) hypertensive kidney; (20) hypertensive kidney; (21) hypertensive kidney; (22) skeletal muscle; (23) skeletal muscle; (24) skeletal muscle; (25) liver; (26) liver; (27) normal monkey heart; (28) normal monkey heart; (29) normal monkey heart; (30) normal monkey heart; (31) smooth muscle cells (SMC); (32) confluent human microvascular endothelial cells (HMVECs); (33) M human umbilical vein endothelial cells (HUVECs); (34) human umbilical vein endothelial cells (HUVECs)-vehicle; (35) M human amniotic endothelial cells (HAECs); and (36) human amniotic endothelial cells (HAECs)-vehicle, was then determined using the TAQMAN™ procedure. These experiments demonstrated that HEAT-3 is expressed in the heart, kidney, and skeletal muscle.

In another experiment, the expression levels of human HEAT-3 mRNA in various vessels, namely (1) aortic smooth muscle cells; (2) microvascular endothelial cells; (3) adipose tissue; (4) normal artery; (5) normal artery; (6) normal artery; (7) diseased artery; (8) diseased artery; (9) diseased aorta; (10) normal vein; (11) normal vein; (12) normal vein; (13) normal vein; (14) diseased vein; (15) normal vein; (16) normal vein; (17) normal vein; (18) LC smooth muscle cells; (19) LC smooth muscle cells; (20) aortic smooth muscle cells; (21) human microvascular endothelial cells; (22) normal human carotid artery; (23) normal human carotid artery; (24) normal human muscular artery; (25) human diseased iliac artery; (26) human diseased tibial artery; (27) human diseased aorta; (28) human normal saphenous vein; (29) human normal saphenous vein; (30) human normal saphenous vein; (31) human normal saphenous vein; (32) human diseased saphenous vein; (33) human normal vein; and (34) human normal saphenous vein, was determined using the TAQMAN™ procedure. These experiments demonstrated that HEAT-3 is highly expressed in vessels such as arteries and veins.

The expression levels of human HEAT-3 mRNA in various human coronary vascular cell types, namely (1) aortic smooth muscle cells; (2) aortic smooth muscle cells; (3) coronary smooth muscle cells; (4) coronary smooth muscle cells; (5) coronary smooth muscle cells; (6) coronary smooth muscle cells; (7) macrophages; (8) macrophages treated with IFNγ; (9) macrophages treated with CD40; (10) macrophages treated with LPS; (11) microvascular endothelial cells; (12) aortic endothelial cells; (13) coronary artery endothelial cells; (14) cortex renal epithelium; (15) renal proximal tubule epithelium; (16) mesangial cells; and (17) skeletal muscle, was also determined using the TAQMAN™ procedure. These experiments demonstrate that HEAT-3 is highly expressed in coronary and aortic vascular smooth muscle cells, as well as in renal proximal tubule epithelium, as compared to other cell types.

Tissue Distribution of HEAT mRNA Using In Situ Analysis

Using in situ hybridization analysis, HEAT-2 mRNA was found to be expressed in human endothelial cells and myocytes in the heart and in endothelial cells and inflammatory cells in ApoE knockout mice diseased aortic roots.

### Assessment of Microvessel Contraction

The following describes the assessment of microvessel contraction using rat microvessels, as described in, for example, Bischoff, A. et al. (2000) Br. J. Pharmacol. 130:1871-1877. Microvessels (e.g., mesenteric or renal microvessels such as interlobar arteries) are prepared from rats (e.g., adult Wistar rats) as described in Chen et al. (1996) Naunyn-Schmiedeberg's Arch. Pharmacol. 353:314-323 and Chen et al. (1997) J. Auton. Pharmacol. 17:137-146. Rats are killed by either decapitation or an overdose of thiobutabarbitone. The vessels are mounted on 40 μm diameter stainless steel wires in a myograph chamber for isometric recording of tension development. The vessels are then bathed in Krebs-Henseleit buffer of the following composition: 119 mM NaCl, 25 mM NaHCO₃, 4.7 mM KCl, 1.18 mM KH₂PO₄, 1.17 mM MgSO₄, 2.5 mM CaCl₂, 0.026 mM EDTA, and 5.5 mM D-glucose. The buffer temperature is maintained at 37° C., and the chamber is gassed with 5% CO2/95% O2 to maintain a pH of 7.4. Additionally, 5 μM cocaine and 1 μM (±)-propranolol may be added to block neuronal catecholoamine uptake and β-adrenoceptor activation by high noradrenaline concentration. Following equilibration, the vessels are challenged several times with 125 mM KCl and 10 μM noradrenaline. The vessels are then treated with 100 μM carbachol; vessels with a relaxation response of at least 50% indicate a functionally intact epithelium.

### Assessment of Intracellular Free Calcium Concentrations in Cultured Rat Aortic Smooth Muscle Cells

The following describes the assessment of intracellular free calcium concentrations in cultured rat aortic smooth muscle cells, as described in, for example, Bischoff, A. et al. (2000) Br. J. Pharmacol. 130:1871-1877. Vascular smooth muscle cells are prepared from rat thoracic aorta according to Rosskoph et al. (1995) Cell Physiol. Biochem. 5:276-285). Briefly, freshly prepared aortae are incubated for 30 minutes at room temperature with 125 U/ml collagenase I in Hank's balanced salt solution (HBSS) of the following composition: 118 mM NaCl, 5 mM KCl, 1 mM CaCl₂, 1 mM MgCl₂, 5 mM D-glucose, and 15 mM HEPES pH 7.4. Thereafter, remaining connective tissue and endothelium are removed, the aortae are cut into small pieces and incubated for 4-6 hours at 37° C. in DMEM/F12 medium with 100 U/ml penicillin, 100 μg/ml streptomycin, and 250 ng/ml amphotericin B. Treatment with collagenase (125 U/ml) and elastase (0.5 mg/ml) in HBSS without Ca²⁺ and Mg²⁺ follow for 2 hours at 37° C. The reaction is stopped by addition of DMEM/F12 medium containing 20% fetal calf serum and penicillin, streptomycin, and amphotericin B, and the cells are plated onto 60-mm cell culture plates. The cells are used between passage 3 and 6. The Ca²⁺ concentration measurements are performed as described in Meyer zu Heringdorf et al. (1996) Naunyn-Schmiedeberg's Arch. Pharmacol. 354:397-403. Briefly, the cells are loaded with 1 μM fura2/AM for 1 hour at room temperature in HBSS, washed with HBSS, and used for fluorescence measurements within the next hour. Ca²⁺ concentrations are measured in a continuously stirred cell suspension at room temperature in a Hitachi F2000 spectrofluorometer as described in Meyer zu Heringdorf et al. (1996) supra.

### Calcium Transport Assay

The following describes the assessment of calcium transport by HEAT molecules in cultured COS-1 cells, as described in, for example, Maruyama, K. and MacLennan, D. H. (1988) Proc. Natl. Acad. Sci. USA 85:3314-3318.

### Cell Culture and DNA Transfection

COS-1 or HEK-293 cells are maintained in Dulbecco's modified Eagle's medium (DMEM) with 0.1 mM α-MEM nonessential amino acids, 4 mM L-glutamine, 100 units of penicillin per ml, 100 μg of streptomycin per ml, and 10% fetal calf serum under 5% CO₂/95% air at 37° C. Transfection of HEAT-containing DNA is carried out by the DEAE dextran-chloroquine shock method (Sompayrac, L. M. and Danna, K. J. (1981) Proc. Natl. Acad. Sci. USA 78:7575-7578; Gorman, C. (1985) in DNA Cloning: A Practical Approach, ed. Gover, D. M. (IRL, Washington, D.C.), Vol. 2, pp. 143-190) with 25 μg of cesium chloride gradient-purified DNA and 1.5 mg of DEAE dextran per 10 cm Petri dish. Cells are then incubated for 3 hours at 37° C. in 6 ml of DMEM containing 300 μg of chloroquine, washed, and cultured in DMEM for 48 or 72 hours. Control cells are treated in the same way with vector DNA or with no added DNA.

### Isolation of Microsomal Fraction

For isolation of a microsomal fraction (Resh, M. D. and Erikson, R. L. (1985) J. Cell Biol. 100:409-417; Yamada, S, and Ikemoto, N. (1980) J. Biol. Chem. 255:3108-3119), cells from five 10 cm Petri dishes are washed twice with 5 ml of a solution of 0.137 M NaCl/2.7 mM KCl/8 mM Na₂HPO₄/1.5 mM KH₂PO₄ (PBS), harvested in a solution of 5 mM EDTA in PBS and washed with 5 ml of PBS. The cells are swollen at 0° C. for 10 minutes in 2 ml of a hypotonic solution of 10 mM Tris-HCl, pH 7.5/0.5 mM MgCl₂, and then phenylmethylsolfonyl fluoride and Trasylol are added to 0.1 mM and 100 units/ml, respectively. The cells are homogenized with 30 strokes in a glass Dounce homogenizer, and the homogenate is diluted with an equal volume of a solution of 0.5 M sucrose/6 mM 2-mercaptoenthanol, 40 μM CaCl₂/300 mM KCl/10 mM Tris-HCl, pH 7.5. The suspension is centrifuged at 10,000×g for 20 minutes to pellet nuclei and mitochondria. The supernatant is brought to a concentration of 0.6 M KCl by the addition of 0.9 ml of a 2.5 M solution. The suspension is centrifuged at 100,000×g for 60 minutes to sediment the microsomal fraction. The pellet is suspended in a solution containing 0.25 M sucrose, 0.15 M KCl, 3 mM 2-mercaptoethanol, 20 μM CaCl₂, 10 mM Tris-HCl (pH 7.5), and centrifuged again at 100,000×g for 60 minutes. The final pellet, containing approximately 100 μg of protein, is suspended in the same solution at a protein concentration of 1 mg/ml.

### Ca²⁺ Transport Assay

Ca²⁺ transport activity is assayed in a reaction mixture containing 20 μM Mops-KOH (pH 6.8), 100 mM KCl, 5 mM CaCl₂, 5 mM ATP, 0.45 mM CaCl₂ (containing ⁴⁵Ca at a specific activity of 10⁶ cpm/μmol), 0.5 mM EGTA, and 5 mM potassium oxalate. The uptake reaction is initiated by the addition of 10 μg of microsomal protein to 1 ml of reaction mixture at room temperature. At different time points, 0.15 ml samples are filtered through a 0.3 μm Millipore filter and washed with 10 ml of 0.15 M KCl. Radioactivity on the filter is measured by liquid scintillation counting. For the measurement of Ca²⁺ ion dependency, free Ca²⁺ concentration is calculated by the computer program of Fabiato and Fabiato ((1979) J. Physiol. (London) 75:463-505). For the measurement of ATP dependency, an ATP regenerating system consisting of 2.5 mM phosphoenolpyruvate and 50 μg of pyruvate kinase per mM is used.

### Measurement of Phosphorylated HEAT Intermediate

Microsomal protein (5 μg) is added to 0.1 ml of a solution of 20 mM Mops, pH 6.8/100 mM KCl/5 mM MgCl₂/0.5 mM EGTA in the presence or absence of 0.5 mM CaCl₂. The reaction, at ice temperature, is started by the addition of 5 μM ATP (10⁶ cpm/nmol) and stopped after 5 seconds by the addition of 0.6 ml of a mixture of 5% trichloroacetic acid and 5 mM potassium phosphate. Incorporation of ³²P is determined either by collecting the protein on a filter for scintillation counting or by separating the protein in acidic NaDodSO₄/polyacrylamide gels for autoradiography Sarkadi, B. et al. (1986) J. Biol. Chem. 261:9552-9557).

### Analysis of HEAT-3 Activity

The full-length HEAT-3 was inserted into the multiple cloning site in the pCDNA3 vector. The DNA for the clone was amplified and transfected into HEK-293 cells using calcium phosphate precipitation. After 72 hours, the cells were harvested, microsomal fractions isolated, and ⁴⁵Ca-uptake measured as a function of calcium concentration using a filter assay.

Two different HEAT-3 fusion proteins were generated. One HEAT-3 fusion protein was created by inserting the 3× Flag epitope at the 3′ end of the HEAT-3 gene. Another HEAT-3 fusion protein was created by inserting the green fluorescent protein (GFP) at the 3′ end of the HEAT-3 gene. Fluorescence of this protein could be observed with the naked eye and by confocal microscopy. Measurement of expression using Western blotting with an anti-GFP antibody showed that HEAT-3 is well expressed in the microsomal fraction.

Confocal microscopy showed that the expression pattern of HEAT-3 is similar to SERCA1, indicating that HEAT-3 is targeted to and localized in the endoplasmic reticulum of HEK-293 cells.

Ca²⁺ uptake experiments (as described above) were performed using both the Flag and GFP fusion proteins. An increase in Ca²⁺ uptake of HEAT-3 was shown over GFP vector alone. Five independent experiments were performed to confirm the increase of calcium uptake with HEAT-3 as compared to vector alone. The Flag fusion protein showed an increase of calcium uptake as compared to Flag vector alone. In these experiments, the Vmax for HEAT-3 was lower (about 20×) than the Vmax for SERCA1 under the same conditions. The KCa for HEAT-3 was about 6.00 pCa units, as compared with about 6.38 pCa units for SERCA1. In the presence of ATP, there is 2-3 fold more calcium uptake compared to uptake in the absence of ATP, indicating that the calcium uptake by HEAT-3 is ATP dependent.

### DEFINITIONS

The 25869, 25934, 26335, 50365, 21117, 38692, 46508, 16816, 16839, 49937, 49931 or 49933 protein, fragments thereof, and derivatives and other variants of the sequence in SEQ ID NO:2, 8, 17, 21, 26, 29, 36, 40, 43, 64, 68 or 71 thereof are collectively referred to as “polypeptides or proteins of the invention” or “25869, 25934, 26335, 50365, 21117, 38692, 46508, 16816, 16839, 49937, 49931 or 49933 polypeptides or proteins”. Nucleic acid molecules encoding such polypeptides or proteins are collectively referred to as “nucleic acids of the invention” or “25869, 25934, 26335, 50365, 21117, 38692, 46508, 16816, 16839, 49937, 49931 or 49933 nucleic acids.”

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

As used herein, the term “hybridizes under low stringency, medium stringency, high stringency, or very high stringency conditions” describes conditions for hybridization and washing. Guidance for performing hybridization reactions can be found in Current Protocols in Molecular Biology (1989) John Wiley & Sons, N.Y., 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules which include an open reading frame encoding a 25869, 25934, 26335, 50365, 21117, 38692, 46508, 16816, 16839, 49937, 49931 or 49933 protein, preferably a mammalian 25869, 25934, 26335, 50365, 21117, 38692, 46508, 16816, 16839, 49937, 49931 or 49933 protein, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of 25869, 25934, 26335, 50365, 21117, 38692, 46508, 16816, 16839, 49937, 49931 or 49933 protein having less than about 30%, 20%, 10% and more preferably 5% (by dry weight), of non-25869, 25934, 26335, 50365, 21117, 38692, 46508, 16816, 16839, 49937, 49931 or 49933 protein (also referred to herein as a “contaminating protein”), or of chemical precursors or non-25869, 25934, 26335, 50365, 21117, 38692, 46508, 16816, 16839, 49937, 49931 or 49933 chemicals. When the 25869, 25934, 26335, 50365, 21117, 38692, 46508, 16816, 16839, 49937, 49931 or 49933 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of 25869, 25934, 26335, 50365, 21117, 38692, 46508, 16816, 16839, 49937, 49931 or 49933 (e.g., the sequence of SEQ ID NO:1, 3, 7, 9, 16, 18, 20, 22, 25, 27, 28, 30, 35, 37, 39, 41, 42, 44, 63, 65, 67, 69, 70 or 72) without abolishing or more preferably, without substantially altering a biological activity, whereas an “essential” amino acid residue results in such a change. For example, amino acid residues that are conserved among the polypeptides of the present invention, e.g., those present in the conserved domains, are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 25869, 25934, 26335, 50365, 21117, 38692, 46508, 16816, 16839, 49937, 49931 or 49933 protein is preferably replaced with another amino acid residue from the same side chain family. Alternatively, in another embodiment, mutations can be introduced randomly along all or part of a 25869, 25934, 26335, 50365, 21117, 38692, 46508, 16816, 16839, 49937, 49931 or 49933 coding sequence, such as by saturation mutagenesis, and the resultant mutants can be screened for 25869, 25934, 26335, 50365, 21117, 38692, 46508, 16816, 16839, 49937, 49931 or 49933 biological activity to identify mutants that retain activity. Following mutagenesis of SEQ ID NO:1, 3, 7, 9, 16, 18, 20, 22, 25, 27, 28, 30, 35, 37, 39, 41, 42, 44, 63, 65, 67, 69, 70 or 72, the encoded protein can be expressed recombinantly and the activity of the protein can be determined.

As used herein, a “biologically active portion” of a 25869, 25934, 26335, 50365, 21117, 38692, 46508, 16816, 16839, 49937, 49931 or 49933 protein includes a fragment of a 25869, 25934, 26335, 50365, 21117, 38692, 46508, 16816, 16839, 49937, 49931 or 49933 protein which participates in an interaction between a 25869, 25934, 26335, 50365, 21117, 38692, 46508, 16816, 16839, 49937, 49931 or 49933 molecule and a non-25869, 25934, 26335, 50365, 21117, 38692, 46508, 16816, 16839, 49937, 49931 or 49933 molecule. Biologically active portions of a 25869, 25934, 26335, 50365, 21117, 38692, 46508, 16816, 16839, 49937, 49931 or 49933 protein include peptides comprising amino acid sequences sufficiently homologous to or derived from the amino acid sequence of the 25869, 25934, 26335, 50365, 21117, 38692, 46508, 16816, 16839, 49937, 49931 or 49933 protein, e.g., the amino acid sequence shown in SEQ ID NO:2, 8, 17, 21, 26, 29, 36, 40, 43, 64, 68 or 71, which include fewer amino acids than the full length 25869, 25934, 26335, 50365, 21117, 38692, 46508, 16816, 16839, 49937, 49931 or 49933 protein, and exhibit at least one activity of a 25869, 25934, 26335, 50365, 21117, 38692, 46508, 16816, 16839, 49937, 49931 or 49933 protein. Typically, biologically active portions comprise a domain or motif with at least one activity of the 25869, 25934, 26335, 50365, 21117, 38692, 46508, 16816, 16839, 49937, 49931 or 49933 protein. A biologically active portion of a 25869, 25934, 26335, 50365, 21117, 38692, 46508, 16816, 16839, 49937, 49931 or 49933 protein can be a polypeptide which is, for example, 10, 25, 50, 100, 200 or more amino acids in length. Biologically active portions of a 25869, 25934, 26335, 50365, 21117, 38692, 46508, 16816, 16839, 49937, 49931 or 49933 protein can be used as targets for developing agents which modulate a 25869, 25934, 26335, 50365, 21117, 38692, 46508, 16816, 16839, 49937, 49931 or 49933 mediated activity.

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin or alternatively, can contain homologues of non-human origin, e.g., rat or mouse proteins. Members of a family can also have common functional characteristics.

Calculations of homology or sequence identity (the terms “homology” and “identity” are used interchangeably herein) between sequences are performed as follows:

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (1970) J. Mol. Biol. 48:444-453 algorithm which has been incorporated into the GAP program in the GCG software package using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to 25869, 25934, 26335, 50365, 21117, 38692, 46508, 16816, 16839, 49937, 49931 or 49933 nucleic acid molecules of the invention. BLAST protein searches can be performed with the XBLAST program, score=50, wordlength=3 to obtain amino acid sequences homologous to 25869, 25934, 26335, 50365, 21117, 38692, 46508, 16816, 16839, 49937, 49931 or 49933 protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

Particular 25869, 25934, 26335, 50365, 21117, 38692, 46508, 16816, 16839, 49937, 49931 or 49933 polypeptides of the present invention have an amino acid sequence substantially identical to the amino acid sequence of SEQ ID NO:2, 8, 17, 21, 26, 29, 36, 40, 43, 64, 68 or 71. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94%, 95%, 96%, 97%, 98% or 99% identity to SEQ ID NO:2, 8, 17, 21, 26, 29, 36, 40, 43, 64, 68 or 71 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97%, 98% or 99% identity to SEQ ID NO:1, 3, 7, 9, 16, 18, 20, 22, 25, 27, 28, 30, 35, 37, 39, 41, 42, 44, 63, 65, 67, 69, 70 or 72 are termed substantially identical.

“Misexpression or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject”, as used herein, can refer to a mammal, e.g., a human, or to an experimental or animal or disease model. The subject can also be a non-human animal, e.g., a horse, cow, goat, or other domestic animal.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

As used herein, cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the term “cancer” (also used interchangeably with the terms, “hyperproliferative” and “neoplastic”) refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Examples of cellular proliferative and/or differentiative disorders of the lung include, but are not limited to, tumors such as bronchogenic carcinoma, including paraneoplastic syndromes, bronchioloalveolar carcinoma, neuroendocrine tumors, such as bronchial carcinoid, miscellaneous tumors, metastatic tumors, and pleural tumors, including solitary fibrous tumors (pleural fibroma) and malignant mesothelioma.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders involving the colon include, but are not limited to, tumors of the colon, such as non-neoplastic polyps, adenomas, familial syndromes, colorectal carcinogenesis, colorectal carcinoma, and carcinoid tumors.

Examples of cancers or neoplastic conditions, in addition to the ones described above, include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Other disorders related to angiogenesis and which may, therefore, be treated using the molecules described herein, include diabetic retinopathy, neovascularization (e.g., intraocular neovascularization), psoriasis, endometriosis, Grave's disease, ischemic disease, chronic inflammatory diseases, macular degeneration, neovascular glaucoma, retinal fibroplasia, uveitis, eye diseases associated with choroidal neovascularization and iris neovascularization, hereditary hemorrhagic telangiectasia, fibrodysplasia ossificans progressiva, idiopathic pulmonary fibrosis, autosomal dominant polycystic kidney disease, synovitis, familial exudative vitreoretinopathy (FEVR), Alagille syndrome, Knobloch syndrome, disseminated lymphangiomatosis, toxic epidermal necrolysis, Von Hippel Lindau disease (VHL), microbial-related dysplastic and neoplastic angiomatous proliferative processes (e.g., verruga peruana (VP)), Proteus syndrome (PS), Castleman's disease, and Klippel-Trenaunay-Weber syndrome.

Proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

As used herein, disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-bome (Arbo) viral encephalitis, Herpes simplex virus Type 1, Herpes simplex virus Type 2, Varicella-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degenration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

As used herein, neurological disorders include disorders of the central nervous system (CNS) and the peripheral nervous system, e.g., cognitive and neurodegenerative disorders, Examples of neurological disorders include, but are not limited to, autonomic function disorders such as hypertension and sleep disorders, and neuropsychiatric disorders, such as depression, schizophrenia, schizoaffective disorder, Korsakoff's psychosis, alcoholism, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Such neurological disorders include, for example, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicella-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer's disease and Pick's disease, degenerative diseases of basal ganglia and brain stem, including Parkinsonism, idiopathic Parkinson's disease (paralysis agitans) and other Lewy diffuse body diseases, progressive supranuclear palsy, corticobasal degenration, multiple system atrophy, including striatonigral degenration, Shy-Drager syndrome, and olivopontocerebellar atrophy, and Huntington's disease, senile dementia, Gilles de la Tourette's syndrome, epilepsy, and Jakob-Creutzfieldt disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease. Further CNS-related disorders include, for example, those listed in the American Psychiatric Association's Diagnostic and Statistical manual of Mental Disorders (DSM), the most current version of which is incorporated herein by reference in its entirety.

As used herein, disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

As used herein, disorders of the breast include, but are not limited to, disorders of development; inflammations, including but not limited to, acute mastitis, periductal mastitis, periductal mastitis (recurrent subareolar abscess, squamous metaplasia of lactiferous ducts), mammary duct ectasia, fat necrosis, granulomatous mastitis, and pathologies associated with silicone breast implants; fibrocystic changes; proliferative breast disease including, but not limited to, epithelial hyperplasia, sclerosing adenosis, and small duct papillomas; tumors including, but not limited to, stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, no special type, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

As used herein, disorders involving the colon include, but are not limited to, congenital anomalies, such as atresia and stenosis, Meckel diverticulum, congenital aganglionic megacolon-Hirschsprung disease; enterocolitis, such as diarrhea and dysentery, infectious enterocolitis, including viral gastroenteritis, bacterial enterocolitis, necrotizing enterocolitis, antibiotic-associated colitis (pseudomembranous colitis), and collagenous and lymphocytic colitis, miscellaneous intestinal inflammatory disorders, including parasites and protozoa, acquired immunodeficiency syndrome, transplantation, drug-induced intestinal injury, radiation enterocolitis, neutropenic colitis (typhlitis), and diversion colitis; idiopathic inflammatory bowel disease, such as Crohn disease and ulcerative colitis; tumors of the colon, such as non-neoplastic polyps, adenomas, familial syndromes, colorectal carcinogenesis, colorectal carcinoma, and carcinoid tumors.

As used herein, disorders involving the kidney include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schönlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypemephroma, adenocarcinoma of kidney), which includes urothelial carcinomas of renal pelvis.

Examples of disorders of the lung include, but are not limited to, congenital anomalies; atelectasis; diseases of vascular origin, such as pulmonary congestion and edema, including hemodynamic pulmonary edema and edema caused by microvascular injury, adult respiratory distress syndrome (diffuse alveolar damage), pulmonary embolism, hemorrhage, and infarction, and pulmonary hypertension and vascular sclerosis; chronic obstructive pulmonary disease, such as emphysema, chronic bronchitis, bronchial asthma, and bronchiectasis; diffuse interstitial (infiltrative, restrictive) diseases, such as pneumoconioses, sarcoidosis, idiopathic pulmonary fibrosis, desquamative interstitial pneumonitis, hypersensitivity pneumonitis, pulmonary eosinophilia (pulmonary infiltration with eosinophilia), Bronchiolitis obliterans-organizing pneumonia, diffuse pulmonary hemorrhage syndromes, including Goodpasture syndrome, idiopathic pulmonary hemosiderosis and other hemorrhagic syndromes, pulmonary involvement in collagen vascular disorders, and pulmonary alveolar proteinosis; complications of therapies, such as drug-induced lung disease, radiation-induced lung disease, and lung transplantation; tumors, such as bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

As used herein, disorders involving the ovary include, for example, polycystic ovarian disease, Stein-leventhal syndrome, Pseudomyxoma peritonei and stromal hyperthecosis; ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecoma-fibromas, androblastomas, hill cell tumors, and gonadoblastoma; and metastatic tumors such as Krukenberg tumors.

As used herein, “a prostate disorder” refers to an abnormal condition occurring in the male pelvic region characterized by, e.g., male sexual dysfunction and/or urinary symptoms. This disorder may be manifested in the form of genitourinary inflammation (e.g., inflammation of smooth muscle cells) as in several common diseases of the prostate including prostatitis, benign prostatic hyperplasia and cancer, e.g., adenocarcinoma or carcinoma, of the prostate.

As used herein, the term “hematopoietic disorder” includes neoplastic and non-neoplastic hematopoietic or immune disorders. Examples of neoplastic immune disorders include, but are not limited to, erythroid leukemias, or leukemias of erythroid precursor cells, e.g., poorly differentiated acute leukemias such as erythroblastic leukemia and acute megakaryoblastic leukemia; acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97). In particular, AML can include the uncontrolled proliferation of CD34+ cells such as AML subtypes M1 and M2, myeloblastic leukemias with and without maturation, and AML subtype M6, erythroleukemia (Di Guglielmo's disease). Additional neoplastic disorders include a myelodysplastic syndrome or preleukemic disorder, e.g., oligoblastic leukemia, smoldering leukemia. Additional cancers of the erythroid lineage include erythroblastosis, and other relevant diseases of the bone marrow.

The term “leukemia” or “leukemic cancer” is intended to have its clinical meaning, namely, a neoplastic disease in which white corpuscle maturation is arrested at a primitive stage of cell development. The disease is characterized by an increased number of leukemic blast cells in the bone marrow, and by varying degrees of failure to produce normal hematopoietic cells. The condition may be either acute or chronic. Leukemias are further typically categorized as being either lymphocytic i.e., being characterized by cells which have properties in common with normal lymphocytes, or myelocytic (or myelogenous), i.e., characterized by cells having some characteristics of normal granulocytic cells. Acute lymphocytic leukemia (“ALL”) arises in lymphoid tissue, and ordinarily first manifests its presence in bone marrow. Acute myelocytic leukemia (“AML”) arises from bone marrow hematopoietic stem cells or their progeny. The term acute myelocytic leukemia subsumes several subtypes of leukemia: myeloblastic leukemia, promyelocytic leukemia, and myelomonocytic leukemia. In addition, leukemias with erythroid or megakaryocytic properties are considered myelogenous leukemias as well.

Examples of non-neoplastic hematopoietic disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjögren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

As used herein, the term “erythroid associated disorders” include disorders involving aberrant (increased or deficient) erythroblast proliferation, e.g., an erythroleukemia, and aberrant (increased or deficient) erythroblast differentiation, e.g., an anemia. Erythrocyte-associated disorders include anemias such as, for example, hemolytic anemias due to hereditary cell membrane abnormalities, such as hereditary spherocytosis, hereditary elliptocytosis, and hereditary pyropoikilocytosis; hemolytic anemias due to acquired cell membrane defects, such as paroxysmal nocturnal hemoglobinuria and spur cell anemia; hemolytic anemias caused by antibody reactions, for example to the RBC antigens, or antigens of the ABO system, Lewis system, Ii system, Rh system, Kidd system, Duffy system, and Kell system; methemoglobinemia; a failure of erythropoiesis, for example, as a result of aplastic anemia, pure red cell aplasia, myelodysplastic syndromes, sideroblastic anemias, and congenital dyserythropoietic anemia; secondary anemia in nonhematolic disorders, for example, as a result of chemotherapy, alcoholism, or liver disease; anemia of chronic disease, such as chronic renal failure; and endocrine deficiency diseases.

Agents that modulate polypeptide or nucleic acid activity or expression of the molecules of the invention can be used to treat anemias, in particular, anemias associated with cancer chemotherapy, chronic renal failure, malignancies, adult and juvenile rheumatoid arthritis, disorders of haemoglobin synthesis, prematurity, and zidovudine treatment of HIV infection. A subject receiving the treatment can be additionally treated with a second agent, e.g., erythropoietin, to further ameliorate the condition.

As used herein, the term “erythropoietin” or “EPO” refers to a glycoprotein produced in the kidney, which is the principal hormone responsible for stimulating red blood cell production (erythrogenesis). EPO stimulates the division and differentiation of committed erythroid progenitors in the bone marrow. Normal plasma erythropoietin levels range from 0.01 to 0.03 Units/mL, and can increase up to 100 to 1.000-fold during hypoxia or anemia. Graber and Krantz, Ann. Rev. Med. 29:51 (1978); Eschbach and Adamson, Kidney Intl. 28:1 (1985). Recombinant human erythropoietin (rHuEpo or epoetin alfa) is commercially available as EPOGEN® (epoetin alfa, recombinant human erythropoietin) (Amgen Inc., Thousand Oaks, Calif.) and as PROCRIT® (epoetin alfa, recombinant human erythropoietin) (Ortho Biotech Inc., Raritan, N.J.).

Another example of an erythroid-associated disorder is erythrocytosis. Erythrocytosis, a disorder of red blood cell overproduction caused by excessive and/or ectopic erythropoietin production, can be caused by cancers, e.g., a renal cell cancer, a hepatocarcinoma, and a central nervous system cancer. Diseases associated with erythrocytosis include polycythemias, e.g., polycythemia vera, secondary polycythemia, and relative polycythemia.

As used herein, disorders involving the pancreas include those of the exocrine pancreas such as congenital anomalies, including but not limited to, ectopic pancreas; pancreatitis, including but not limited to, acute pancreatitis; cysts, including but not limited to, pseudocysts; tumors, including but not limited to, cystic tumors and carcinoma of the pancreas; and disorders of the endocrine pancreas such as, diabetes mellitus; islet cell tumors, including but not limited to, insulinomas, gastrinomas, and other rare islet cell tumors.

As used herein, skeletal muscle disorders include, but are not limited to, muscular dystrophy (e.g., Duchenne muscular dystrophy, Becker muscular dystrophy, Emery-Dreifuss muscular dystrophy, limb-girdle muscular dystrophy, facioscapulohumeral muscular dystrophy, myotonic dystrophy, oculopharyngeal muscular dystrophy, distal muscular dystrophy, and congenital muscular dystrophy), motor neuron diseases (e.g., amyotrophic lateral sclerosis, infantile progressive spinal muscular atrophy, intermediate spinal muscular atrophy, spinal bulbar muscular atrophy, and adult spinal muscular atrophy), myopathies (e.g., inflammatory myopathies (e.g., dermatomyositis and polymyositis), myotonia congenita, paramyotonia congenita, central core disease, nemaline myopathy, myotubular myopathy, and periodic paralysis), tumors such as rhabdomyosarcoma, and metabolic diseases of muscle (e.g., phosphorylase deficiency, acid maltase deficiency, phosphofructokinase deficiency, debrancher enzyme deficiency, mitochondrial myopathy, carnitine deficiency, carnitine palmityl transferase deficiency, phosphoglycerate kinase deficiency, phosphoglycerate mutase deficiency, lactate dehydrogenase deficiency, and myoadenylate deaminase deficiency).

Diseases of the skin, include but are not limited to, disorders of pigmentation and melanocytes, including but not limited to, vitiligo, freckle, melasma, lentigo, nevocellular nevus, dysplastic nevi, and malignant melanoma; benign epithelial tumors, including but not limited to, seborrheic keratoses, acanthosis nigricans, fibroepithelial polyp, epithelial cyst, keratoacanthoma, and adnexal (appendage) tumors; premalignant and malignant epidermal tumors, including but not limited to, actinic keratosis, squamous cell carcinoma, basal cell carcinoma, and merkel cell carcinoma; tumors of the dermis, including but not limited to, benign fibrous histiocytoma, dermatofibrosarcoma protuberans, xanthomas, and dermal vascular tumors; tumors of cellular immigrants to the skin, including but not limited to, histiocytosis X, mycosis fungoides (cutaneous T-cell lymphoma), and mastocytosis; disorders of epidermal maturation, including but not limited to, ichthyosis; acute inflammatory dermatoses, including but not limited to, urticaria, acute eczematous dermatitis, and erythema multiforme; chronic inflammatory dermatoses, including but not limited to, psoriasis, lichen planus, and lupus erythematosus; blistering (bullous) diseases, including but not limited to, pemphigus, bullous pemphigoid, dermatitis herpetiformis, and noninflammatory blistering diseases: epidermolysis bullosa and porphyria; disorders of epidermal appendages, including but not limited to, acne vulgaris; panniculitis, including but not limited to, erythema nodosum and erythema induratum; and infection and infestation, such as verrucae, molluscum contagiosum, impetigo, superficial fungal infections, and arthropod bites, stings, and infestations.

As used herein, hormonal disorders and diseases include type I and type II diabetes mellitus, pituitary disorders (e.g., growth disorders), thyroid disorders (e.g., hypothyroidism or hyperthyroidism), and reproductive or fertility disorders (e.g., disorders which affect the organs of the reproductive system, e.g., the prostate gland, the uterus, or the vagina; disorders which involve an imbalance in the levels of a reproductive hormone in a subject; disorders affecting the ability of a subject to reproduce; and disorders affecting secondary sex characteristic development, e.g., adrenal hyperplasia).

Examples of immune, e.g., inflammatory, (e.g. respiratory inflammatory) disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjögren's Syndrome, inflammatory bowel disease, e.g. Crohn's disease and ulcerative colitis, aphthous ulcer, iritis, conjunctivitis, keratoconjunctivitis, asthma, allergic asthma, chronic obstructive pulmonary disease, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

As used herein, disorders involving the heart, or “cardiovascular disease” or a “cardiovascular disorder” includes a disease or disorder which affects the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. A cardiovascular disorder includes, but is not limited to disorders such as arteriosclerosis, atherosclerosis, cardiac hypertrophy, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, congestive heart failure, sinus node dysfunction, angina, heart failure, hypertension, atrial fibrillation, atrial flutter, pericardial disease, including but not limited to, pericardial effusion and pericarditis; cardiomyopathies, e.g., dilated cardiomyopathy or idiopathic cardiomyopathy, myocardial infarction, coronary artery disease, coronary artery spasm, ischemic disease, arrhythmia, sudden cardiac death, and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects). A cardiovascular disease or disorder also can include an endothelial cell disorder.

As used herein, the term “atherosclerosis” is intended to have its clinical meaning. This term refers to a cardiovascular condition occurring as a result of narrowing down of the arterial walls. The narrowing is due to the formation of plaques (raised patches) or streaks in the inner lining of the arteries. These plaques consist of foam cells of low-density lipoproteins, oxidized-LDL, decaying muscle cells, fibrous tissue, clumps of blood platelets, cholesterol, and sometimes calcium. They tend to form in regions of turbulent blood flow and are found most often in people with high concentrations of cholesterol in the bloodstream. The number and thickness of plaques increase with age, causing loss of the smooth lining of the blood vessels and encouraging the formation of thrombi (blood clots). Sometimes fragments of thrombi break off and form emboli, which travel through the bloodstream and block smaller vessels. The blood supply is restricted to the heart, eventually forming a blood clot leading to death. The major causes of atherosclerosis are hypercholesterolemia (and low HDL), hypoalphoproteinemia, and hyperlipidemia marked by high circulating cholesterol and high lipids like LDL-cholesterol and triglycerides in the blood. These lipids are deposited in the arterial walls, obstructing the blood flow and forming atherosclerotic plaques leading to death.

As used herein the term “hypercholesterolemia” is a condition with elevated levels of circulating total cholesterol, LDL-cholesterol and VLDL-cholesterol as per the guidelines of the Expert Panel Report of the National Cholesterol Educational Program (NCEP) of Detection, Evaluation of Treatment of high cholesterol in adults (see, Arch. Int. Med. (1988) 148, 36-39).

As used herein the term “hyperlipidemia” or “hyperlipemia” is a condition where the blood lipid parameters are elevated in the blood. This condition manifests an abnormally high concentration of fats. The lipid fractions in the circulating blood are, total cholesterol, low density lipoproteins, very low density lipoproteins and triglycerides.

As used herein the term “lipoprotein” such as VLDL, LDL and HDL, refers to a group of proteins found in the serum, plasma and lymph and are important for lipid transport. The chemical composition of each lipoprotein differs in that the HDL has a higher proportion of protein versus lipid, whereas the VLDL has a lower proportion of protein versus lipid.

As used herein, the term “triglyceride” means a lipid or neutral fat consisting of glycerol combined with three fatty acid molecules.

As used herein the term “xanthomatosis” is a disease evidenced by a yellowish swelling or plaques in the skin resulting from deposits of fat. The presence of xanthomas are usually accompanied by raised blood cholesterol levels.

As used herein the term “apolipoprotein B” or “apoprotein B” or “Apo B” refers to the protein component of the LDL cholesterol transport proteins. Cholesterol synthesized de novo is transported from the liver and intestine to peripheral tissues in the form of lipoproteins. Most of the apolipoprotein B is secreted into the circulatory system as VLDL.

As used herein the term “apolipoprotein A” or “apoprotein A” or “Apo A” refers to the protein component of the HDL cholesterol transport proteins.

“Procedural vascular trauma” includes the effects of surgical/medical-mechanical interventions into mammalian vasculature, but does not include vascular trauma due to the organic vascular pathologies listed hereinabove, or to unintended traumas, such as due to an accident. Thus, procedural vascular traumas within the scope of the present treatment method include (1) organ grafting or transplantation, such as transplantation and grafting of heart, kidney, liver and the like, e.g., involving vessel anastomosis; (2) vascular surgery, such as coronary bypass surgery, biopsy, heart valve replacement, atheroectomy, thrombectomy, and the like; (3) transcatheter vascular therapies (TVT) including angioplasty, e.g., laser angioplasty and PTCA procedures discussed hereinbelow, employing balloon catheters, or indwelling catheters; (4) vascular grafting using natural or synthetic materials, such as in saphenous vein coronary bypass grafts, dacron and venous grafts used for peripheral arterial reconstruction, etc.; (5) placement of a mechanical shunt, such as a PTFE hemodialysis shunt used for arteriovenous communications; and (6) placement of an intravascular stent, which may be metallic, plastic or a biodegradable polymer. See U.S. patent application Ser. No. 08/389,712, filed Feb. 15, 1995, which is incorporated by reference herein. For a general discussion of implantable devices and biomaterials from which they can be formed, see H. Kambic et al., “Biomaterials in Artificial Organs”, Chem. Eng. News, 30 (Apr. 14, 1986), the disclosure of which is incorporated by reference herein.

As used herein, “cholesterol lowering agents” include agents which are useful for lowering serum cholesterol such as for example bile acid sequestering resins (e.g. colestipol hydrochloride or cholestyramine), fish oil, stanol esters, an ApoAII-lowering agent, a VLDL lowering agent, an ApoAI-stimulating agent, fibric acid derivatives (e.g. clofibrate, fenofibrate, or gemfibrozil), thiazolidenediones (e.g. troglitazone), or HMG-CoA reductase inhibitors (e.g. statins, such as fluvastatin sodium, lovastatin, pravastatin sodium, or simvastatin), as well as nicotinic acid, niacin, or probucol.

“VLDL-lowering agent” includes an agent which decreases the hepatic synthesis of triglyceride-rich lipoproteins or increases the catabolism of triglyceride-rich lipoproteins, e.g., fibrates such as gemfibrozil, or the statins, increases the expression of the apoE-mediated clearance pathway, or improves insulin sensitivity in diabetics, e.g., the thiazolidene diones.

As used herein, a “lipid homeostasis disorder” includes a disorder, disease, or condition associated with, caused by, and/or linked to abnormal regulation (e.g., upregulation or downregulation) of lipid metabolism. Lipid homeostasis disorders may be caused by or associated with aberrant lipolysis, aberrant lipid uptake, aberrant lipid synthesis and/or secretion, aberrant intracellular lipid release and/or turnover, aberrant intracellular triglyceride release and/or turnover, aberrant intracellular lipid and/or triglyceride mass, and/or aberrant secreted lipid and/or triglyceride mass within or from a cell, e.g., a liver cell. Lipid homeostasis disorders include, but are not limited to, atherosclerosis, obesity, diabetes, insulin resistance, hyperlipidemia, hypolipidemia, dyslipidemia, hypercholesterolemia, hypocholesterolemia, triglyceride storage disease, cardiovascular disease, coronary artery disease, hypertension, stroke, overweight, anorexia, cachexia, hyperlipoproteinemia, hypolipoproteinemia, Niemann Pick disease, hypertriglyceridemia, hypotriglyceridemia, pancreatitis, diffuse idiopathic skeletal hyperostosis (DISH), atherogenic lipoprotein phenotype (ALP), epilepsy, liver disease, fatty liver, steatohepatitis, and polycystic ovarian syndrome.

Disorders which can be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolism, for example, fibrosis resulting from a storage disorder such as Gaucher's disease (lipid abnormalities) or a glycogen storage disease, A1-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein can be used for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

Disorders involving the small intestine include the malabsorption syndromes such as, celiac sprue, tropical sprue (postinfectious sprue), whipple disease, disaccharidase (lactase) deficiency, abetalipoproteinemia, and tumors of the small intestine including adenomas and adenocarcinoma.

Examples of pain conditions include, but are not limited to, pain elicited during various forms of tissue injury, e.g., inflammation, infection, and ischemia; pain associated with musculoskeletal disorders, e.g., joint pain, or arthritis; tooth pain; headaches, e.g., migrane; pain associated with surgery; pain related to inflammation, e.g., irritable bowel syndrome; chest pain; or hyperalgesia, e.g., excessive sensitivity to pain (described in, for example, Fields (1987) Pain, New York: McGraw-Hill). Other examples of pain disorders or pain syndromes include, but are not limited to, complex regional pain syndrome (CRPS), reflex sympathetic dystrophy (RSD), causalgia, neuralgia, central pain and dysesthesia syndrome, carotidynia, neurogenic pain, refractory cervicobrachial pain syndrome, myofascial pain syndrome, craniomandibular pain dysfunction syndrome, chronic idiopathic pain syndrome, Costen's pain-dysfunction, acute chest pain syndrome, nonulcer dyspepsia, interstitial cystitis, gynecologic pain syndrome, patellofemoral pain syndrome, anterior knee pain syndrome, recurrent abdominal pain in children, colic, low back pain syndrome, neuropathic pain, phantom pain from amputation, phantom tooth pain, or pain asymbolia (the inability to feel pain). Other examples of pain conditions include pain induced by parturition, or post partum pain.

As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Additionally, the molecules of the invention can play an important role in the etiology of certain viral diseases, including but not limited to Hepatitis B, Hepatitis C and Herpes Simplex Virus (HSV). Modulators of the activity of the molecules of the invention could be used to control viral diseases. The modulators can be used in the treatment and/or diagnosis of viral infected tissue or virus-associated tissue fibrosis, especially liver and liver fibrosis. Also, such modulators can be used in the treatment and/or diagnosis of virus-associated carcinoma, especially hepatocellular cancer.

Additionally, molecules of the invention can play an important role in the regulation of metabolism or pain disorders. Diseases of metabolic imbalance include, but are not limited to, obesity, anorexia nervosa, cachexia, lipid disorders, and diabetes. Examples of pain disorders include, but are not limited to, pain response elicited during various forms of tissue injury, e.g., inflammation, infection, and ischemia, usually referred to as hyperalgesia (described in, for example, Fields (1987) Pain, New York: McGraw-Hill); pain associated with musculoskeletal disorders, e.g., joint pain; tooth pain; headaches; pain associated with surgery; pain related to irritable bowel syndrome; or chest pain.

Aberrant expression and/or activity of the molecules of the invention can mediate disorders associated with bone metabolism. “Bone metabolism” refers to direct or indirect effects in the formation or degeneration of bone structures, e.g., bone formation, bone resorption, etc., which can ultimately affect the concentrations in serum of calcium and phosphate. This term also includes activities mediated by the molecules of the invention in bone cells, e.g. osteoclasts and osteoblasts, that can in turn result in bone formation and degeneration. For example, molecules of the invention can support different activities of bone resorbing osteoclasts such as the stimulation of differentiation of monocytes and mononuclear phagocytes into osteoclasts. Accordingly, molecules of the invention that modulate the production of bone cells can influence bone formation and degeneration, and thus can be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

As used herein, platelet disorders include, but are not limited to, thrombocytopenia, include idiopathic thrombocytopenic purpura, including acute idiopathic thrombocytopenic purpura, drug-induced thrombocytopenia, HIV-associated thrombocytopenia, and thrombotic microangiopathies: thrombotic thrombocytopenic purpura and hemolytic-uremic syndrome.

Various aspects of the invention are described in further detail below.

### Isolated Nucleic Acid Molecules

In one aspect, the invention provides, an isolated or purified, nucleic acid molecule that encodes a 25869, 25934, 26335, 50365, 21117, 38692, 46508, 16816, 16839, 49937, 49931 or 49933 polypeptide described herein, e.g., a full length 25869, 25934, 26335, 50365, 21117, 38692, 46508, 16816, 16839, 49937, 49931 or 49933 protein or a fragment thereof, e.g., a biologically active portion of 25869, 25934, 26335, 50365, 21117, 38692, 46508, 16816, 16839, 49937, 49931 or 49933 protein. Also included is a nucleic acid fragment suitable for use as a hybridization probe, which can be used, e.g., to identify a nucleic acid molecule encoding a polypeptide of the invention, 25869, 25934, 26335, 50365, 21117, 38692, 46508, 16816, 16839, 49937, 49931 or 49933 mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO:1, 3, 7, 9, 16, 18, 20, 22, 25, 27, 28, 30, 35, 37, 39, 41, 42, 44, 63, 65, 67, 69, 70 or 72, or a portion of any of this nucleotide sequence. In one embodiment, the nucleic acid molecule includes sequences encoding the human 25869, 25934, 26335, 50365, 21117, 38692, 46508, 16816, 16839, 49937, 49931 or 49933 protein (i.e., “the coding region” of SEQ ID NO:1, 7, 16, 20, 25, 28, 35, 39, 42, 63, 67 or 70, as shown in SEQ ID NO:3, 9, 18, 22, 27, 30, 37, 41, 44, 65, 69 or 72, respectively), as well as 5′ untranslated sequences and 3′ untranslated sequences. Alternatively, the nucleic acid molecule can include only the coding region of SEQ ID NO:1, 7, 16, 20, 25, 28, 35, 39, 42, 63, 67 or 70 (e.g., SEQ ID NO:3, 9, 18, 22, 27, 30, 37, 41, 44, 65, 69 or 72) and, e.g., no flanking sequences which normally accompany the subject sequence. In another embodiment, the nucleic acid molecule encodes a sequence corresponding to a fragment of the protein corresponding to conserved domains identified within SEQ ID NO:2, 8, 17, 21, 26, 29, 36, 40, 43, 64, 68 or 71.

In another embodiment, an isolated nucleic acid molecule of the invention includes a nucleic acid molecule which is a complement of the nucleotide sequence shown in SEQ ID NO:1, 3, 7, 9, 16, 18, 20, 22, 25, 27, 28, 30, 35, 37, 39, 41, 42, 44, 63, 65, 67, 69, 70 or 72, or a portion of any of these nucleotide sequences. In other embodiments, the nucleic acid molecule of the invention is sufficiently complementary to the nucleotide sequence shown in SEQ ID NO:1, 3, 7, 9, 16, 18, 20, 22, 25, 27, 28, 30, 35, 37, 39, 41, 42, 44, 63, 65, 67, 69, 70 or 72 such that it can hybridize to the nucleotide sequence shown in SEQ ID NO:1, 3, 7, 9, 16, 18, 20, 22, 25, 27, 28, 30, 35, 37, 39, 41, 42, 44, 63, 65, 67, 69, 70 or 72, thereby forming a stable duplex.

In one embodiment, an isolated nucleic acid molecule of the present invention includes a nucleotide sequence which is at least about: 60%, 65%, 70%, 75%, 80%, 85%, 90%, 91%, 92%, 93%, 94%, 95%, 96%, 97%, 98%, 99%, or more homologous to the entire length of the nucleotide sequence shown in SEQ ID NO:1, 3, 7, 9, 16, 18, 20, 22, 25, 27, 28, 30, 35, 37, 39, 41, 42, 44, 63, 65, 67, 69, 70 or 72, or a portion, preferably of the same length, of any of these nucleotide sequences.

### 25869, 25934, 26335, 50365, 21117, 38692, 46508, 16816, 16839, 49937, 49931 or 49933 Nucleic Acid Fragments

A nucleic acid molecule of the invention can include only a portion of the nucleic acid sequence of SEQ ID NO:1, 3, 7, 9, 16, 18, 20, 22, 25, 27, 28, 30, 35, 37, 39, 41, 42, 44, 63, 65, 67, 69, 70 or 72. For example, such a nucleic acid molecule can include a fragment which can be used as a probe or primer or a fragment encoding a portion of a 25869, 25934, 26335, 50365, 21117, 38692, 46508, 16816, 16839, 49937, 49931 or 49933 protein, e.g., an immunogenic or biologically active portion of a 25869, 25934, 26335, 50365, 21117, 38692, 46508, 16816, 16839, 49937, 49931 or 49933 protein. A fragment can comprise those nucleotides of SEQ ID NO:1, 3, 7, 9, 16, 18, 20, 22, 25, 27, 28, 30, 35, 37, 39, 41, 42, 44, 63, 65, 67, 69, 70 or 72, which encode a domain of human 25869, 25934, 26335, 50365, 21117, 38692, 46508, 16816, 16839, 49937, 49931 or 49933. The nucleotide sequence determined from the cloning of the 25869, 25934, 26335, 50365, 21117, 38692, 46508, 16816, 16839, 49937, 49931 or 49933 gene allows for the generation of probes and primers designed for use in identifying and/or cloning other 25869, 25934, 26335, 50365, 21117, 38692, 46508, 16816, 16839, 49937, 49931 or 49933 family members, or fragments thereof, as well as 25869, 25934, 26335, 50365, 21117, 38692, 46508, 16816, 16839, 49937, 49931 or 49933 homologs, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 100 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a 25869, 25934, 26335, 50365, 21117, 38692, 46508, 16816, 16839, 49937, 49931 or 49933 nucleic acid fragment can include a sequence corresponding to a domain, as described herein.

25869, 25934, 26335, 50365, 21117, 38692, 46508, 16816, 16839, 49937, 49931 or 49933 probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of SEQ ID NO:1, 3, 7, 9, 16, 18, 20, 22, 25, 27, 28, 30, 35, 37, 39, 41, 42, 44, 63, 65, 67, 69, 70 or 72, or of a naturally occurring allelic variant or mutant of SEQ ID NO:1, 3, 7, 9, 16, 18, 20, 22, 25, 27, 28, 30, 35, 37, 39, 41, 42, 44, 63, 65, 67, 69, 70 or 72.

In a preferred embodiment the nucleic acid is a probe which is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes a domain identified in the 25869, 25934, 26335, 50365, 21117, 38692, 46508, 16816, 16839, 49937, 49931 or 49933 sequences.

In another embodiment a set of primers is provided, e.g., primers suitable for use in a PCR, which can be used to amplify a selected region of a 25869, 25934, 26335, 50365, 21117, 38692, 46508, 16816, 16839, 49937, 49931 or 49933 sequence, e.g., a domain, region, site or other sequence described herein. The primers should be at least 5, 10, or 50 base pairs in length and less than 100, or less than 200, base pairs in length. The primers should be identical, or differ by one base from a sequence disclosed herein or from a naturally occurring variant.

A nucleic acid fragment can encode an epitope bearing region of a polypeptide described herein.

A nucleic acid fragment encoding a “biologically active portion of a 25869, 25934, 26335, 50365, 21117, 38692, 46508, 16816, 16839, 49937, 49931 or 49933 polypeptide” can be prepared by isolating a portion of the nucleotide sequence of SEQ ID NO:1, 3, 7, 9, 16, 18, 20, 22, 25, 27, 28, 30, 35, 37, 39, 41, 42, 44, 63, 65, 67, 69, 70 or 72, which encodes a polypeptide having a 25869, 25934, 26335, 50365, 21117, 38692, 46508, 16816, 16839, 49937, 49931 or 49933 biological activity (e.g., the biological activities of the 25869, 25934, 26335, 50365, 21117, 38692, 46508, 16816, 16839, 49937, 49931 or 49933 proteins are described herein), expressing the encoded portion of the 25869, 25934, 26335, 50365, 21117, 38692, 46508, 16816, 16839, 49937, 49931 or 49933 protein (e.g., by recombinant expression in vitro) and assessing the activity of the encoded portion of the 25869, 25934, 26335, 50365, 21117, 38692, 46508, 16816, 16839, 49937, 49931 or 49933 protein. A nucleic acid fragment encoding a biologically active portion of a 25869, 25934, 26335, 50365, 21117, 38692, 46508, 16816, 16839, 49937, 49931 or 49933 polypeptide, can comprise a nucleotide sequence which is greater than 300 or more nucleotides in length.

In preferred embodiments, a nucleic acid includes a nucleotide sequence which is about 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7249 or more nucleotides in length and hybridizes under stringent hybridization conditions to a nucleic acid molecule of SEQ ID NO:1, 3, 7, 9, 16, 18, 20, 22, 25, 27, 28, 30, 35, 37, 39, 41, 42, 44, 63, 65, 67, 69, 70 or 72.

### 25869, 25934, 26335, 50365, 21117, 38692, 46508, 16816, 16839, 49937, 49931 or 49933 Nucleic Acid Variants

The invention further encompasses nucleic acid molecules that differ from the nucleotide sequence shown in SEQ ID NO:1, 3, 7, 9, 16, 18, 20, 22, 25, 27, 28, 30, 35, 37, 39, 41, 42, 44, 63, 65, 67, 69, 70 or 72. Such differences can be due to degeneracy of the genetic code (and result in a nucleic acid which encodes the same 25869, 25934, 26335, 50365, 21117, 38692, 46508, 16816, 16839, 49937, 49931 or 49933 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:2, 8, 17, 21, 26, 29, 36, 40, 43, 64, 68 or 71. If alignment is needed for this comparison the sequences should be aligned for maximum homology. “Looped” out sequences from deletions or insertions, or mismatches, are considered differences.

Nucleic acids of the inventor can be chosen for having codons, which are preferred, or non-preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO:1, 3, 7, 9, 16, 18, 20, 22, 25, 27, 28, 30, 35, 37, 39, 41, 42, 44, 63, 65, 67, 69, 70 or 72, e.g., as follows: by at least one but less than 10, 20, 30, or 40 nucleotides; at least one but less than 1%, 5%, 10% or 20% of the nucleotides in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in SEQ ID NO:2, 8, 17, 21, 26, 29, 36, 40, 43, 64, 68 or 71 or a fragment of this sequence. Such nucleic acid molecules can readily be identified as being able to hybridize under stringent conditions, to the nucleotide sequence shown in SEQ ID NO:2, 8, 17, 21, 26, 29, 36, 40, 43, 64, 68 or 71 or a fragment of the sequence. Nucleic acid molecules corresponding to orthologs, homologs, and allelic variants of the 25869, 25934, 26335, 50365, 21117, 38692, 46508, 16816, 16839, 49937, 49931 or 49933 cDNAs of the invention can further be isolated by mapping to the same chromosome or locus as the 25869, 25934, 26335, 50365, 21117, 38692, 46508, 16816, 16839, 49937, 49931 or 49933 gene.

Preferred variants include those that are correlated with activities specific to the molecules of the invention, i.e. carboxylase activity, fatty acid desaturase activity, serine/threonine dehydratase activity, hexokinase activity, peptidyl tRNA hydrolase activity, dual specificity phosphatase activity, phospholipase activity, transporter activity, or other.

Allelic variants of 25869, 25934, 26335, 50365, 21117, 38692, 46508, 16816, 16839, 49937, 49931 or 49933, e.g., human 25869, 25934, 26335, 50365, 21117, 38692, 46508, 16816, 16839, 49937, 49931 or 49933, include both functional and non-functional proteins. Functional allelic variants are naturally occurring amino acid sequence variants of the 25869, 25934, 26335, 50365, 21117, 38692, 46508, 16816, 16839, 49937, 49931 or 49933 protein within a population that maintain the ability to (1) hydrolyze an ester linkage and/or liberate the free acid form of a substrate, e.g., hydrolysis of a triglyceride and/or liberation of free fatty acid(s) and glycerol; (2) catalyze the formation of a double bond, preferably, at positions up to 9 carbons from the carboxyl end of a molecule, e.g., a fatty acid, such as a polyunsaturated fatty acid; (3) catalyze the phosphorylation of a sugar, e.g., an aldohexoses and a ketohexoses (e.g., glucose, mannose, fructose, sorbitol and glucosamine); (4) catalyze sugar metabolism; (5) transfer a phosphate from a phosphate donor (e.g., ATP) to a sugar, e.g., an aldohexoses and a ketohexoses (e.g., glucose, mannose, fructose, sorbitol and glucosamine) to form a phosphorylated sugar, e.g., glucose-6-phosphate; (6) catalyze the removal of a phosphate group attached to a tyrosine residue in a protein target, e.g., a growth factor receptor; (7) catalyze the removal of a phosphate group attached to a serine or threonine residue in a protein e.g., a growth factor receptor; (8) hydrolyze covalent bond between peptide and tRNA within peptidyl-tRNAs; (9) catalyze the hydrolysis of phosphatidyl-inositol-4,5-bisphosphate (PIP2) producing diacylglycerol and inositol 1,4,5-trisphosphate; (10) transport a substrate or target molecule (e.g., a Ca²⁺ ion) from one side of a biological membrane to the other; or (11) be phosphorylated or dephosphorylated. Functional allelic variants will typically contain only conservative substitution of one or more amino acids of SEQ ID NO:2, 8, 17, 21, 26, 29, 36, 40, 43, 64, 68 or 71, or substitution, deletion or insertion of non-critical residues in non-critical regions of the protein. Non-functional allelic variants are naturally-occurring amino acid sequence variants of the 25869, 25934, 26335, 50365, 21117, 38692, 46508, 16816, 16839, 49937, 49931 or 49933, e.g., human 25869, 25934, 26335, 50365, 21117, 38692, 46508, 16816, 16839, 49937, 49931 or 49933, protein within a population that do not have the ability to (1) hydrolyze an ester linkage and/or liberate the free acid form of a substrate, e.g., hydrolysis of a triglyceride and/or liberation of free fatty acid(s) and glycerol; (2) catalyze the formation of a double bond, preferably, at positions up to 9 carbons from the carboxyl end of a molecule, e.g., a fatty acid, such as a polyunsaturated fatty acid; (3) catalyze the phosphorylation of a sugar, e.g., an aldohexoses and a ketohexoses (e.g., glucose, mannose, fructose, sorbitol and glucosamine); (4) catalyze sugar metabolism; (5) transfer a phosphate from a phosphate donor (e.g., ATP) to a sugar, e.g., an aldohexoses and a ketohexoses (e.g., glucose, mannose, fructose, sorbitol and glucosamine) to form a phosphorylated sugar, e.g., glucose-6-phosphate; (6) catalyze the removal of a phosphate group attached to a tyrosine residue in a protein target, e.g., a growth factor receptor; (7) catalyze the removal of a phosphate group attached to a serine or threonine residue in a protein e.g., a growth factor receptor; (8) hydrolyze covalent bond between peptide and tRNA within peptidyl-tRNAs; (9) catalyze the hydrolysis of phosphatidyl-inositol-4,5-bisphosphate (PIP2) producing diacylglycerol and inositol 1,4,5-trisphosphate; (10) transport a substrate or target molecule (e.g., a Ca²⁺ ion) from one side of a biological membrane to the other; or (11) be phosphorylated or dephosphorylated. Non-functional allelic variants will typically contain a non-conservative substitution, a deletion, or insertion, or premature truncation of the amino acid sequence of SEQ ID NO:2, 8, 17, 21, 26, 29, 36, 40, 43, 64, 68 or 71, or a substitution, insertion, or deletion in critical residues or critical regions of the protein.

Moreover, nucleic acid molecules encoding other 25869, 25934, 26335, 50365, 21117, 38692, 46508, 16816, 16839, 49937, 49931 or 49933 family members and, thus, which have a nucleotide sequence which differs from the 25869, 25934, 26335, 50365, 21117, 38692, 46508, 16816, 16839, 49937, 49931 or 49933 sequences of SEQ ID NO:1, 3, 7, 9, 16, 18, 20, 22, 25, 27, 28, 30, 35, 37, 39, 41, 42, 44, 63, 65, 67, 69, 70 or 72 are intended to be within the scope of the invention.

### Antisense Nucleic Acid Molecules, Ribozymes and Modified 25869, 25934, 26335, 50365, 21117, 38692, 46508, 16816, 16839, 49937, 49931 or 49933 Nucleic Acid Molecules

In another aspect, the invention features, an isolated nucleic acid molecule which is antisense to 25869, 25934, 26335, 50365, 21117, 38692, 46508, 16816, 16839, 49937, 49931 or 49933. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire 25869, 25934, 26335, 50365, 21117, 38692, 46508, 16816, 16839, 49937, 49931 or 49933 coding strand, or to only a portion thereof (e.g., the coding region of human 25869, 25934, 26335, 50365, 21117, 38692, 46508, 16816, 16839, 49937, 49931 or 49933 corresponding to SEQ ID NO:3, 9, 18, 22, 27, 30, 37, 41, 44, 65, 69 or 72, respectively). In another embodiment, the antisense nucleic acid molecule is antisense to a “noncoding region” of the coding strand of a nucleotide sequence encoding 25869, 25934, 26335, 50365, 21117, 38692, 46508, 16816, 16839, 49937, 49931 or 49933 (e.g., the 5′ and 3′ untranslated regions).

An antisense nucleic acid can be designed such that it is complementary to the entire coding region of 25869, 25934, 26335, 50365, 21117, 38692, 46508, 16816, 16839, 49937, 49931 or 49933 mRNA, but more preferably is an oligonucleotide which is antisense to only a portion of the coding or noncoding region of 25869, 25934, 26335, 50365, 21117, 38692, 46508, 16816, 16839, 49937, 49931 or 49933 mRNA. For example, the antisense oligonucleotide can be complementary to the region surrounding the translation start site of 25869, 25934, 26335, 50365, 21117, 38692, 46508, 16816, 16839, 49937, 49931 or 49933 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a 25869, 25934, 26335, 50365, 21117, 38692, 46508, 16816, 16839, 49937, 49931 or 49933 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically or selectively bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a 25869, 25934, 26335, 50365, 21117, 38692, 46508, 16816, 16839, 49937, 49931 or 49933-encoding nucleic acid can include one or more sequences complementary to the nucleotide sequence of a 25869, 25934, 26335, 50365, 21117, 38692, 46508, 16816, 16839, 49937, 49931 or 49933 cDNA disclosed herein (i.e., SEQ ID NO:1, 3, 7, 9, 16, 18, 20, 22, 25, 27, 28, 30, 35, 37, 39, 41, 42, 44, 63, 65, 67, 69, 70 or 72), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a 25869, 25934, 26335, 50365, 21117, 38692, 46508, 16816, 16839, 49937, 49931 or 49933-encoding mRNA. See, e.g., Cech et al. U.S. Pat. No. 4,987,071; and Cech et al. U.S. Pat. No. 5,116,742. Alternatively, 25869, 25934, 26335, 50365, 21117, 38692, 46508, 16816, 16839, 49937, 49931 or 49933 mRNA can be used to select a catalytic RNA having a specific ribonuclease activity from a pool of RNA molecules. See, e.g., Bartel and Szostak (1993) Science 261:1411-1418.

25869, 25934, 26335, 50365, 21117, 38692, 46508, 16816, 16839, 49937, 49931 or 49933 gene expression can be inhibited by targeting nucleotide sequences complementary to the regulatory region of the 25869, 25934, 26335, 50365, 21117, 38692, 46508, 16816, 16839, 49937, 49931 or 49933 (e.g., the 25869, 25934, 26335, 50365, 21117, 38692, 46508, 16816, 16839, 49937, 49931 or 49933 promoter and/or enhancers) to form triple helical structures that prevent transcription of the 25869, 25934, 26335, 50365, 21117, 38692, 46508, 16816, 16839, 49937, 49931 or 49933 gene in target cells. See generally, Helene (1991) Anticancer Drug Des. 6:569-84; Helene (1992) Ann. N.Y. Acad. Sci. 660:27-36; and Maher (1992) Bioassays 14:807-15. The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or calorimetric.

A 25869, 25934, 26335, 50365, 21117, 38692, 46508, 16816, 16839, 49937, 49931 or 49933 nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup et al. (1996) Bioorganic & Medicinal Chemistry 4: 5-23).

As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. 93: 14670-675.

PNAs of 25869, 25934, 26335, 50365, 21117, 38692, 46508, 16816, 16839, 49937, 49931 or 49933 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 25869, 25934, 26335, 50365, 21117, 38692, 46508, 16816, 16839, 49937, 49931 or 49933 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup et al. (1996) supra)); or as probes or primers for DNA sequencing or hybridization (Hyrup et al. (1996) supra; Perry-O'Keefe supra).

In other embodiments, the oligonucleotide can include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (see, e.g., Krol et al. (1988) Bio-Techniques 6:958-976) or intercalating agents. (see, e.g., Zon (1988) Pharm. Res. 5:539-549). To this end, the oligonucleotide can be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a 25869, 25934, 26335, 50365, 21117, 38692, 46508, 16816, 16839, 49937, 49931 or 49933 nucleic acid of the invention, two complementary regions one having a fluorophore and one a quencher such that the molecular beacon is useful for quantitating the presence of the 25869, 25934, 26335, 50365, 21117, 38692, 46508, 16816, 16839, 49937, 49931 or 49933 nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

### Isolated 25869, 25934, 26335, 50365, 21117, 38692, 46508, 16816, 16839, 49937, 49931 or 49933 Polypeptides

In another aspect, the invention features, an isolated 25869, 25934, 26335, 50365, 21117, 38692, 46508, 16816, 16839, 49937, 49931 or 49933 protein, or fragment, e.g., a biologically active portion, for use as immunogens or antigens to raise or test (or more generally to bind) anti-25869, 25934, 26335, 50365, 21117, 38692, 46508, 16816, 16839, 49937, 49931 or 49933 antibodies. 25869, 25934, 26335, 50365, 21117, 38692, 46508, 16816, 16839, 49937, 49931 or 49933 protein can be isolated from cells or tissue sources using standard protein purification techniques. 25869, 25934, 26335, 50365, 21117, 38692, 46508, 16816, 16839, 49937, 49931 or 49933 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when the polypeptide is expressed in a native cell, or in systems which result in the alteration or omission of post-translational modifications, e.g., glycosylation or cleavage, present in a native cell.

In a preferred embodiment, a 25869, 25934, 26335, 50365, 21117, 38692, 46508, 16816, 16839, 49937, 49931 or 49933 polypeptide has one or more of the following characteristics: it has the ability to (1) hydrolyze an ester linkage and/or liberate the free acid form of a substrate, e.g., hydrolysis of a triglyceride and/or liberation of free fatty acid(s) and glycerol; (2) catalyze the formation of a double bond, preferably, at positions up to 9 carbons from the carboxyl end of a molecule, e.g., a fatty acid, such as a polyunsaturated fatty acid; (3) catalyze the phosphorylation of a sugar, e.g., an aldohexoses and a ketohexoses (e.g., glucose, mannose, fructose, sorbitol and glucosamine); (4) catalyze sugar metabolism; (5) transfer a phosphate from a phosphate donor (e.g., ATP) to a sugar, e.g., an aldohexoses and a ketohexoses (e.g., glucose, mannose, fructose, sorbitol and glucosamine) to form a phosphorylated sugar, e.g., glucose-6-phosphate; (6) catalyze the removal of a phosphate group attached to a tyrosine residue in a protein target, e.g., a growth factor receptor; (7) catalyze the removal of a phosphate group attached to a serine or threonine residue in a protein e.g., a growth factor receptor; (8) hydrolyze covalent bond between peptide and tRNA within peptidyl-tRNAs; (9) catalyze the hydrolysis of phosphatidyl-inositol-4,5-bisphosphate (PIP2) producing diacylglycerol and inositol 1,4,5-trisphosphate; (10) transport a substrate or target molecule (e.g., a Ca²⁺ ion) from one side of a biological membrane to the other; (11) be phosphorylated or dephosphorylated; (12) it has a molecular weight, e.g., a deduced molecular weight, preferably ignoring any contribution of post translational modifications, amino acid composition or other physical characteristic of a 25869, 25934, 26335, 50365, 21117, 38692, 46508, 16816, 16839, 49937, 49931 or 49933 polypeptide, e.g., a polypeptide of SEQ ID NO:2, 8, 17, 21, 26, 29, 36, 40, 43, 64, 68 or 71; (13) it has an overall sequence similarity of at least 60%, preferably at least 70%, more preferably at least 80, 90, or 95%, with a polypeptide of SEQ ID NO:2, 8, 17, 21, 26, 29, 36, 40, 43, 64, 68 or 71; (14) it is expressed in a multitude of human tissues and cell lines (refer to section for each molecule of the invention); and (15) it has specific domains which are preferably about 70%, 80%, 90% or 95% identical to the identified amino acid residues of SEQ ID NO:2, 8, 17, 21, 26, 29, 36, 40, 43, 64, 68 or 71 (refer to section for each molecule of the invention for domain names and locations within amino acid sequence).

In a preferred embodiment the 25869, 25934, 26335, 50365, 21117, 38692, 46508, 16816, 16839, 49937, 49931 or 49933 protein, or fragment thereof, differs from the corresponding sequence in SEQ ID NO:2, 8, 17, 21, 26, 29, 36, 40, 43, 64, 68 or 71. In one embodiment it differs by at least one but by less than 15, 10 or 5 amino acid residues. In another it differs from the corresponding sequence in SEQ ID NO:2, 8, 17, 21, 26, 29, 36, 40, 43, 64, 68 or 71 by at least one residue but less than 20%, 15%, 10% or 5% of the residues in it differ from the corresponding sequence in SEQ ID NO:2, 8, 17, 21, 26, 29, 36, 40, 43, 64, 68 or 71. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the identified or conserved domain(s) within SEQ ID NO:2, 8, 17, 21, 26, 29, 36, 40, 43, 64, 68 or 71. In another embodiment one or more differences are in the identified or conserved domain(s) within SEQ ID NO:2, 8, 17, 21, 26, 29, 36, 40, 43, 64, 68 or 71.

Other embodiments include a protein that contains one or more changes in amino acid sequence, e.g., a change in an amino acid residue which is not essential for activity. Such 25869, 25934, 26335, 50365, 21117, 38692, 46508, 16816, 16839, 49937, 49931 or 49933 proteins differ in amino acid sequence from SEQ ID NO:2, 8, 17, 21, 26, 29, 36, 40, 43, 64, 68 or 71, yet retain biological activity.

In one embodiment, the protein includes an amino acid sequence at least about 60%, 65%, 70%, 75%, 80%, 85%, 90%, 95%, 98% or more homologous to SEQ ID NO:2, 8, 17, 21, 26, 29, 36, 40, 43, 64, 68 or 71.

A 25869, 25934, 26335, 50365, 21117, 38692, 46508, 16816, 16839, 49937, 49931 or 49933 protein or fragment is provided which varies from the sequence of SEQ ID NO:2, 8, 17, 21, 26, 29, 36, 40, 43, 64, 68 or 71 in regions defined by amino acids that are not within identified or conserved domains or regions by at least one but by less than 15, 10 or 5 amino acid residues in the protein or fragment but which does not differ from SEQ ID NO:2, 8, 17, 21, 26, 29, 36, 40, 43, 64, 68 or 71 in regions defined by amino acids that are within identified or conserved domains or regions. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 25869, 25934, 26335, 50365, 21117, 38692, 46508, 16816, 16839, 49937, 49931 or 49933 protein includes an identified domain (refer to section for each molecule of the invention). Moreover, other biologically active portions, in which other regions of the protein are deleted, can be prepared by recombinant techniques and evaluated for one or more of the functional activities of a native 25869, 25934, 26335, 50365, 21117, 38692, 46508, 16816, 16839, 49937, 49931 or 49933 protein.

In a preferred embodiment, the 25869, 25934, 26335, 50365, 21117, 38692, 46508, 16816, 16839, 49937, 49931 or 49933 protein has an amino acid sequence shown in SEQ ID NO:2, 8, 17, 21, 26, 29, 36, 40, 43, 64, 68 or 71. In other embodiments, the 25869, 25934, 26335, 50365, 21117, 38692, 46508, 16816, 16839, 49937, 49931 or 49933 protein is sufficiently or substantially identical to SEQ ID NO:2, 8, 17, 21, 26, 29, 36, 40, 43, 64, 68 or 71. In yet another embodiment, the 25869, 25934, 26335, 50365, 21117, 38692, 46508, 16816, 16839, 49937, 49931 or 49933 protein is sufficiently or substantially identical to SEQ ID NO:2, 8, 17, 21, 26, 29, 36, 40, 43, 64, 68 or 71 and retains the functional activity of the protein of SEQ ID NO:2, 8, 17, 21, 26, 29, 36, 40, 43, 64, 68 or 71, as described in detail in the subsections above.

### 25869, 25934, 26335, 50365, 21117, 38692, 46508, 16816, 16839, 49937, 49931 or 49933 Chimeric or Fusion Proteins

In another aspect, the invention provides 25869, 25934, 26335, 50365, 21117, 38692, 46508, 16816, 16839, 49937, 49931 or 49933 chimeric or fusion proteins. As used herein, a 25869, 25934, 26335, 50365, 21117, 38692, 46508, 16816, 16839, 49937, 49931 or 49933 “chimeric protein” or “fusion protein” includes a 25869, 25934, 26335, 50365, 21117, 38692, 46508, 16816, 16839, 49937, 49931 or 49933 polypeptide linked to a non-25869, 25934, 26335, 50365, 21117, 38692, 46508, 16816, 16839, 49937, 49931 or 49933 polypeptide. A “non-25869, 25934, 26335, 50365, 21117, 38692, 46508, 16816, 16839, 49937, 49931 or 49933 polypeptide” refers to a polypeptide having an amino acid sequence corresponding to a protein which is not substantially homologous to the 25869, 25934, 26335, 50365, 21117, 38692, 46508, 16816, 16839, 49937, 49931 or 49933 protein, e.g., a protein which is different from the 25869, 25934, 26335, 50365, 21117, 38692, 46508, 16816, 16839, 49937, 49931 or 49933 protein and which is derived from the same or a different organism. The 25869, 25934, 26335, 50365, 21117, 38692, 46508, 16816, 16839, 49937, 49931 or 49933 polypeptide of the fusion protein can correspond to all or a portion e.g., a fragment described herein of a 25869, 25934, 26335, 50365, 21117, 38692, 46508, 16816, 16839, 49937, 49931 or 49933 amino acid sequence. In a preferred embodiment, a 25869, 25934, 26335, 50365, 21117, 38692, 46508, 16816, 16839, 49937, 49931 or 49933 fusion protein includes at least one (or two) biologically active portion of a 25869, 25934, 26335, 50365, 21117, 38692, 46508, 16816, 16839, 49937, 49931 or 49933 protein. The non-25869, 25934, 26335, 50365, 21117, 38692, 46508, 16816, 16839, 49937, 49931 or 49933 polypeptide can be fused to the N-terminus or C-terminus of the 25869, 25934, 26335, 50365, 21117, 38692, 46508, 16816, 16839, 49937, 49931 or 49933 polypeptide.

The fusion protein can include a moiety which has a high affinity for a ligand. For example, the fusion protein can be a GST-25869, 25934, 26335, 50365, 21117, 38692, 46508, 16816, 16839, 49937, 49931 or 49933 fusion protein in which the 25869, 25934, 26335, 50365, 21117, 38692, 46508, 16816, 16839, 49937, 49931 or 49933 sequences are fused to the C-terminus of the GST sequences. Such fusion proteins can facilitate the purification of recombinant 25869, 25934, 26335, 50365, 21117, 38692, 46508, 16816, 16839, 49937, 49931 or 49933. Alternatively, the fusion protein can be a 25869, 25934, 26335, 50365, 21117, 38692, 46508, 16816, 16839, 49937, 49931 or 49933 protein containing a heterologous signal sequence at its N-terminus. In certain host cells (e.g., mammalian host cells), expression and/or secretion of 25869, 25934, 26335, 50365, 21117, 38692, 46508, 16816, 16839, 49937, 49931 or 49933 can be increased through use of a heterologous signal sequence.

Fusion proteins can include all or a part of a serum protein, e.g., a portion of an immunoglobulin (e.g., IgG, IgA, or IgE), e.g., an Fc region and/or the hinge C1 and C2 sequences of an immunoglobulin or human serum albumin.

The 25869, 25934, 26335, 50365, 21117, 38692, 46508, 16816, 16839, 49937, 49931 or 49933 fusion proteins of the invention can be incorporated into pharmaceutical compositions and administered to a subject in vivo. The 25869, 25934, 26335, 50365, 21117, 38692, 46508, 16816, 16839, 49937, 49931 or 49933 fusion proteins can be used to affect the bioavailability of a 25869, 25934, 26335, 50365, 21117, 38692, 46508, 16816, 16839, 49937, 49931 or 49933 substrate. 25869, 25934, 26335, 50365, 21117, 38692, 46508, 16816, 16839, 49937, 49931 or 49933 fusion proteins can be useful therapeutically for the treatment of disorders caused by, for example, (i) aberrant modification or mutation of a gene encoding a 25869, 25934, 26335, 50365, 21117, 38692, 46508, 16816, 16839, 49937, 49931 or 49933 protein; (ii) mis-regulation of the 25869, 25934, 26335, 50365, 21117, 38692, 46508, 16816, 16839, 49937, 49931 or 49933 gene; and (iii) aberrant post-translational modification of a 25869, 25934, 26335, 50365, 21117, 38692, 46508, 16816, 16839, 49937, 49931 or 49933 protein.

Moreover, the 25869, 25934, 26335, 50365, 21117, 38692, 46508, 16816, 16839, 49937, 49931 or 49933-fusion proteins of the invention can be used as immunogens to produce anti-25869, 25934, 26335, 50365, 21117, 38692, 46508, 16816, 16839, 49937, 49931 or 49933 antibodies in a subject, to purify 25869, 25934, 26335, 50365, 21117, 38692, 46508, 16816, 16839, 49937, 49931 or 49933 ligands and in screening assays to identify molecules which inhibit the interaction of 25869, 25934, 26335, 50365, 21117, 38692, 46508, 16816, 16839, 49937, 49931 or 49933 with a 25869, 25934, 26335, 50365, 21117, 38692, 46508, 16816, 16839, 49937, 49931 or 49933 substrate.

Expression vectors are commercially available that already encode a fusion moiety (e.g., a GST polypeptide). A 25869, 25934, 26335, 50365, 21117, 38692, 46508, 16816, 16839, 49937, 49931 or 49933-encoding nucleic acid can be cloned into such an expression vector such that the fusion moiety is linked in-frame to the 25869, 25934, 26335, 50365, 21117, 38692, 46508, 16816, 16839, 49937, 49931 or 49933 protein.

### Variants of 25869, 25934, 26335, 50365, 21117, 38692, 46508, 16816, 16839, 49937, 49931 or 49933 Proteins

In another aspect, the invention also features a variant of a 25869, 25934, 26335, 50365, 21117, 38692, 46508, 16816, 16839, 49937, 49931 or 49933 polypeptide, e.g., which functions as an agonist (mimetics) or as an antagonist. Variants of the 25869, 25934, 26335, 50365, 21117, 38692, 46508, 16816, 16839, 49937, 49931 or 49933 proteins can be generated by mutagenesis, e.g., discrete point mutation, the insertion or deletion of sequences or the truncation of a 25869, 25934, 26335, 50365, 21117, 38692, 46508, 16816, 16839, 49937, 49931 or 49933 protein. An agonist of the 25869, 25934, 26335, 50365, 21117, 38692, 46508, 16816, 16839, 49937, 49931 or 49933 proteins can retain substantially the same, or a subset, of the biological activities of the naturally occurring form of a 25869, 25934, 26335, 50365, 21117, 38692, 46508, 16816, 16839, 49937, 49931 or 49933 protein. An antagonist of a 25869, 25934, 26335, 50365, 21117, 38692, 46508, 16816, 16839, 49937, 49931 or 49933 protein can inhibit one or more of the activities of the naturally occurring form of the 25869, 25934, 26335, 50365, 21117, 38692, 46508, 16816, 16839, 49937, 49931 or 49933 protein by, for example, competitively modulating a 25869, 25934, 26335, 50365, 21117, 38692, 46508, 16816, 16839, 49937, 49931 or 49933-mediated activity of a 25869, 25934, 26335, 50365, 21117, 38692, 46508, 16816, 16839, 49937, 49931 or 49933 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 25869, 25934, 26335, 50365, 21117, 38692, 46508, 16816, 16839, 49937, 49931 or 49933 protein.

Variants of a 25869, 25934, 26335, 50365, 21117, 38692, 46508, 16816, 16839, 49937, 49931 or 49933 protein can be identified by screening combinatorial libraries of mutants, e.g., truncation mutants, of a 25869, 25934, 26335, 50365, 21117, 38692, 46508, 16816, 16839, 49937, 49931 or 49933 protein for agonist or antagonist activity.

Libraries of fragments e.g., N terminal, C terminal, or internal fragments, of a 25869, 25934, 26335, 50365, 21117, 38692, 46508, 16816, 16839, 49937, 49931 or 49933 protein coding sequence can be used to generate a variegated population of fragments for screening and subsequent selection of variants of a 25869, 25934, 26335, 50365, 21117, 38692, 46508, 16816, 16839, 49937, 49931 or 49933 protein.

Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Recursive ensemble mutagenesis (REM), a new technique which enhances the frequency of functional mutants in the libraries, can be used in combination with the screening assays to identify 25869, 25934, 26335, 50365, 21117, 38692, 46508, 16816, 16839, 49937, 49931 or 49933 variants (Arkin and Yourvan (1992) Proc. Natl. Acad. Sci. USA 89:7811-7815; Delgrave et al. (1993) Protein Engineering 6:327-331).

Cell based assays can be exploited to analyze a variegated 25869, 25934, 26335, 50365, 21117, 38692, 46508, 16816, 16839, 49937, 49931 or 49933 library. For example, a library of expression vectors can be transfected into a cell line, e.g., a cell line, which ordinarily responds to 25869, 25934, 26335, 50365, 21117, 38692, 46508, 16816, 16839, 49937, 49931 or 49933 in a substrate-dependent manner. The transfected cells are then contacted with 25869, 25934, 26335, 50365, 21117, 38692, 46508, 16816, 16839, 49937, 49931 or 49933 and the effect of the expression of the mutant on signaling by the 25869, 25934, 26335, 50365, 21117, 38692, 46508, 16816, 16839, 49937, 49931 or 49933 substrate can be detected, e.g., by measuring either carboxylase activity, fatty acid desaturase activity, serine/threonine dehydratase activity, hexokinase activity, peptidyl tRNA hydrolase activity, dual specificity phosphatase activity, phospholipase activity, transporter activity, or other activity disclosed herein. Plasmid DNA can then be recovered from the cells which score for inhibition, or alternatively, potentiation of signaling by the 25869, 25934, 26335, 50365, 21117, 38692, 46508, 16816, 16839, 49937, 49931 or 49933 substrate, and the individual clones further characterized.

In another aspect, the invention features a method of making a 25869, 25934, 26335, 50365, 21117, 38692, 46508, 16816, 16839, 49937, 49931 or 49933 polypeptide, e.g., a peptide having a non-wild type activity, e.g., an antagonist, agonist, or super agonist of a naturally occurring 25869, 25934, 26335, 50365, 21117, 38692, 46508, 16816, 16839, 49937, 49931 or 49933 polypeptide, e.g., a naturally occurring 25869, 25934, 26335, 50365, 21117, 38692, 46508, 16816, 16839, 49937, 49931 or 49933 polypeptide. The method includes altering the sequence of a 25869, 25934, 26335, 50365, 21117, 38692, 46508, 16816, 16839, 49937, 49931 or 49933 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a 25869, 25934, 26335, 50365, 21117, 38692, 46508, 16816, 16839, 49937, 49931 or 49933 polypeptide a biological activity of a naturally occurring 25869, 25934, 26335, 50365, 21117, 38692, 46508, 16816, 16839, 49937, 49931 or 49933 polypeptide. The method includes altering the sequence, e.g., by substitution or deletion of one or more residues, of a 25869, 25934, 26335, 50365, 21117, 38692, 46508, 16816, 16839, 49937, 49931 or 49933 polypeptide, e.g., altering the sequence of a non-conserved region, or a domain or residue described herein, and testing the altered polypeptide for the desired activity.

### Anti-25869, 25934, 26335, 50365, 21117, 38692, 46508, 16816, 16839, 49937, 49931 or 49933 Antibodies

In another aspect, the invention provides an anti-25869, 25934, 26335, 50365, 21117, 38692, 46508, 16816, 16839, 49937, 49931 or 49933 antibody. The term “antibody” as used herein refers to an immunoglobulin molecule or immunologically active portion thereof, i.e., an antigen-binding portion. Examples of immunologically active portions of immunoglobulin molecules include scFV and dcFV fragments, Fab and F(ab′)₂ fragments which can be generated by treating the antibody with an enzyme such as papain or pepsin, respectively.

The antibody can be a polyclonal, monoclonal, recombinant, e.g., a chimeric or humanized, fully human, non-human, e.g., murine, or single chain antibody. In a preferred embodiment it has effector function and can fix complement. The antibody can be coupled to a toxin or imaging agent.

A full-length 25869, 25934, 26335, 50365, 21117, 38692, 46508, 16816, 16839, 49937, 49931 or 49933 protein or, antigenic peptide fragment of 25869, 25934, 26335, 50365, 21117, 38692, 46508, 16816, 16839, 49937, 49931 or 49933 can be used as an immunogen or can be used to identify anti-25869, 25934, 26335, 50365, 21117, 38692, 46508, 16816, 16839, 49937, 49931 or 49933 antibodies made with other immunogens, e.g., cells, membrane preparations, and the like. The antigenic peptide of 25869, 25934, 26335, 50365, 21117, 38692, 46508, 16816, 16839, 49937, 49931 or 49933 should include at least 8 amino acid residues of the amino acid sequence shown in SEQ ID NO:2, 8, 17, 21, 26, 29, 36, 40, 43, 64, 68 or 71 and encompasses an epitope of 25869, 25934, 26335, 50365, 21117, 38692, 46508, 16816, 16839, 49937, 49931 or 49933. Preferably, the antigenic peptide includes at least 10 amino acid residues, more preferably at least 15 amino acid residues, even more preferably at least 20 amino acid residues, and most preferably at least 30 amino acid residues.

Fragments of 25869, 25934, 26335, 50365, 21117, 38692, 46508, 16816, 16839, 49937, 49931 or 49933 which include hydrophilic regions of SEQ ID NO:2, 8, 17, 21, 26, 29, 36, 40, 43, 64, 68 or 71 can be used to make, e.g., used as immunogens or used to characterize the specificity of an antibody, antibodies against hydrophilic regions of the 25869, 25934, 26335, 50365, 21117, 38692, 46508, 16816, 16839, 49937, 49931 or 49933 protein. Similarly, fragments of 25869, 25934, 26335, 50365, 21117, 38692, 46508, 16816, 16839, 49937, 49931 or 49933 which include hydrophobic regions of SEQ ID NO:2, 8, 17, 21, 26, 29, 36, 40, 43, 64, 68 or 71 can be used to make an antibody against a hydrophobic region of the 25869, 25934, 26335, 50365, 21117, 38692, 46508, 16816, 16839, 49937, 49931 or 49933 protein; fragments of 25869, 25934, 26335, 50365, 21117, 38692, 46508, 16816, 16839, 49937, 49931 or 49933 which include residues within extra cellular domain(s) of SEQ ID NO:2, 8, 17, 21, 26, 29, 36, 40, 43, 64, 68 or 71 can be used to make an antibody against an extracellular or non-cytoplasmic region of the 25869, 25934, 26335, 50365, 21117, 38692, 46508, 16816, 16839, 49937, 49931 or 49933 protein; fragments of 25869, 25934, 26335, 50365, 21117, 38692, 46508, 16816, 16839, 49937, 49931 or 49933 which include residues within intracellular regions of SEQ ID NO:2, 8, 17, 21, 26, 29, 36, 40, 43, 64, 68 or 71 can be used to make an antibody against an intracellular region of the 25869, 25934, 26335, 50365, 21117, 38692, 46508, 16816, 16839, 49937, 49931 or 49933 protein; a fragment of 25869, 25934, 26335, 50365, 21117, 38692, 46508, 16816, 16839, 49937, 49931 or 49933 which include residues within identified or conserved domains of SEQ ID NO:2, 8, 17, 21, 26, 29, 36, 40, 43, 64, 68 or 71 can be used to make an antibody against the identified or conserved domain of the 25869, 25934, 26335, 50365, 21117, 38692, 46508, 16816, 16839, 49937, 49931 or 49933 protein.

Antibodies reactive with, or specific or selective for, any of these regions, or other regions or domains described herein are provided.

Preferred epitopes encompassed by the antigenic peptide are regions of 25869, 25934, 26335, 50365, 21117, 38692, 46508, 16816, 16839, 49937, 49931 or 49933 located on the surface of the protein, e.g., hydrophilic regions, as well as regions with high antigenicity. For example, an Emini surface probability analysis of the human 25869, 25934, 26335, 50365, 21117, 38692, 46508, 16816, 16839, 49937, 49931 or 49933 protein sequence can be used to indicate the regions that have a particularly high probability of being localized to the surface of the 25869, 25934, 26335, 50365, 21117, 38692, 46508, 16816, 16839, 49937, 49931 or 49933 protein and are thus likely to constitute surface residues useful for targeting antibody production.

In a preferred embodiment the antibody can bind to the extracellular portion of the 25869, 25934, 26335, 50365, 21117, 38692, 46508, 16816, 16839, 49937, 49931 or 49933 protein, e.g., it can bind to a whole cell which expresses the 25869, 25934, 26335, 50365, 21117, 38692, 46508, 16816, 16839, 49937, 49931 or 49933 protein. In another embodiment, the antibody binds an intracellular portion of the 25869, 25934, 26335, 50365, 21117, 38692, 46508, 16816, 16839, 49937, 49931 or 49933 protein.

In a preferred embodiment the antibody binds an epitope on any domain or region on 25869, 25934, 26335, 50365, 21117, 38692, 46508, 16816, 16839, 49937, 49931 or 49933 proteins described herein.

Additionally, chimeric, humanized, and completely human antibodies are also within the scope of the invention. Chimeric, humanized, but most preferably, completely human antibodies are desirable for applications which include repeated administration, e.g., therapeutic treatment of human patients, and some diagnostic applications.

Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559).

A humanized or complementarity determining region (CDR)-grafted antibody will have at least one or two, but generally all three recipient CDR's (of heavy and or light immuoglobulin chains) replaced with a donor CDR. The antibody may be replaced with at least a portion of a non-human CDR or only some of the CDR's may be replaced with non-human CDR's. It is only necessary to replace the number of CDR's required for binding of the humanized antibody to a 25869, 25934, 26335, 50365, 21117, 38692, 46508, 16816, 16839, 49937, 49931 or 49933 or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, (1987) From Genes to Clones (Verlagsgesellschaft, Weinheim, Germany). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison (1985) Science 229:1202-1207, by Oi et al. (1986) BioTechniques 4:214, and by Queen et al. U.S. Pat. Nos. 5,585,089, 5,693,761 and 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a 25869, 25934, 26335, 50365, 21117, 38692, 46508, 16816, 16839, 49937, 49931 or 49933 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. (1986) Nature 321:552-525; Verhoeyan et al. (1988) Science 239:1534; Beidler et al. (1988) J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

The anti-25869, 25934, 26335, 50365, 21117, 38692, 46508, 16816, 16839, 49937, 49931 or 49933 antibody can be a single chain antibody. A single-chain antibody (scFV) can be engineered as described in, for example, Colcher et al. (1999) Ann. NY Acad. Sci. 880:263-80; and Reiter (1996) Clin. Cancer Res. 2:245-52. The single chain antibody can be dimerized or multimerized to generate multivalent antibodies having specificities for different epitopes of the same target 25869, 25934, 26335, 50365, 21117, 38692, 46508, 16816, 16839, 49937, 49931 or 49933 protein.

In a preferred embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

Radioactive ions include, but are not limited to iodine, yttrium and praseodymium.

The conjugates of the invention can be used for modifying a given biological response, the therapeutic moiety is not to be construed as limited to classical chemical therapeutic agents. For example, the therapeutic moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

An anti-25869, 25934, 26335, 50365, 21117, 38692, 46508, 16816, 16839, 49937, 49931 or 49933 antibody (e.g., monoclonal antibody) can be used to isolate 25869, 25934, 26335, 50365, 21117, 38692, 46508, 16816, 16839, 49937, 49931 or 49933 by standard techniques, such as affinity chromatography or immunoprecipitation. Moreover, an anti-25869, 25934, 26335, 50365, 21117, 38692, 46508, 16816, 16839, 49937, 49931 or 49933 antibody can be used to detect 25869, 25934, 26335, 50365, 21117, 38692, 46508, 16816, 16839, 49937, 49931 or 49933 protein (e.g., in a cellular lysate or cell supernatant) in order to evaluate the abundance and pattern of expression of the protein. Anti-25869, 25934, 26335, 50365, 21117, 38692, 46508, 16816, 16839, 49937, 49931 or 49933 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In preferred embodiments, an antibody can be made by immunizing with a purified 25869, 25934, 26335, 50365, 21117, 38692, 46508, 16816, 16839, 49937, 49931 or 49933 antigen, or a fragment thereof, e.g., a fragment described herein, a membrane associated antigen, tissues, e.g., crude tissue preparations, whole cells, preferably living cells, lysed cells, or cell fractions, e.g., membrane fractions.

Antibodies which bind only a native 25869, 25934, 26335, 50365, 21117, 38692, 46508, 16816, 16839, 49937, 49931 or 49933 protein, only denatured or otherwise non-native 25869, 25934, 26335, 50365, 21117, 38692, 46508, 16816, 16839, 49937, 49931 or 49933 protein, or which bind both, are within the invention. Antibodies with linear or conformational epitopes are within the invention. Conformational epitopes sometimes can be identified by identifying antibodies which bind to native but not denatured 25869, 25934, 26335, 50365, 21117, 38692, 46508, 16816, 16839, 49937, 49931 or 49933 protein.

### Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a 25869, 25934, 26335, 50365, 21117, 38692, 46508, 16816, 16839, 49937, 49931 or 49933 nucleic acid in a form suitable for expression of the nucleic acid in a host cell.

Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., 25869, 25934, 26335, 50365, 21117, 38692, 46508, 16816, 16839, 49937, 49931 or 49933 proteins, mutant forms of 25869, 25934, 26335, 50365, 21117, 38692, 46508, 16816, 16839, 49937, 49931 or 49933 proteins, fusion proteins, and the like).

The recombinant expression vectors of the invention can be designed for expression of 25869, 25934, 26335, 50365, 21117, 38692, 46508, 16816, 16839, 49937, 49931 or 49933 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in 25869, 25934, 26335, 50365, 21117, 38692, 46508, 16816, 16839, 49937, 49931 or 49933 activity assays, (e.g., direct assays or competitive assays described in detail below), or to generate antibodies specific or selective for 25869, 25934, 26335, 50365, 21117, 38692, 46508, 16816, 16839, 49937, 49931 or 49933 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The 25869, 25934, 26335, 50365, 21117, 38692, 46508, 16816, 16839, 49937, 49931 or 49933 expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub et al., (1986) Reviews—Trends in Genetics 1:1.

Another aspect the invention provides a host cell which includes a nucleic acid molecule described herein, e.g., a 25869, 25934, 26335, 50365, 21117, 38692, 46508, 16816, 16839, 49937, 49931 or 49933 nucleic acid molecule within a recombinant expression vector or a 25869, 25934, 26335, 50365, 21117, 38692, 46508, 16816, 16839, 49937, 49931 or 49933 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications can occur in succeeding generations due to either mutation or environmental influences, such progeny may not, in fact, be identical to the parent cell, but are still included within the scope of the term as used herein.

A host cell can be any prokaryotic or eukaryotic cell. For example, a 25869, 25934, 26335, 50365, 21117, 38692, 46508, 16816, 16839, 49937, 49931 or 49933 protein can be expressed in bacterial cells such as E. coli, insect cells, yeast or mammalian cells (such as Chinese hamster ovary (CHO) cells or CV-1 origin, SV-40 (COS) cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a 25869, 25934, 26335, 50365, 21117, 38692, 46508, 16816, 16839, 49937, 49931 or 49933 protein. Accordingly, the invention further provides methods for producing a 25869, 25934, 26335, 50365, 21117, 38692, 46508, 16816, 16839, 49937, 49931 or 49933 protein using the host cells of the invention. In one embodiment, the method includes culturing the host cell of the invention (into which a recombinant expression vector encoding a 25869, 25934, 26335, 50365, 21117, 38692, 46508, 16816, 16839, 49937, 49931 or 49933 protein has been introduced) in a suitable medium such that a 25869, 25934, 26335, 50365, 21117, 38692, 46508, 16816, 16839, 49937, 49931 or 49933 protein is produced. In another embodiment, the method further includes isolating a 25869, 25934, 26335, 50365, 21117, 38692, 46508, 16816, 16839, 49937, 49931 or 49933 protein from the medium or the host cell.

In another aspect, the invention features, a cell or purified preparation of cells which include a 25869, 25934, 26335, 50365, 21117, 38692, 46508, 16816, 16839, 49937, 49931 or 49933 transgene, or which otherwise misexpress 25869, 25934, 26335, 50365, 21117, 38692, 46508, 16816, 16839, 49937, 49931 or 49933. The cell preparation can consist of human or non-human cells, e.g., rodent cells, e.g., mouse or rat cells, rabbit cells, or pig cells. In preferred embodiments, the cell or cells include a 25869, 25934, 26335, 50365, 21117, 38692, 46508, 16816, 16839, 49937, 49931 or 49933 transgene, e.g., a heterologous form of a 25869, 25934, 26335, 50365, 21117, 38692, 46508, 16816, 16839, 49937, 49931 or 49933, e.g., a gene derived from humans (in the case of a non-human cell). The 25869, 25934, 26335, 50365, 21117, 38692, 46508, 16816, 16839, 49937, 49931 or 49933 transgene can be misexpressed, e.g., overexpressed or underexpressed. In other preferred embodiments, the cell or cells include a gene which misexpresses an endogenous 25869, 25934, 26335, 50365, 21117, 38692, 46508, 16816, 16839, 49937, 49931 or 49933, e.g., a gene the expression of which is disrupted, e.g., a knockout. Such cells can serve as a model for studying disorders which are related to mutated or misexpressed 25869, 25934, 26335, 50365, 21117, 38692, 46508, 16816, 16839, 49937, 49931 or 49933 alleles or for use in drug screening.

In another aspect, the invention features, a human cell, e.g., a hematopoietic stem cell, transformed with nucleic acid which encodes a subject 25869, 25934, 26335, 50365, 21117, 38692, 46508, 16816, 16839, 49937, 49931 or 49933 polypeptide.

Also provided are cells, preferably human cells, e.g., human hematopoietic or fibroblast cells, in which an endogenous 25869, 25934, 26335, 50365, 21117, 38692, 46508, 16816, 16839, 49937, 49931 or 49933 is under the control of a regulatory sequence that does not normally control the expression of the endogenous 25869, 25934, 26335, 50365, 21117, 38692, 46508, 16816, 16839, 49937, 49931 or 49933 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 25869, 25934, 26335, 50365, 21117, 38692, 46508, 16816, 16839, 49937, 49931 or 49933 gene. For example, an endogenous 25869, 25934, 26335, 50365, 21117, 38692, 46508, 16816, 16839, 49937, 49931 or 49933 gene which is “transcriptionally silent,” e.g., not normally expressed, or expressed only at very low levels, can be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

### Transgenic Animals

The invention provides non-human transgenic animals. Such animals are useful for studying the function and/or activity of a 25869, 25934, 26335, 50365, 21117, 38692, 46508, 16816, 16839, 49937, 49931 or 49933 protein and for identifying and/or evaluating modulators of 25869, 25934, 26335, 50365, 21117, 38692, 46508, 16816, 16839, 49937, 49931 or 49933 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 25869, 25934, 26335, 50365, 21117, 38692, 46508, 16816, 16839, 49937, 49931 or 49933 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a 25869, 25934, 26335, 50365, 21117, 38692, 46508, 16816, 16839, 49937, 49931 or 49933 protein to particular cells. A transgenic founder animal can be identified based upon the presence of a 25869, 25934, 26335, 50365, 21117, 38692, 46508, 16816, 16839, 49937, 49931 or 49933 transgene in its genome and/or expression of 25869, 25934, 26335, 50365, 21117, 38692, 46508, 16816, 16839, 49937, 49931 or 49933 mRNA in tissues or cells of the animals. A transgenic founder animal can then be used to breed additional animals carrying the transgene. Moreover, transgenic animals carrying a transgene encoding a 25869, 25934, 26335, 50365, 21117, 38692, 46508, 16816, 16839, 49937, 49931 or 49933 protein can further be bred to other transgenic animals carrying other transgenes.

25869, 25934, 26335, 50365, 21117, 38692, 46508, 16816, 16839, 49937, 49931 or 49933 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

For example, the following animal models may be used in the methods of the invention: the hypertensive transgenic mouse model that lacks fat and has lipoatrophic diabetes (Reitmann, M. L. et al. (1999) Ann. N.Y. Acad. Sci. 192:289-96; Moitra J. et al. (1998) Genes Dev. 12:3168-81); a VEGF transgenic animal model for atherosclerosis and angiogenesis (Sueishi, K. et al. (1997) Ann. N.Y. Acad. Sci. 811:311-324); guinea pigs, which are used as models for cholesterol and lipoprotein metabolism, as well as early atherosclerosis development (Fernandez, M. L. (2001) J. Nutr. 131(1):10-20); the macrophage scavenger receptor class A (SR-A) transgenic mouse, which shows reduced atherosclerosis (De Winther, M. P. et al. (2000) Int. J. Tissue React. 22(2-3):85-91); the St. Thomas' Hospital rabbit strain, an animal model with genetically elevated plasma levels of VLDL, IDL, and low-density lipoprotein (LDL) (Nordestgaard, B. G. et al. (1992) Eur. J. Epidemiol. 8 Suppl 1:92-8); the Watanabe heritable hyperlipidemic (WHHL) rabbit, the animal model for familial hypercholesterolemia (Buja, L. M. et al. (1990) Eur. Heart J. 11 Suppl E:41-52); atherosclerosis induced in Cynomolgus macaque monkeys by feeding cholesterol (Weingand, K. W. (1989) Exp. Mol. Pathol. 50(1):1-15); atherosclerosis induced by infection with Marek's disease herpesvirus in chickens (1999) Am. Heart J. 138(5 Pt 2):S465-8); genetically selected lines of Japanese quail, highly susceptible (SUS) and resistant (RES) to atherosclerosis (1987) Atherosclerosis 68(1-2):77-8); the atherosclerotic and hypertensive strain of male broad-breasted white turkeys (BBWT) (Pagnan, A. (1980) Artery 6(4):320-7); diet-induced, apoE deficiency-induced, or LDL receptor-deficiency induced atherosclerosis in mice (Smith, J. D. (1997) J. Intern. Med. 242(2):99-109); the JCR:LA-corpulent rat, an experimental model for the obese-diabetic-dyslipidemic syndrome that mimics the human condition and exhibits spontaneous development of atherosclerosis and myocardial lesions (Brindley, D. N. (1995) Metabolism 44(2 Suppl 2):23-7); marmosets or other animals treated with cholestyramine or other cholesterol and/or lipid lowering drugs; and numerous other animal models of atherosclerosis (reviewed in Bocan, T. M. (1998) Curr. Pharm. Des. 4(1):37-52; Fekete, S. (1993) Acta Vet. Hung. 41(1-2):3-9).

Further examples of animals that can be used include the transgenic mouse described in U.S. Pat. No. 5,932,779 that contains a mutation in an endogenous melanocortin-4-receptor (MC4-R) gene; animals having mutations which lead to syndromes that include obesity symptoms (described in, for example, Friedman, J. M. et al. (1991) Mamm. Genome 1:130-144; Friedman, J. M. and Liebel, R. L. (1992) Cell 69:217-220; Bray, G. A. (1992) Prog. Brain Res. 93:333-341; and Bray, G. A. (1989) Amer. J. Clin. Nutr. 5:891-902); the animals described in Stubdal, H. et al. (2000) Mol. Cell. Biol. 20(3):878-82 (the mouse tubby phenotype characterized by maturity-onset obesity); the animals described in Abadie, J. M. et al. (2000) Lipids 35(6):613-20 (the obese Zucker rat (ZR), a genetic model of human youth-onset obesity and type 2 diabetes mellitus); the animals described in Shaughnessy, S. et al. (2000) Diabetes 49(6):904-11 (mice null for the adipocyte fatty acid binding protein); or the animals described in Loskutoff, D. J. et al. (2000) Ann. N.Y. Acad. Sci. 902:272-81 (the fat mouse). Other examples of animals that may be used include non-recombinant, non-genetic animal models of obesity such as, for example, rabbit, mouse, or rat models in which the animal has been exposed to long-term over-eating.

The invention also includes a population of cells from a transgenic animal, as discussed, e.g., below.

### Uses

The nucleic acid molecules, proteins, protein homologs, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a 25869, 25934, 26335, 50365, 21117, 38692, 46508, 16816, 16839, 49937, 49931 or 49933 protein (e.g., via a recombinant expression vector in a host cell in gene therapy applications), to detect a 25869, 25934, 26335, 50365, 21117, 38692, 46508, 16816, 16839, 49937, 49931 or 49933 mRNA (e.g., in a biological sample) or a genetic alteration in a 25869, 25934, 26335, 50365, 21117, 38692, 46508, 16816, 16839, 49937, 49931 or 49933 gene, and to modulate 25869, 25934, 26335, 50365, 21117, 38692, 46508, 16816, 16839, 49937, 49931 or 49933 activity, as described further below. The 25869, 25934, 26335, 50365, 21117, 38692, 46508, 16816, 16839, 49937, 49931 or 49933 proteins can be used to treat disorders characterized by insufficient, aberrant or excessive production of a 25869, 25934, 26335, 50365, 21117, 38692, 46508, 16816, 16839, 49937, 49931 or 49933 substrate or production of 25869, 25934, 26335, 50365, 21117, 38692, 46508, 16816, 16839, 49937, 49931 or 49933 inhibitors. In addition, the 25869, 25934, 26335, 50365, 21117, 38692, 46508, 16816, 16839, 49937, 49931 or 49933 proteins can be used to screen for naturally occurring 25869, 25934, 26335, 50365, 21117, 38692, 46508, 16816, 16839, 49937, 49931 or 49933 substrates, to screen for drugs or compounds which modulate 25869, 25934, 26335, 50365, 21117, 38692, 46508, 16816, 16839, 49937, 49931 or 49933 activity, as well as to treat disorders characterized by insufficient, aberrant or excessive production of 25869, 25934, 26335, 50365, 21117, 38692, 46508, 16816, 16839, 49937, 49931 or 49933 protein or production of 25869, 25934, 26335, 50365, 21117, 38692, 46508, 16816, 16839, 49937, 49931 or 49933 protein forms which have decreased, aberrant or unwanted activity compared to 25869, 25934, 26335, 50365, 21117, 38692, 46508, 16816, 16839, 49937, 49931 or 49933 wild type protein (e.g., aberrant or deficient carboxylase activity, fatty acid desaturase activity, serine/threonine dehydratase activity, hexokinase activity, peptidyl tRNA hydrolase activity, dual specificity phosphatase activity, phospholipase activity, transporter activity, or other activity disclosed herein). Moreover, the anti-25869, 25934, 26335, 50365, 21117, 38692, 46508, 16816, 16839, 49937, 49931 or 49933 antibodies of the invention can be used to detect and isolate 25869, 25934, 26335, 50365, 21117, 38692, 46508, 16816, 16839, 49937, 49931 or 49933 proteins, regulate the bioavailability of 25869, 25934, 26335, 50365, 21117, 38692, 46508, 16816, 16839, 49937, 49931 or 49933 proteins, and modulate 25869, 25934, 26335, 50365, 21117, 38692, 46508, 16816, 16839, 49937, 49931 or 49933 activity.

A method of evaluating a compound for the ability to interact with, e.g., bind, a subject 25869, 25934, 26335, 50365, 21117, 38692, 46508, 16816, 16839, 49937, 49931 or 49933 polypeptide is provided. The method includes: contacting the compound with the subject 25869, 25934, 26335, 50365, 21117, 38692, 46508, 16816, 16839, 49937, 49931 or 49933 polypeptide; and evaluating ability of the compound to interact with, e.g., to bind or form a complex with the subject 25869, 25934, 26335, 50365, 21117, 38692, 46508, 16816, 16839, 49937, 49931 or 49933 polypeptide. This method can be performed in vitro, e.g., in a cell free system, or in vivo, e.g., in a two-hybrid interaction trap assay. This method can be used to identify naturally occurring molecules which interact with subject 25869, 25934, 26335, 50365, 21117, 38692, 46508, 16816, 16839, 49937, 49931 or 49933 polypeptide. It can also be used to find natural or synthetic inhibitors of subject 25869, 25934, 26335, 50365, 21117, 38692, 46508, 16816, 16839, 49937, 49931 or 49933 polypeptide. Screening methods are discussed in more detail below.

### Screening Assays:

The invention provides methods (also referred to herein as “screening assays”) for identifying modulators, i.e., candidate or test compounds or agents (e.g., proteins, peptides, peptidomimetics, peptoids, small molecules or other drugs) which bind to 25869, 25934, 26335, 50365, 21117, 38692, 46508, 16816, 16839, 49937, 49931 or 49933 proteins, have a stimulatory or inhibitory effect on, for example, 25869, 25934, 26335, 50365, 21117, 38692, 46508, 16816, 16839, 49937, 49931 or 49933 expression or 25869, 25934, 26335, 50365, 21117, 38692, 46508, 16816, 16839, 49937, 49931 or 49933 activity, or have a stimulatory or inhibitory effect on, for example, the expression or activity of a 25869, 25934, 26335, 50365, 21117, 38692, 46508, 16816, 16839, 49937, 49931 or 49933 substrate. Compounds thus identified can be used to modulate the activity of target gene products (e.g., 25869, 25934, 26335, 50365, 21117, 38692, 46508, 16816, 16839, 49937, 49931 or 49933 genes) in a therapeutic protocol, to elaborate the biological function of the target gene product, or to identify compounds that disrupt normal target gene interactions.

In one embodiment, the invention provides assays for screening candidate or test compounds which are substrates of a 25869, 25934, 26335, 50365, 21117, 38692, 46508, 16816, 16839, 49937, 49931 or 49933 protein or polypeptide or a biologically active portion thereof. In another embodiment, the invention provides assays for screening candidate or test compounds which bind to or modulate the activity of a 25869, 25934, 26335, 50365, 21117, 38692, 46508, 16816, 16839, 49937, 49931 or 49933 protein or polypeptide or a biologically active portion thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909-13; Erb et al. (1994) Proc. Natl. Acad. Sci. USA 91:11422-426; Zuckermann et al. (1994). J. Med. Chem. 37:2678-85; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233-51.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a 25869, 25934, 26335, 50365, 21117, 38692, 46508, 16816, 16839, 49937, 49931 or 49933 protein or biologically active portion thereof is contacted with a test compound, and the ability of the test compound to modulate 25869, 25934, 26335, 50365, 21117, 38692, 46508, 16816, 16839, 49937, 49931 or 49933 activity is determined. Determining the ability of the test compound to modulate 25869, 25934, 26335, 50365, 21117, 38692, 46508, 16816, 16839, 49937, 49931 or 49933 activity can be accomplished by monitoring, for example, carboxylase activity, fatty acid desaturase activity, serine/threonine dehydratase activity, hexokinase activity, peptidyl tRNA hydrolase activity, dual specificity phosphatase activity, phospholipase activity, transporter activity, or other activity disclosed herein. The cell, for example, can be of mammalian origin, e.g., human.

The ability of the test compound to modulate 25869, 25934, 26335, 50365, 21117, 38692, 46508, 16816, 16839, 49937, 49931 or 49933 binding to a compound, e.g., a 25869, 25934, 26335, 50365, 21117, 38692, 46508, 16816, 16839, 49937, 49931 or 49933 substrate, or to bind to 25869, 25934, 26335, 50365, 21117, 38692, 46508, 16816, 16839, 49937, 49931 or 49933 can also be evaluated. This can be accomplished, for example, by coupling the compound, e.g., the substrate, with a radioisotope or enzymatic label such that binding of the compound, e.g., the substrate, to 25869, 25934, 26335, 50365, 21117, 38692, 46508, 16816, 16839, 49937, 49931 or 49933 can be determined by detecting the labeled compound, e.g., substrate, in a complex. Alternatively, 25869, 25934, 26335, 50365, 21117, 38692, 46508, 16816, 16839, 49937, 49931 or 49933 could be coupled with a radioisotope or enzymatic label to monitor the ability of a test compound to modulate 25869, 25934, 26335, 50365, 21117, 38692, 46508, 16816, 16839, 49937, 49931 or 49933 binding to a 25869, 25934, 26335, 50365, 21117, 38692, 46508, 16816, 16839, 49937, 49931 or 49933 substrate in a complex. For example, compounds (e.g., 25869, 25934, 26335, 50365, 21117, 38692, 46508, 16816, 16839, 49937, 49931 or 49933 substrates) can be labeled with ¹²⁵I, ¹⁴C, ³⁵S or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 25869, 25934, 26335, 50365, 21117, 38692, 46508, 16816, 16839, 49937, 49931 or 49933 substrate) to interact with 25869, 25934, 26335, 50365, 21117, 38692, 46508, 16816, 16839, 49937, 49931 or 49933 with or without the labeling of any of the interactants can be evaluated. For example, a microphysiometer can be used to detect the interaction of a compound with 25869, 25934, 26335, 50365, 21117, 38692, 46508, 16816, 16839, 49937, 49931 or 49933 without the labeling of either the compound or the 25869, 25934, 26335, 50365, 21117, 38692, 46508, 16816, 16839, 49937, 49931 or 49933. McConnell et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 25869, 25934, 26335, 50365, 21117, 38692, 46508, 16816, 16839, 49937, 49931 or 49933.

In yet another embodiment, a cell-free assay is provided in which a 25869, 25934, 26335, 50365, 21117, 38692, 46508, 16816, 16839, 49937, 49931 or 49933 protein or biologically active portion thereof is contacted with a test compound and the ability of the test compound to bind to the 25869, 25934, 26335, 50365, 21117, 38692, 46508, 16816, 16839, 49937, 49931 or 49933 protein or biologically active portion thereof is evaluated. Preferred biologically active portions of the 25869, 25934, 26335, 50365, 21117, 38692, 46508, 16816, 16839, 49937, 49931 or 49933 proteins to be used in assays of the present invention include fragments which participate in interactions with non-25869, 25934, 26335, 50365, 21117, 38692, 46508, 16816, 16839, 49937, 49931 or 49933 molecules, e.g., fragments with high surface probability scores.

Soluble and/or membrane-bound forms of isolated proteins (e.g., 25869, 25934, 26335, 50365, 21117, 38692, 46508, 16816, 16839, 49937, 49931 or 49933 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 25869, 25934, 26335, 50365, 21117, 38692, 46508, 16816, 16839, 49937, 49931 or 49933 protein to bind to a target molecule can be accomplished using real-time Biomolecular Interaction Analysis (BIA) (see, e.g., Sjolander and Urbaniczky (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either 25869, 25934, 26335, 50365, 21117, 38692, 46508, 16816, 16839, 49937, 49931 or 49933, an anti-25869, 25934, 26335, 50365, 21117, 38692, 46508, 16816, 16839, 49937, 49931 or 49933 antibody or its target molecule to facilitate separation of complexed from uncomplexed forms of one or both of the proteins, as well as to accommodate automation of the assay. Binding of a test compound to a 25869, 25934, 26335, 50365, 21117, 38692, 46508, 16816, 16839, 49937, 49931 or 49933 protein, or interaction of a 25869, 25934, 26335, 50365, 21117, 38692, 46508, 16816, 16839, 49937, 49931 or 49933 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/25869, 25934, 26335, 50365, 21117, 38692, 46508, 16816, 16839, 49937, 49931 or 49933 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or 25869, 25934, 26335, 50365, 21117, 38692, 46508, 16816, 16839, 49937, 49931 or 49933 protein, and the mixture incubated under conditions conducive to complex formation (e.g., at physiological conditions for salt and pH).

Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 25869, 25934, 26335, 50365, 21117, 38692, 46508, 16816, 16839, 49937, 49931 or 49933 binding or activity determined using standard techniques.

Other techniques for immobilizing either a 25869, 25934, 26335, 50365, 21117, 38692, 46508, 16816, 16839, 49937, 49931 or 49933 protein or a target molecule on matrices include using conjugation of biotin and streptavidin. Biotinylated 25869, 25934, 26335, 50365, 21117, 38692, 46508, 16816, 16839, 49937, 49931 or 49933 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific or selective for the immobilized component (the antibody, in turn, can be directly labeled or indirectly labeled with, e.g., a labeled anti-Ig antibody).

In one embodiment, this assay is performed utilizing antibodies reactive with 25869, 25934, 26335, 50365, 21117, 38692, 46508, 16816, 16839, 49937, 49931 or 49933 protein or target molecules but which do not interfere with binding of the 25869, 25934, 26335, 50365, 21117, 38692, 46508, 16816, 16839, 49937, 49931 or 49933 protein to its target molecule. Such antibodies can be derivatized to the wells of the plate, and unbound target or 25869, 25934, 26335, 50365, 21117, 38692, 46508, 16816, 16839, 49937, 49931 or 49933 protein trapped in the wells by antibody conjugation. Methods for detecting such complexes, in addition to those described above for the GST-immobilized complexes, include immunodetection of complexes using antibodies reactive with the 25869, 25934, 26335, 50365, 21117, 38692, 46508, 16816, 16839, 49937, 49931 or 49933 protein or target molecule, as well as enzyme-linked assays which rely on detecting an enzymatic activity associated with the 25869, 25934, 26335, 50365, 21117, 38692, 46508, 16816, 16839, 49937, 49931 or 49933 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas and Minton (1993) Trends Biochem Sci 18:284-7); chromatography (gel filtration chromatography, ion-exchange chromatography); electrophoresis (see, e.g., Ausubel et al., eds. (1999) Current Protocols in Molecular Biology, J. Wiley, New York.); and immunoprecipitation (see, for example, Ausubel et al., eds. (1999) Current Protocols in Molecular Biology, J. Wiley, New York). Such resins and chromatographic techniques are known to one skilled in the art (see, e.g., Heegaard (1998) J Mol Recognit 11:141-8; Hage and Tweed (1997) J Chromatogr B Biomed Sci Appl. 699:499-525). Further, fluorescence energy transfer can also be conveniently utilized, as described herein, to detect binding without further purification of the complex from solution.

In a preferred embodiment, the assay includes contacting the 25869, 25934, 26335, 50365, 21117, 38692, 46508, 16816, 16839, 49937, 49931 or 49933 protein or biologically active portion thereof with a known compound which binds 25869, 25934, 26335, 50365, 21117, 38692, 46508, 16816, 16839, 49937, 49931 or 49933 to form an assay mixture, contacting the assay mixture with a test compound, and determining the ability of the test compound to interact with a 25869, 25934, 26335, 50365, 21117, 38692, 46508, 16816, 16839, 49937, 49931 or 49933 protein, wherein determining the ability of the test compound to interact with a 25869, 25934, 26335, 50365, 21117, 38692, 46508, 16816, 16839, 49937, 49931 or 49933 protein includes determining the ability of the test compound to preferentially bind to 25869, 25934, 26335, 50365, 21117, 38692, 46508, 16816, 16839, 49937, 49931 or 49933 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the 25869, 25934, 26335, 50365, 21117, 38692, 46508, 16816, 16839, 49937, 49931 or 49933 genes herein identified. In an alternative embodiment, the invention provides methods for determining the ability of the test compound to modulate the activity of a 25869, 25934, 26335, 50365, 21117, 38692, 46508, 16816, 16839, 49937, 49931 or 49933 protein through modulation of the activity of a downstream effector of a 25869, 25934, 26335, 50365, 21117, 38692, 46508, 16816, 16839, 49937, 49931 or 49933 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner.

Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific or selective for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific or selective for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific or selective for one of the binding components to anchor any complexes formed in solution, and a labeled antibody specific or selective for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the 25869, 25934, 26335, 50365, 21117, 38692, 46508, 16816, 16839, 49937, 49931 or 49933 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 25869, 25934, 26335, 50365, 21117, 38692, 46508, 16816, 16839, 49937, 49931 or 49933 (“25869, 25934, 26335, 50365, 21117, 38692, 46508, 16816, 16839, 49937, 49931 or 49933-binding proteins” or “25869, 25934, 26335, 50365, 21117, 38692, 46508, 16816, 16839, 49937, 49931 or 49933-bp”) and are involved in 25869, 25934, 26335, 50365, 21117, 38692, 46508, 16816, 16839, 49937, 49931 or 49933 activity. Such 25869, 25934, 26335, 50365, 21117, 38692, 46508, 16816, 16839, 49937, 49931 or 49933-bps can be activators or inhibitors of signals by the 25869, 25934, 26335, 50365, 21117, 38692, 46508, 16816, 16839, 49937, 49931 or 49933 proteins or 25869, 25934, 26335, 50365, 21117, 38692, 46508, 16816, 16839, 49937, 49931 or 49933 targets as, for example, downstream elements of a 25869, 25934, 26335, 50365, 21117, 38692, 46508, 16816, 16839, 49937, 49931 or 49933-mediated signaling pathway.

The two-hybrid system is based on the modular nature of most transcription factors, which consist of separable DNA-binding and activation domains. Briefly, the assay utilizes two different DNA constructs. In one construct, the gene that codes for a 25869, 25934, 26335, 50365, 21117, 38692, 46508, 16816, 16839, 49937, 49931 or 49933 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: 25869, 25934, 26335, 50365, 21117, 38692, 46508, 16816, 16839, 49937, 49931 or 49933 protein can be the fused to the activator domain.) If the “bait” and the “prey” proteins are able to interact, in vivo, forming a 25869, 25934, 26335, 50365, 21117, 38692, 46508, 16816, 16839, 49937, 49931 or 49933-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 25869, 25934, 26335, 50365, 21117, 38692, 46508, 16816, 16839, 49937, 49931 or 49933 protein.

In another embodiment, modulators of 25869, 25934, 26335, 50365, 21117, 38692, 46508, 16816, 16839, 49937, 49931 or 49933 expression are identified. For example, a cell or cell free mixture is contacted with a candidate compound and the expression of 25869, 25934, 26335, 50365, 21117, 38692, 46508, 16816, 16839, 49937, 49931 or 49933 mRNA or protein evaluated relative to the level of expression of 25869, 25934, 26335, 50365, 21117, 38692, 46508, 16816, 16839, 49937, 49931 or 49933 mRNA or protein in the absence of the candidate compound. When expression of 25869, 25934, 26335, 50365, 21117, 38692, 46508, 16816, 16839, 49937, 49931 or 49933 mRNA or protein is greater in the presence of the candidate compound than in its absence, the candidate compound is identified as a stimulator of 25869, 25934, 26335, 50365, 21117, 38692, 46508, 16816, 16839, 49937, 49931 or 49933 mRNA or protein expression. Alternatively, when expression of 25869, 25934, 26335, 50365, 21117, 38692, 46508, 16816, 16839, 49937, 49931 or 49933 mRNA or protein is less (statistically significantly less) in the presence of the candidate compound than in its absence, the candidate compound is identified as an inhibitor of 25869, 25934, 26335, 50365, 21117, 38692, 46508, 16816, 16839, 49937, 49931 or 49933 mRNA or protein expression. The level of 25869, 25934, 26335, 50365, 21117, 38692, 46508, 16816, 16839, 49937, 49931 or 49933 mRNA or protein expression can be determined by methods described herein for detecting 25869, 25934, 26335, 50365, 21117, 38692, 46508, 16816, 16839, 49937, 49931 or 49933 mRNA or protein.

In another aspect, the invention pertains to a combination of two or more of the assays described herein. For example, a modulating agent can be identified using a cell-based or a cell free assay, and the ability of the agent to modulate the activity of a 25869, 25934, 26335, 50365, 21117, 38692, 46508, 16816, 16839, 49937, 49931 or 49933 protein can be confirmed in vivo, e.g., in an animal such as an animal model for aberrant or deficient carboxylase activity, fatty acid desaturase activity, serine/threonine dehydratase activity, hexokinase activity, peptidyl tRNA hydrolase activity; dual specificity phosphatase activity, phospholipase activity, transporter activity, or other activity disclosed herein.

This invention further pertains to novel agents identified by the above-described screening assays. Accordingly, it is within the scope of this invention to further use an agent identified as described herein (e.g., a 25869, 25934, 26335, 50365, 21117, 38692, 46508, 16816, 16839, 49937, 49931 or 49933 modulating agent, an antisense 25869, 25934, 26335, 50365, 21117, 38692, 46508, 16816, 16839, 49937, 49931 or 49933 nucleic acid molecule, a 25869, 25934, 26335, 50365, 21117, 38692, 46508, 16816, 16839, 49937, 49931 or 49933-specific antibody, or a 25869, 25934, 26335, 50365, 21117, 38692, 46508, 16816, 16839, 49937, 49931 or 49933-binding partner) in an appropriate animal model to determine the efficacy, toxicity, side effects, or mechanism of action, of treatment with such an agent. Furthermore, novel agents identified by the above-described screening assays can be used for treatments as described herein.

### Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate 25869, 25934, 26335, 50365, 21117, 38692, 46508, 16816, 16839, 49937, 49931 or 49933 with a disease; (ii) identify an individual from a minute biological sample (tissue typing); and (iii) aid in forensic identification of a biological sample. These applications are described in the subsections below.

### Chromosome Mapping

The 25869, 25934, 26335, 50365, 21117, 38692, 46508, 16816, 16839, 49937, 49931 or 49933 nucleotide sequences or portions thereof can be used to map the location of the 25869, 25934, 26335, 50365, 21117, 38692, 46508, 16816, 16839, 49937, 49931 or 49933 genes on a chromosome. This process is called chromosome mapping. Chromosome mapping is useful in correlating the 25869, 25934, 26335, 50365, 21117, 38692, 46508, 16816, 16839, 49937, 49931 or 49933 sequences with genes associated with disease.

Briefly, 25869, 25934, 26335, 50365, 21117, 38692, 46508, 16816, 16839, 49937, 49931 or 49933 genes can be mapped to chromosomes by preparing PCR primers (preferably 15-25 bp in length) from the 25869, 25934, 26335, 50365, 21117, 38692, 46508, 16816, 16839, 49937, 49931 or 49933 nucleotide sequences. These primers can then be used for PCR screening of somatic cell hybrids containing individual human chromosomes. Only those hybrids containing the human gene corresponding to the 25869, 25934, 26335, 50365, 21117, 38692, 46508, 16816, 16839, 49937, 49931 or 49933 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio et al. (1983) Science 220:919-924).

Other mapping strategies e.g., in situ hybridization (described in Fan et al. (1990) Proc. Natl. Acad. Sci. USA, 87:6223-27), pre-screening with labeled flow-sorted chromosomes, and pre-selection by hybridization to chromosome specific cDNA libraries can be used to map 25869, 25934, 26335, 50365, 21117, 38692, 46508, 16816, 16839, 49937, 49931 or 49933 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al. (1988) Human Chromosomes: A Manual of Basic Techniques, Pergamon Press, New York).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland et al. (1987) Nature, 325:783-787.

Moreover, differences in the DNA sequences between individuals affected and unaffected with a disease associated with the 25869, 25934, 26335, 50365, 21117, 38692, 46508, 16816, 16839, 49937, 49931 or 49933 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### Tissue Typing

25869, 25934, 26335, 50365, 21117, 38692, 46508, 16816, 16839, 49937, 49931 or 49933 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 25869, 25934, 26335, 50365, 21117, 38692, 46508, 16816, 16839, 49937, 49931 or 49933 nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, 7, 16, 20, 25, 28, 35, 39, 42, 63, 67 or 70 can provide positive individual identification with a panel of perhaps 10 to 1,000 primers which each yield a noncoding amplified sequence of 100 bases. If predicted coding sequences, such as those in SEQ ID NO:3, 9, 18, 22, 27, 30, 37, 41, 44, 65, 69 or 72 are used, a more appropriate number of primers for positive individual identification would be 500-2,000.

If a panel of reagents from 25869, 25934, 26335, 50365, 21117, 38692, 46508, 16816, 16839, 49937, 49931 or 49933 nucleotide sequences described herein is used to generate a unique identification database for an individual, those same reagents can later be used to identify tissue from that individual. Using the unique identification database, positive identification of the individual, living or dead, can be made from extremely small tissue samples.

### Use of Partial 25869, 25934, 26335, 50365, 21117, 38692, 46508, 16816, 16839, 49937, 49931 or 49933 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1, 7, 16, 20, 25, 28, 35, 39, 42, 63, 67 or 70 (e.g., fragments derived from the noncoding regions of SEQ ID NO:1, 7, 16, 20, 25, 28, 35, 39, 42, 63, 67 or 70 having a length of at least 20 bases, preferably at least 30 bases) are particularly appropriate for this use.

The 25869, 25934, 26335, 50365, 21117, 38692, 46508, 16816, 16839, 49937, 49931 or 49933 nucleotide sequences described herein can further be used to provide polynucleotide reagents, e.g., labeled or labelable probes which can be used in, for example, an in situ hybridization technique, to identify a specific tissue. This can be very useful in cases where a forensic pathologist is presented with a tissue of unknown origin. Panels of such 25869, 25934, 26335, 50365, 21117, 38692, 46508, 16816, 16839, 49937, 49931 or 49933 probes can be used to identify tissue by species and/or by organ type.

In a similar fashion, these reagents, e.g., 25869, 25934, 26335, 50365, 21117, 38692, 46508, 16816, 16839, 49937, 49931 or 49933 primers or probes can be used to screen tissue culture for contamination (i.e. screen for the presence of a mixture of different types of cells in a culture).

### Predictive Medicine

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes 25869, 25934, 26335, 50365, 21117, 38692, 46508, 16816, 16839, 49937, 49931 or 49933. Such disorders include, e.g., a disorder associated with the misexpression of 25869, 25934, 26335, 50365, 21117, 38692, 46508, 16816, 16839, 49937, 49931 or 49933 gene; cellular proliferative and/or differentiative disorders, angiogenic disorders, brain disorders, neurological disorders, blood vessel disorders, breast disorders, colon disorders, kidney disorders, lung disorders, ovarian disorders, prostate disorders, hematopoeitic disorders, pancreatic disorders, skeletal muscle disorders, skin disorders, hormonal disorders, immune e.g., inflammatory, disorders, cardiovascular disorders, lipid homeostasis disorders, endothelial cell disorders, liver disorders, disorders of the small intestine, pain disorders, viral diseases, metabolic disorders, bone metabolism disorders or platelet disorders.

The method includes one or more of the following: detecting, in a tissue of the subject, the presence or absence of a mutation which affects the expression of the 25869, 25934, 26335, 50365, 21117, 38692, 46508, 16816, 16839, 49937, 49931 or 49933 gene, or detecting the presence or absence of a mutation in a region which controls the expression of the gene, e.g., a mutation in the 5′ control region; detecting, in a tissue of the subject, the presence or absence of a mutation which alters the structure of the 25869, 25934, 26335, 50365, 21117, 38692, 46508, 16816, 16839, 49937, 49931 or 49933 gene; detecting, in a tissue of the subject, the misexpression of the 25869, 25934, 26335, 50365, 21117, 38692, 46508, 16816, 16839, 49937, 49931 or 49933 gene, at the mRNA level, e.g., detecting a non-wild type level of an mRNA; or detecting, in a tissue of the subject, the misexpression of the gene, at the protein level, e.g., detecting a non-wild type level of a 25869, 25934, 26335, 50365, 21117, 38692, 46508, 16816, 16839, 49937, 49931 or 49933 polypeptide.

In preferred embodiments the method includes: ascertaining the existence of at least one of: a deletion of one or more nucleotides from the 25869, 25934, 26335, 50365, 21117, 38692, 46508, 16816, 16839, 49937, 49931 or 49933 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO:1, 7, 16, 20, 25, 28, 35, 39, 42, 63, 67 or 70, or naturally occurring mutants thereof or 5′ or 3′ flanking sequences naturally associated with the 25869, 25934, 26335, 50365, 21117, 38692, 46508, 16816, 16839, 49937, 49931 or 49933 gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the 25869, 25934, 26335, 50365, 21117, 38692, 46508, 16816, 16839, 49937, 49931 or 49933 gene; the presence of a non-wild type splicing pattern of a messenger RNA transcript of the gene; or a non-wild type level of 25869, 25934, 26335, 50365, 21117, 38692, 46508, 16816, 16839, 49937, 49931 or 49933.

Methods of the invention can be used prenatally or to determine if a subject's offspring will be at risk for a disorder.

In preferred embodiments the method includes determining the structure of a 25869, 25934, 26335, 50365, 21117, 38692, 46508, 16816, 16839, 49937, 49931 or 49933 gene, an abnormal structure being indicative of risk for the disorder.

In preferred embodiments the method includes contacting a sample from the subject with an antibody to the 25869, 25934, 26335, 50365, 21117, 38692, 46508, 16816, 16839, 49937, 49931 or 49933 protein or a nucleic acid, which hybridizes specifically with the gene. These and other embodiments are discussed below.

### Diagnostic and Prognostic Assays

The presence, level, or absence of 25869, 25934, 26335, 50365, 21117, 38692, 46508, 16816, 16839, 49937, 49931 or 49933 protein or nucleic acid in a biological sample can be evaluated by obtaining a biological sample from a test subject and contacting the biological sample with a compound or an agent capable of detecting 25869, 25934, 26335, 50365, 21117, 38692, 46508, 16816, 16839, 49937, 49931 or 49933 protein or nucleic acid (e.g., mRNA, genomic DNA) that encodes 25869, 25934, 26335, 50365, 21117, 38692, 46508, 16816, 16839, 49937, 49931 or 49933 protein such that the presence of 25869, 25934, 26335, 50365, 21117, 38692, 46508, 16816, 16839, 49937, 49931 or 49933 protein or nucleic acid is detected in the biological sample. The term “biological sample” includes tissues, cells and biological fluids isolated from a subject, as well as tissues, cells and fluids present within a subject. A preferred biological sample is serum. The level of expression of the 25869, 25934, 26335, 50365, 21117, 38692, 46508, 16816, 16839, 49937, 49931 or 49933 gene can be measured in a number of ways, including, but not limited to: measuring the mRNA encoded by the 25869, 25934, 26335, 50365, 21117, 38692, 46508, 16816, 16839, 49937, 49931 or 49933 genes; measuring the amount of protein encoded by the 25869, 25934, 26335, 50365, 21117, 38692, 46508, 16816, 16839, 49937, 49931 or 49933 genes; or measuring the activity of the protein encoded by the 25869, 25934, 26335, 50365, 21117, 38692, 46508, 16816, 16839, 49937, 49931 or 49933 genes.

The level of mRNA corresponding to the 25869, 25934, 26335, 50365, 21117, 38692, 46508, 16816, 16839, 49937, 49931 or 49933 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 25869, 25934, 26335, 50365, 21117, 38692, 46508, 16816, 16839, 49937, 49931 or 49933 nucleic acid, such as the nucleic acid of SEQ ID NO:1, 7, 16, 20, 25, 28, 35, 39, 42, 63, 67 or 70, or a portion thereof, such as an oligonucleotide of at least 7, 15, 30, 50, 100, 250 or 500 nucleotides in length and sufficient to specifically hybridize under stringent conditions to 25869, 25934, 26335, 50365, 21117, 38692, 46508, 16816, 16839, 49937, 49931 or 49933 mRNA or genomic DNA. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by the 25869, 25934, 26335, 50365, 21117, 38692, 46508, 16816, 16839, 49937, 49931 or 49933 genes.

The level of mRNA in a sample that is encoded by one of 25869, 25934, 26335, 50365, 21117, 38692, 46508, 16816, 16839, 49937, 49931 or 49933 can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the 25869, 25934, 26335, 50365, 21117, 38692, 46508, 16816, 16839, 49937, 49931 or 49933 gene being analyzed.

In another embodiment, the methods further contacting a control sample with a compound or agent capable of detecting 25869, 25934, 26335, 50365, 21117, 38692, 46508, 16816, 16839, 49937, 49931 or 49933 mRNA, or genomic DNA, and comparing the presence of 25869, 25934, 26335, 50365, 21117, 38692, 46508, 16816, 16839, 49937, 49931 or 49933 mRNA or genomic DNA in the control sample with the presence of 25869, 25934, 26335, 50365, 21117, 38692, 46508, 16816, 16839, 49937, 49931 or 49933 mRNA or genomic DNA in the test sample.

A variety of methods can be used to determine the level of protein encoded by 25869, 25934, 26335, 50365, 21117, 38692, 46508, 16816, 16839, 49937, 49931 or 49933. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 25869, 25934, 26335, 50365, 21117, 38692, 46508, 16816, 16839, 49937, 49931 or 49933 protein in a biological sample in vitro as well as in vivo. In vitro techniques for detection of 25869, 25934, 26335, 50365, 21117, 38692, 46508, 16816, 16839, 49937, 49931 or 49933 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 25869, 25934, 26335, 50365, 21117, 38692, 46508, 16816, 16839, 49937, 49931 or 49933 protein include introducing into a subject a labeled anti-25869, 25934, 26335, 50365, 21117, 38692, 46508, 16816, 16839, 49937, 49931 or 49933 antibody. For example, the antibody can be labeled with a radioactive marker whose presence and location in a subject can be detected by standard imaging techniques.

In another embodiment, the methods further include contacting the control sample with a compound or agent capable of detecting 25869, 25934, 26335, 50365, 21117, 38692, 46508, 16816, 16839, 49937, 49931 or 49933 protein, and comparing the presence of 25869, 25934, 26335, 50365, 21117, 38692, 46508, 16816, 16839, 49937, 49931 or 49933 protein in the control sample with the presence of 25869, 25934, 26335, 50365, 21117, 38692, 46508, 16816, 16839, 49937, 49931 or 49933 protein in the test sample.

The invention also includes kits for detecting the presence of 25869, 25934, 26335, 50365, 21117, 38692, 46508, 16816, 16839, 49937, 49931 or 49933 in a biological sample. For example, the kit can include a compound or agent capable of detecting 25869, 25934, 26335, 50365, 21117, 38692, 46508, 16816, 16839, 49937, 49931 or 49933 protein or mRNA in a biological sample; and a standard. The compound or agent can be packaged in a suitable container. The kit can further comprise instructions for using the kit to detect 25869, 25934, 26335, 50365, 21117, 38692, 46508, 16816, 16839, 49937, 49931 or 49933 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted 25869, 25934, 26335, 50365, 21117, 38692, 46508, 16816, 16839, 49937, 49931 or 49933 expression or activity. As used herein, the term “unwanted” includes an unwanted phenomenon involved in a biological response such as pain or deregulated cell proliferation.

In one embodiment, a disease or disorder associated with aberrant or unwanted 25869, 25934, 26335, 50365, 21117, 38692, 46508, 16816, 16839, 49937, 49931 or 49933 expression or activity is identified. A test sample is obtained from a subject and 25869, 25934, 26335, 50365, 21117, 38692, 46508, 16816, 16839, 49937, 49931 or 49933 protein or nucleic acid (e.g., mRNA or genomic DNA) is evaluated, wherein the level, e.g., the presence or absence, of 25869, 25934, 26335, 50365, 21117, 38692, 46508, 16816, 16839, 49937, 49931 or 49933 protein or nucleic acid is diagnostic for a subject having or at risk of developing a disease or disorder associated with aberrant or unwanted 25869, 25934, 26335, 50365, 21117, 38692, 46508, 16816, 16839, 49937, 49931 or 49933 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 25869, 25934, 26335, 50365, 21117, 38692, 46508, 16816, 16839, 49937, 49931 or 49933 expression or activity. For example, such methods can be used to determine whether a subject can be effectively treated with an agent for a cellular proliferative and/or differentiative disorders, angiogenic disorders, brain disorders, neurological disorders, blood vessel disorders, breast disorders, colon disorders, kidney disorders, lung disorders, ovarian disorders, prostate disorders, hematopoeitic disorders, pancreatic disorders, skeletal muscle disorders, skin disorders, hormonal disorders, immune e.g., inflammatory, disorders, cardiovascular disorders, lipid homeostasis disorders, endothelial cell disorders, liver disorders, disorders of the small intestine, pain disorders, viral diseases, metabolic disorders, bone metabolism disorders or platelet disorders.

The methods of the invention can also be used to detect genetic alterations in a 25869, 25934, 26335, 50365, 21117, 38692, 46508, 16816, 16839, 49937, 49931 or 49933 gene, thereby determining if a subject with the altered gene is at risk for a disorder characterized by misregulation in 25869, 25934, 26335, 50365, 21117, 38692, 46508, 16816, 16839, 49937, 49931 or 49933 protein activity or nucleic acid expression, such as a cellular proliferative and/or differentiative disorders, angiogenic disorders, brain disorders, neurological disorders, blood vessel disorders, breast disorders, colon disorders, kidney disorders, lung disorders, ovarian disorders, prostate disorders, hematopoeitic disorders, pancreatic disorders, skeletal muscle disorders, skin disorders, hormonal disorders, immune e.g., inflammatory, disorders, cardiovascular disorders, lipid homeostasis disorders, endothelial cell disorders, liver disorders, disorders of the small intestine, pain disorders, viral diseases, metabolic disorders, bone metabolism disorders or platelet disorders. In preferred embodiments, the methods include detecting, in a sample from the subject, the presence or absence of a genetic alteration characterized by at least one of an alteration affecting the integrity of a gene encoding a 25869, 25934, 26335, 50365, 21117, 38692, 46508, 16816, 16839, 49937, 49931 or 49933-protein, or the mis-expression of the 25869, 25934, 26335, 50365, 21117, 38692, 46508, 16816, 16839, 49937, 49931 or 49933 gene. For example, such genetic alterations can be detected by ascertaining the existence of at least one of 1) a deletion of one or more nucleotides from a 25869, 25934, 26335, 50365, 21117, 38692, 46508, 16816, 16839, 49937, 49931 or 49933 gene; 2) an addition of one or more nucleotides to a 25869, 25934, 26335, 50365, 21117, 38692, 46508, 16816, 16839, 49937, 49931 or 49933 gene; 3) a substitution of one or more nucleotides of a 25869, 25934, 26335, 50365, 21117, 38692, 46508, 16816, 16839, 49937, 49931 or 49933 gene, 4) a chromosomal rearrangement of a 25869, 25934, 26335, 50365, 21117, 38692, 46508, 16816, 16839, 49937, 49931 or 49933 gene; 5) an alteration in the level of a messenger RNA transcript of a 25869, 25934, 26335, 50365, 21117, 38692, 46508, 16816, 16839, 49937, 49931 or 49933 gene, 6) aberrant modification of a 25869, 25934, 26335, 50365, 21117, 38692, 46508, 16816, 16839, 49937, 49931 or 49933 gene, such as of the methylation pattern of the genomic DNA, 7) the presence of a non-wild type splicing pattern of a messenger RNA transcript of a 25869, 25934, 26335, 50365, 21117, 38692, 46508, 16816, 16839, 49937, 49931 or 49933 gene, 8) a non-wild type level of a 25869, 25934, 26335, 50365, 21117, 38692, 46508, 16816, 16839, 49937, 49931 or 49933-protein, 9) allelic loss of a 25869, 25934, 26335, 50365, 21117, 38692, 46508, 16816, 16839, 49937, 49931 or 49933 gene, and 10) inappropriate post-translational modification of a 25869, 25934, 26335, 50365, 21117, 38692, 46508, 16816, 16839, 49937, 49931 or 49933-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the 25869, 25934, 26335, 50365, 21117, 38692, 46508, 16816, 16839, 49937, 49931 or 49933-gene. This method can include the steps of collecting a sample of cells from a subject, isolating nucleic acid (e.g., genomic, mRNA or both) from the sample, contacting the nucleic acid sample with one or more primers which specifically hybridize to a 25869, 25934, 26335, 50365, 21117, 38692, 46508, 16816, 16839, 49937, 49931 or 49933 gene under conditions such that hybridization and amplification of the 25869, 25934, 26335, 50365, 21117, 38692, 46508, 16816, 16839, 49937, 49931 or 49933 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a 25869, 25934, 26335, 50365, 21117, 38692, 46508, 16816, 16839, 49937, 49931 or 49933 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 25869, 25934, 26335, 50365, 21117, 38692, 46508, 16816, 16839, 49937, 49931 or 49933 can be identified by hybridizing a sample and control nucleic acids, e.g., DNA or RNA, two dimensional arrays, e.g., chip based arrays. Such arrays include a plurality of addresses, each of which is positionally distinguishable from the other. A different probe is located at each address of the plurality. The arrays can have a high density of addresses, e.g., can contain hundreds or thousands of oligonucleotides probes (Cronin et al. (1996) Human Mutation 7: 244-255; Kozal et al. (1996) Nature Medicine 2: 753-759). For example, genetic mutations in 25869, 25934, 26335, 50365, 21117, 38692, 46508, 16816, 16839, 49937, 49931 or 49933 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 25869, 25934, 26335, 50365, 21117, 38692, 46508, 16816, 16839, 49937, 49931 or 49933 gene and detect mutations by comparing the sequence of the sample 25869, 25934, 26335, 50365, 21117, 38692, 46508, 16816, 16839, 49937, 49931 or 49933 with the corresponding wild-type (control) sequence. Automated sequencing procedures can be utilized when performing the diagnostic assays (Naeve et al. (1995) Biotechniques 19:448-53), including sequencing by mass spectrometry.

Other methods for detecting mutations in the 25869, 25934, 26335, 50365, 21117, 38692, 46508, 16816, 16839, 49937, 49931 or 49933 gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 25869, 25934, 26335, 50365, 21117, 38692, 46508, 16816, 16839, 49937, 49931 or 49933 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in 25869, 25934, 26335, 50365, 21117, 38692, 46508, 16816, 16839, 49937, 49931 or 49933 genes. For example, single strand conformation polymorphism (SSCP) can be used to detect differences in electrophoretic mobility between mutant and wild type nucleic acids (Orita et al. (1989) Proc Natl. Acad. Sci. USA: 86:2766, see also Cotton (1993) Mutat. Res. 285:125-144; and Hayashi (1992) Genet. Anal. Tech. Appl. 9:73-79). Single-stranded DNA fragments of sample and control 25869, 25934, 26335, 50365, 21117, 38692, 46508, 16816, 16839, 49937, 49931 or 49933 nucleic acids will be denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl. Acad. Sci USA 86:6230).

Alternatively, allele specific amplification technology which depends on selective PCR amplification can be used in conjunction with the instant invention. Oligonucleotides used as primers for specific amplification can carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification can also be performed using Taq ligase for amplification (Barany (1991) Proc. Natl. Acad Sci USA 88:189-93). In such cases, ligation will occur only if there is a perfect match at the 3′ end of the 5′ sequence making it possible to detect the presence of a known mutation at a specific site by looking for the presence or absence of amplification.

The methods described herein can be performed, for example, by utilizing pre-packaged diagnostic kits comprising at least one probe nucleic acid or antibody reagent described herein, which can be conveniently used, e.g., in clinical settings to diagnose patients exhibiting symptoms or family history of a disease or illness involving a 25869, 25934, 26335, 50365, 21117, 38692, 46508, 16816, 16839, 49937, 49931 or 49933 gene.

### Use of 25869, 25934, 26335, 50365, 21117, 38692, 46508, 16816, 16839, 49937, 49931 or 49933 Molecules as Surrogate Markers

The 25869, 25934, 26335, 50365, 21117, 38692, 46508, 16816, 16839, 49937, 49931 or 49933 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 25869, 25934, 26335, 50365, 21117, 38692, 46508, 16816, 16839, 49937, 49931 or 49933 molecules of the invention can be detected, and can be correlated with one or more biological states in vivo. For example, the 25869, 25934, 26335, 50365, 21117, 38692, 46508, 16816, 16839, 49937, 49931 or 49933 molecules of the invention can serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers can serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease can be made using cholesterol levels as a surrogate marker, and an analysis of HIV infection can be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 25869, 25934, 26335, 50365, 21117, 38692, 46508, 16816, 16839, 49937, 49931 or 49933 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker can be indicative of the concentration of the drug in a biological tissue, in that the marker is either expressed or transcribed or not expressed or transcribed in that tissue in relationship to the level of the drug. In this fashion, the distribution or uptake of the drug can be monitored by the pharmacodynamic marker. Similarly, the presence or quantity of the pharmacodynamic marker can be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug can be sufficient to activate multiple rounds of marker (e.g., a 25869, 25934, 26335, 50365, 21117, 38692, 46508, 16816, 16839, 49937, 49931 or 49933 marker) transcription or expression, the amplified marker can be in a quantity which is more readily detectable than the drug itself. Also, the marker can be more easily detected due to the nature of the marker itself, for example, using the methods described herein, anti-25869, 25934, 26335, 50365, 21117, 38692, 46508, 16816, 16839, 49937, 49931 or 49933 antibodies can be employed in an immune-based detection system for a 25869, 25934, 26335, 50365, 21117, 38692, 46508, 16816, 16839, 49937, 49931 or 49933 protein marker, or 25869, 25934, 26335, 50365, 21117, 38692, 46508, 16816, 16839, 49937, 49931 or 49933-specific radiolabeled probes can be used to detect a 25869, 25934, 26335, 50365, 21117, 38692, 46508, 16816, 16839, 49937, 49931 or 49933 mRNA marker. Furthermore, the use of a pharmacodynamic marker can offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am. J. Health-Syst. Pharm. 56 Suppl. 3: S16-S20.

The 25869, 25934, 26335, 50365, 21117, 38692, 46508, 16816, 16839, 49937, 49931 or 49933 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, can be selected. For example, based on the presence or quantity of RNA, or protein (e.g., 25869, 25934, 26335, 50365, 21117, 38692, 46508, 16816, 16839, 49937, 49931 or 49933 protein or RNA) for specific tumor markers in a subject, a drug or course of treatment can be selected that is optimized for the treatment of the specific tumor likely to be present in the subject. Similarly, the presence or absence of a specific sequence mutation in 25869, 25934, 26335, 50365, 21117, 38692, 46508, 16816, 16839, 49937, 49931 or 49933 DNA can correlate with a 25869, 25934, 26335, 50365, 21117, 38692, 46508, 16816, 16839, 49937, 49931 or 49933 drug response. The use of pharmacogenomic markers therefore permits the application of the most appropriate treatment for each subject without having to administer the therapy.

### Pharmaceutical Compositions

The nucleic acid and polypeptides, fragments thereof, as well as anti-25869, 25934, 26335, 50365, 21117, 38692, 46508, 16816, 16839, 49937, 49931 or 49933 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody, unconjugated or conjugated as described herein, can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent can, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### Methods of Treatment:

The present invention provides for both prophylactic and therapeutic methods of treating a subject at risk of (or susceptible to) a disorder or having a disorder associated with aberrant or unwanted 25869, 25934, 26335, 50365, 21117, 38692, 46508, 16816, 16839, 49937, 49931 or 49933 expression or activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

With regards to both prophylactic and therapeutic methods of treatment, such treatments can be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 25869, 25934, 26335, 50365, 21117, 38692, 46508, 16816, 16839, 49937, 49931 or 49933 molecules of the present invention or 25869, 25934, 26335, 50365, 21117, 38692, 46508, 16816, 16839, 49937, 49931 or 49933 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted 25869, 25934, 26335, 50365, 21117, 38692, 46508, 16816, 16839, 49937, 49931 or 49933 expression or activity, by administering to the subject a 25869, 25934, 26335, 50365, 21117, 38692, 46508, 16816, 16839, 49937, 49931 or 49933 or an agent which modulates 25869, 25934, 26335, 50365, 21117, 38692, 46508, 16816, 16839, 49937, 49931 or 49933 expression or at least one 25869, 25934, 26335, 50365, 21117, 38692, 46508, 16816, 16839, 49937, 49931 or 49933 activity. Subjects at risk for a disease which is caused or contributed to by aberrant or unwanted 25869, 25934, 26335, 50365, 21117, 38692, 46508, 16816, 16839, 49937, 49931 or 49933 expression or activity can be identified by, for example, any or a combination of diagnostic or prognostic assays as described herein. Administration of a prophylactic agent can occur prior to the manifestation of symptoms characteristic of the 25869, 25934, 26335, 50365, 21117, 38692, 46508, 16816, 16839, 49937, 49931 or 49933 aberrance, such that a disease or disorder is prevented or, alternatively, delayed in its progression. Depending on the type of 25869, 25934, 26335, 50365, 21117, 38692, 46508, 16816, 16839, 49937, 49931 or 49933 aberrance, for example, a 25869, 25934, 26335, 50365, 21117, 38692, 46508, 16816, 16839, 49937, 49931 or 49933, 25869, 25934, 26335, 50365, 21117, 38692, 46508, 16816, 16839, 49937, 49931 or 49933 agonist or 25869, 25934, 26335, 50365, 21117, 38692, 46508, 16816, 16839, 49937, 49931 or 49933 antagonist agent can be used for treating the subject. The appropriate agent can be determined based on screening assays described herein.

It is possible that some 25869, 25934, 26335, 50365, 21117, 38692, 46508, 16816, 16839, 49937, 49931 or 49933 disorders can be caused, at least in part, by an abnormal level of gene product, or by the presence of a gene product exhibiting abnormal activity. As such, the reduction in the level and/or activity of such gene products would bring about the amelioration of disorder symptoms.

The 25869, 25934, 26335, 50365, 21117, 38692, 46508, 16816, 16839, 49937, 49931 or 49933 molecules can act as novel diagnostic targets and therapeutic agents for controlling one or more of a cellular proliferative and/or differentiative disorder, angiogenic disorder, brain disorder, neurological disorder, blood vessel disorder, breast disorder, colon disorder, kidney disorder, lung disorder, ovarian disorder, prostate disorder, hematopoeitic disorder, pancreatic disorder, skeletal muscle disorder, skin disorder, hormonal disorder, immune e.g., inflammatory, disorder, cardiovascular disorder, lipid homeostasis disorder, endothelial cell disorder, liver disorder, disorder of the small intestine, pain disorder, viral disease, metabolic disorder bone metabolism disorders or platelet disorders, all of which are described above.

As discussed, successful treatment of 25869, 25934, 26335, 50365, 21117, 38692, 46508, 16816, 16839, 49937, 49931 or 49933 disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of 25869, 25934, 26335, 50365, 21117, 38692, 46508, 16816, 16839, 49937, 49931 or 49933 disorders. Such molecules can include, but are not limited to peptides, phosphopeptides, small organic or inorganic molecules, or antibodies (including, for example, polyclonal, monoclonal, humanized, human, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules can be utilized in treating or preventing a disease characterized by 25869, 25934, 26335, 50365, 21117, 38692, 46508, 16816, 16839, 49937, 49931 or 49933 expression is through the use of aptamer molecules specific for 25869, 25934, 26335, 50365, 21117, 38692, 46508, 16816, 16839, 49937, 49931 or 49933 protein. Aptamers are nucleic acid molecules having a tertiary structure which permits them to specifically or selectively bind to protein ligands (see, e.g., Osborne et al. (1997) Curr. Opin. Chem Biol. 1: 5-9; and Patel (1997) Curr Opin Chem Biol 1:32-46). Since nucleic acid molecules can in many cases be more conveniently introduced into target cells than therapeutic protein molecules can be, aptamers offer a method by which 25869, 25934, 26335, 50365, 21117, 38692, 46508, 16816, 16839, 49937, 49931 or 49933 protein activity can be specifically decreased without the introduction of drugs or other molecules which can have pluripotent effects.

Antibodies can be generated that are both specific for target gene product and that reduce target gene product activity. Such antibodies can, therefore, by administered in instances whereby negative modulatory techniques are appropriate for the treatment of 25869, 25934, 26335, 50365, 21117, 38692, 46508, 16816, 16839, 49937, 49931 or 49933 disorders. For a description of antibodies, see the Antibody section above.

In circumstances wherein injection of an animal or a human subject with a 25869, 25934, 26335, 50365, 21117, 38692, 46508, 16816, 16839, 49937, 49931 or 49933 protein or epitope for stimulating antibody production is harmful to the subject, it is possible to generate an immune response against 25869, 25934, 26335, 50365, 21117, 38692, 46508, 16816, 16839, 49937, 49931 or 49933 through the use of anti-idiotypic antibodies (see, for example, Herlyn (1999) Ann Med 31:66-78; and Bhattacharya-Chatterjee and Foon (1998) Cancer Treat Res. 94:51-68). If an anti-idiotypic antibody is introduced into a mammal or human subject, it should stimulate the production of anti-anti-idiotypic antibodies, which should be specific to the 25869, 25934, 26335, 50365, 21117, 38692, 46508, 16816, 16839, 49937, 49931 or 49933 protein.

Vaccines directed to a disease characterized by 25869, 25934, 26335, 50365, 21117, 38692, 46508, 16816, 16839, 49937, 49931 or 49933 expression can also be generated in this fashion.

In instances where the target antigen is intracellular and whole antibodies are used, internalizing antibodies can be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate 25869, 25934, 26335, 50365, 21117, 38692, 46508, 16816, 16839, 49937, 49931 or 49933 disorders. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays can utilize antibody mimics and/or “biosensors” that have been created through molecular imprinting techniques. The compound which is able to modulate 25869, 25934, 26335, 50365, 21117, 38692, 46508, 16816, 16839, 49937, 49931 or 49933 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al (1996) Current Opinion in Biotechnology 7:89-94 and in Shea (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis et al (1993) Nature 361:645-647. Through the use of isotope-labeling, the “free” concentration of compound which modulates the expression or activity of 25869, 25934, 26335, 50365, 21117, 38692, 46508, 16816, 16839, 49937, 49931 or 49933 can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. An rudimentary example of such a “biosensor” is discussed in Kriz et al (1995) Analytical Chemistry 67:2142-2144.

Another aspect of the invention pertains to methods of modulating 25869, 25934, 26335, 50365, 21117, 38692, 46508, 16816, 16839, 49937, 49931 or 49933 expression or activity for therapeutic purposes. Accordingly, in an exemplary embodiment, the modulatory method of the invention involves contacting a cell with a 25869, 25934, 26335, 50365, 21117, 38692, 46508, 16816, 16839, 49937, 49931 or 49933 or agent that modulates one or more of the activities of 25869, 25934, 26335, 50365, 21117, 38692, 46508, 16816, 16839, 49937, 49931 or 49933 protein activity associated with the cell. An agent that modulates 25869, 25934, 26335, 50365, 21117, 38692, 46508, 16816, 16839, 49937, 49931 or 49933 protein activity can be an agent as described herein, such as a nucleic acid or a protein, a naturally-occurring target molecule of a 25869, 25934, 26335, 50365, 21117, 38692, 46508, 16816, 16839, 49937, 49931 or 49933 protein (e.g., a 25869, 25934, 26335, 50365, 21117, 38692, 46508, 16816, 16839, 49937, 49931 or 49933 substrate or receptor), a 25869, 25934, 26335, 50365, 21117, 38692, 46508, 16816, 16839, 49937, 49931 or 49933 antibody, a 25869, 25934, 26335, 50365, 21117, 38692, 46508, 16816, 16839, 49937, 49931 or 49933 agonist or antagonist, a peptidomimetic of a 25869, 25934, 26335, 50365, 21117, 38692, 46508, 16816, 16839, 49937, 49931 or 49933 agonist or antagonist, or other small molecule.

In one embodiment, the agent stimulates one or 25869, 25934, 26335, 50365, 21117, 38692, 46508, 16816, 16839, 49937, 49931 or 49933 activities. Examples of such stimulatory agents include active 25869, 25934, 26335, 50365, 21117, 38692, 46508, 16816, 16839, 49937, 49931 or 49933 protein and a nucleic acid molecule encoding 25869, 25934, 26335, 50365, 21117, 38692, 46508, 16816, 16839, 49937, 49931 or 49933. In another embodiment, the agent inhibits one or more 25869, 25934, 26335, 50365, 21117, 38692, 46508, 16816, 16839, 49937, 49931 or 49933 activities. Examples of such inhibitory agents include antisense 25869, 25934, 26335, 50365, 21117, 38692, 46508, 16816, 16839, 49937, 49931 or 49933 nucleic acid molecules, anti-25869, 25934, 26335, 50365, 21117, 38692, 46508, 16816, 16839, 49937, 49931 or 49933 antibodies, and 25869, 25934, 26335, 50365, 21117, 38692, 46508, 16816, 16839, 49937, 49931 or 49933 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 25869, 25934, 26335, 50365, 21117, 38692, 46508, 16816, 16839, 49937, 49931 or 49933 protein or nucleic acid molecule. In one embodiment, the method involves administering an agent (e.g., an agent identified by a screening assay described herein), or combination of agents that modulates (e.g., up regulates or down regulates) 25869, 25934, 26335, 50365, 21117, 38692, 46508, 16816, 16839, 49937, 49931 or 49933 expression or activity. In another embodiment, the method involves administering a 25869, 25934, 26335, 50365, 21117, 38692, 46508, 16816, 16839, 49937, 49931 or 49933 protein or nucleic acid molecule as therapy to compensate for reduced, aberrant, or unwanted 25869, 25934, 26335, 50365, 21117, 38692, 46508, 16816, 16839, 49937, 49931 or 49933 expression or activity.

Stimulation of 25869, 25934, 26335, 50365, 21117, 38692, 46508, 16816, 16839, 49937, 49931 or 49933 activity is desirable in situations in which 25869, 25934, 26335, 50365, 21117, 38692, 46508, 16816, 16839, 49937, 49931 or 49933 is abnormally downregulated and/or in which increased 25869, 25934, 26335, 50365, 21117, 38692, 46508, 16816, 16839, 49937, 49931 or 49933 activity is likely to have a beneficial effect. For example, stimulation of 25869, 25934, 26335, 50365, 21117, 38692, 46508, 16816, 16839, 49937, 49931 or 49933 activity is desirable in situations in which a 25869, 25934, 26335, 50365, 21117, 38692, 46508, 16816, 16839, 49937, 49931 or 49933 is downregulated and/or in which increased 25869, 25934, 26335, 50365, 21117, 38692, 46508, 16816, 16839, 49937, 49931 or 49933 activity is likely to have a beneficial effect. Likewise, inhibition of 25869, 25934, 26335, 50365, 21117, 38692, 46508, 16816, 16839, 49937, 49931 or 49933 activity is desirable in situations in which 25869, 25934, 26335, 50365, 21117, 38692, 46508, 16816, 16839, 49937, 49931 or 49933 is abnormally upregulated and/or in which decreased 25869, 25934, 26335, 50365, 21117, 38692, 46508, 16816, 16839, 49937, 49931 or 49933 activity is likely to have a beneficial effect.

### Pharmacogenomics

The 25869, 25934, 26335, 50365, 21117, 38692, 46508, 16816, 16839, 49937, 49931 or 49933 molecules of the present invention, as well as agents, or modulators which have a stimulatory or inhibitory effect on 25869, 25934, 26335, 50365, 21117, 38692, 46508, 16816, 16839, 49937, 49931 or 49933 activity (e.g., 25869, 25934, 26335, 50365, 21117, 38692, 46508, 16816, 16839, 49937, 49931 or 49933 gene expression) as identified by a screening assay described herein can be administered to individuals to treat (prophylactically or therapeutically) 25869, 25934, 26335, 50365, 21117, 38692, 46508, 16816, 16839, 49937, 49931 or 49933-associated disorders (e.g., aberrant or deficient carboxylase activity, fatty acid desaturase activity, serine/threonine dehydratase activity, hexokinase activity, peptidyl tRNA hydrolase activity, dual specificity phosphatase activity, phospholipase activity, transporter activity, or other activity disclosed herein) associated with aberrant or unwanted 25869, 25934, 26335, 50365, 21117, 38692, 46508, 16816, 16839, 49937, 49931 or 49933 activity.

In conjunction with such treatment, pharmacogenomics (i.e., the study of the relationship between an individual's genotype and that individual's response to a foreign compound or drug) can be considered. Differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a 25869, 25934, 26335, 50365, 21117, 38692, 46508, 16816, 16839, 49937, 49931 or 49933 molecule or 25869, 25934, 26335, 50365, 21117, 38692, 46508, 16816, 16839, 49937, 49931 or 49933 modulator as well as tailoring the dosage and/or therapeutic regimen of treatment with a 25869, 25934, 26335, 50365, 21117, 38692, 46508, 16816, 16839, 49937, 49931 or 49933 molecule or 25869, 25934, 26335, 50365, 21117, 38692, 46508, 16816, 16839, 49937, 49931 or 49933 modulator.

Pharmacogenomics deals with clinically significant hereditary variations in the response to drugs due to altered drug disposition and abnormal action in affected persons. See, for example, Eichelbaum et al. (1996) Clin. Exp. Pharmacol. Physiol. 23:983-985 and Linder et al. (1997) Clin. Chem. 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP can occur once per every 1000 bases of DNA. A SNP can be involved in a disease process, however, the vast majority can not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that can be common among such genetically similar individuals.

Alternatively, a method termed the “candidate gene approach”, can be utilized to identify genes that predict drug response. According to this method, if a gene that encodes a drug's target is known (e.g., a 25869, 25934, 26335, 50365, 21117, 38692, 46508, 16816, 16839, 49937, 49931 or 49933 protein of the present invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a 25869, 25934, 26335, 50365, 21117, 38692, 46508, 16816, 16839, 49937, 49931 or 49933 molecule or 25869, 25934, 26335, 50365, 21117, 38692, 46508, 16816, 16839, 49937, 49931 or 49933 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 25869, 25934, 26335, 50365, 21117, 38692, 46508, 16816, 16839, 49937, 49931 or 49933 molecule or 25869, 25934, 26335, 50365, 21117, 38692, 46508, 16816, 16839, 49937, 49931 or 49933 modulator, such as a modulator identified by one of the exemplary screening assays described herein.

The present invention further provides methods for identifying new agents, or combinations, that are based on identifying agents that modulate the activity of one or more of the gene products encoded by one or more of the 25869, 25934, 26335, 50365, 21117, 38692, 46508, 16816, 16839, 49937, 49931 or 49933 genes of the present invention, wherein these products can be associated with resistance of the cells to a therapeutic agent. Specifically, the activity of the proteins encoded by the 25869, 25934, 26335, 50365, 21117, 38692, 46508, 16816, 16839, 49937, 49931 or 49933 genes of the present invention can be used as a basis for identifying agents for overcoming agent resistance. By blocking the activity of one or more of the resistance proteins, target cells, e.g., human cells, will become sensitive to treatment with an agent to which the unmodified target cells were resistant.

Monitoring the influence of agents (e.g., drugs) on the expression or activity of a 25869, 25934, 26335, 50365, 21117, 38692, 46508, 16816, 16839, 49937, 49931 or 49933 protein can be applied in clinical trials. For example, the effectiveness of an agent determined by a screening assay as described herein to increase 25869, 25934, 26335, 50365, 21117, 38692, 46508, 16816, 16839, 49937, 49931 or 49933 gene expression, protein levels, or upregulate 25869, 25934, 26335, 50365, 21117, 38692, 46508, 16816, 16839, 49937, 49931 or 49933 activity, can be monitored in clinical trials of subjects exhibiting decreased 25869, 25934, 26335, 50365, 21117, 38692, 46508, 16816, 16839, 49937, 49931 or 49933 gene expression, protein levels, or downregulated 25869, 25934, 26335, 50365, 21117, 38692, 46508, 16816, 16839, 49937, 49931 or 49933 activity. Alternatively, the effectiveness of an agent determined by a screening assay to decrease 25869, 25934, 26335, 50365, 21117, 38692, 46508, 16816, 16839, 49937, 49931 or 49933 gene expression, protein levels, or downregulate 25869, 25934, 26335, 50365, 21117, 38692, 46508, 16816, 16839, 49937, 49931 or 49933 activity, can be monitored in clinical trials of subjects exhibiting increased 25869, 25934, 26335, 50365, 21117, 38692, 46508, 16816, 16839, 49937, 49931 or 49933 gene expression, protein levels, or upregulated 25869, 25934, 26335, 50365, 21117, 38692, 46508, 16816, 16839, 49937, 49931 or 49933 activity. In such clinical trials, the expression or activity of a 25869, 25934, 26335, 50365, 21117, 38692, 46508, 16816, 16839, 49937, 49931 or 49933 gene, and preferably, other genes that have been implicated in, for example, a [FAMILYNAME]-associated or another 25869, 25934, 26335, 50365, 21117, 38692, 46508, 16816, 16839, 49937, 49931 or 49933-associated disorder can be used as a “read out” or markers of the phenotype of a particular cell.

### Other Embodiments

In another aspect, the invention features a method of analyzing a plurality of capture probes. The method is useful, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence, wherein the capture probes are from a cell or subject which expresses 25869, 25934, 26335, 50365, 21117, 38692, 46508, 16816, 16839, 49937, 49931 or 49933 or from a cell or subject in which a 25869, 25934, 26335, 50365, 21117, 38692, 46508, 16816, 16839, 49937, 49931 or 49933 mediated response has been elicited; contacting the array with a 25869, 25934, 26335, 50365, 21117, 38692, 46508, 16816, 16839, 49937, 49931 or 49933 nucleic acid (preferably purified), a 25869, 25934, 26335, 50365, 21117, 38692, 46508, 16816, 16839, 49937, 49931 or 49933 polypeptide (preferably purified), or an anti-25869, 25934, 26335, 50365, 21117, 38692, 46508, 16816, 16839, 49937, 49931 or 49933 antibody, and thereby evaluating the plurality of capture probes. Binding, e.g., in the case of a nucleic acid, hybridization with a capture probe at an address of the plurality, is detected, e.g., by a signal generated from a label attached to the 25869, 25934, 26335, 50365, 21117, 38692, 46508, 16816, 16839, 49937, 49931 or 49933 nucleic acid, polypeptide, or antibody.

The capture probes can be a set of nucleic acids from a selected sample, e.g., a sample of nucleic acids derived from a control or non-stimulated tissue or cell.

The method can include contacting the 25869, 25934, 26335, 50365, 21117, 38692, 46508, 16816, 16839, 49937, 49931 or 49933 nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of 25869, 25934, 26335, 50365, 21117, 38692, 46508, 16816, 16839, 49937, 49931 or 49933. Such methods can be used to diagnose a subject, e.g., to evaluate risk for a disease or disorder, to evaluate suitability of a selected treatment for a subject, to evaluate whether a subject has a disease or disorder.

The method can be used to detect SNPs, as described above.

In another aspect, the invention features, a method of analyzing 25869, 25934, 26335, 50365, 21117, 38692, 46508, 16816, 16839, 49937, 49931 or 49933, e.g., analyzing structure, function, or relatedness to other nucleic acid or amino acid sequences. The method includes: providing a 25869, 25934, 26335, 50365, 21117, 38692, 46508, 16816, 16839, 49937, 49931 or 49933 nucleic acid or amino acid sequence; comparing the 25869, 25934, 26335, 50365, 21117, 38692, 46508, 16816, 16839, 49937, 49931 or 49933 sequence with one or more preferably a plurality of sequences from a collection of sequences, e.g., a nucleic acid or protein sequence database; to thereby analyze 25869, 25934, 26335, 50365, 21117, 38692, 46508, 16816, 16839, 49937, 49931 or 49933.

The method can include evaluating the sequence identity between a 25869, 25934, 26335, 50365, 21117, 38692, 46508, 16816, 16839, 49937, 49931 or 49933 sequence and a database sequence. The method can be performed by accessing the database at a second site, e.g., over the internet. Preferred databases include GenBank™ and SwissProt.

In another aspect, the invention features, a set of oligonucleotides, useful, e.g., for identifying SNP's, or identifying specific alleles of 25869, 25934, 26335, 50365, 21117, 38692, 46508, 16816, 16839, 49937, 49931 or 49933. The set includes a plurality of oligonucleotides, each of which has a different nucleotide at an interrogation position, e.g., an SNP or the site of a mutation. In a preferred embodiment, the oligonucleotides of the plurality identical in sequence with one another (except for differences in length). The oligonucleotides can be provided with differential labels, such that an oligonucleotide which hybridizes to one allele provides a signal that is distinguishable from an oligonucleotides which hybridizes to a second allele.

The sequences of 25869, 25934, 26335, 50365, 21117, 38692, 46508, 16816, 16839, 49937, 49931 or 49933 molecules are provided in a variety of mediums to facilitate use thereof. A sequence can be provided as a manufacture, other than an isolated nucleic acid or amino acid molecule, which contains a 25869, 25934, 26335, 50365, 21117, 38692, 46508, 16816, 16839, 49937, 49931 or 49933 molecule. Such a manufacture can provide a nucleotide or amino acid sequence, e.g., an open reading frame, in a form which allows examination of the manufacture using means not directly applicable to examining the nucleotide or amino acid sequences, or a subset thereof, as they exist in nature or in purified form.

A 25869, 25934, 26335, 50365, 21117, 38692, 46508, 16816, 16839, 49937, 49931 or 49933 nucleotide or amino acid sequence can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as compact disc and CD-ROM; electrical storage media such as RAM, ROM, EPROM, EEPROM, and the like; and general hard disks and hybrids of these categories such as magnetic/optical storage media. The medium is adapted or configured for having thereon 25869, 25934, 26335, 50365, 21117, 38692, 46508, 16816, 16839, 49937, 49931 or 49933 sequence information of the present invention.

As used herein, the term “electronic apparatus” is intended to include any suitable computing or processing apparatus of other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as personal digital assistants (PDAs), cellular phones, pagers, and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the 25869, 25934, 26335, 50365, 21117, 38692, 46508, 16816, 16839, 49937, 49931 or 49933 sequence information.

A variety of data storage structures are available to a skilled artisan for creating a computer readable medium having recorded thereon a 25869, 25934, 26335, 50365, 21117, 38692, 46508, 16816, 16839, 49937, 49931 or 49933 nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

By providing the 25869, 25934, 26335, 50365, 21117, 38692, 46508, 16816, 16839, 49937, 49931 or 49933 nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention which match a particular target sequence or target motif.

The present invention therefore provides a medium for holding instructions for performing a method for determining whether a subject has a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder or a pre-disposition to a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder, wherein the method comprises the steps of determining 25869, 25934, 26335, 50365, 21117, 38692, 46508, 16816, 16839, 49937, 49931 or 49933 sequence information associated with the subject and based on the 25869, 25934, 26335, 50365, 21117, 38692, 46508, 16816, 16839, 49937, 49931 or 49933 sequence information, determining whether the subject has a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder and/or recommending a particular treatment for the disease, disorder, or pre-disease condition.

The present invention further provides in an electronic system and/or in a network, a method for determining whether a subject has a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder or a pre-disposition to a disease associated with 25869, 25934, 26335, 50365, 21117, 38692, 46508, 16816, 16839, 49937, 49931 or 49933, wherein the method comprises the steps of determining 25869, 25934, 26335, 50365, 21117, 38692, 46508, 16816, 16839, 49937, 49931 or 49933 sequence information associated with the subject, and based on the 25869, 25934, 26335, 50365, 21117, 38692, 46508, 16816, 16839, 49937, 49931 or 49933 sequence information, determining whether the subject has a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder or a pre-disposition to a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder, and/or recommending a particular treatment for the disease, disorder, or pre-disease condition. The method may further comprise the step of receiving phenotypic information associated with the subject and/or acquiring from a network phenotypic information associated with the subject.

The present invention also provides in a network, a method for determining whether a subject has a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder or a pre-disposition to a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder, said method comprising the steps of receiving 25869, 25934, 26335, 50365, 21117, 38692, 46508, 16816, 16839, 49937, 49931 or 49933 sequence information from the subject and/or information related thereto, receiving phenotypic information associated with the subject, acquiring information from the network corresponding to 25869, 25934, 26335, 50365, 21117, 38692, 46508, 16816, 16839, 49937, 49931 or 49933 and/or corresponding to a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder, and based on one or more of the phenotypic information, the 25869, 25934, 26335, 50365, 21117, 38692, 46508, 16816, 16839, 49937, 49931 or 49933 information (e.g., sequence information and/or information related thereto), and the acquired information, determining whether the subject has a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder or a pre-disposition to a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder. The method may further comprise the step of recommending a particular treatment for the disease, disorder, or pre-disease condition.

The present invention also provides a business method for determining whether a subject has a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder or a pre-disposition to a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder, said method comprising the steps of receiving information related to 25869, 25934, 26335, 50365, 21117, 38692, 46508, 16816, 16839, 49937, 49931 or 49933 (e.g., sequence information and/or information related thereto), receiving phenotypic information associated with the subject, acquiring information from the network related to 25869, 25934, 26335, 50365, 21117, 38692, 46508, 16816, 16839, 49937, 49931 or 49933 and/or related to a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder, and based on one or more of the phenotypic information, the 25869, 25934, 26335, 50365, 21117, 38692, 46508, 16816, 16839, 49937, 49931 or 49933 information, and the acquired information, determining whether the subject has a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder or a pre-disposition to a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder. The method may further comprise the step of recommending a particular treatment for the disease, disorder, or pre-disease condition.

The invention also includes an array comprising a 25869, 25934, 26335, 50365, 21117, 38692, 46508, 16816, 16839, 49937, 49931 or 49933 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be 25869, 25934, 26335, 50365, 21117, 38692, 46508, 16816, 16839, 49937, 49931 or 49933. This allows a profile to be developed showing a battery of genes specifically expressed in one or more tissues.

In addition to such qualitative information, the invention allows the quantitation of gene expression. Thus, not only tissue specificity, but also the level of expression of a battery of genes in the tissue if ascertainable. Thus, genes can be grouped on the basis of their tissue expression per se and level of expression in that tissue. This is useful, for example, in ascertaining the relationship of gene expression in that tissue. Thus, one tissue can be perturbed and the effect on gene expression in a second tissue can be determined. In this context, the effect of one cell type on another cell type in response to a biological stimulus can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder, progression of protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder, and processes, such a cellular transformation associated with the protein kinase, methyltransferase, acyl-CoA dehydrogenase, short chain dehyrdogenase, reductase, acyltransferase, phosphatase, transferase, ATP-ase or synthase-associated or another 25869, 25934, 26335, 50365, 21117, 38692, 46508, 16816, 16839, 49937, 49931 or 49933-associated disease or disorder.

The array is also useful for ascertaining the effect of the expression of a gene on the expression of other genes in the same cell or in different cells (e.g., acertaining the effect of 25869, 25934, 26335, 50365, 21117, 38692, 46508, 16816, 16839, 49937, 49931 or 49933 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including 25869, 25934, 26335, 50365, 21117, 38692, 46508, 16816, 16839, 49937, 49931 or 49933) that could serve as a molecular target for diagnosis or therapeutic intervention.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBI).

Thus, the invention features a method of making a computer readable record of a sequence of a 25869, 25934, 26335, 50365, 21117, 38692, 46508, 16816, 16839, 49937, 49931 or 49933 sequence which includes recording the sequence on a computer readable matrix. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features a method of analyzing a sequence. The method includes: providing a 25869, 25934, 26335, 50365, 21117, 38692, 46508, 16816, 16839, 49937, 49931 or 49933 sequence, or record, in computer readable form; comparing a second sequence to the 25869, 25934, 26335, 50365, 21117, 38692, 46508, 16816, 16839, 49937, 49931 or 49933 sequence; thereby analyzing a sequence. Comparison can include comparing to sequences for sequence identity or determining if one sequence is included within the other, e.g., determining if the 25869, 25934, 26335, 50365, 21117, 38692, 46508, 16816, 16839, 49937, 49931 or 49933 sequence includes a sequence being compared. In a preferred embodiment the 25869, 25934, 26335, 50365, 21117, 38692, 46508, 16816, 16839, 49937, 49931 or 49933 or second sequence is stored on a first computer, e.g., at a first site and the comparison is performed, read, or recorded on a second computer, e.g., at a second site. E.g., the 25869, 25934, 26335, 50365, 21117, 38692, 46508, 16816, 16839, 49937, 49931 or 49933 or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

### EXEMPLIFICATION

### Example 1

### Tissue Distribution of 25869, 25934, 26335, 50365, 21117, 38692, 46508, 16816, 16839, 49937, 49931 or 49933 mRNA

Northern blot hybridizations with various RNA samples can be performed under standard conditions and washed under stringent conditions, i.e., 0.2×SSC at 65° C. A DNA probe corresponding to all or a portion of the 25869, 25934, 26335, 50365, 21117, 38692, 46508, 16816, 16839, 49937, 49931 or 49933 cDNA (SEQ ID NO:1, 3, 7, 9, 16, 18, 20, 22, 25, 27, 28, 30, 35, 37, 39, 41, 42, 44, 63, 65, 67, 69, 70 or 72) or 25869, 25934, 26335, 50365, 21117, 38692, 46508, 16816, 16839, 49937, 49931 or 49933 cDNA can be used. The DNA was radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations.

### Example 2

### TaqMan™ Procedure

The Taqman™ procedure is a quantitative, real-time PCR-based approach to detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest and served as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe included an oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separated the reporter dye and the quencher dye, resulting in increased fluorescence of the reporter. Accumulation of PCR products was detected directly by monitoring the increase in fluorescence of the reporter dye. When the probe was intact, the proximity of the reporter dye to the quencher dye resulted in suppression of the reporter fluorescence. During PCR, if the target of interest was present, the probe specifically annealed between the forward and reverse primer sites. The 5′-3′ nucleolytic activity of the AmpliTaq™ Gold DNA Polymerase cleaved the probe between the reporter and the quencher only if the probe hybridized to the target. The probe fragments were then displaced from the target, and polymerization of the strand continued. The 3′ end of the probe was blocked to prevent extension of the probe during PCR. This process occurred in every cycle and did not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control GAPDH or β-actin gene confirming efficient removal of genomic DNA contamination.

### Example 3

### In Situ Hybridization Procedure

For in situ analysis, various tissues, e.g. tissues obtained from brain, are first frozen on dry ice. Ten-micrometer-thick sections of the tissues are postfixed with 4% formaldehyde in DEPC treated 1× phosphate-buffered saline at room temperature for 10 minutes before being rinsed twice in DEPC 1× phosphate-buffered saline and once in 0.1 M triethanolamine-HCl (pH 8.0). Following incubation in 0.25% acetic anhydride-0.1 M triethanolamine-HCl for 10 minutes, sections are rinsed in DEPC 2×SSC (1×SSC is 0.15M NaCl plus 0.015M sodium citrate). Tissue is then dehydrated through a series of ethanol washes, incubated in 100% chloroform for 5 minutes, and then rinsed in 100% ethanol for 1 minute and 95% ethanol for 1 minute and allowed to air dry.

Hybridizations are performed with ³⁵S-radiolabeled (5×10⁷ cpm/ml) cRNA probes. Probes are incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1×Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides are washed with 2×SSC. Sections are then sequentially incubated at 37° C. in TNE (a solution containing 10 mM Tris-HCl (pH 7.6), 500 mM NaCl, and 1 mM EDTA), for 10 minutes, in TNE with 10 ug of RNase A per ml for 30 minutes, and finally in TNE for 10 minutes. Slides are then rinsed with 2×SSC at room temperature, washed with 2×SSC at 50° C. for 1 hour, washed with 0.2×SSC at 55° C. for 1 hour, and 0.2×SSC at 60° C. for 1 hour. Sections are then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

### Example 4

### Recombinant Expression of 25869, 25934, 26335, 50365, 21117, 38692, 46508, 16816, 16839, 49937, 49931 or 49933 in Bacterial Cells

In this example, 25869, 25934, 26335, 50365, 21117, 38692, 46508, 16816, 16839, 49937, 49931 or 49933 is expressed as a recombinant glutathione-S-transferase (GST) fusion polypeptide in E. coli and the fusion polypeptide is isolated and characterized. Specifically, 25869, 25934, 26335, 50365, 21117, 38692, 46508, 16816, 16839, 49937, 49931 or 49933 is fused to GST and this fusion polypeptide is expressed in E. coli, e.g., strain PEB199. Expression of the GST-25869, -25934, -26335, -50365, -21117, -38692, -46508, -16816, -16839, -49937, -49931 or -49933 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 5

### Expression of Recombinant 25869, 25934, 26335, 50365, 21117, 38692, 46508, 16816, 16839, 49937, 49931 or 49933 Protein in COS Cells

To express the 25869, 25934, 26335, 50365, 21117, 38692, 46508, 16816, 16839, 49937, 49931 or 49933 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 25869, 25934, 26335, 50365, 21117, 38692, 46508, 16816, 16839, 49937, 49931 or 49933 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 25869, 25934, 26335, 50365, 21117, 38692, 46508, 16816, 16839, 49937, 49931 or 49933 DNA sequence is amplified by PCR using two primers. The 5′ primer contains the restriction site of interest followed by approximately twenty nucleotides of the 25869, 25934, 26335, 50365, 21117, 38692, 46508, 16816, 16839, 49937, 49931 or 49933 coding sequence starting from the initiation codon; the 3′ end sequence contains complementary sequences to the other restriction site of interest, a translation stop codon, the HA tag or FLAG tag and the last 20 nucleotides of the 25869, 25934, 26335, 50365, 21117, 38692, 46508, 16816, 16839, 49937, 49931 or 49933 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the 25869, 25934, 26335, 50365, 21117, 38692, 46508, 16816, 16839, 49937, 49931 or 49933 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 25869-, 25934-, 26335-, 50365-, 21117-, 38692-, 46508-, 16816-, 16839-, 49937-, 49931- or 49933-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 25869, 25934, 26335, 50365, 21117, 38692, 46508, 16816, 16839, 49937, 49931 or 49933 polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 25869, 25934, 26335, 50365, 21117, 38692, 46508, 16816, 16839, 49937, 49931 or 49933 coding sequence is cloned directly into the polylinker of the pCDNA/Amp vector using the appropriate restriction sites. The resulting plasmid is transfected into COS cells in the manner described above, and the expression of the 25869, 25934, 26335, 50365, 21117, 38692, 46508, 16816, 16839, 49937, 49931 or 49933 polypeptide is detected by radiolabelling and immunoprecipitation using a 25869, 25934, 26335, 50365, 21117, 38692, 46508, 16816, 16839, 49937, 49931 or 49933 specific monoclonal antibody.

The contents of all references, patents and published patent applications cited throughout this application are incorporated herein by reference.

### EQUIVALENTS

Those skilled in the art will recognize, or be able to ascertain using no more than routine experimentation, many equivalents to the specific embodiments of the invention described herein.

What is claimed is:

1. An isolated 25869, 25934, 26335, 50365, 21117, 38692, 46508, 16816, 16839, 49937, 49931 or 49933 nucleic acid molecule selected from the group consisting of:
a) a nucleic acid molecule comprising a nucleotide sequence which is at least 60% identical to the nucleotide sequence of SEQ ID NO:1, 3, 7, 9, 16, 18, 20, 22, 25, 27, 28, 30, 35, 37, 39, 41, 42, 44, 63, 65, 67, 69, 70 or 72, or the nucleotide sequence of the DNA insert of the plasmid deposited with ATCC Accession Number ______;
b) a nucleic acid molecule comprising a fragment of at least 15 nucleotides of the nucleotide sequence of SEQ ID NO:1, 3, 7, 9, 16, 18, 20, 22, 25, 27, 28, 30, 35, 37, 39, 41, 42, 44, 63, 65, 67, 69, 70 or 72, or the nucleotide sequence of the DNA insert of the plasmid deposited with ATCC Accession Number ______;
c) a nucleic acid molecule which encodes a polypeptide comprising the amino acid sequence of SEQ ID NO:2, 8, 17, 21, 26, 29, 36, 40, 43, 64, 68 or 71, or the amino acid sequence encoded by the cDNA insert of the plasmid deposited with the ATCC Accession Number ______;
d) a nucleic acid molecule which encodes a fragment of a polypeptide comprising the amino acid sequence of SEQ ID NO:2, 8, 17, 21, 26, 29, 36, 40, 43, 64, 68 or 71, or the amino acid sequence encoded by the cDNA insert of the plasmid deposited with the ATCC Accession Number ______, wherein the fragment comprises at least 15 contiguous amino acids of SEQ ID NO:2, 8, 17, 21, 26, 29, 36, 40, 43, 64, 68 or 71, or the amino acid sequence encoded by the cDNA insert of the plasmid deposited with the ATCC Accession Number ______;
e) a nucleic acid molecule which encodes a naturally occurring allelic variant of a polypeptide comprising the amino acid sequence of SEQ ID NO:2, 8, 17, 21, 26, 29, 36, 40, 43, 64, 68 or 71, or the amino acid sequence encoded by the cDNA insert of the plasmid deposited with the ATCC Accession Number ______, wherein the nucleic acid molecule hybridizes to a nucleic acid molecule comprising SEQ ID NO:1, 3, 7, 9, 16, 18, 20, 22, 25, 27, 28, 30, 35, 37, 39, 41, 42, 44, 63, 65, 67, 69, 70 or 72, or a complement thereof, under stringent conditions;
f) a nucleic acid molecule comprising the nucleotide sequence of SEQ ID NO:1, 3, 7, 9, 16, 18, 20, 22, 25, 27, 28, 30, 35, 37, 39, 41, 42, 44, 63, 65, 67, 69, 70 or 72, or the nucleotide sequence of the DNA insert of the plasmid deposited with ATCC Accession Number ______; and
g) a nucleic acid molecule which encodes a polypeptide comprising the amino acid sequence of SEQ ID NO:2, 8, 17, 21, 26, 29, 36, 40, 43, 64, 68 or 71, or the amino acid sequence encoded by the cDNA insert of the plasmid deposited with the ATCC Accession Number ______.

2. The isolated nucleic acid molecule of claim 1, which is the nucleotide sequence SEQ ID NO:1, 7, 16, 20, 25, 28, 35, 39, 42, 63, 67 or 70.

3. A host cell which contains the nucleic acid molecule of claim 1.

4. An isolated 25869, 25934, 26335, 50365, 21117, 38692, 46508, 16816, 16839, 49937, 49931 or 49933 polypeptide selected from the group consisting of:
a) a polypeptide which is encoded by a nucleic acid molecule comprising a nucleotide sequence which is at least 60% identical to a nucleic acid comprising the nucleotide sequence of SEQ ID NO:1, 3, 7, 9, 16, 18, 20, 22, 25, 27, 28, 30, 35, 37, 39, 41, 42, 44, 63, 65, 67, 69, 70 or 72, or the nucleotide sequence of the DNA insert of the plasmid deposited with ATCC Accession Number ______, or a complement thereof;
b) a naturally occurring allelic variant of a polypeptide comprising the amino acid sequence of SEQ ID NO:2, 8, 17, 21, 26, 29, 36, 40, 43, 64, 68 or 71, or the amino acid sequence encoded by the cDNA insert of the plasmid deposited with the ATCC Accession Number ______, wherein the polypeptide is encoded by a nucleic acid molecule which hybridizes to a nucleic acid molecule comprising SEQ ID NO:1, 3, 7, 9, 16, 18, 20, 22, 25, 27, 28, 30, 35, 37, 39, 41, 42, 44, 63, 65, 67, 69, 70 or 72, or a complement thereof under stringent conditions;
c) a fragment of a polypeptide comprising the amino acid sequence of SEQ ID NO:2, 8, 17, 21, 26, 29, 36, 40, 43, 64, 68 or 71, or the amino acid sequence encoded by the cDNA insert of the plasmid deposited with the ATCC Accession Number ______, wherein the fragment comprises at least 15 contiguous amino acids of SEQ ID NO:2, 8, 17, 21, 26, 29, 36, 40, 43, 64, 68 or 71; and
d) the amino acid sequence of SEQ ID NO:2, 8, 17, 21, 26, 29, 36, 40, 43, 64, 68 or 71.

5. An antibody which selectively binds to a polypeptide of claim 4.

6. The polypeptide of claim 4, further comprising heterologous amino acid sequences.

7. A method for producing a polypeptide selected from the group consisting of:
a) a polypeptide comprising the amino acid sequence of SEQ ID NO:2, 8, 17, 21, 26, 29, 36, 40, 43, 64, 68 or 71, or the amino acid sequence encoded by the cDNA insert of the plasmid deposited with the ATCC Accession Number
b) a polypeptide comprising a fragment of the amino acid sequence of SEQ ID NO:2, 8, 17, 21, 26, 29, 36, 40, 43, 64, 68 or 71, or the amino acid sequence encoded by the cDNA insert of the plasmid deposited with the ATCC Accession Number ______, wherein the fragment comprises at least 15 contiguous amino acids of SEQ ID NO:2, 8, 17, 21, 26, 29, 36, 40, 43, 64, 68 or 71, or the amino acid sequence encoded by the cDNA insert of the plasmid deposited with the ATCC Accession Number ______;
c) a naturally occurring allelic variant of a polypeptide comprising the amino acid sequence of SEQ ID NO:2, 8, 17, 21, 26, 29, 36, 40, 43, 64, 68 or 71, or the amino acid sequence encoded by the cDNA insert of the plasmid deposited with the ATCC Accession Number ______, wherein the polypeptide is encoded by a nucleic acid molecule which hybridizes to a nucleic acid molecule comprising SEQ ID NO:1, 3, 7, 9, 16, 18, 20, 22, 25, 27, 28, 30, 35, 37, 39, 41, 42, 44, 63, 65, 67, 69, 70 or 72; and
d) the amino acid sequence of SEQ ID NO:2, 8, 17, 21, 26, 29, 36, 40, 43, 64, 68 or 71;
comprising culturing the host cell of claim 3 under conditions in which the nucleic acid molecule is expressed.

8. A method for detecting the presence of a nucleic acid molecule of claim 1 or a polypeptide encoded by the nucleic acid molecule in a sample, comprising:
a) contacting the sample with a compound which selectively hybridizes to the nucleic acid molecule of claim 1 or binds to the polypeptide encoded by the nucleic acid molecule; and
b) determining whether the compound hybridizes to the nucleic acid or binds to the polypeptide in the sample.

9. A kit comprising a compound which selectively hybridizes to a nucleic acid molecule of claim 1 or binds to a polypeptide encoded by the nucleic acid molecule and instructions for use.

10. A method for identifying a compound which binds to a polypeptide or modulates the activity of the polypeptide of claim 4 comprising the steps of:
a) contacting a polypeptide, or a cell expressing a polypeptide of claim 4 with a test compound; and
b) determining whether the polypeptide binds to the test compound or determining the effect of the test compound on the activity of the polypeptide.

11. A method for modulating the activity of a polypeptide of claim 4 comprising contacting the polypeptide or a cell expressing the polypeptide with a compound which binds to the polypeptide in a sufficient concentration to modulate the activity of the polypeptide.

12. A method for identifying a compound capable of treating a disorder characterized by aberrant 25869, 25934, 26335, 50365, 21117, 38692, 46508, 16816, 16839, 49937, 49931 or 49933 activity, comprising assaying the ability of the compound to modulate 25869, 25934, 26335, 50365, 21117, 38692, 46508, 16816, 16839, 49937, 49931 or 49933 nucleic acid expression or 25869, 25934, 26335, 50365, 21117, 38692, 46508, 16816, 16839, 49937, 49931 or 49933 polypeptide activity, thereby identifying a compound capable of treating a disorder characterized by aberrant 25869, 25934, 26335, 50365, 21117, 38692, 46508, 16816, 16839, 49937, 49931 or 49933 activity.

13. A method of identifying a nucleic acid molecule associated with a disorder characterized by aberrant 25869, 25934, 26335, 50365, 21117, 38692, 46508, 16816, 16839, 49937, 49931 or 49933 activity, comprising:
a) contacting a sample from a subject with a disorder characterized by aberrant 25869, 25934, 26335, 50365, 21117, 38692, 46508, 16816, 16839, 49937, 49931 or 49933 activity, comprising nucleic acid molecules with a hybridization probe comprising at least 25 contiguous nucleotides of SEQ ID NO:1, 7, 16, 20, 25, 28, 35, 39, 42, 63, 67 or 70 defined in claim 2; and
b) detecting the presence of a nucleic acid molecule in the sample that hybridizes to the probe, thereby identifying a nucleic acid molecule associated with a disorder characterized by aberrant 25869, 25934, 26335, 50365, 21117, 38692, 46508, 16816, 16839, 49937, 49931 or 49933 activity.

14. A method of identifying a polypeptide associated with a disorder characterized by aberrant 25869, 25934, 26335, 50365, 21117, 38692, 46508, 16816, 16839, 49937, 49931 or 49933 activity, comprising:
a) contacting a sample comprising polypeptides with a 25869, 25934, 26335, 50365, 21117, 38692, 46508, 16816, 16839, 49937, 49931 or 49933 polypeptide defined in claim 4; and
b) detecting the presence of a polypeptide in the sample that binds to the 25869, 25934, 26335, 50365, 21117, 38692, 46508, 16816, 16839, 49937, 49931 or 49933 binding partner, thereby identifying the polypeptide associated with a disorder characterized by aberrant 25869, 25934, 26335, 50365, 21117, 38692, 46508, 16816, 16839, 49937, 49931 or 49933 activity.

15. A method of identifying a subject having a disorder characterized by aberrant 25869, 25934, 26335, 50365, 21117, 38692, 46508, 16816, 16839, 49937, 49931 or 49933 activity, comprising:
a) contacting a sample obtained from the subject comprising nucleic acid molecules with a hybridization probe comprising at least 25 contiguous nucleotides of SEQ ID NO:1, 7, 16, 20, 25, 28, 35, 39, 42, 63, 67 or 70 defined in claim 2; and
b) detecting the presence of a nucleic acid molecule in the sample that hybridizes to the probe, thereby identifying a subject having a disorder characterized by aberrant 25869, 25934, 26335, 50365, 21117, 38692, 46508, 16816, 16839, 49937, 49931 or 49933 activity.

16. A method for treating a subject having a disorder characterized by aberrant 25869, 25934, 26335, 50365, 21117, 38692, 46508, 16816, 16839, 49937, 49931 or 49933 activity, or a subject at risk of developing a disorder characterized by aberrant 25869, 25934, 26335, 50365, 21117, 38692, 46508, 16816, 16839, 49937, 49931 or 49933 activity, comprising administering to the subject a 25869, 25934, 26335, 50365, 21117, 38692, 46508, 16816, 16839, 49937, 49931 or 49933 modulator of the nucleic acid molecule defined in claim 1 or the polypeptide encoded by the nucleic acid molecule or contacting a cell with a 25869, 25934, 26335, 50365, 21117, 38692, 46508, 16816, 16839, 49937, 49931 or 49933 modulator.

17. The method defined in claim 16 wherein said disorder is a cellular proliferative and/or differentiative disorder, an angiogenic disorder, a brain disorder, a neurological disorder, a blood vessel disorder, a breast disorder, a colon disorder, a kidney disorder, a lung disorder, an ovarian disorder, a prostate disorder, a hematopoeitic disorder, a pancreatic disorder, a skeletal muscle disorder, a skin disorder, a hormonal disorder, an immune e.g., inflammatory, disorder, a cardiovascular disorder, a lipid homeostasis disorder, an endothelial cell disorder, a liver disorder, a disorder of the small intestine, a pain disorder, a viral disease, a metabolic disorder, bone metabolism disorders or platelet disorders.

18. The method of claim 16, wherein the 25869, 25934, 26335, 50365, 21117, 38692, 46508, 16816, 16839, 49937, 49931 or 49933 modulator is
a) a small molecule;
b) peptide;
c) phosphopeptide;
d) anti-25869, 25934, 26335, 50365, 21117, 38692, 46508, 16816, 16839, 49937, 49931 or 49933 antibody;
e) a 25869, 25934, 26335, 50365, 21117, 38692, 46508, 16816, 16839, 49937, 49931 or 49933 polypeptide comprising the amino acid sequence of SEQ ID NO:2, 8, 17, 21, 26, 29, 36, 40, 43, 64, 68 or 71, or a fragment thereof;
f) a 25869, 25934, 26335, 50365, 21117, 38692, 46508, 16816, 16839, 49937, 49931 or 49933 polypeptide comprising an amino acid sequence which is at least 90 percent identical to the amino acid sequence of SEQ ID NO:2, 8, 17, 21, 26, 29, 36, 40, 43, 64, 68 or 71, wherein the percent identity is calculated using the ALIGN program for comparing amino acid sequences, a PAM120 weight residue table, a gap length penalty of 12, and a gap penalty of 4; or
g) an isolated naturally occurring allelic variant of a polypeptide consisting of the amino acid sequence of SEQ ID NO:2, 8, 17, 21, 26, 29, 36, 40, 43, 64, 68 or 71, wherein the polypeptide is encoded by a nucleic acid molecule which hybridizes to a complement of a nucleic acid molecule consisting of SEQ ID NO:1, 7, 16, 20, 25, 28, 35, 39, 42, 63, 67 or 70 at 6×SSC at 45° C., followed by one or more washes in 0.2×SSC, 0.1% SDSₐₜ 65° C.

19. The method of claim 16, wherein the 25869, 25934, 26335, 50365, 21117, 38692, 46508, 16816, 16839, 49937, 49931 or 49933 modulator is
a) an antisense 25869, 25934, 26335, 50365, 21117, 38692, 46508, 16816, 16839, 49937, 49931 or 49933 nucleic acid molecule;
b) is a ribozyme;
c) the nucleotide sequence of SEQ ID NO:1, 7, 16, 20, 25, 28, 35, 39, 42, 63, 67 or 70 or a fragment thereof;
d) a nucleic acid molecule encoding a polypeptide comprising an amino acid sequence which is at least 90 percent identical to the amino acid sequence of SEQ ID NO:2, 8, 17, 21, 26, 29, 36, 40, 43, 64, 68 or 71, wherein the percent identity is calculated using the ALIGN program for comparing amino acid sequences, a PAM120 weight residue table, a gap length penalty of 12, and a gap penalty of 4;
e) a nucleic acid molecule encoding a naturally occurring allelic variant of a polypeptide comprising the amino acid sequence of SEQ ID NO:2, 8, 17, 21, 26, 29, 36, 40, 43, 64, 68 or 71, wherein the nucleic acid molecule which hybridizes to a complement of a nucleic acid molecule consisting of SEQ ID NO:1, 7, 16, 20, 25, 28, 35, 39, 42, 63, 67 or 70 at 6×SSC at 45° C., followed by one or more washes in 0.2×SSC, 0.1% SDS at 65° C.; or
f) a gene therapy vector.
